(19) United States
(12) Patent Application Publication
(10) Pub. No.: US 2009/0227588 A1
(43) Pub. Date: Sep. 10, 2009

(54) Substituted pyrazole compounds useful as soluble epoxide hyrolase inhibitors

(76) Inventors: Roman Wolfgang Fleck, Geneva (CH); Xin Guo, Danbury, CT (US); Ho Yin Lo, Bethel, CT (US); Chuk Chui Man, Ridgefield, CT (US)

Correspondence Address: MICHAEL P. MORRIS;BOEHRINGER INGELHEIM USA CORPORATION, 900 RIDGEBURY ROAD, P O BOX 368, RIDGEFIELD, CT, 06877-0368, US

(21) Appl. No.: 12/095,928

(22) PCT Filed: Nov. 14, 2006
(86) PCT No.: PCT/US2006/060863

§ 371 Date: Jun. 23, 2008

Related U.S. Application Data
(60) Provisional application No. 60/742,350, filed on Dec. 5, 2005.

(51) Int. Cl. A61K 31/5377; C07D 401/14; A61K 31/444; A61K 31/4439; C07D 401/04; A61P 3/10; A61K 31/501; C07D 413/14

(52) U.S. Cl. 514/236.5; 546/256; 514/333; 514/341; 546/275.4; 544/238; 514/252.03; 544/131

(57) ABSTRACT

Disclosed are compounds active against soluble epoxide hydrolase (sEH), compositions thereof and methods of using and making same.

This application claims benefit to U.S. provisional application Ser. No. 60/742,350 filed Dec. 5, 2005.

### BACKGROUND OF THE INVENTION

1. Technical Field

This invention relates to compounds possessing anti-sEH activity and methods of using soluble epoxide hydrolase (sEH) inhibitors for diseases related to cardiovascular disease.

2. Background Information

Epoxide hydrolases are a group of enzymes ubiquitous in nature, detected in species ranging from plants to mammals. These enzymes are functionally related in that they all catalyze the addition of water to an epoxide, resulting in a diol. Epoxide hydrolases are important metabolizing enzymes in living systems and their diol products are frequently found as intermediates in the metabolic pathway of xenobiotics. Epoxide hydrolases are therefore important enzymes for the detoxification of epoxides by conversion to their corresponding, non-reactive diols.

In mammals, several types of epoxide hydrolases have been characterized including soluble epoxide hydrolase (sEH), also referred to as cytosolic epoxide hydrolase, cholesterol epoxide hydrolase, LTA₄ hydrolase, hepoxilin hydrolase, and microsomal epoxide hydrolase (Fretland and Omiecinski, Chemico-Biological Interactions, 129: 41-59 (2000)). Epoxide hydrolases have been found in all tissues examined in vertebrates including heart, kidney and liver (Vogel, et al., Eur J. Biochemistry, 126: 425-431 (1982); Schladt et al., Biochem. Pharmacol., 35: 3309-3316 (1986)). Epoxide hydrolases have also been detected in human blood components including lymphocytes (e.g. T-lymphocytes), monocytes, erythrocytes, platelets and plasma. In the blood, most of the sEH detected was present in lymphocytes (Seidegard et al., Cancer Research, 44: 3654-3660 (1984)).

The epoxide hydrolases differ in their specificity towards epoxide substrates. For example, sEH is selective for aliphatic epoxides such as epoxide fatty acids while microsomal epoxide hydrolase (mEH) is more selective for cyclic and arene epoxides. The primary known physiological substrates of sEH are four regioisomeric cis epoxides of arachidonic acid, 5,6-, 8,9-, 11,12-, and 14,15-epoxyeicosatrienoic acid, also known as epoxyeicosatrienoic acids or EETs. Also known to be substrates for sEH are epoxides of linoleic acid known as leukotoxin or isoleukotoxin. Both the EETs and the leukotoxins are generated by members of the cytochrome P450 monooxygenase family (Capdevila, et al., J. Lipid Res., 41: 163-181 (2000)).

EETs function as chemical autocrine and paracrine mediators in the cardiovascular and renal systems (Spector, et al, Progress in Lipid Research, 43: 55-90 (2004); Newman, et al., Progress in Lipid Research 44: 1-51 (2005)). EETs appear to be able to function as endothelial derived hyperpolarizing factor (EDHF) in various vascular beds due to their ability to cause hyperpolarization of the membranes of vascular smooth muscle cells with resultant vasodilation (Weintraub, et al., Circ. Res., 81: 258-267 (1997)). EDHF is synthesized from arachidonic acid by various cytochrome P450 enzymes in endothelial cells proximal to vascular smooth muscle (Quilley, et al., Brit. Pharm., 54: 1059 (1997); Quilley and McGiff, TIPS, 21: 121-124 (2000)); Fleming and Busse, Nephrol. Dial. Transplant, 13: 2721-2723 (1998)). In the vascular smooth muscle cells EETs provoke signaling pathways which lcad to activation of BK_{Ca2+} channels (big Ca²⁺ activated potassium channels) and inhibition of L-type Ca²⁺ channels, ultimately resulting in hyperpolarization of membrane potential, inhibition of Ca²⁺ influx and relaxation (Li et al., Circ. Res., 85: 349-356 (1999)). Endothelium dependent vasodilation has been shown to be impaired in different forms of experimental hypertension as well as in human hypertension (Lind, et al., Blood Pressure, 9: 4-15 (2000)). Impaired endothelium dependent vasorelaxation is also a characteristic feature of the syndrome known as endothelial dysfunction (Goligorsky, et. al., Hypertension, 37 [part 2]:744-748 (2001)). Endothelial dysfunction plays a significant role in a large number of pathological conditions including type 1 and type 2 diabetes, insulin resistance syndrome, hypertension, atherosclerosis, coronary artery disease, angina, ischemia, ischemic stroke, Raynaud's disease and renal disease. Hence, it is likely that enhancement of EETs concentration would have a beneficial therapeutic effect in patients where endothelial dysfunction plays a causative role. Other effects of EETs that may influence hypertension involve effects on kidney function. Levels of various EETs and their hydrolysis products, the DHETs, increase significantly both in the kidneys of spontaneously hypertensive rats (SHR) (Yu, et al., Circ. Res. 87: 992-998 (2000)) and in women suffering from pregnancy induced hypertension (Catella, et al., Proc. Natl. Acad. Sci. U.S.A., 87: 5893-5897 (1990)). In angiotensin II infused rats the treatment with a selective sEH inhibitor attenuated the afferent arteriolar diameter in the kidney and lowered urinary albumin secretion, a marker of compromised renal function, suggesting antihypertensive and renal vascular protective effects of increased EETs levels (Zhao, et al, 15: 1244-1253 (2004)). In the spontaneously hypertensive rat model, both cytochrome P450 and sEH activities were found to increase (Yu et al., Molecular Pharmacology, 57: 1011-1020 (2000)). Addition of a known sEH inhibitor was shown to decrease the blood pressure to normal levels. Furthermore, administration of a selective sEH inhibitor to angiotensin II treated rats was demonstrated to lower systolic blood pressure (Imig, et al, Hypertension, 39: 690-694 (2002)). Finally, male soluble epoxide hydrolase null mice exhibited a phenotype characterized by lower blood pressure than their wild-type counterparts (Sinal, et al., J. Biol. Chem., 275: 40504-40510 (2000)).

EETs, especially 11,12-EET, also have been shown to exhibit anti-inflammatory properties (Node, et al., Science, 285: 1276-1279 (1999); Campbell, TIPS, 21: 125-127 (2000); Zeldin and Liao, TIPS, 21: 127-128 (2000)). Node, et al. have demonstrated 11,12-EET decreases expression of cytokine induced endothelial cell adhesion molecules, especially VCAM-1. They further showed that EETs prevent leukocyte adhesion to the vascular wall and that the mechanism responsible involves inhibition of NF-κB and IκB kinase. Vascular inflammation plays a role in endothelial dysfunction (Kessler, et al., Circulation, 99: 1878-1884 (1999)). Hence, the ability of EETs to inhibit the NF-κB pathway should also help ameliorate this condition. In addition, the administration of EETs and/or the administration of a selective sEH inhibitor was demonstrated to attenuate tobacco smoke induced inflammation, as assessed total bronchoalveolar lavage cell numbers and concomittant reduction in neutrophils, alveolar macrophages, and lymphocytes (Smith, et al, 102: 2186-2191 (2005)).

In addition to the physiological effect of some substrates of sEH (EETs, mentioned above), some diols, i.e. DHETs, produced by sEH may have potent biological effects. For example, sEH metabolism of epoxides produced from linoleic acid (leukotoxin and isoleukotoxin) produces leukotoxin and isoleukotoxin diols (Greene, et al., Arch. Biochem. Biophys. 376(2): 420-432 (2000)). These diols were shown to be toxic to cultured rat alveolar epithelial cells, increasing intracellular calcium levels, increasing intercellular junction permeability and promoting loss of epithelial integrity (Moghaddam et al., Nature Medicine, 3: 562-566 (1997)). Therefore these diols could contribute to the etiology of diseases such as adult respiratory distress syndrome where lung leukotoxin levels have been shown to be elevated (Ishizaki, et al., Pulm. Pharm. & Therap., 12: 145-155 (1999)). Hammock, et al. have disclosed the treatment of inflammatory diseases, in particular adult respiratory distress syndrome and other acute inflammatory conditions mediated by lipid metabolites, by the administration of inhibitors of epoxide hydrolase (WO 98/06261; U.S. Pat. No. 5,955,496).

A number of classes of sEH inhibitors have been identified. Among these are chalcone oxide derivatives (Miyamoto, et al. Arch. Biochem. Biophys., 254: 203-213 (1987)) and various trans-3-phenylglycidols (Dietze, et al., Biochem. Pharm. 42: 1163-1175 (1991); Dietze, et al., Comp. Biochem. Physiol. B, 104: 309-314 (1993)).

More recently, Hammock et al. have disclosed certain biologically stable inhibitors of sEH for the treatment of inflammatory diseases, for use in affinity separations of epoxide hydrolases and in agricultural applications (U.S. Pat. No. 6,150,415). The Hammock '415 patent also generally describes that the disclosed pharmacophores can be used to deliver a reactive functionality to the catalytic site, e.g., alkylating agents or Michael acceptors, and that these reactive functionalities can be used to deliver fluorescent or affinity labels to the enzyme active site for enzyme detection (col. 4, line 66 to col. 5, line 5). Certain urea and carbamate inhibitors of sEH have also been described in the literature (Morisseau et al., Proc. Natl. Acad. Sci., 96: 8849-8854 (1999); Argiriadi et al., J. Biol. Chem., 275 (20): 15265-15270 (2000); Nakagawa et al. Bioorg. Med. Chem., 8: 2663-2673 (2000); US 2005/0026844 and Kim, et al., J. Med. Chem. 47(8): 2110-2122 (2004) both of which describe inhibitors with additional, tethered oxo pharmacophores).

WO 00/23060 discloses a method of treating immunological disorders mediated by T-lymphocytes by administration of an inhibitor of sEH. Several 1-(4-aminophenyl)pyrazoles are given as examples of inhibitors of sEH.

U.S. Pat. No. 6,150,415 to Hammock is directed to a method of inhibiting an epoxide hydrolase, using compounds having the structure

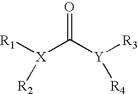

wherein X and Y is each independently nitrogen, oxygen, or sulfur, and X can further be carbon, at least one of R1-R4 is hydrogen, R2 is hydrogen when X is nitrogen but is not present when X is sulfur or oxygen, R4 is hydrogen when Y is nitrogen but is not present when Y is sulfur or oxygen, R1 and R3 is each independently H, C1-20 substituted or unsubstituted alkyl, cycloalkyl, aryl, acyl, or heterocyclic. Related to the Hammock patent is U.S. Pat. No. 6,531,506 to Kroetz et al. which claims a method of treating hypertension using of an inhibitor of epoxide hydrolase, also claimed are methods of treating hypertension using compounds similar to those described in the Hammock patent. Neither of these patents teaches or suggests methods of treating cardiovascular diseases using the particular sEH inhibitors described herein.

As outlined in the discussion above, inhibitors of sEH are useful therefore, in the treatment of cardiovascular diseases such as endothelial dysfunction either by preventing the degradation of sEH substrates that have beneficial effects or by preventing the formation of metabolites that have adverse effects.

All references cited above and throughout this application are incorporated herein by reference in their entirety.

### BRIEF SUMMARY OF THE INVENTION

It is therefore an object of the invention to provide compounds active as sEH inhibitors of the formula I as described herein below.

It is a further object of the invention to provide a method of treating hypertension by administering to a patient a compound of the formula I as described herein below.

It is yet a further object to provide methods of making the compounds described herein below.

### DETAILED DESCRIPTION OF THE INVENTION

In a first generic embodiment, there is provided a compound of the formula (I) wherein:

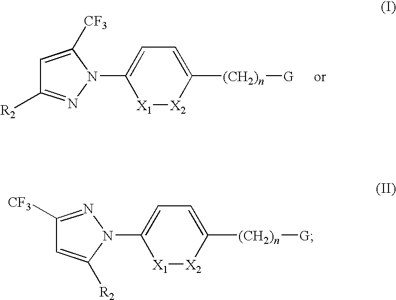

wherein G=

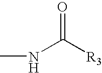

or

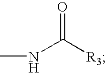

### X₁-X₂ is —CH═CH—, —N═CH—, —C═N— or —N═N—;

R₂ is chosen from heteroaryl and carbocycle optionally substituted by C₁₋₁₀ alkyl, C₁₋₁₀ alkoxy each substituent of R₂ is optionally halogenated;
R₃ is chosen from heteroaryl, heterocycle, carbocycle, Ar₂—Ar₁— and an acyclic moiety chosen from: —NH—(CH₂)ₜ—Ar₁, —NH—(CH₂)ₜ—O—Ar₁, —NH—Ar₁, C₁₋₁₀ alkyl, —C₁₋₁₀ alkyl-Ar₁, O—C₁₋₁₀ alkyl-Ar₁, Ar₂-L-Ar₁- and —C₁₋₁₀ alkyl(phenyl)₂, or R₃ is L;
L is a C₁₋₁₀ alkyl chain optionally interrupted by O, S or NRₓ, and optionally substituted by oxo (═O);
Ar₁ and Ar₂ are each independently heteroaryl, heterocycle or carbocycle, each optionally substituted by one or more C₁₋₁₀ alkyl, C₁₋₁₀ alkoxy, —NRₓR_{y}, —C(O)—NRₓR_{y}, Rₓ—S(O)m-, Het-C(O)—, Het-S(O)ₘ—, NO₂, OH, halogen, C₁₋₁₀ alkoxycarbonyl, CO₂, CN, C₁₋₁₀ acyl, —S(O)ₘ—NRₓR_{y}, Rₓ—S(O)ₘ—NHR_{y}, —(CH₂)ₜ—OH wherein Het is pyrrolidinyl or morpholinyl;
m is 0-2;
n is 0-5;
t is 0-5;
or the pharmaceutically acceptable salts thereof.

In another embodiment, there is provided a compound of the formula (I) as described immediately above and wherein:

R₂ is chosen from pyridinyl, phenyl and cyclohexyl optionally substituted by C₁₋₁₀ alkyl, C₁₋₁₀ alkoxy each substituent of R₂ is optionally halogenated;
R₃ is chosen from phenyl, pyridinone, pyridinyl, —NH—(CH₂)ₜ—Ar₁, —NH—(CH₂)ₜ—O—Ar₁, —NH—Ar₁, C₁₋₁₀ alkyl, —C₁₋₁₀ alkyl-Ar₁ and —C₁₋₁₀ alkyl(phenyl)₂;
Ar₁ and Ar₂ are each independently phenyl, pyridinone, pyridinyl, morpholinyl, benzofuranyl, piperidinyl, cyclohexenyl, benzodioxolanyl, pyrrolidinyl, tetrazolyl, oxazolyl, isoxazolyl, pyrimidinyl or benzodioxolyl.

In another generic aspect of the invention, there is provided a compound of the formula (Ia):

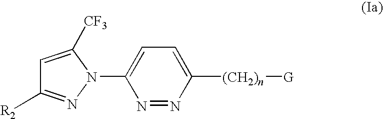

wherein for the Formula (Ia), the component R₂ is:

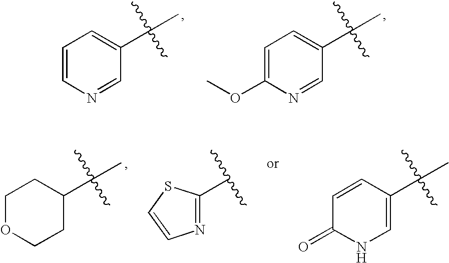

and the component

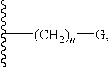

is chosen from those shown in the table I below;

TABLE 1

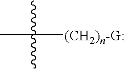

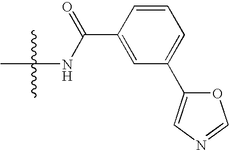

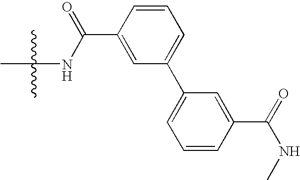

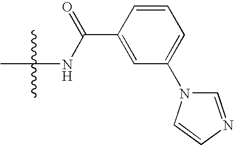

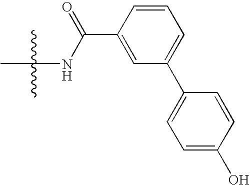

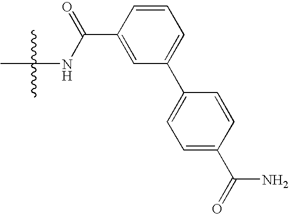

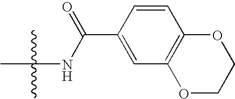

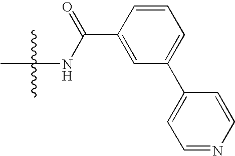

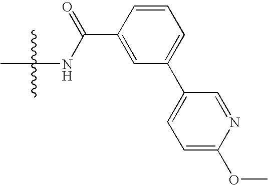

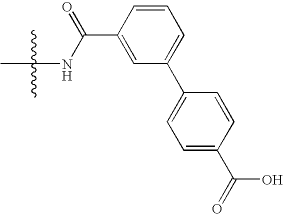

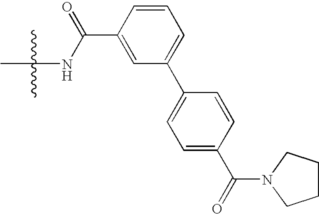

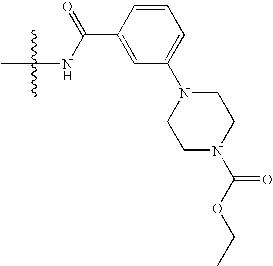

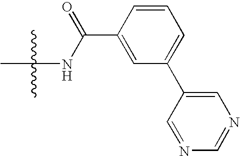

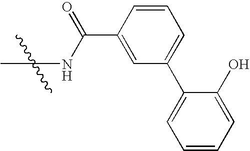

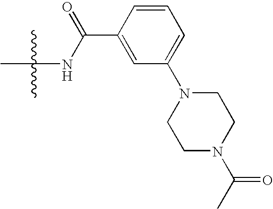

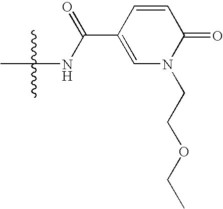

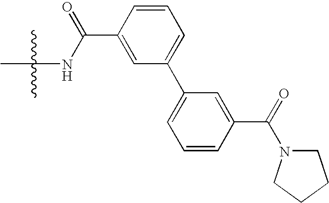

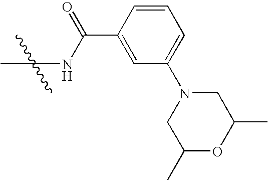

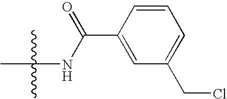

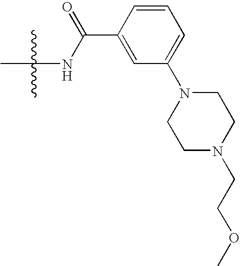

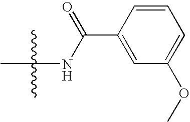

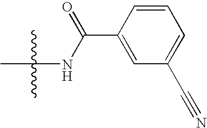

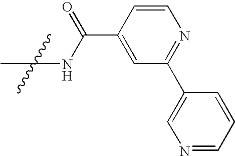

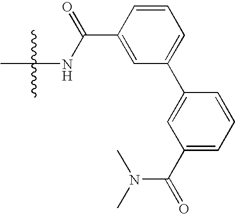

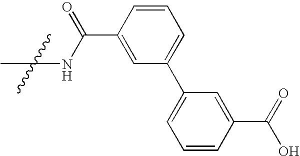

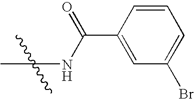

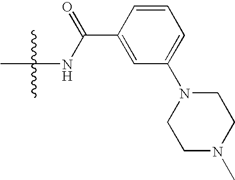

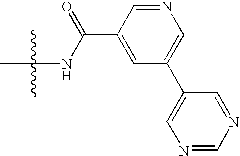

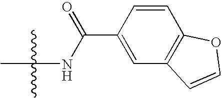

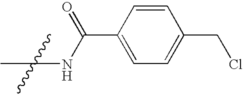

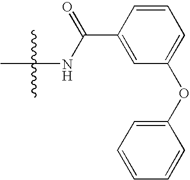

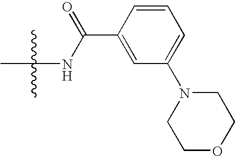

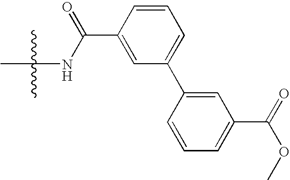

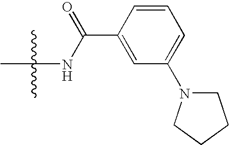

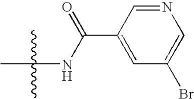

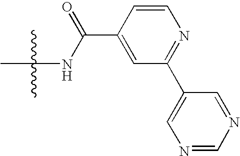

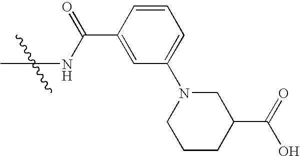

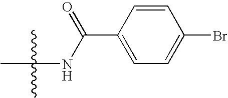

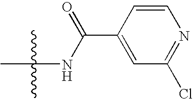

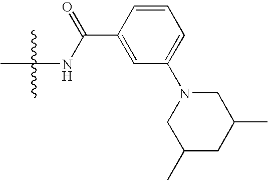

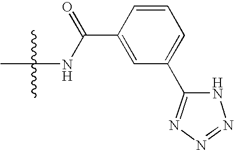

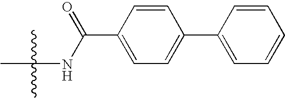

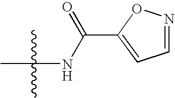

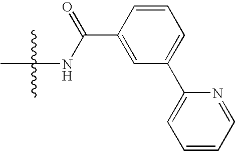

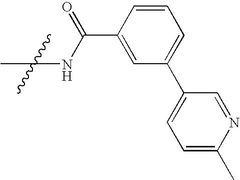

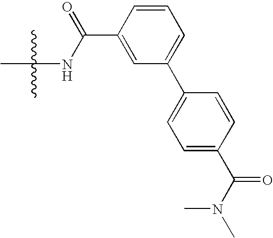

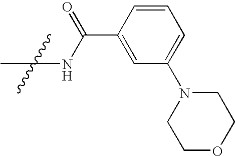

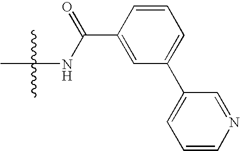

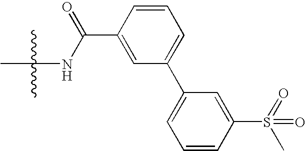

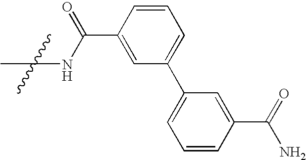

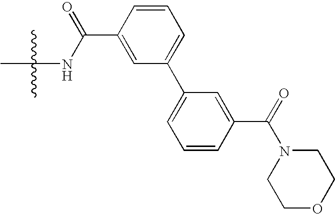

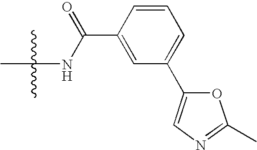

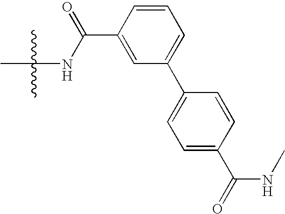

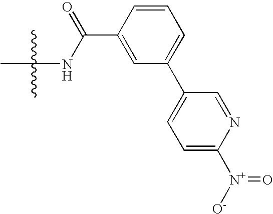

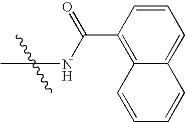

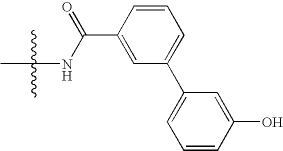

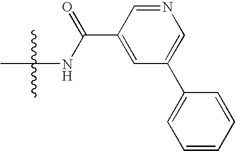

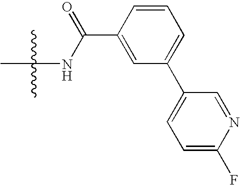

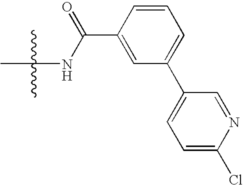

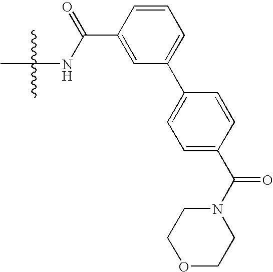

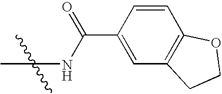

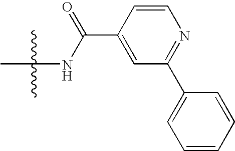

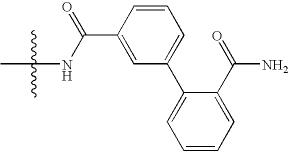

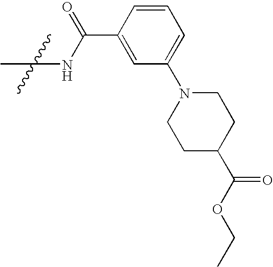

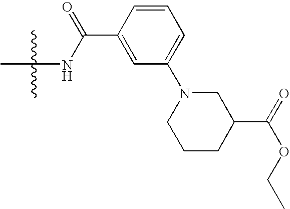

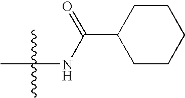

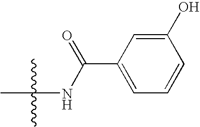

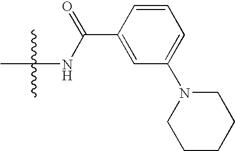

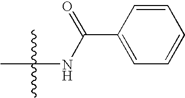

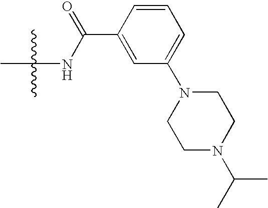

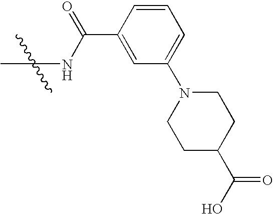

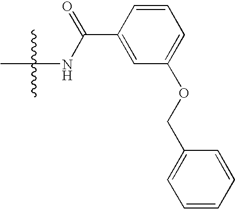

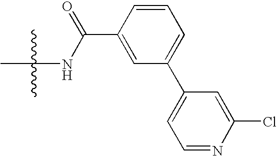

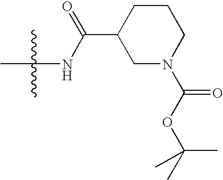

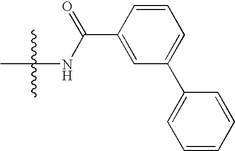

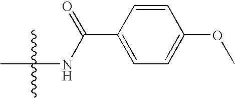

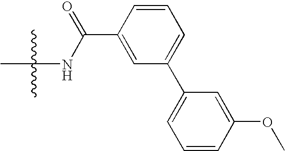

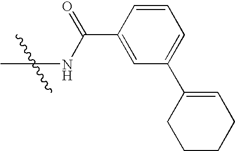

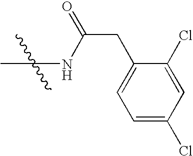

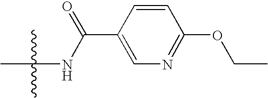

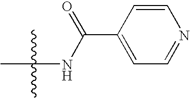

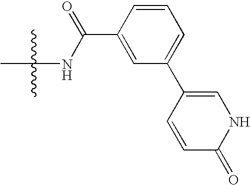

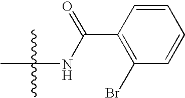

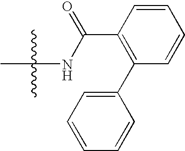

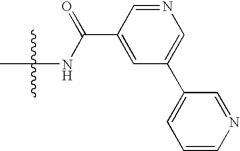

or the pharmaceutically acceptable salts thereof.

In another generic aspect of the invention, there is provided a compound of the formula (Ib):

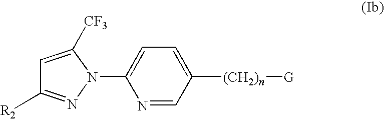

wherein for the Formula (Ib), the component R₂ is:

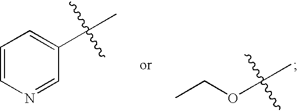

and the component

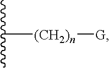

is chosen from those shown in the table II below;

TABLE II

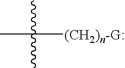

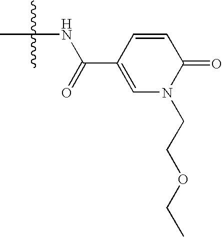

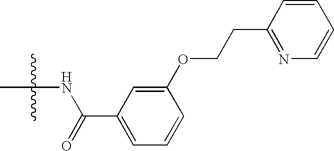

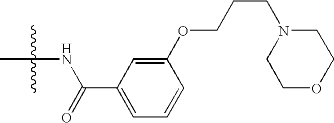

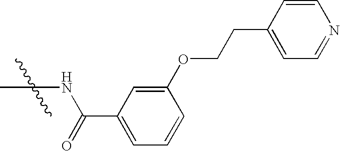

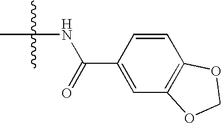

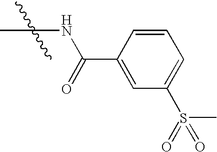

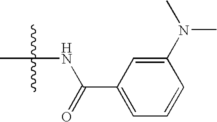

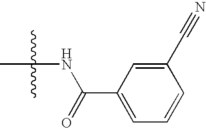

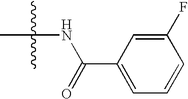

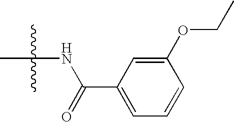

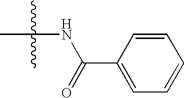

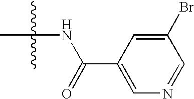

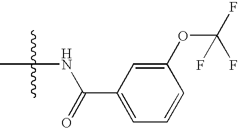

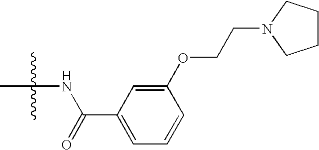

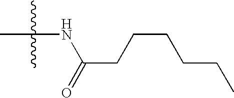

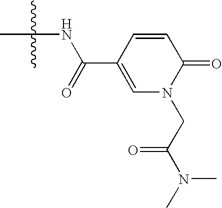

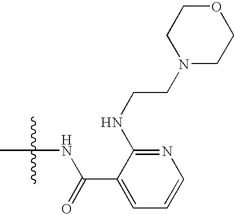

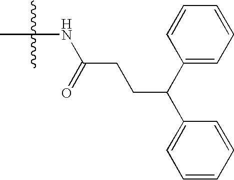

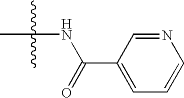

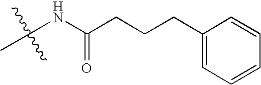

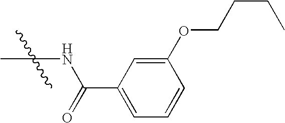

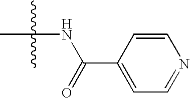

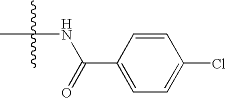

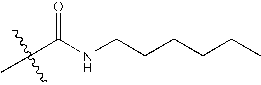

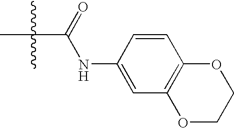

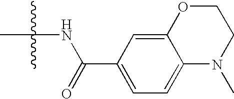

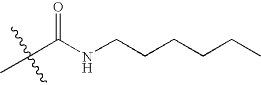

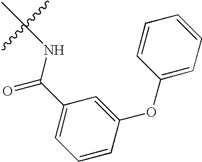

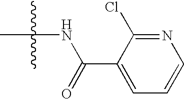

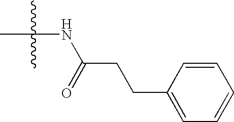

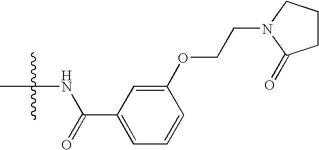

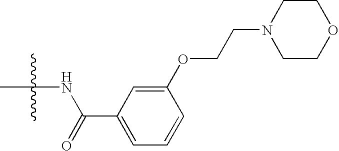

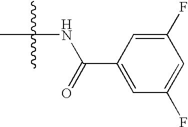

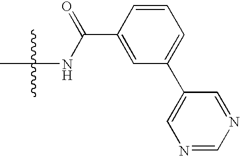

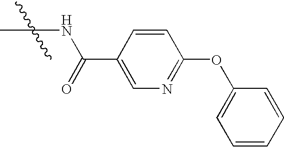

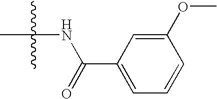

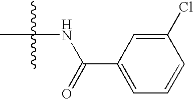

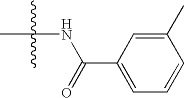

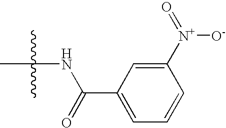

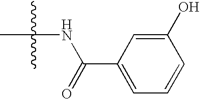

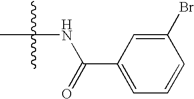

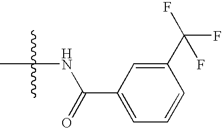

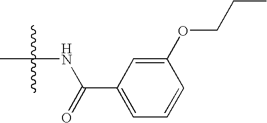

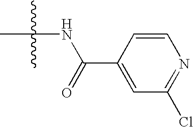

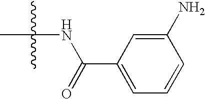

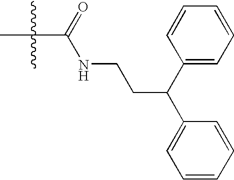

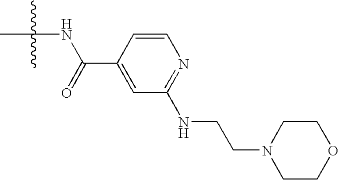

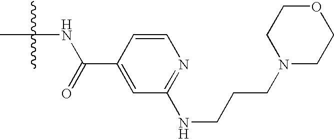

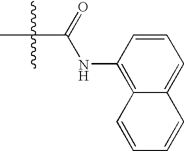

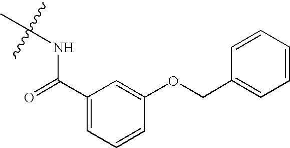

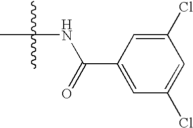

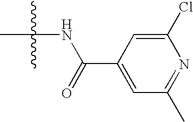

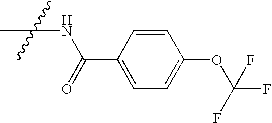

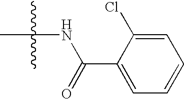

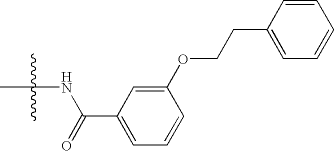

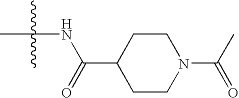

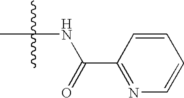

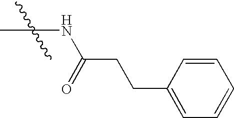

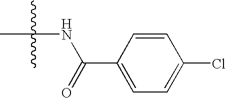

or the pharmaceutically acceptable salts thereof.

In another generic aspect of the invention, there is provided a compound of the formula (Ic) or (Id):

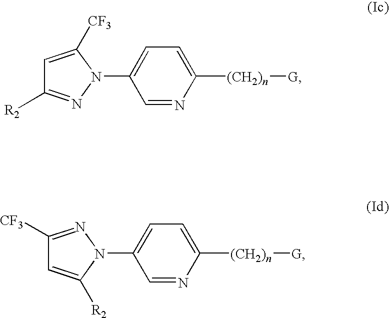

wherein for the Formula (Ic) or (Id), the component R₂ is:

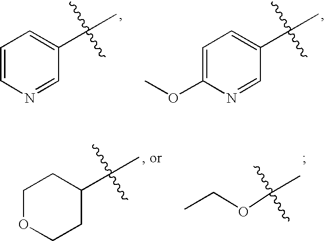

and the component

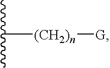

is chosen from those shown in the table III below;

TABLE III

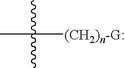

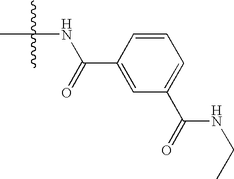

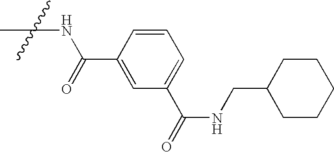

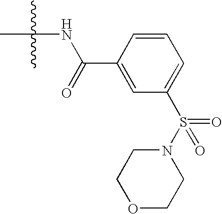

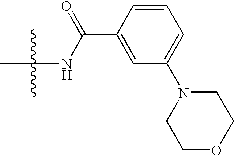

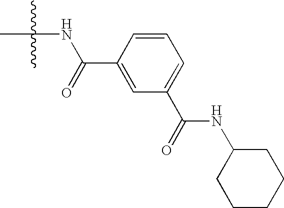

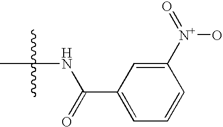

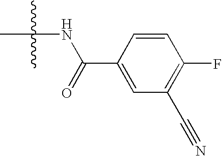

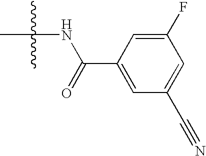

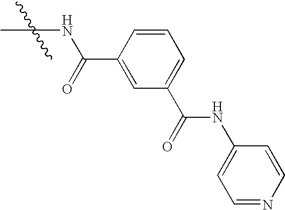

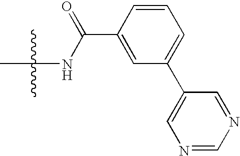

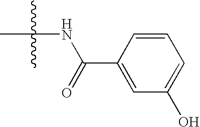

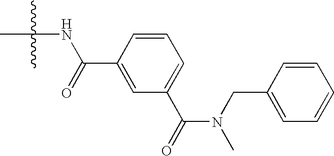

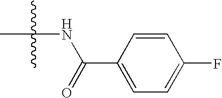

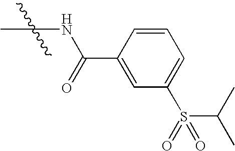

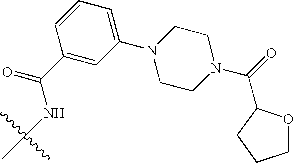

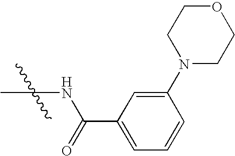

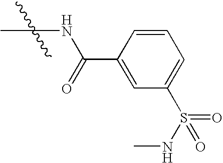

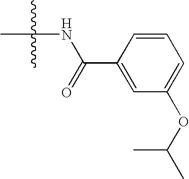

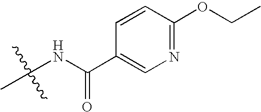

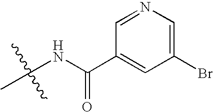

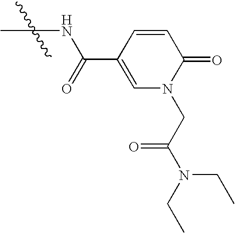

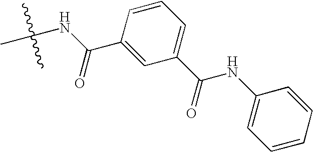

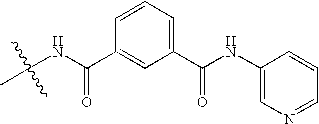

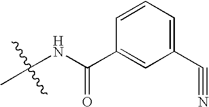

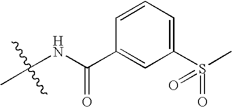

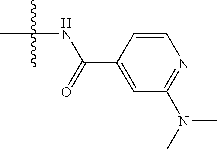

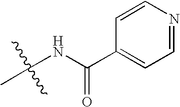

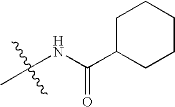

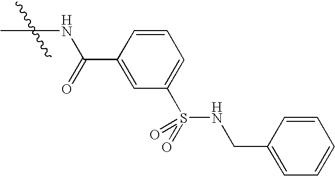

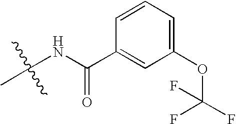

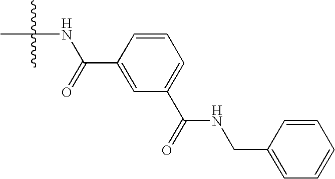

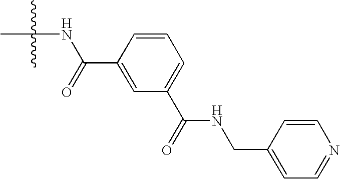

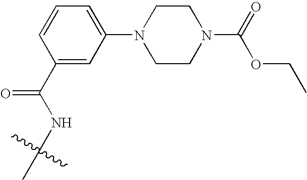

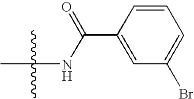

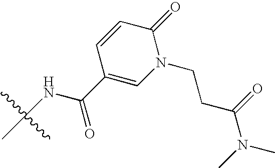

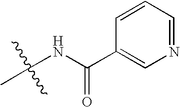

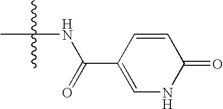

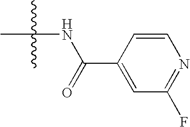

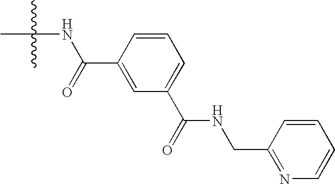

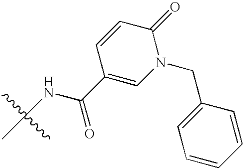

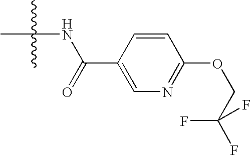

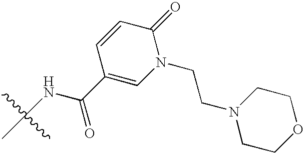

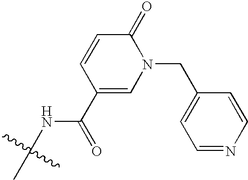

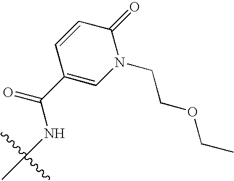

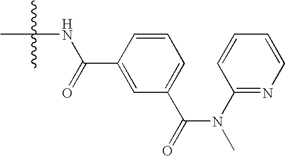

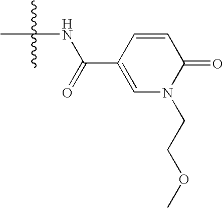

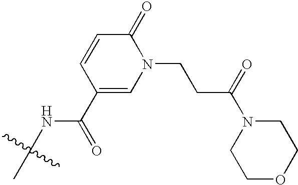

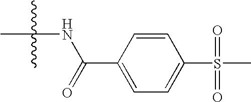

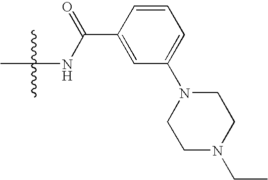

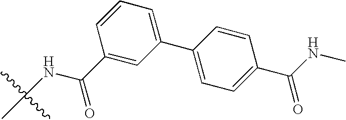

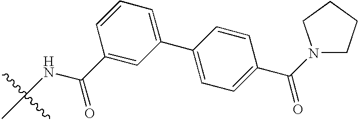

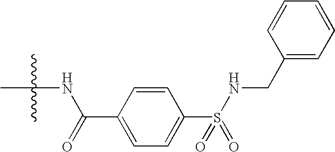

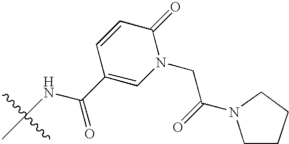

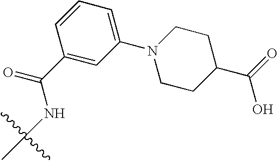

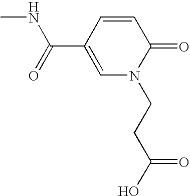

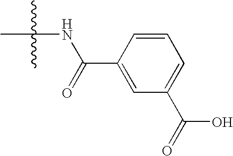

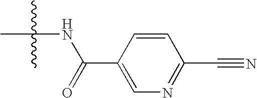

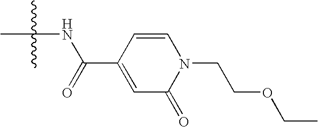

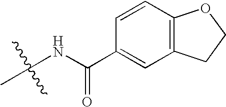

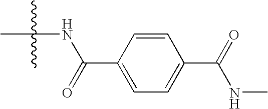

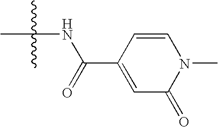

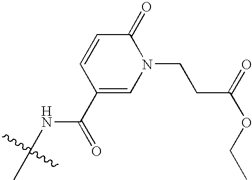

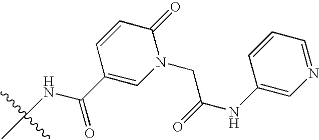

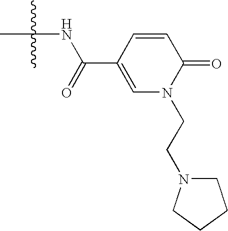

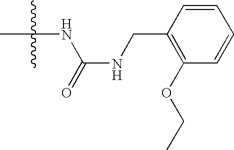

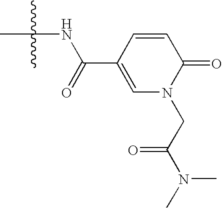

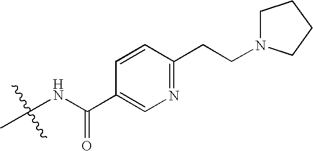

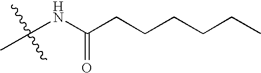

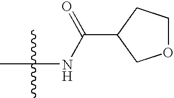

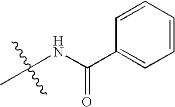

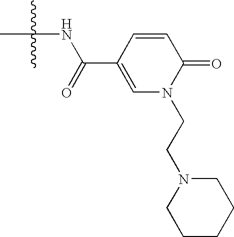

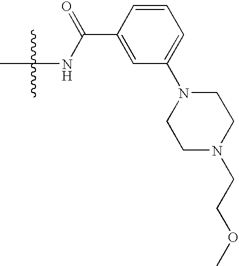

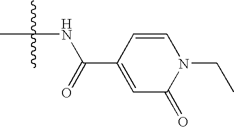

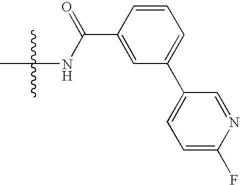

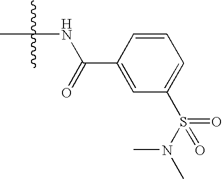

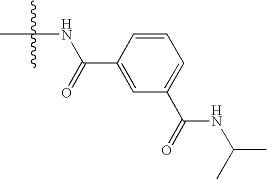

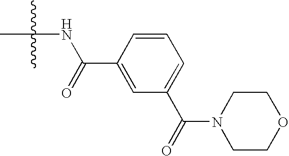

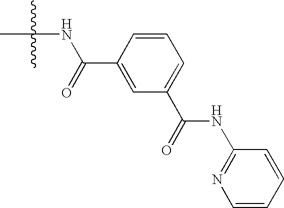

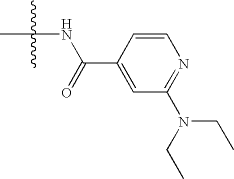

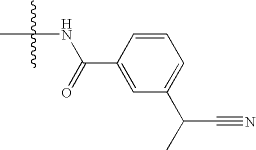

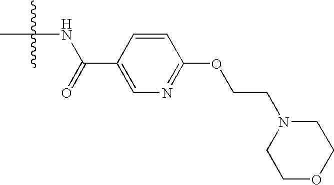

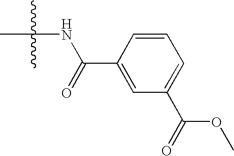

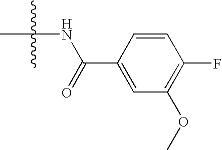

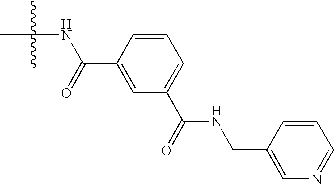

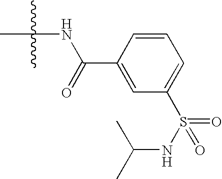

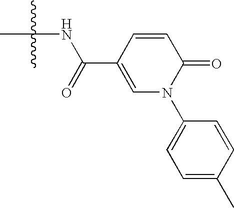

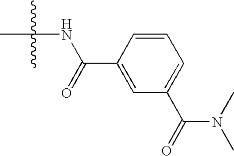

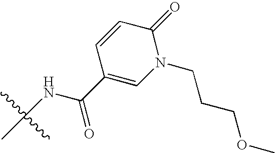

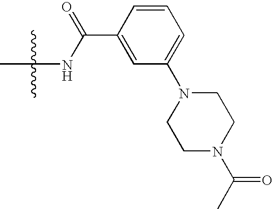

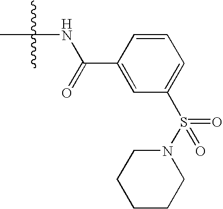

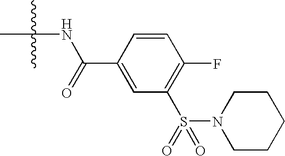

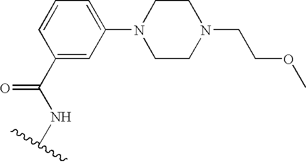

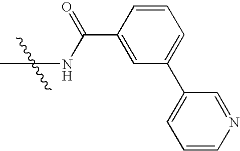

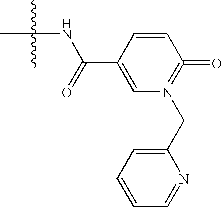

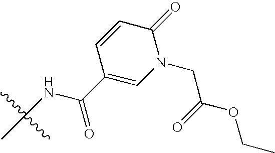

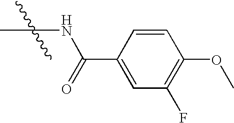

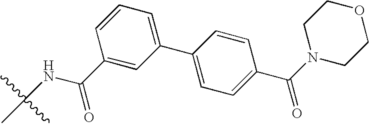

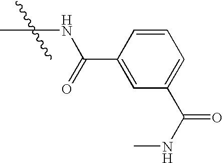

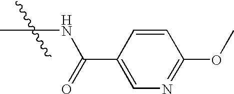

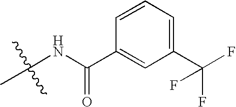

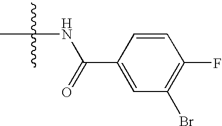

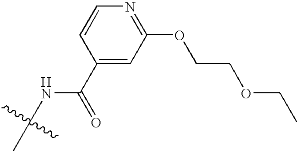

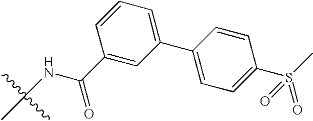

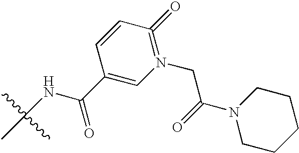

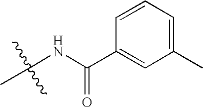

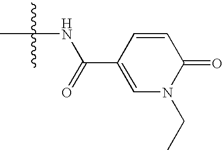

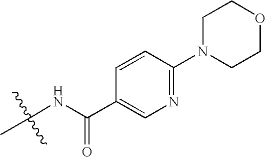

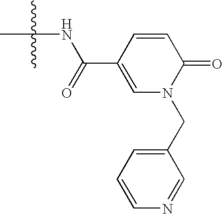

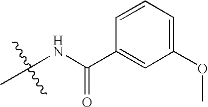

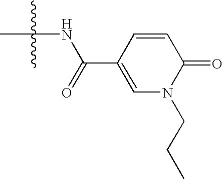

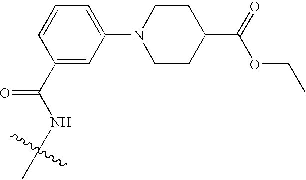

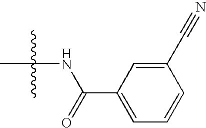

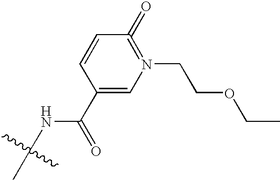

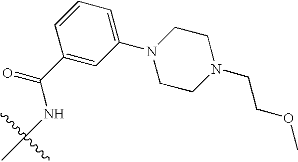

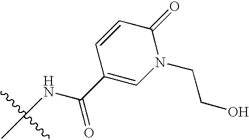

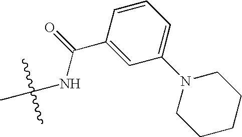

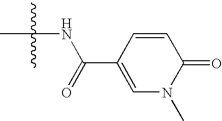

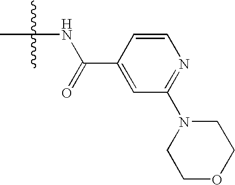

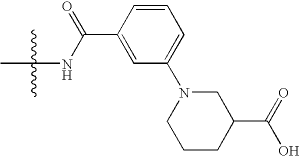

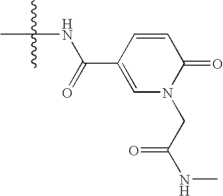

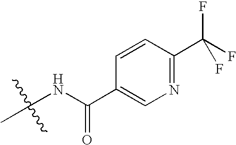

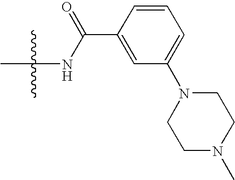

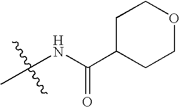

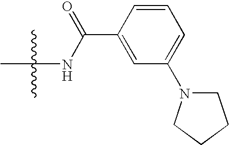

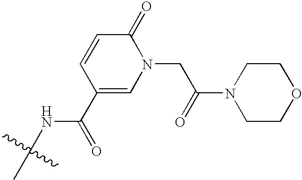

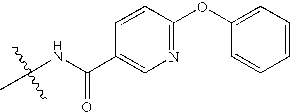

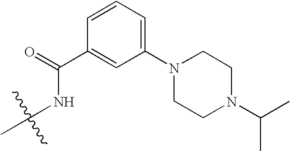

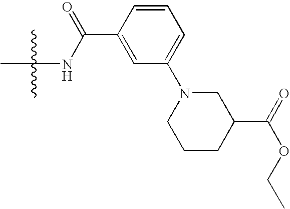

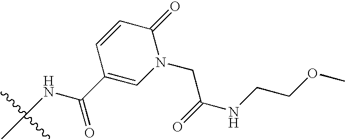

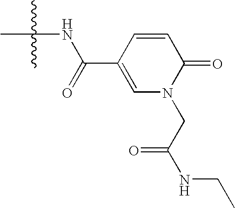

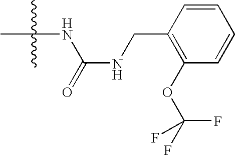

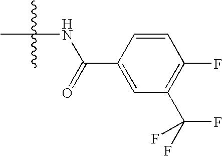

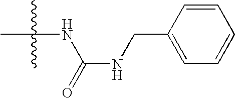

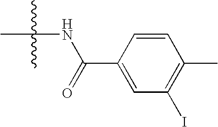

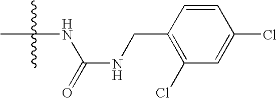

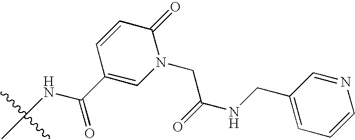

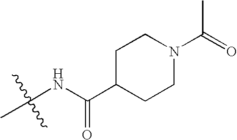

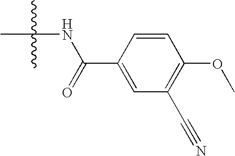

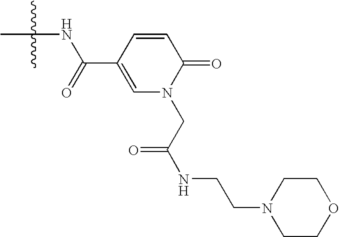

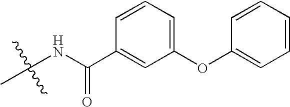

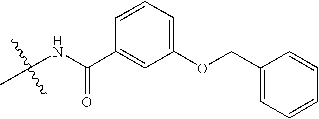

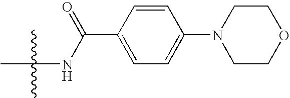

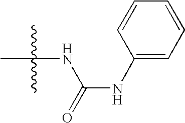

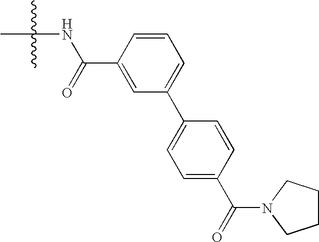

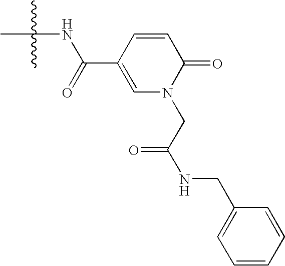

or the pharmaceutically acceptable salts thereof.

In another generic aspect of the invention, there is provided a compound of the formula (Ie), (If), (Ig) or (Ih):

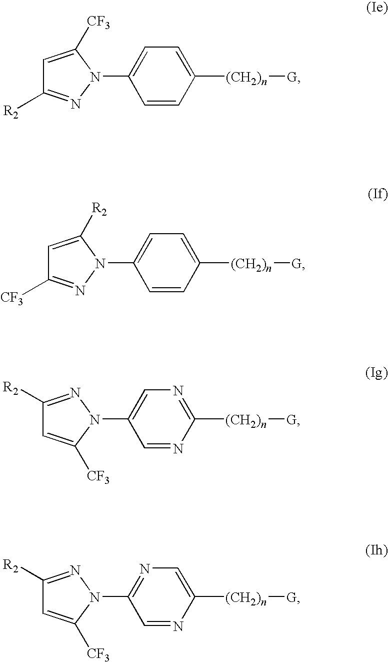

wherein for the Formula (Ie), (If), (Ig) or (Ih), the component R₂ is:

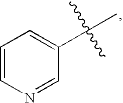

and the component

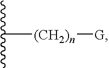

is chosen from those shown in the table IV below;

TABLE IV

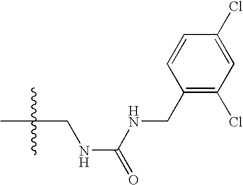

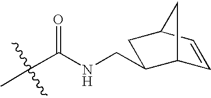

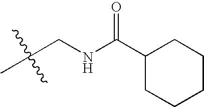

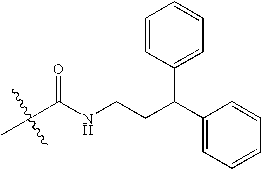

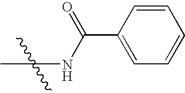

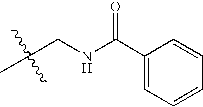

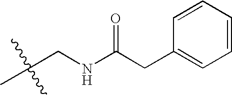

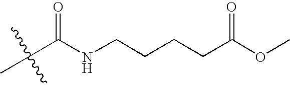

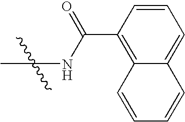

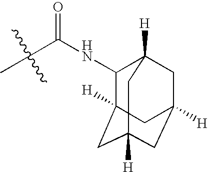

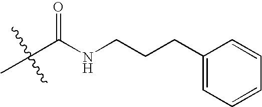

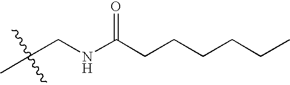

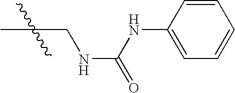

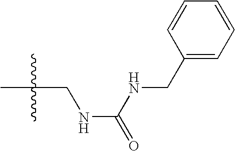

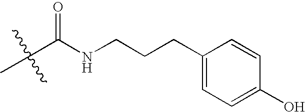

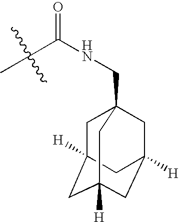

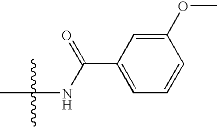

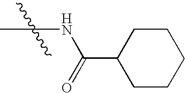

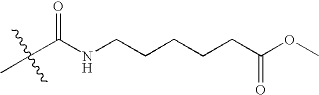

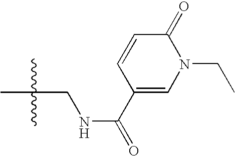

or the pharmaceutically acceptable salts thereof.

In one aspect of the invention, there is provided the following compounds which can be made according to the general synthetic procedures and examples which follow:

TABLE V

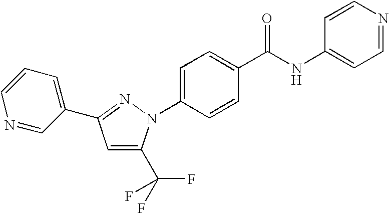

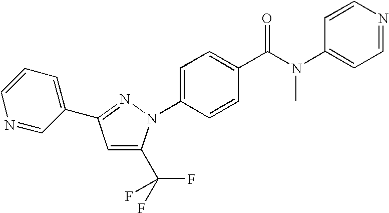

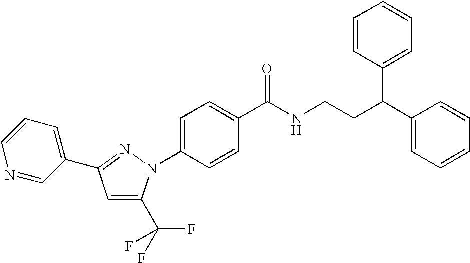

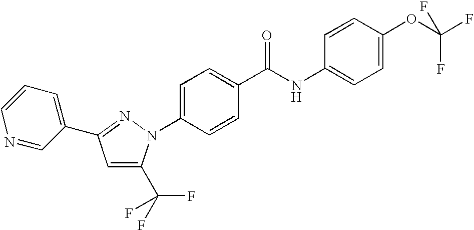

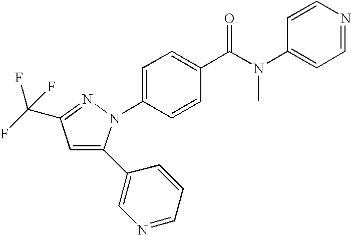

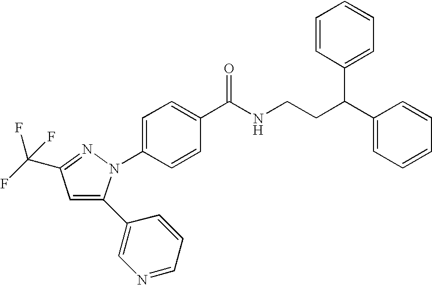

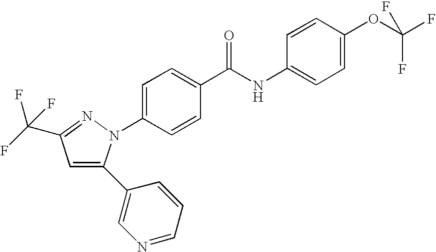

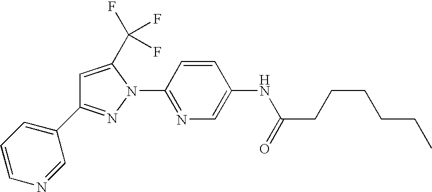

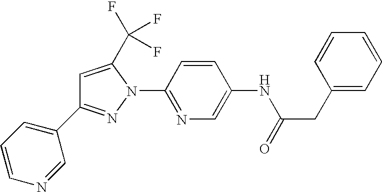

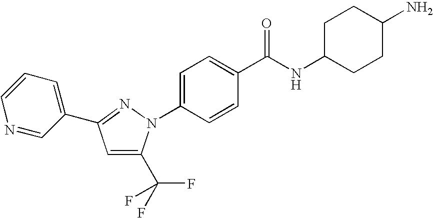

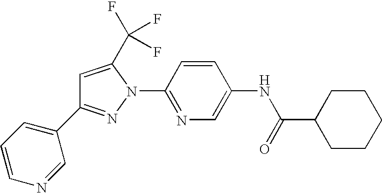

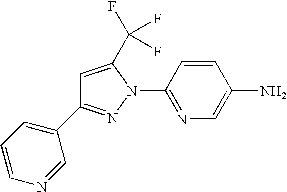

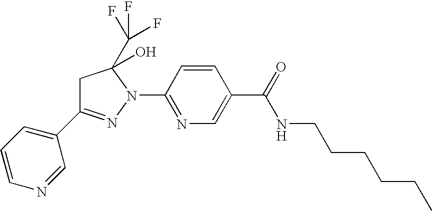

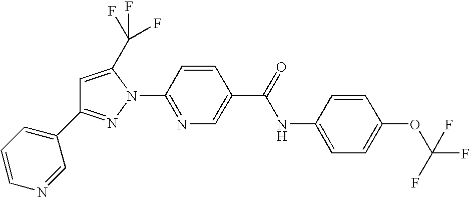

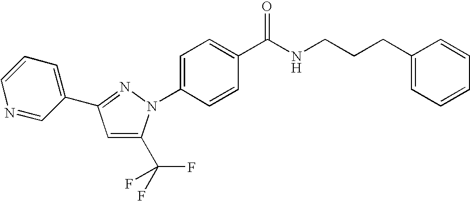

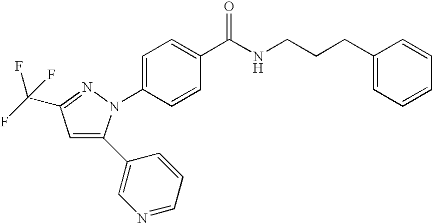

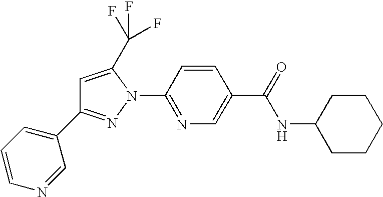

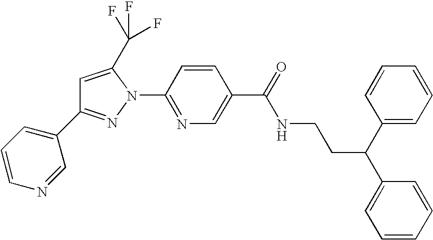

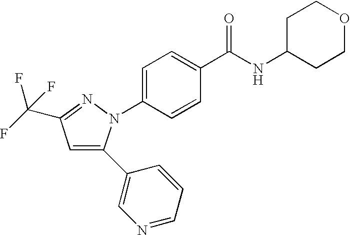

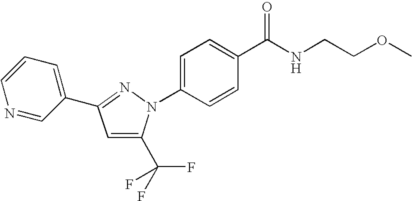

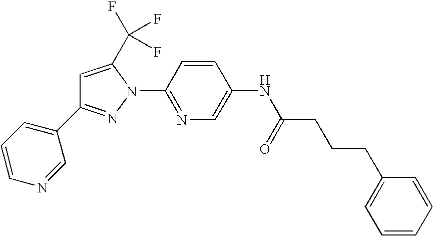

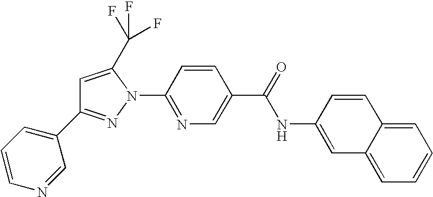

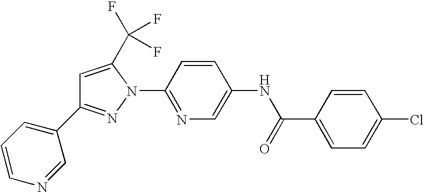

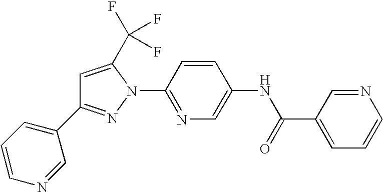

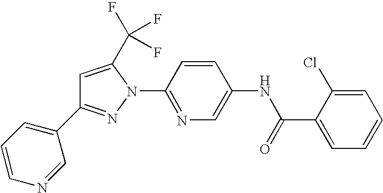

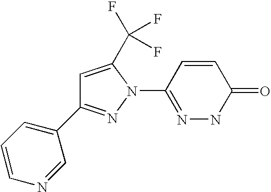

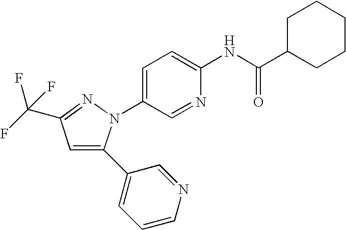

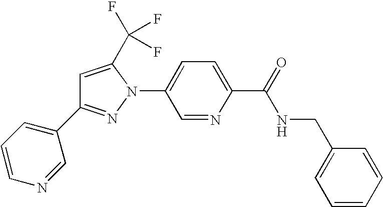

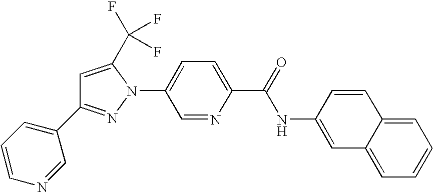

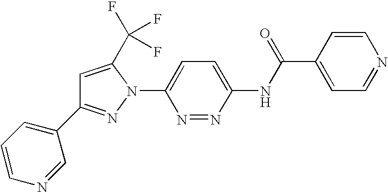

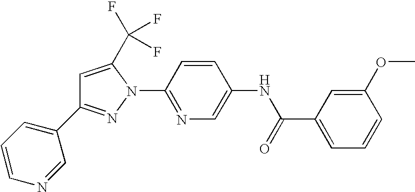

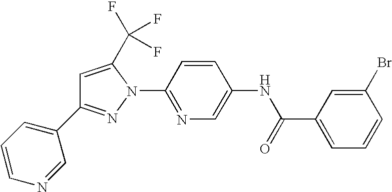

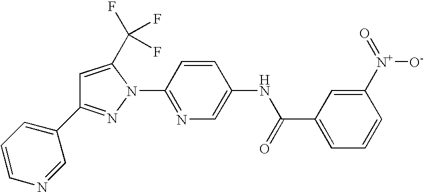

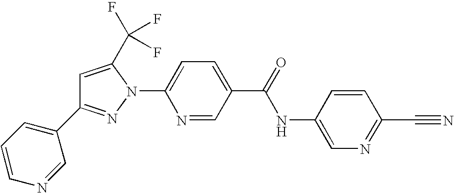

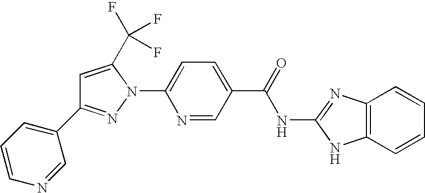

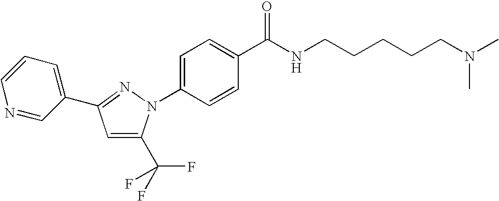

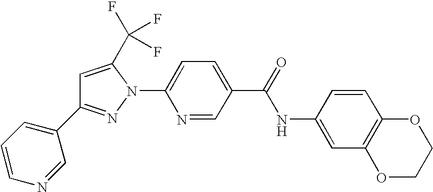

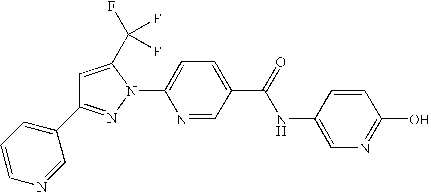

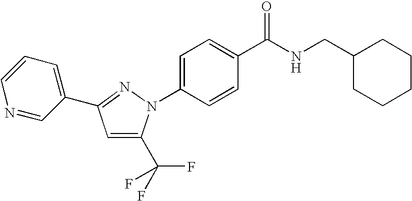

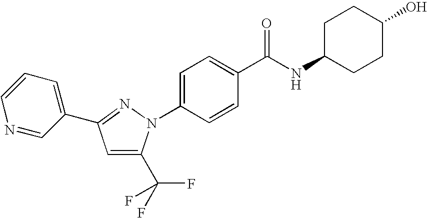

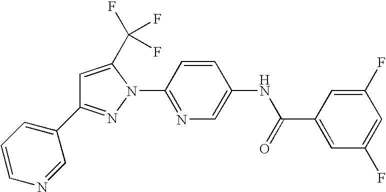

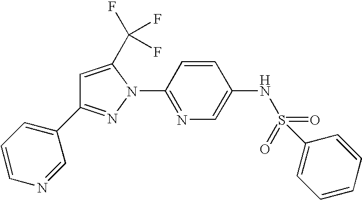

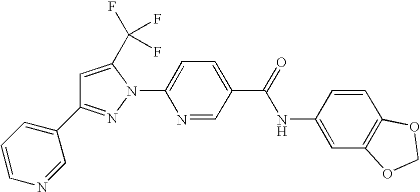

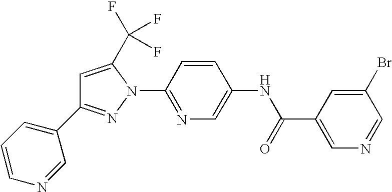

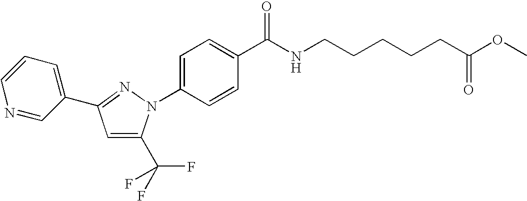

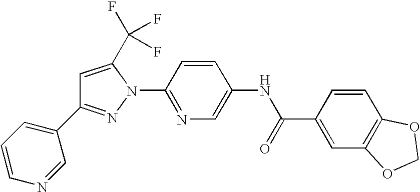

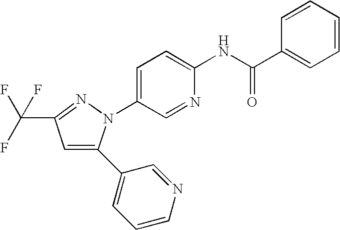

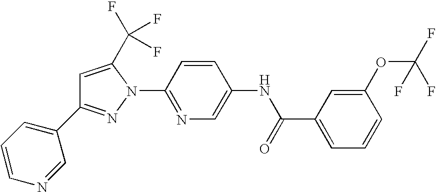

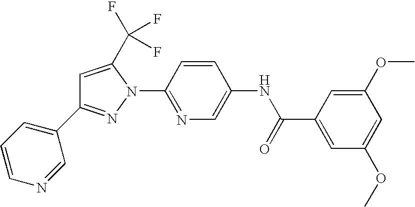

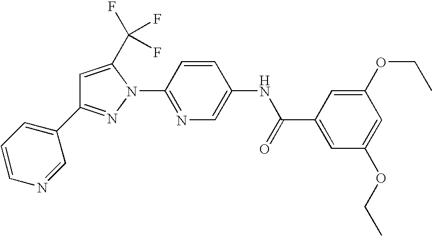

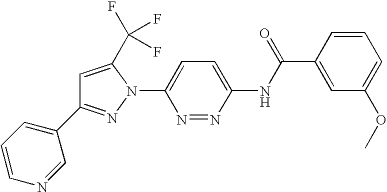

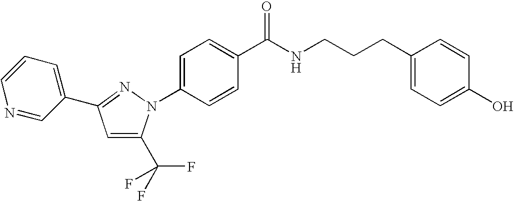

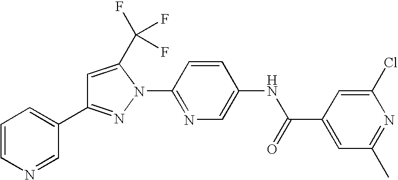

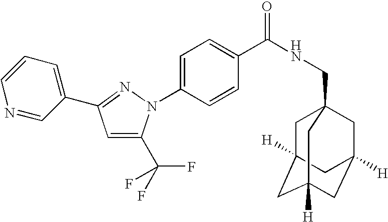

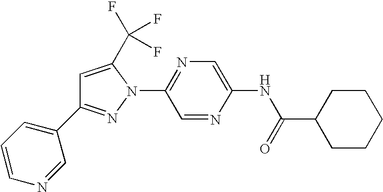

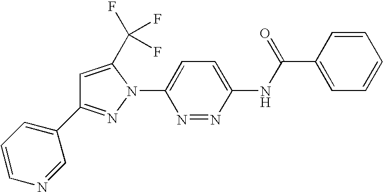

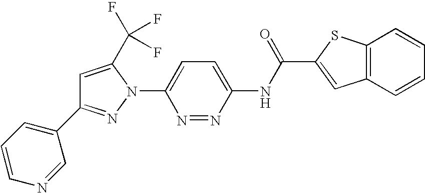

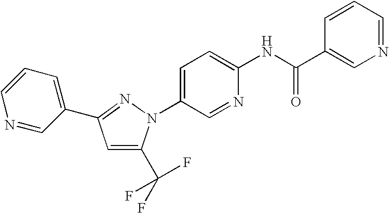

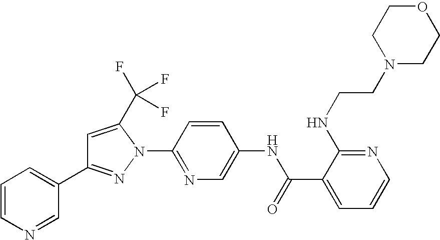

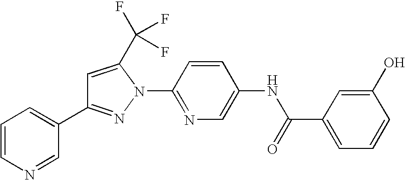

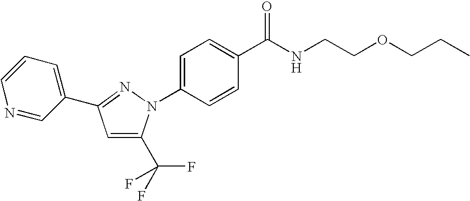

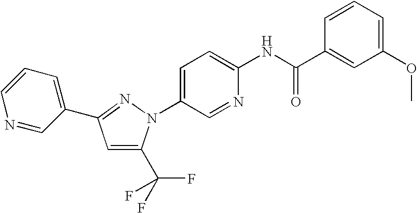

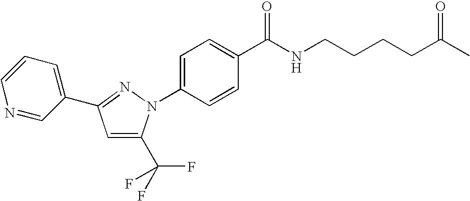

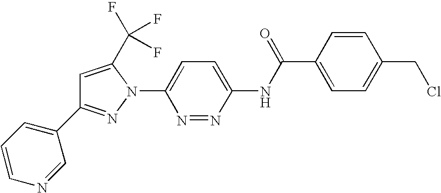

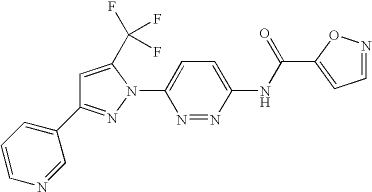

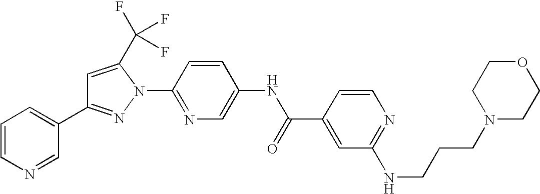

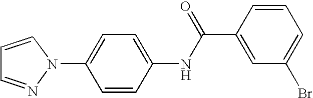

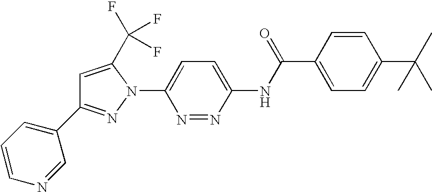

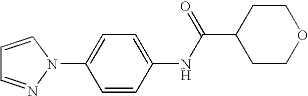

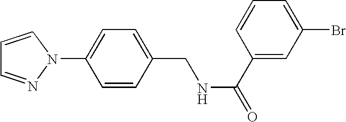

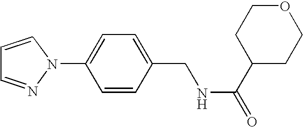

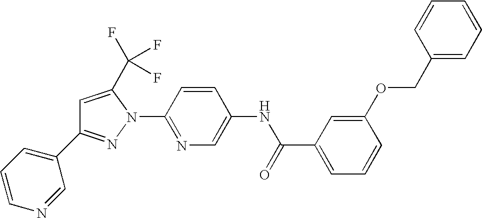

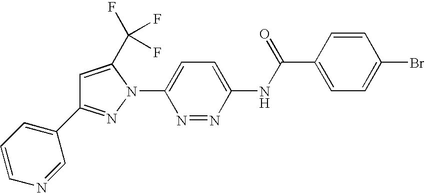

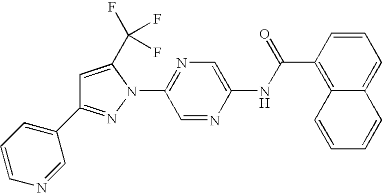

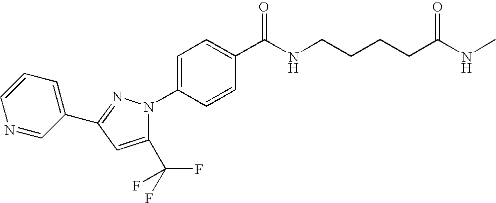

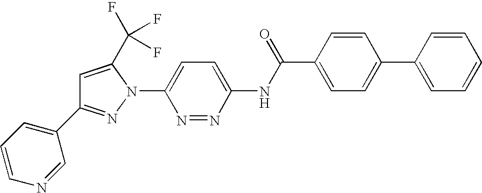

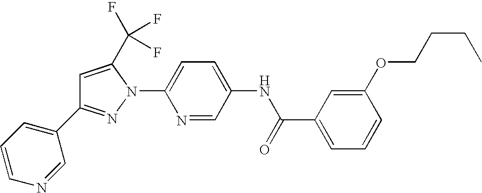

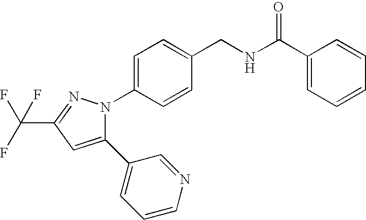

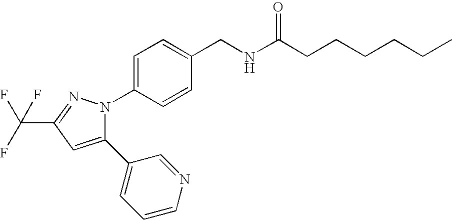

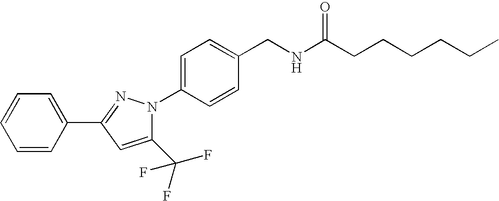

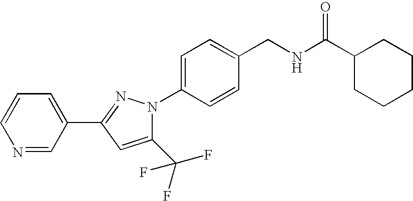

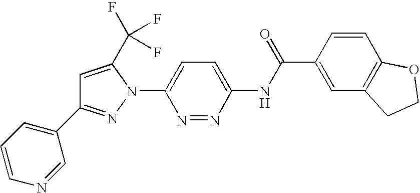

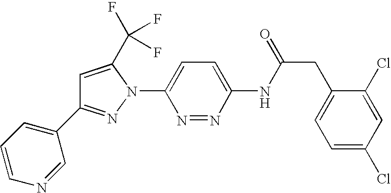

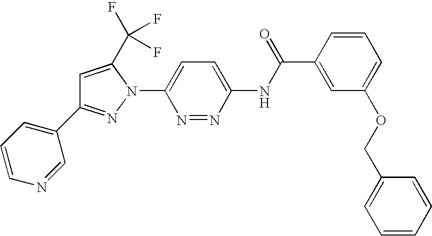

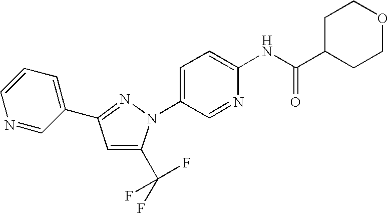

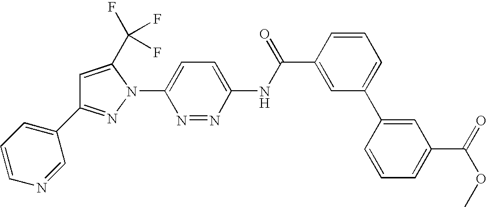

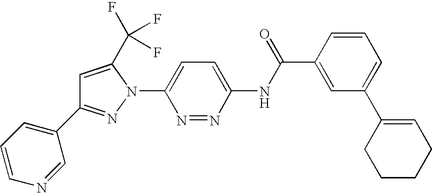

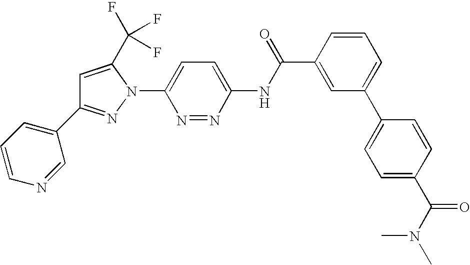

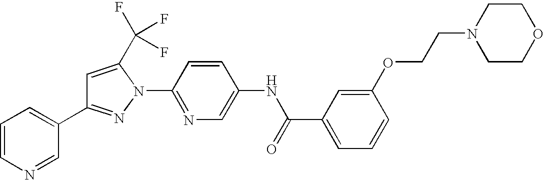

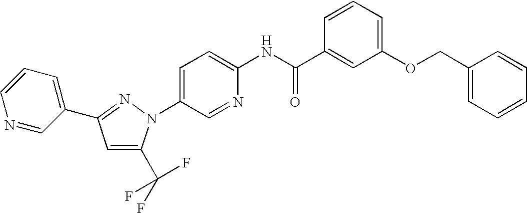

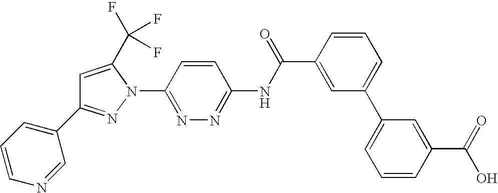

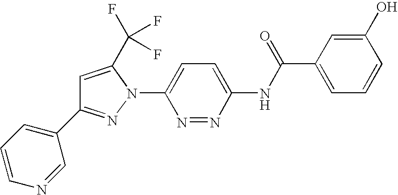

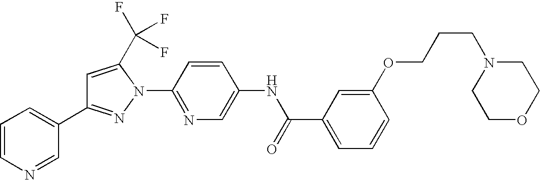

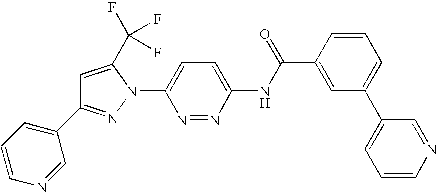

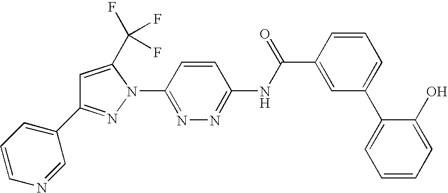

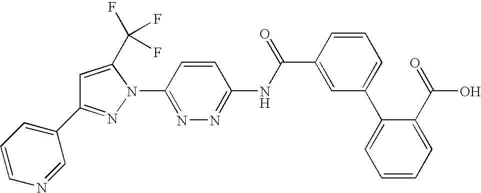

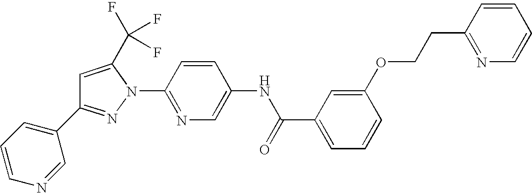

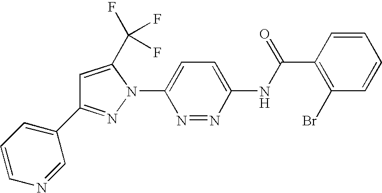

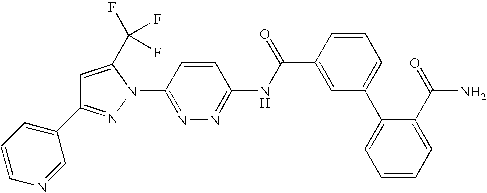

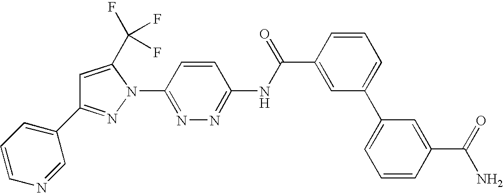

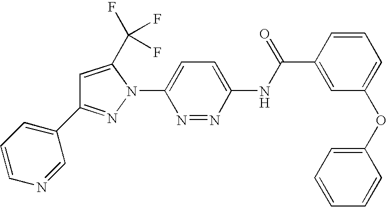

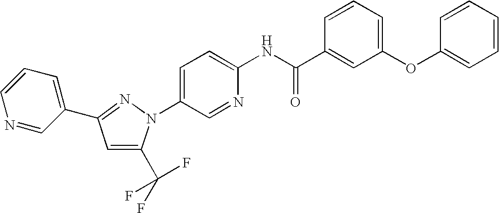

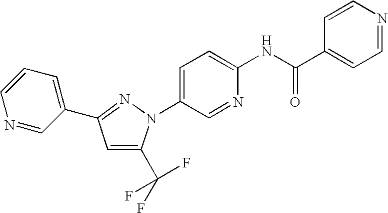

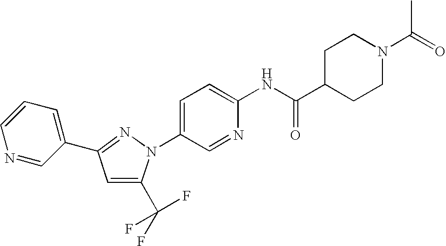

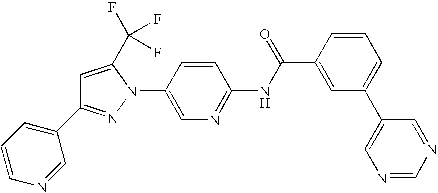

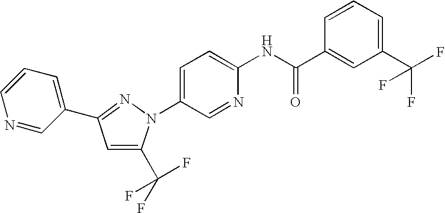

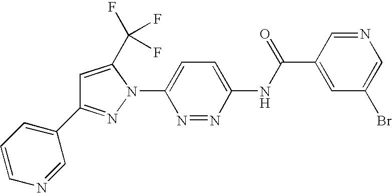

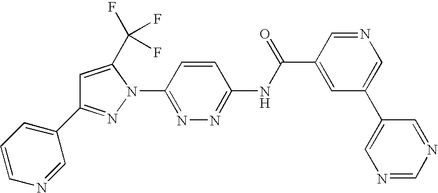

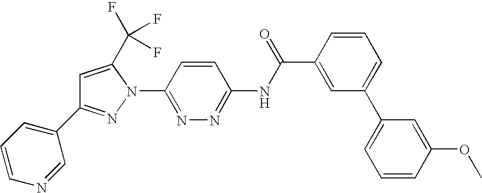

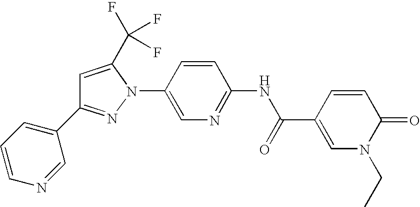

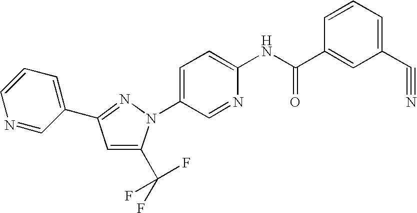

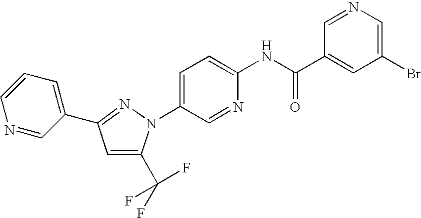

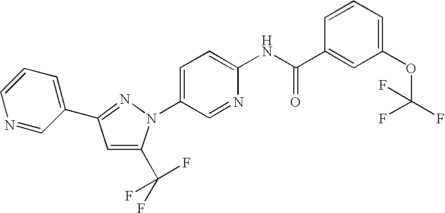

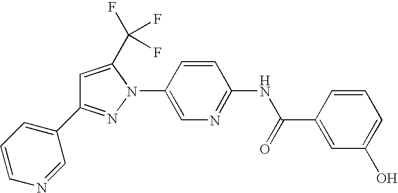

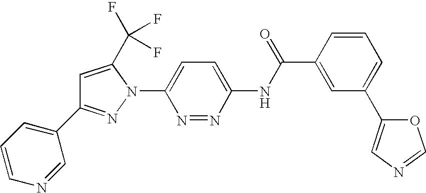

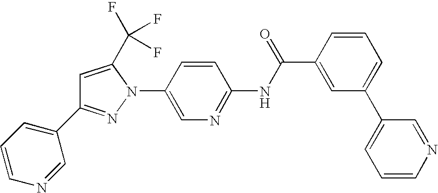

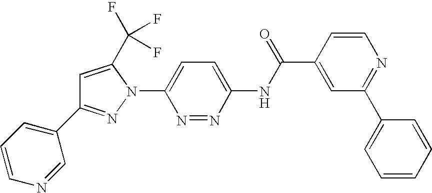

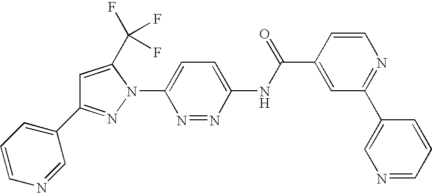

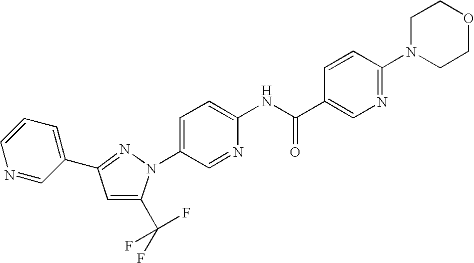

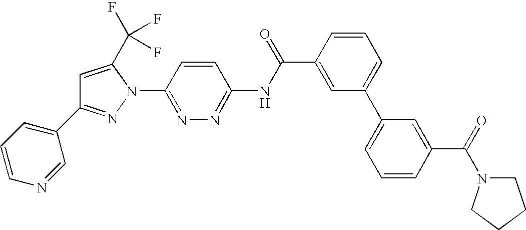

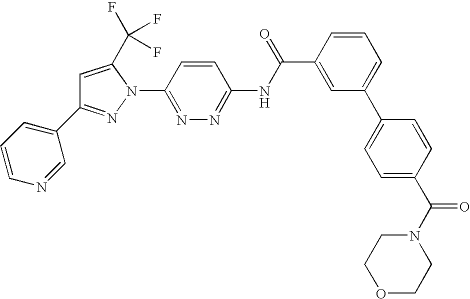

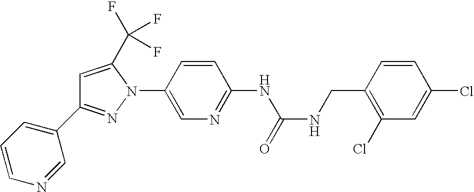

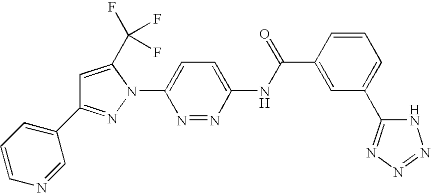

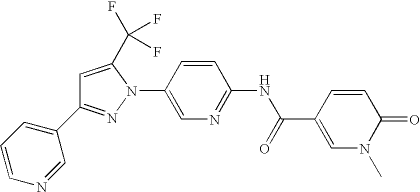

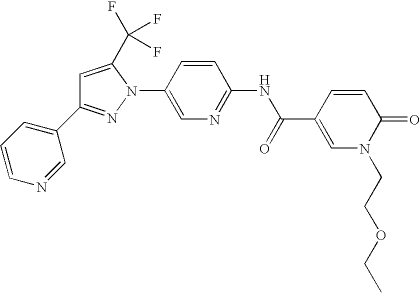

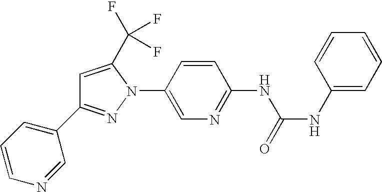

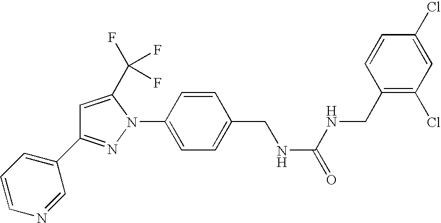

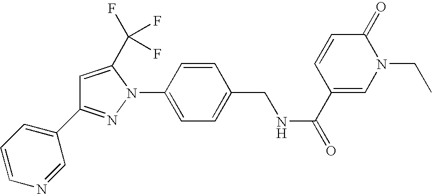

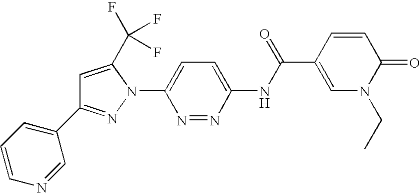

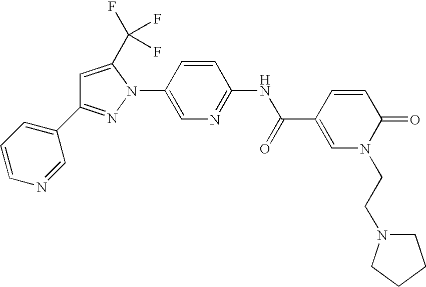

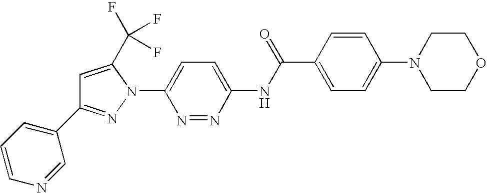

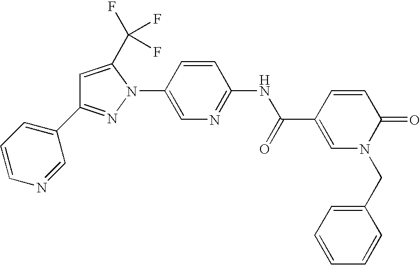

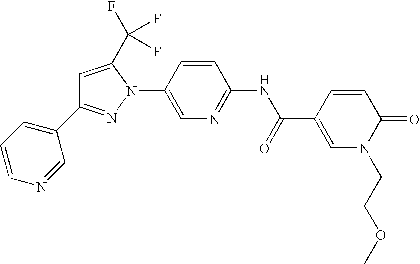

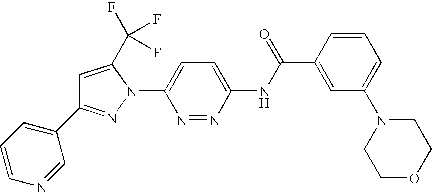

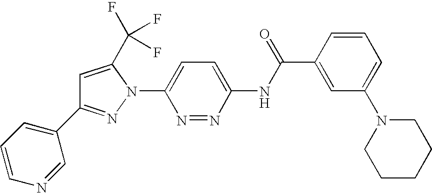

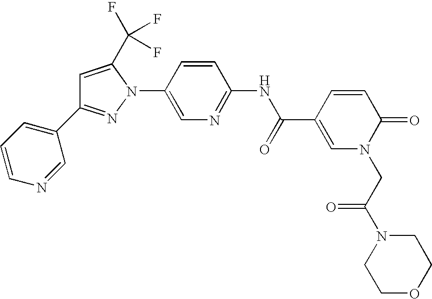

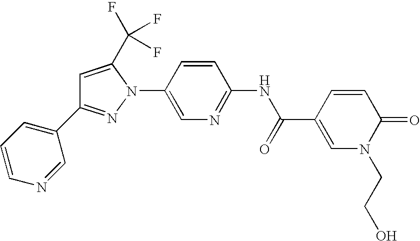

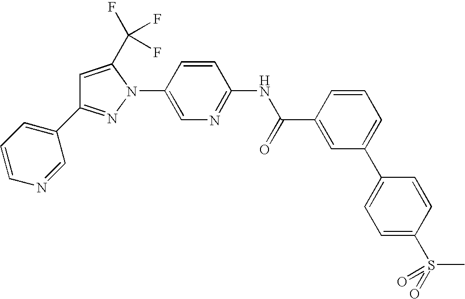

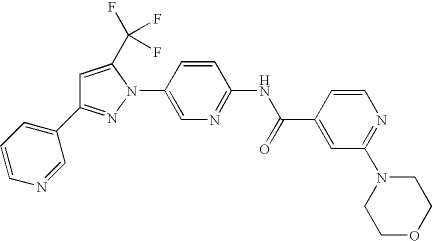

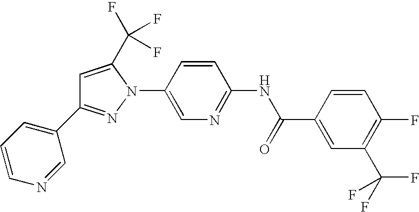

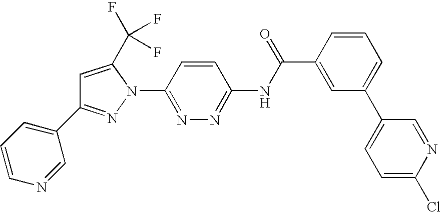

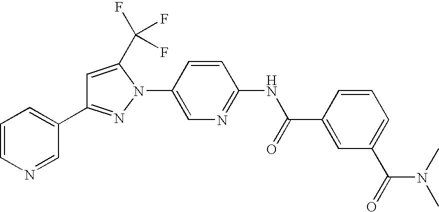

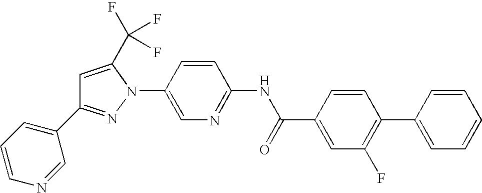

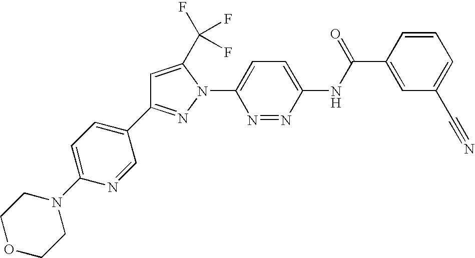

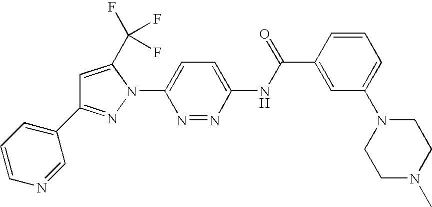

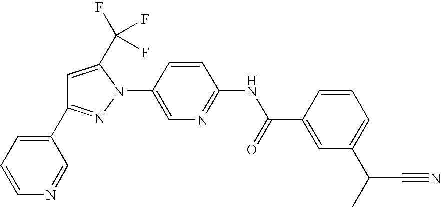

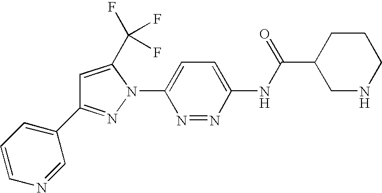

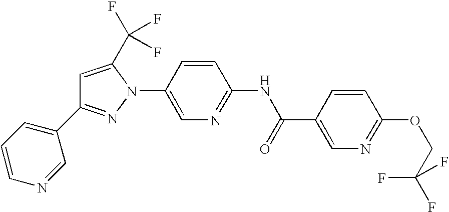

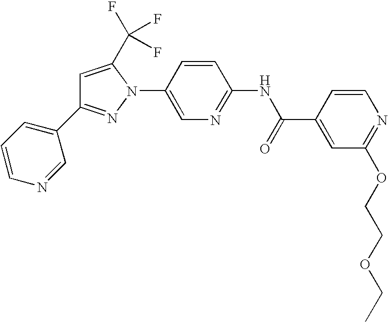

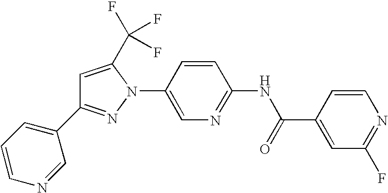

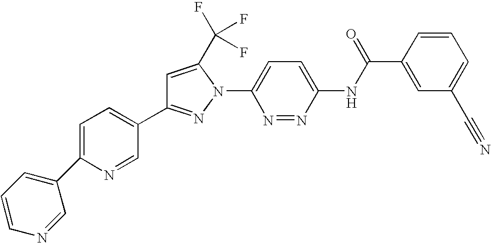

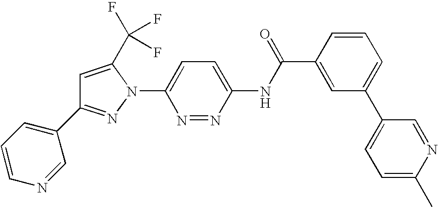

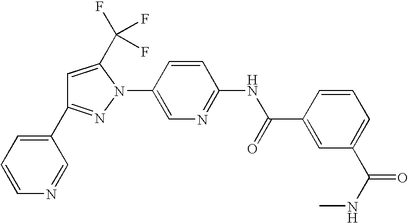

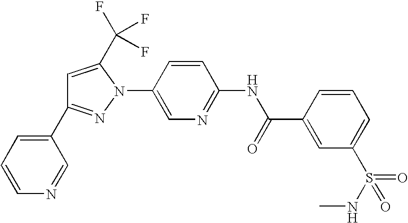

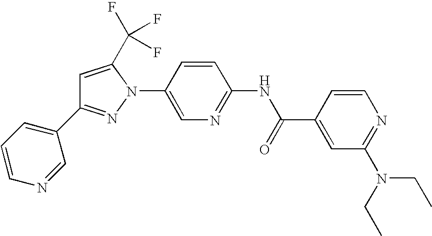

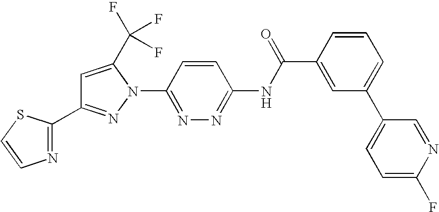

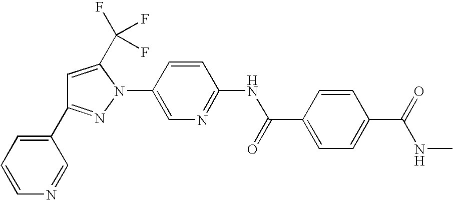

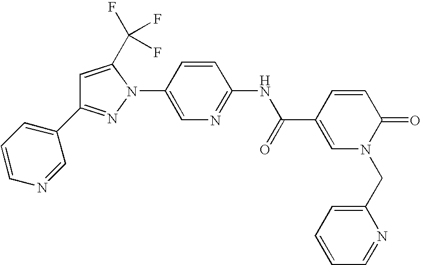

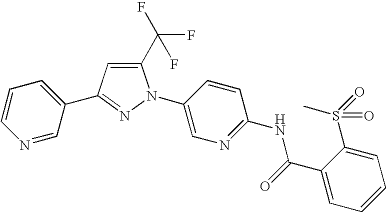

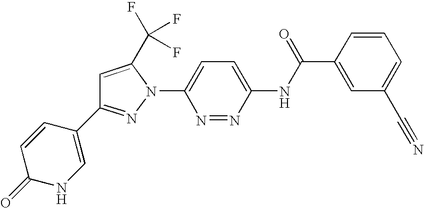

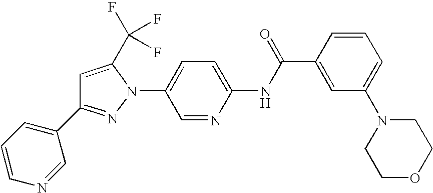

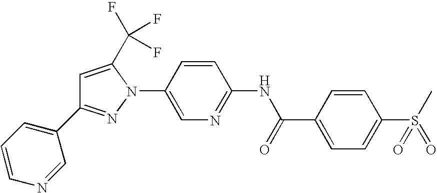

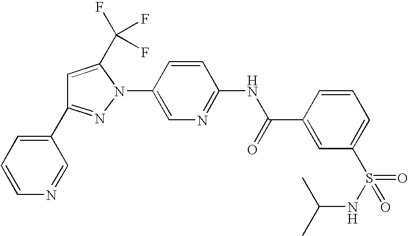

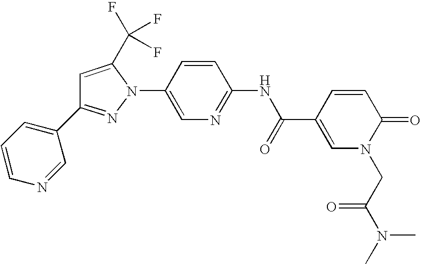

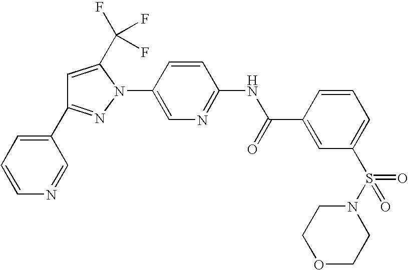

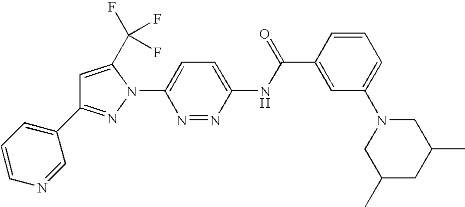

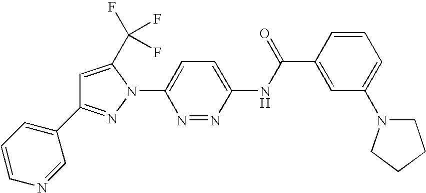

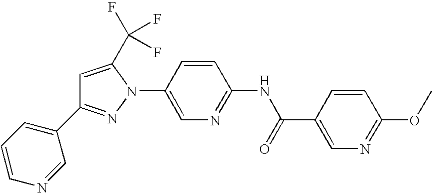

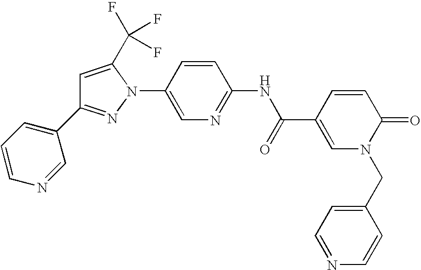

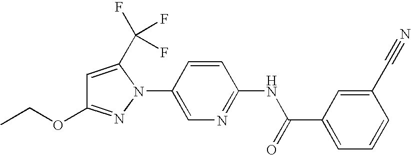

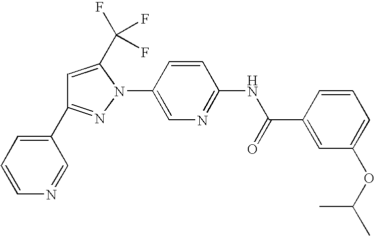

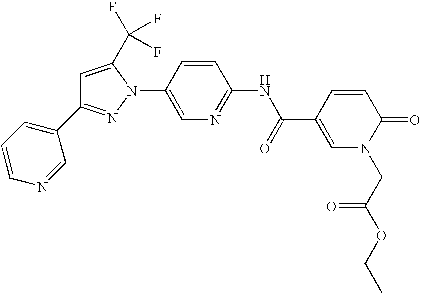

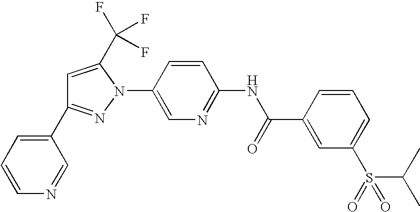

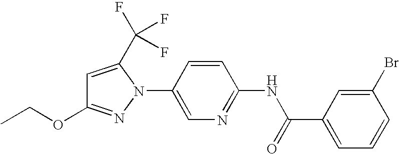

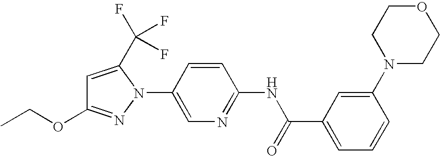

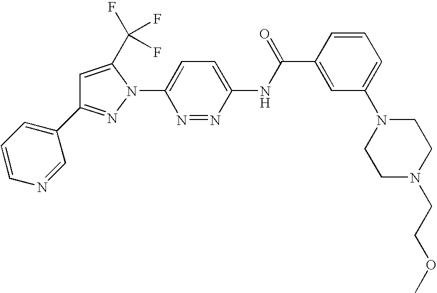

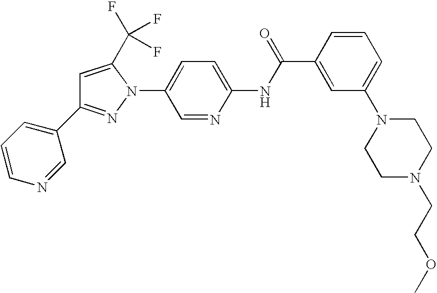

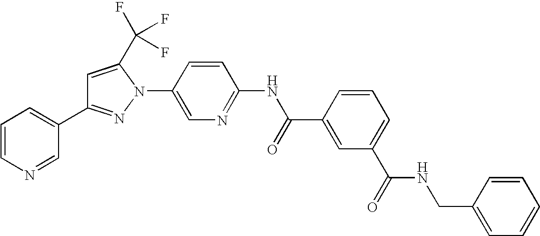

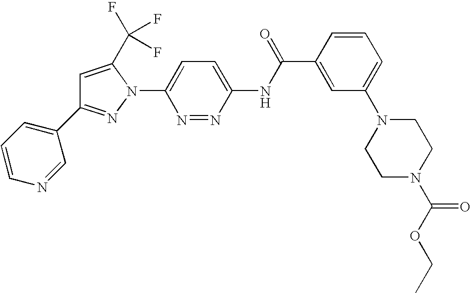

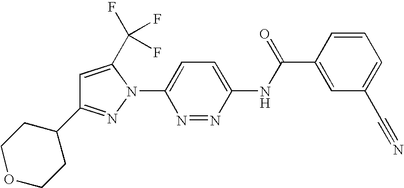

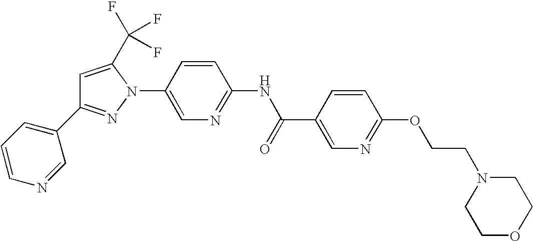

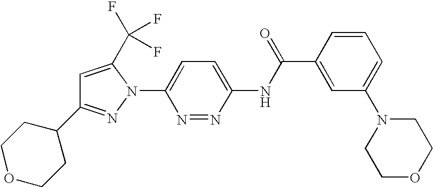

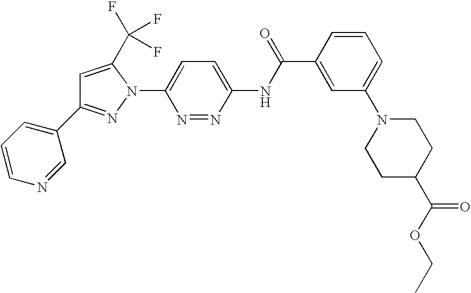

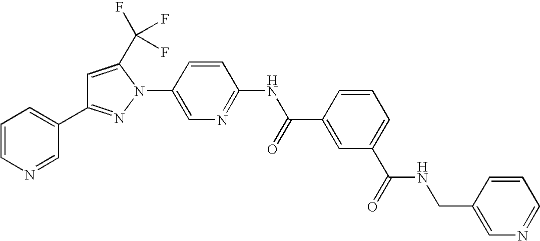

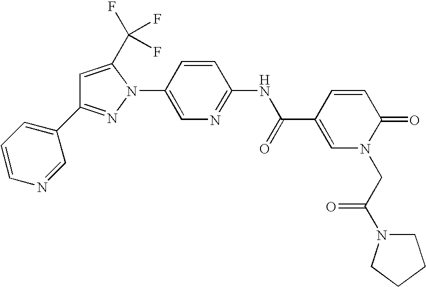

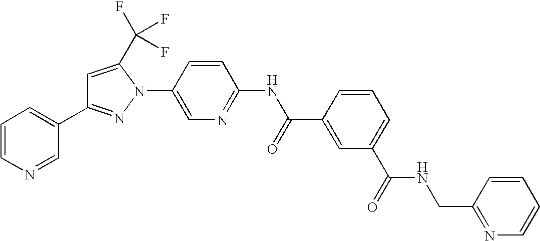

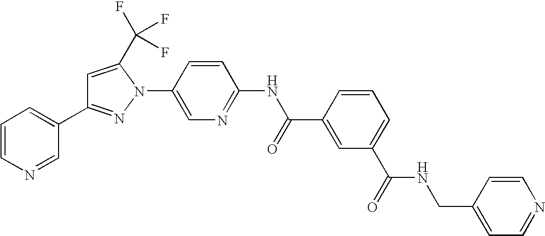

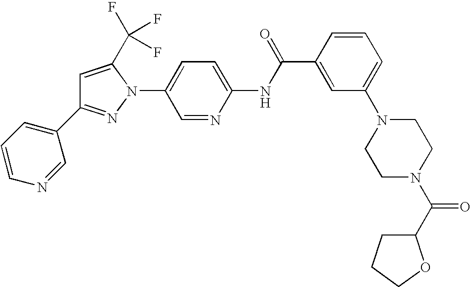

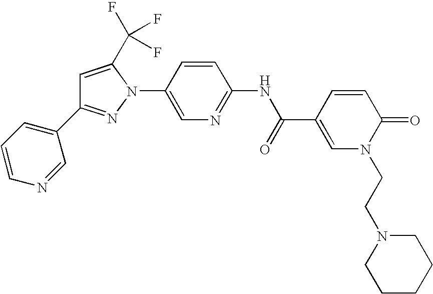

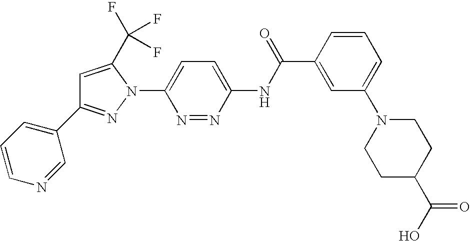

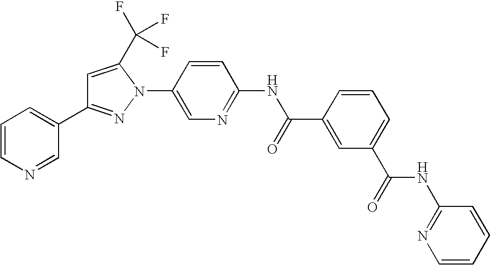

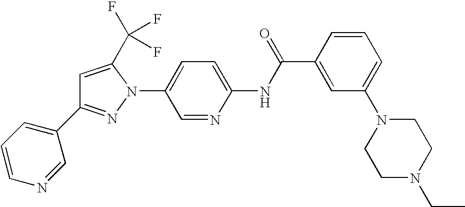

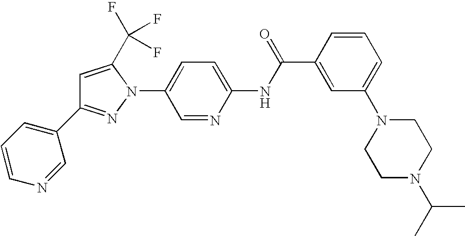

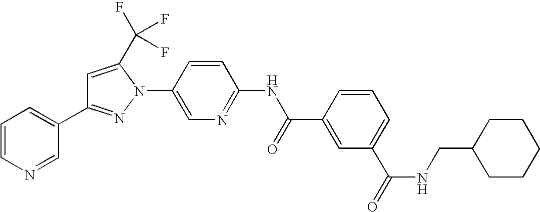

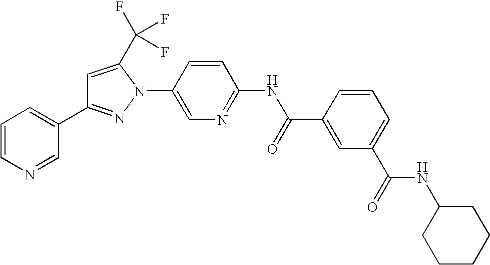

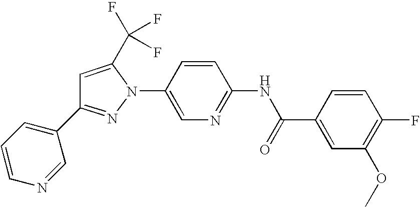

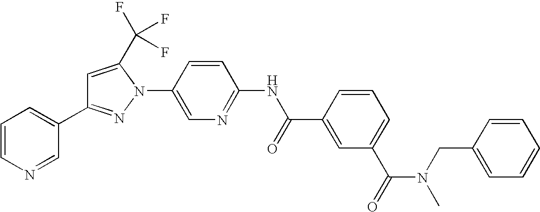

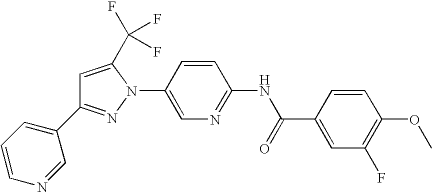

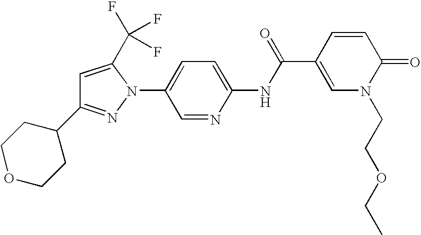

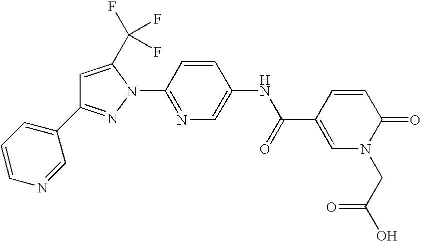

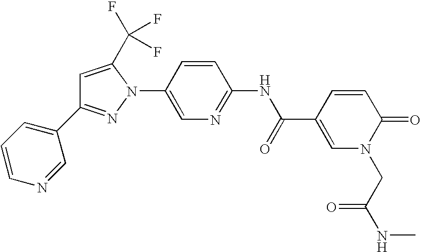

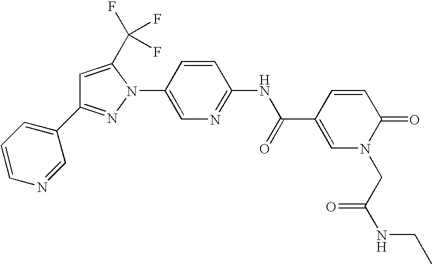

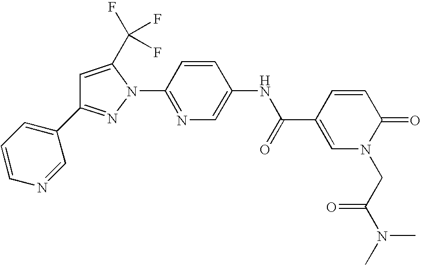

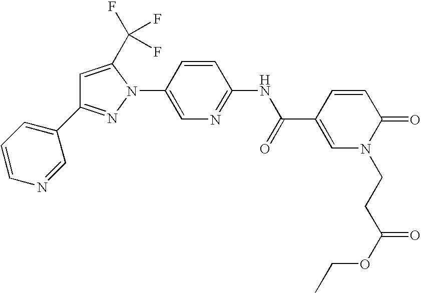

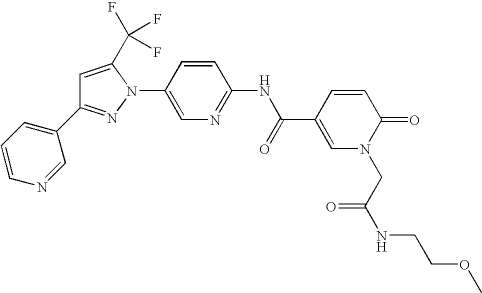

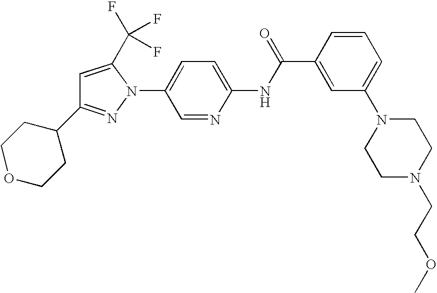

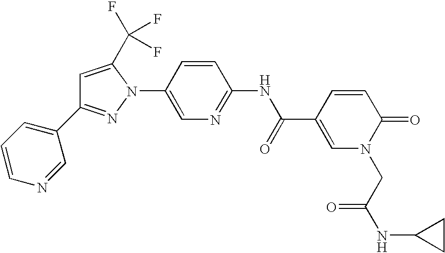

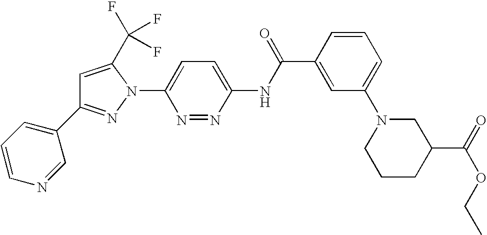

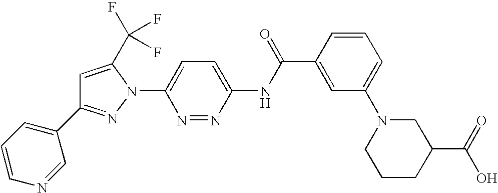

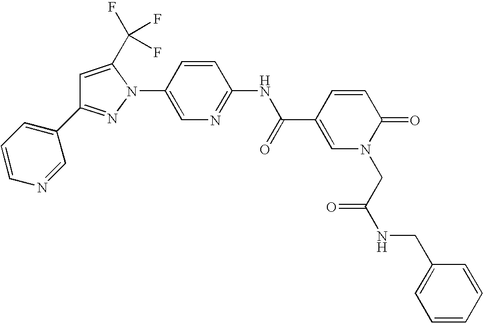

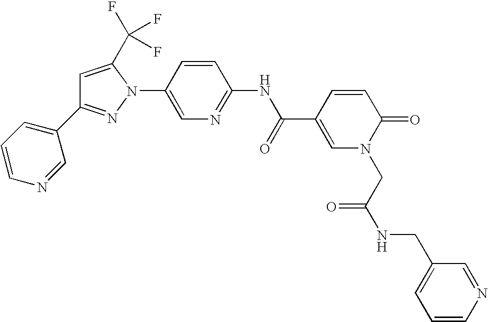

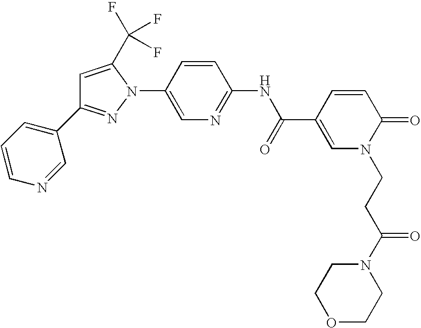

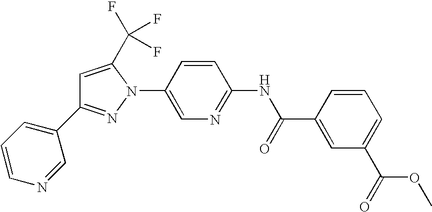

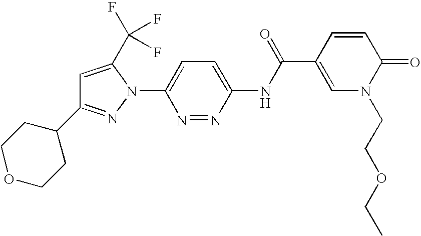

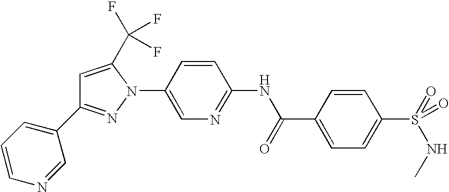

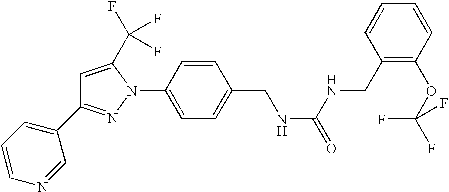

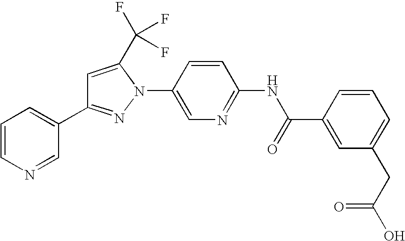

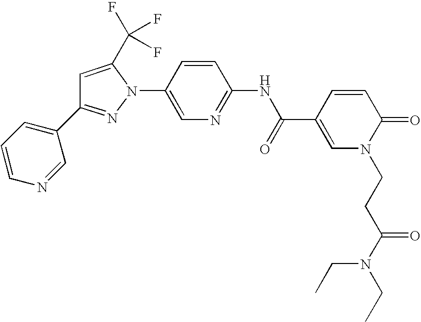

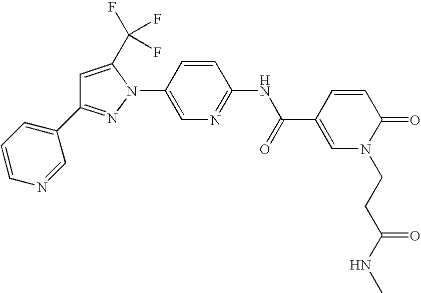

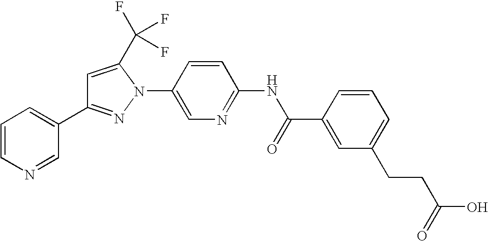

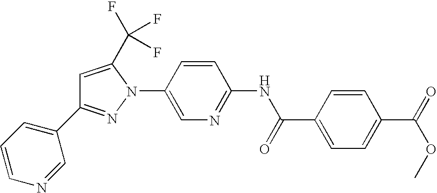

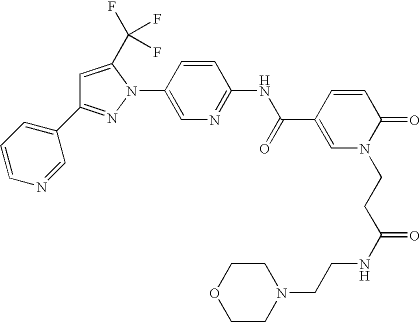

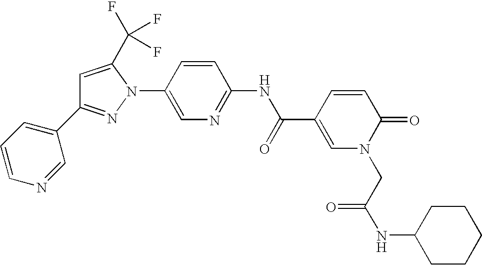

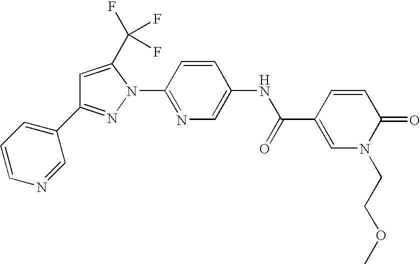

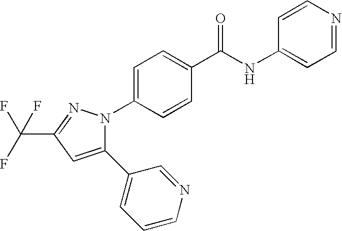

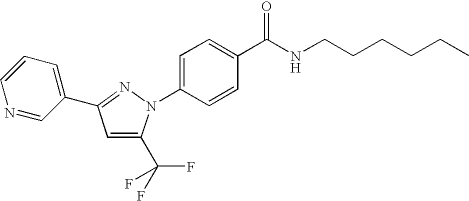

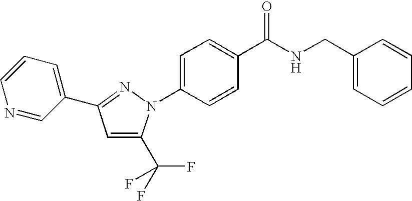

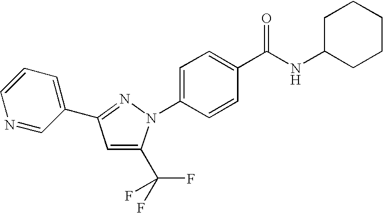

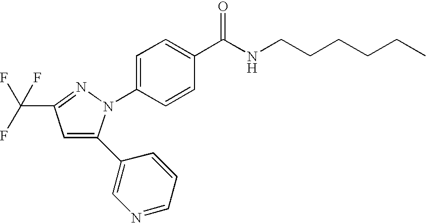

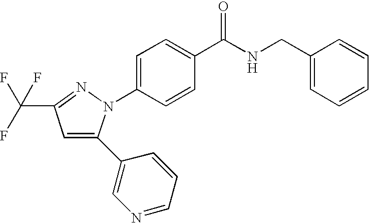

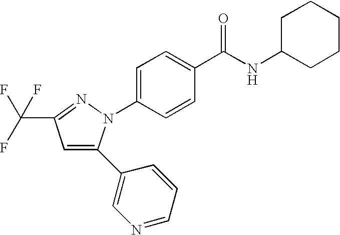

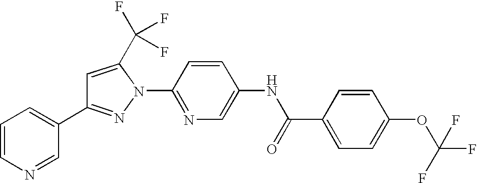

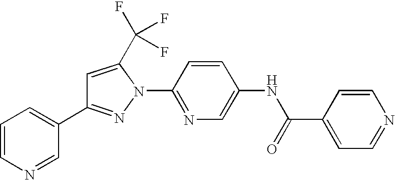

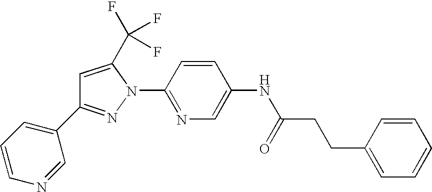

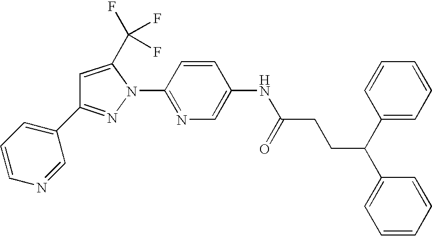

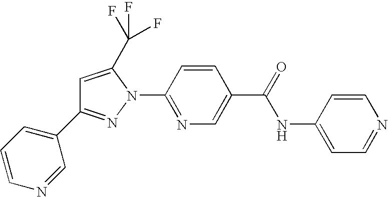

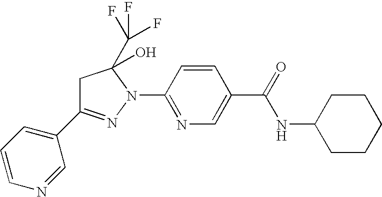

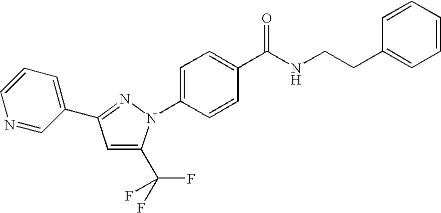

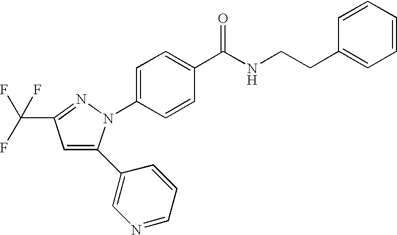

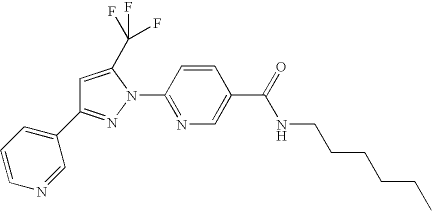

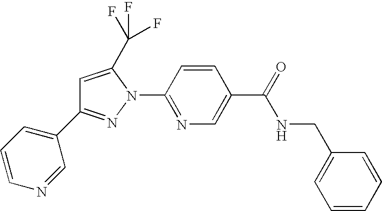

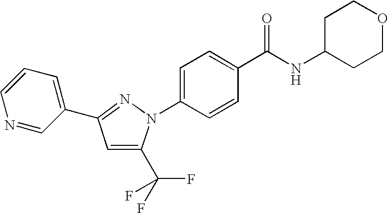

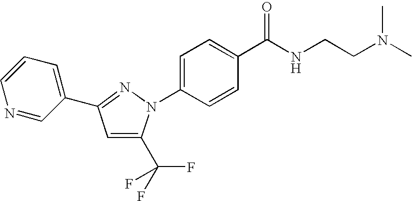

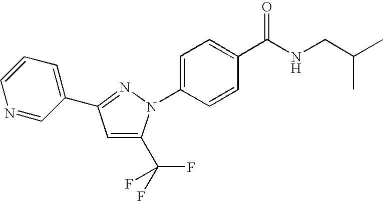

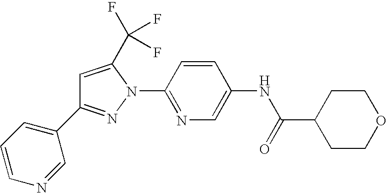

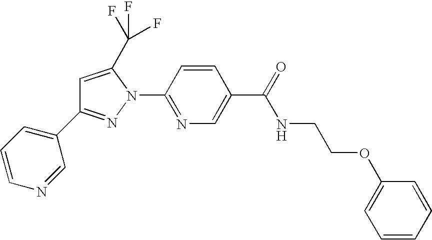

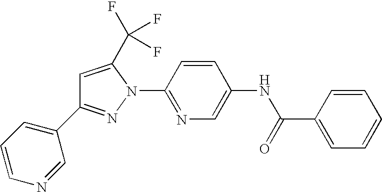

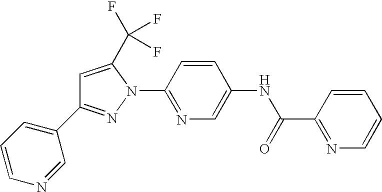

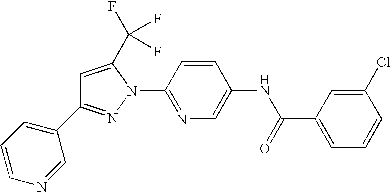

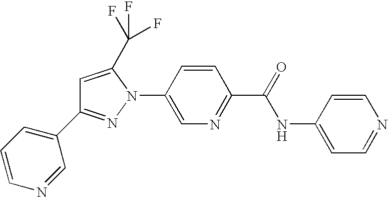

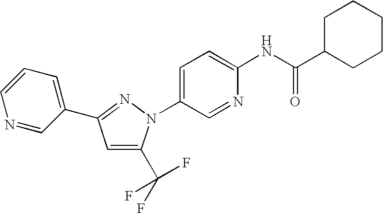

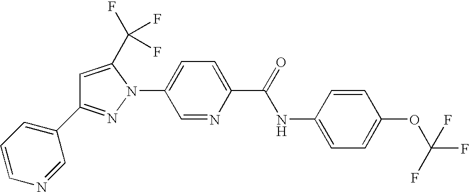

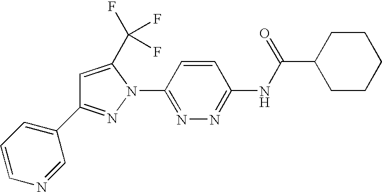

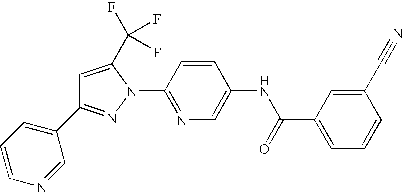

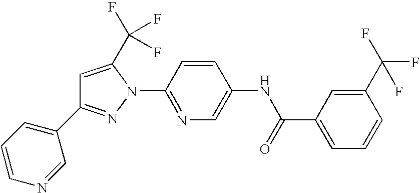

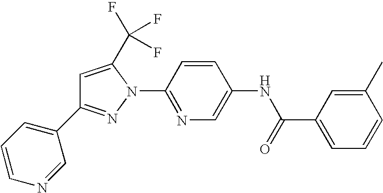

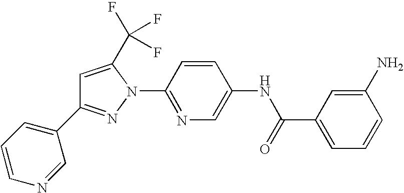

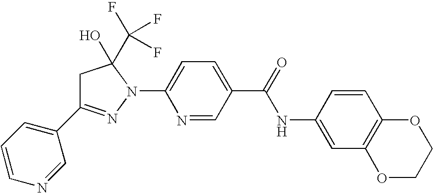

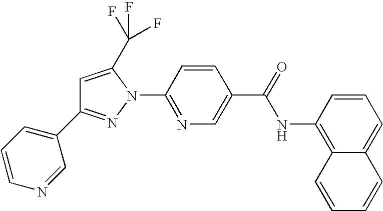

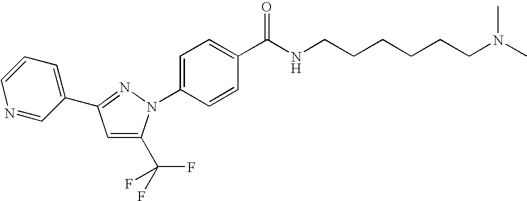

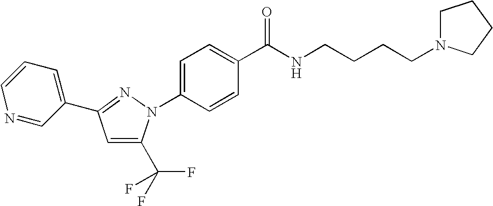

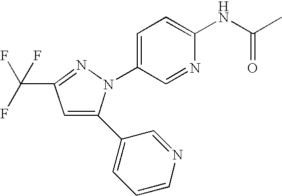

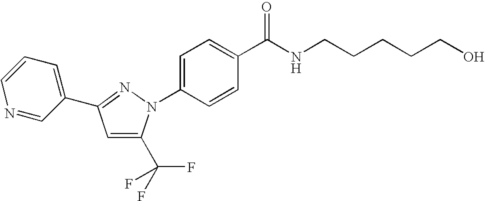

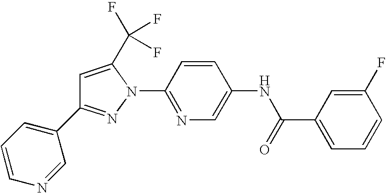

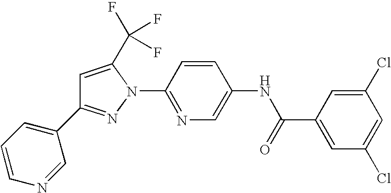

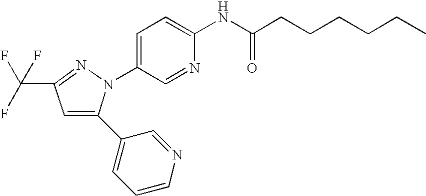

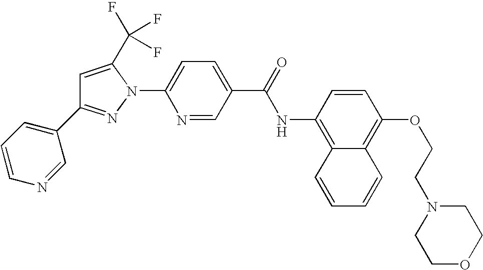

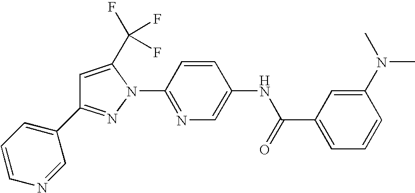

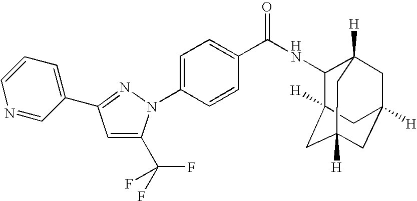

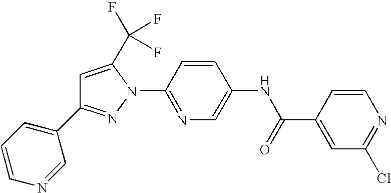

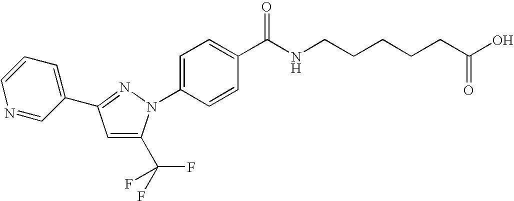

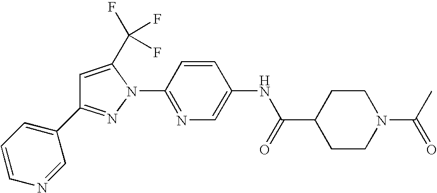

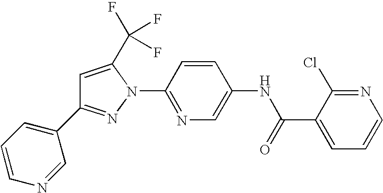

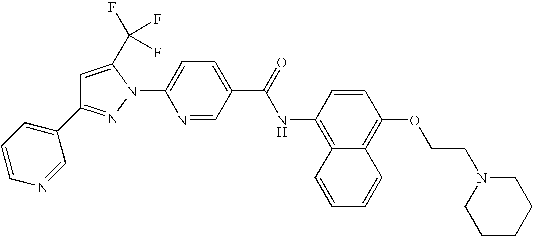

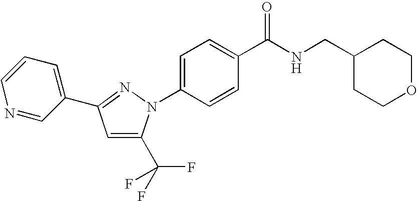

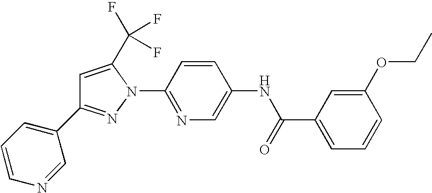

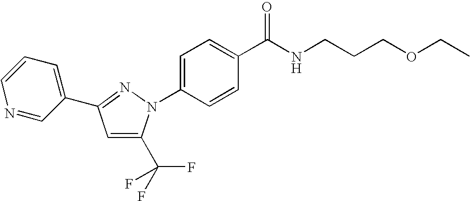

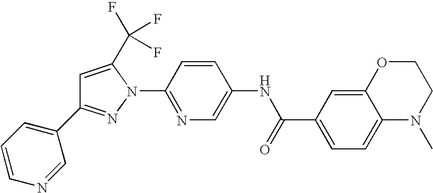

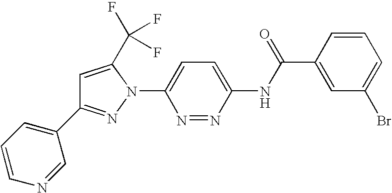

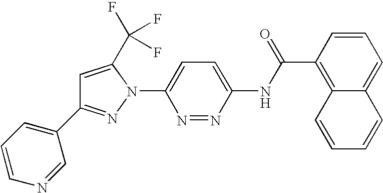

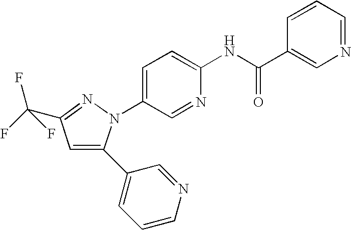

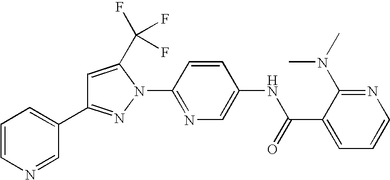

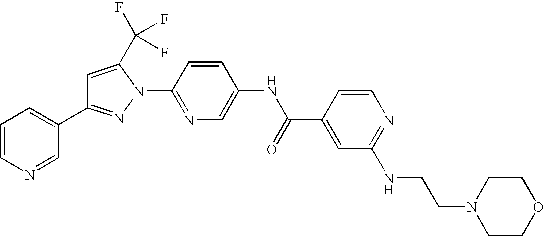

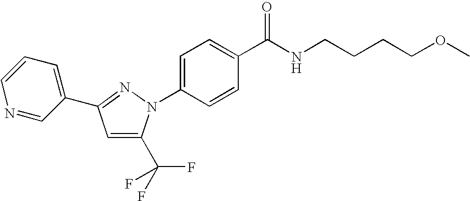

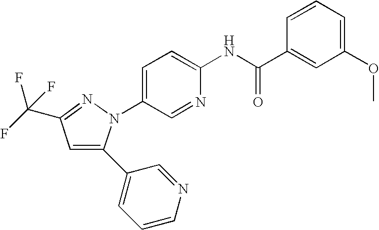

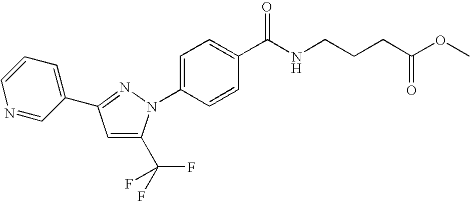

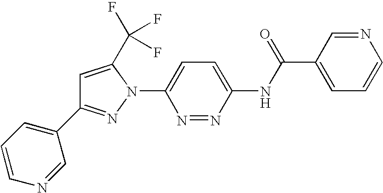

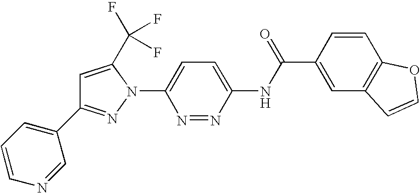

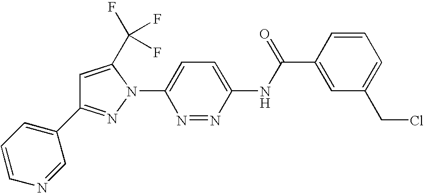

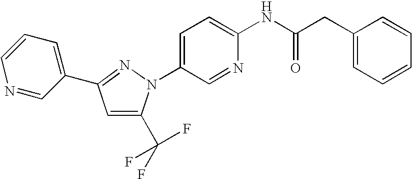

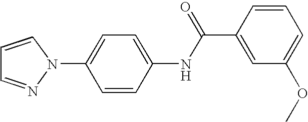

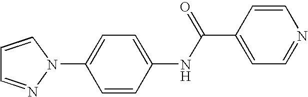

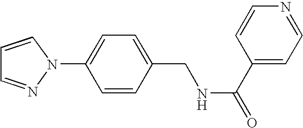

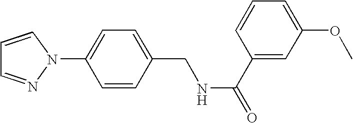

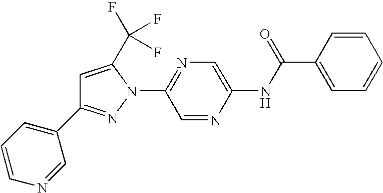

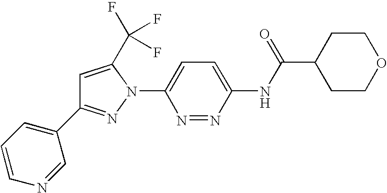

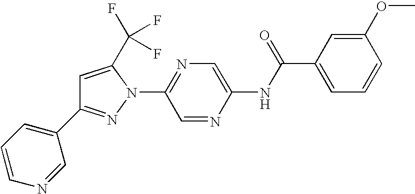

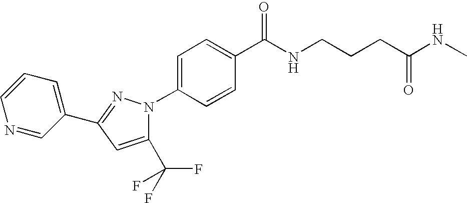

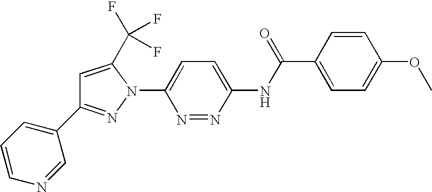

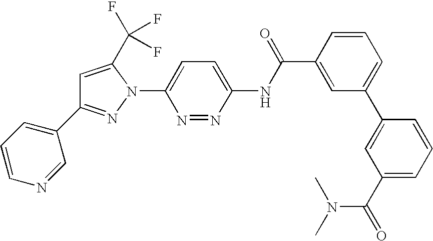

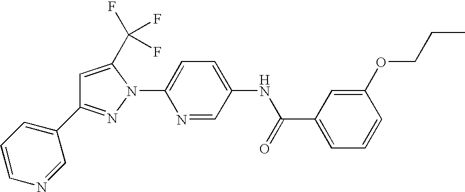

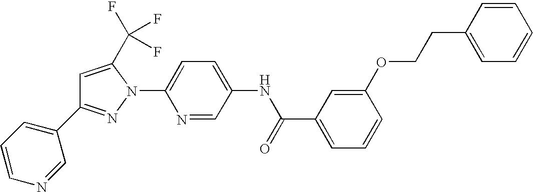

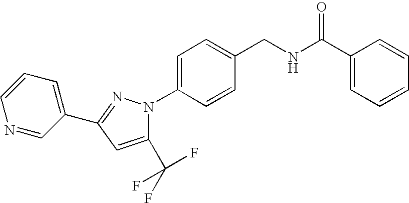

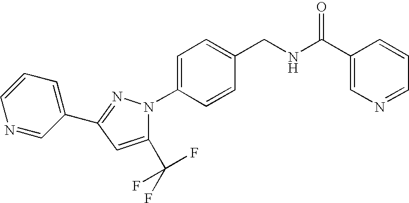

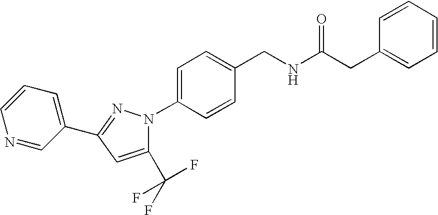

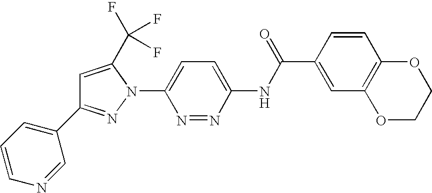

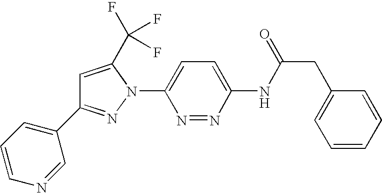

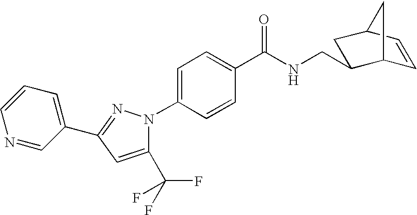

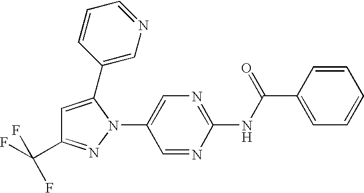

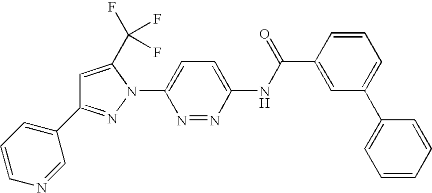

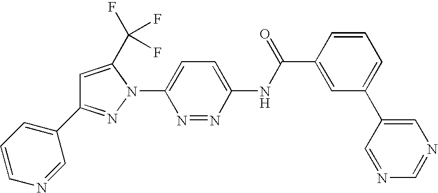

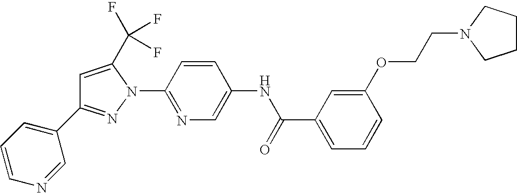

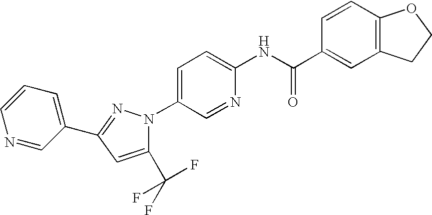

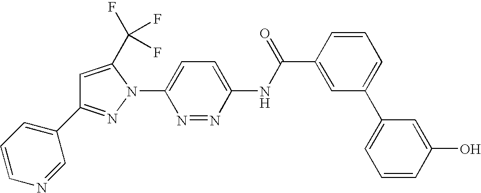

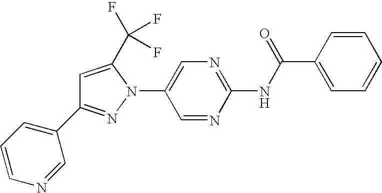

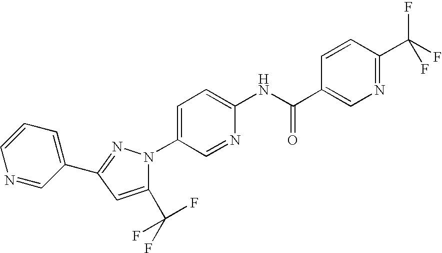

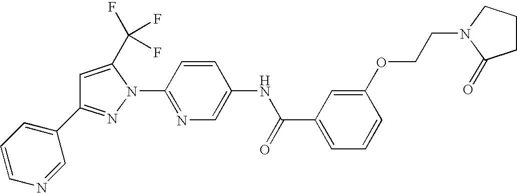

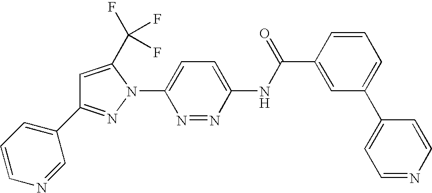

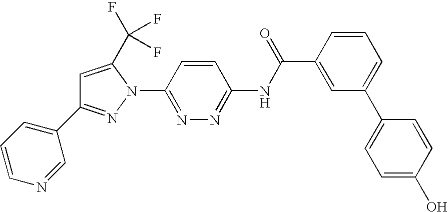

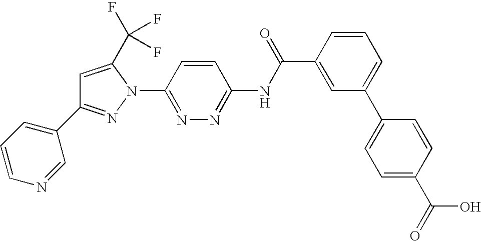

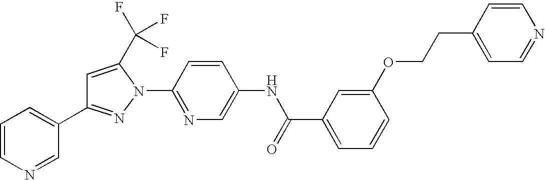

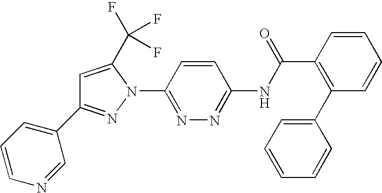

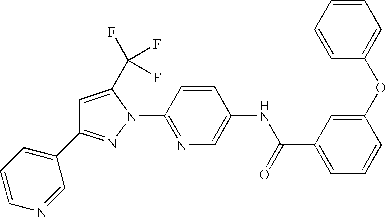

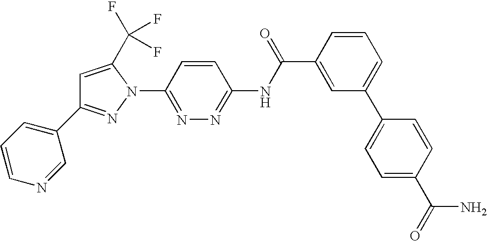

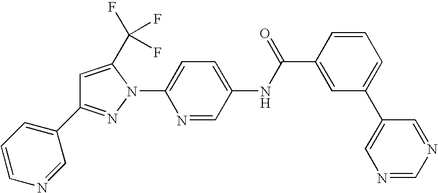

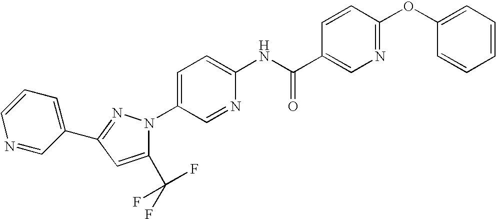

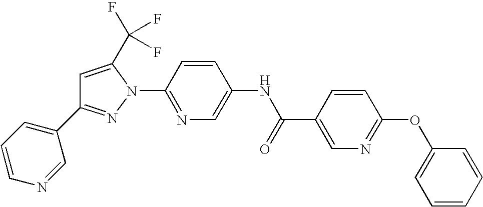

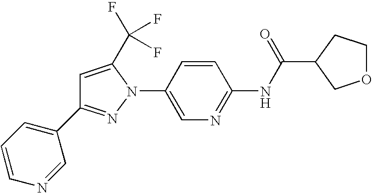

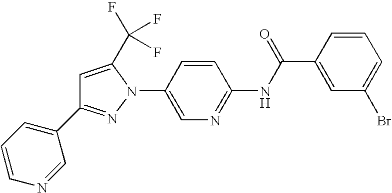

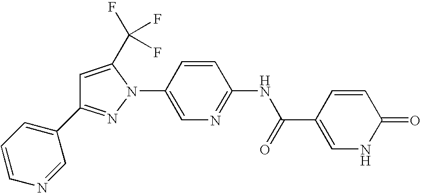

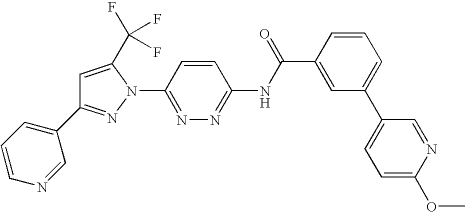

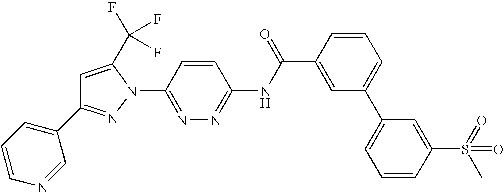

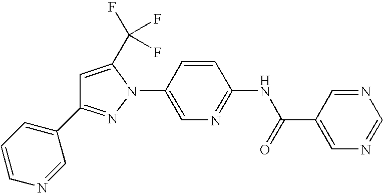

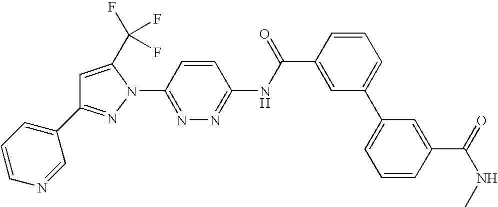

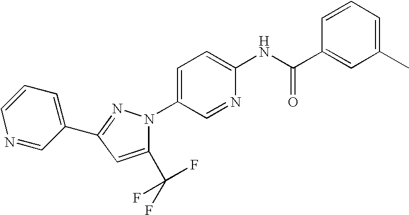

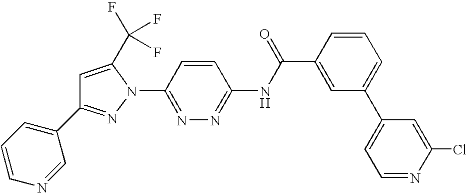

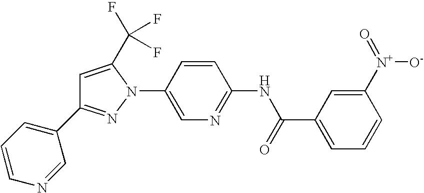

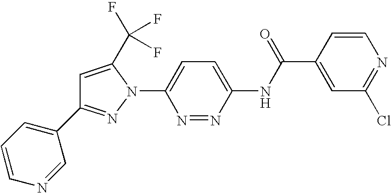

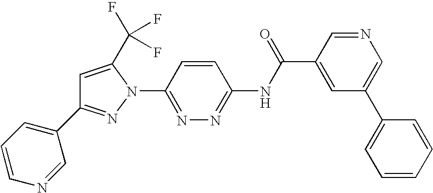

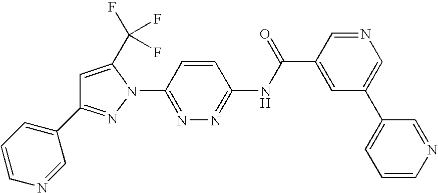

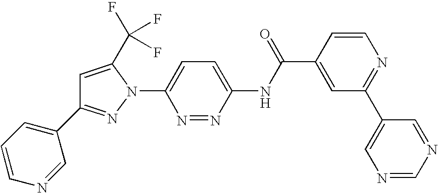

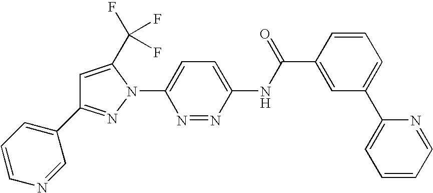

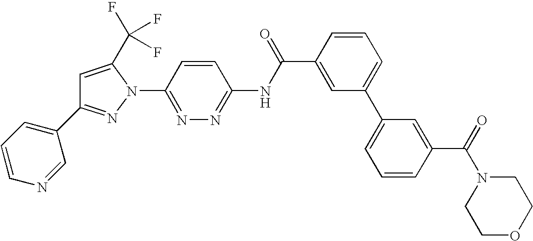

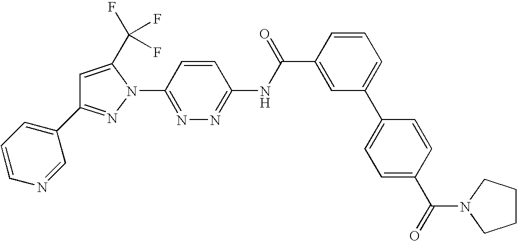

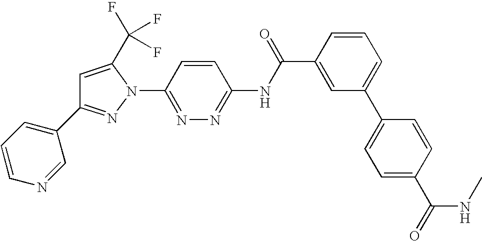

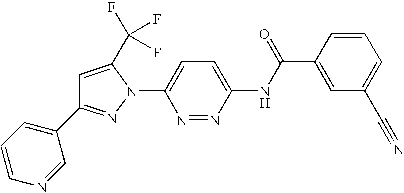

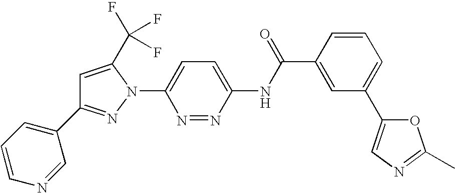

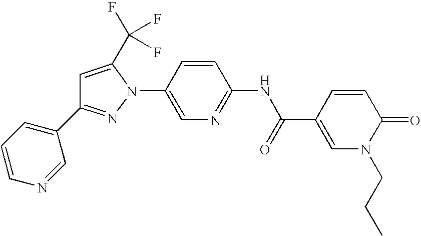

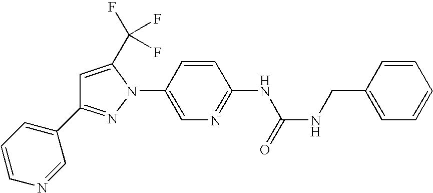

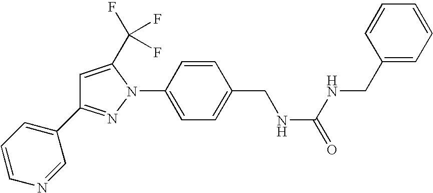

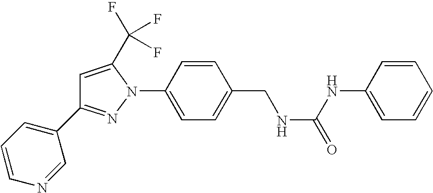

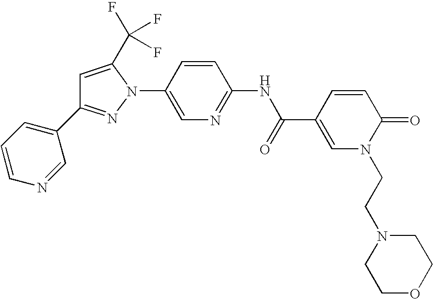

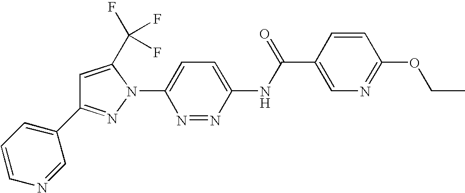

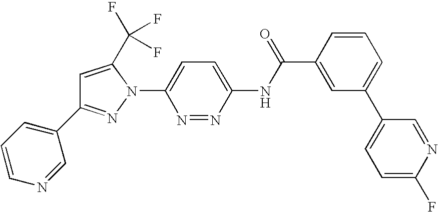

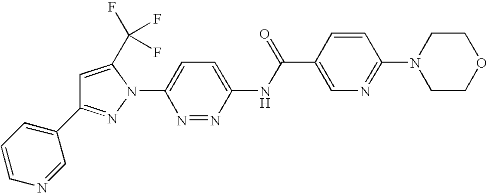

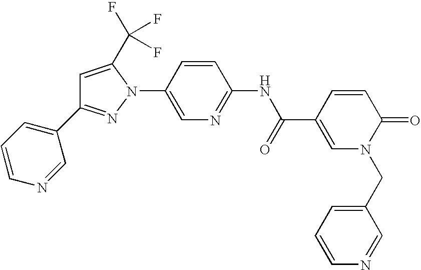

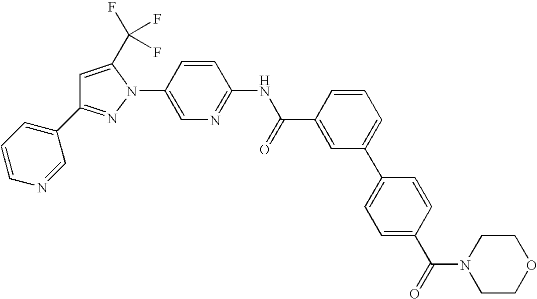

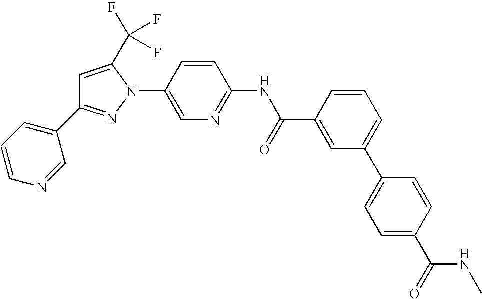

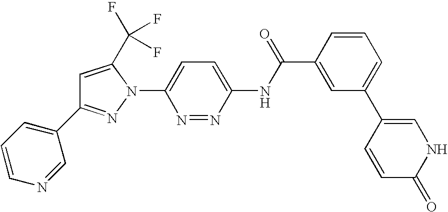

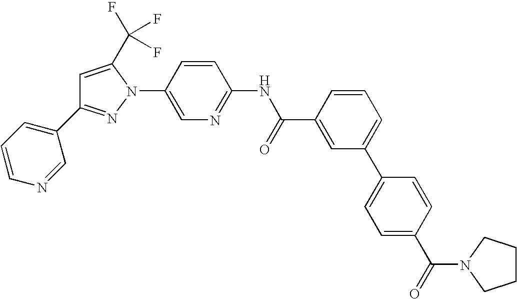

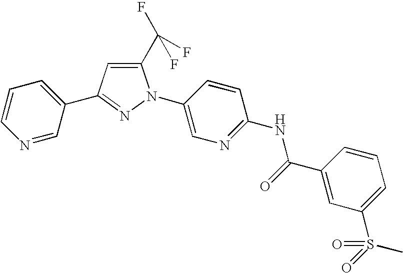

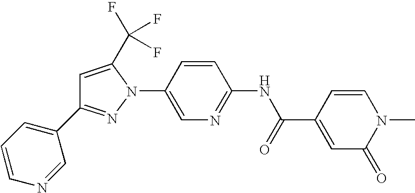

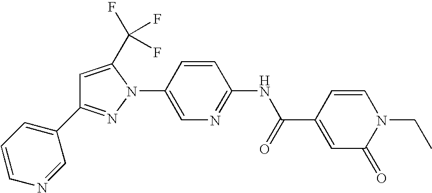

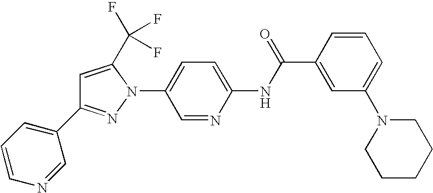

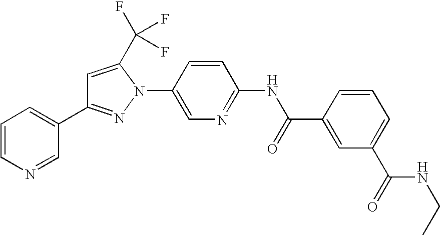

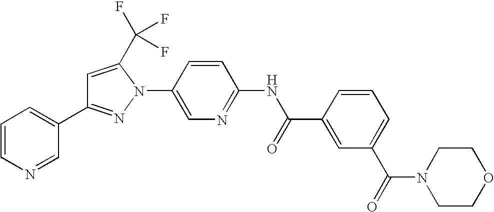

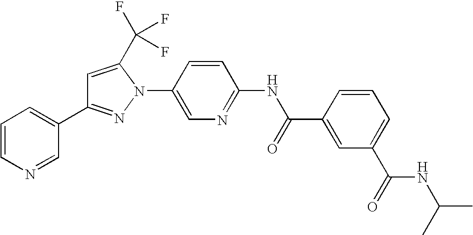

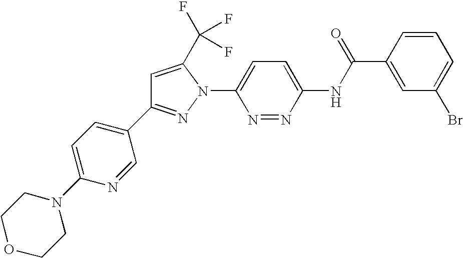

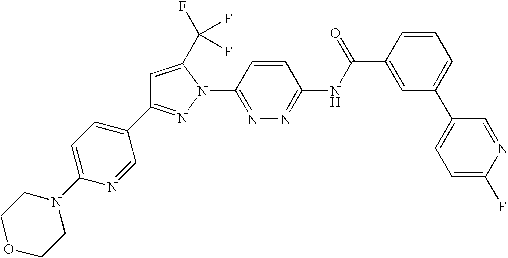

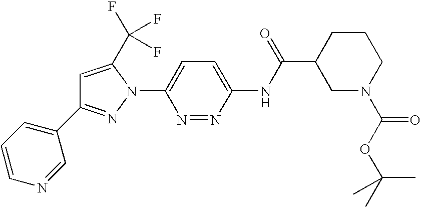

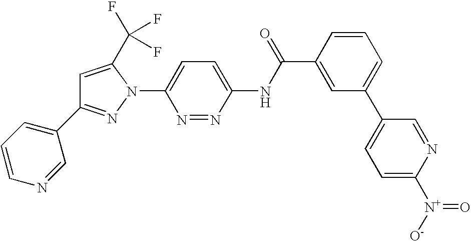

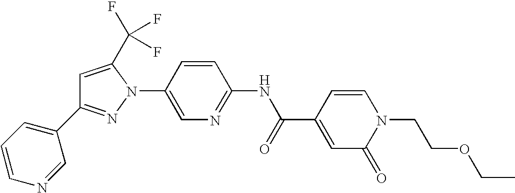

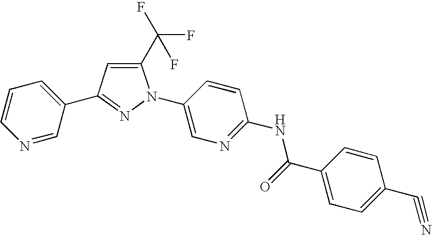

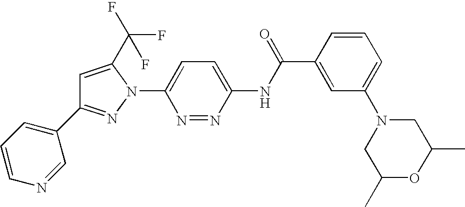

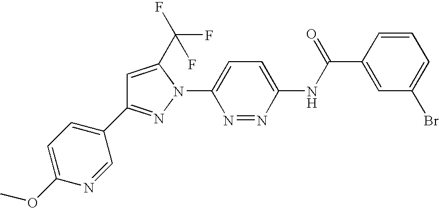

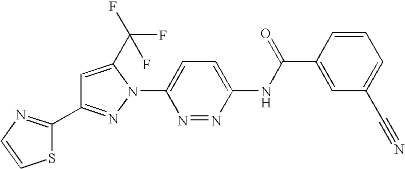

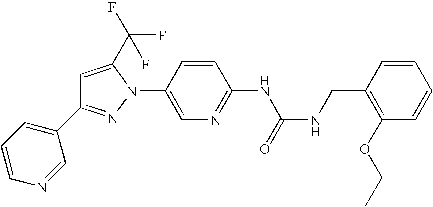

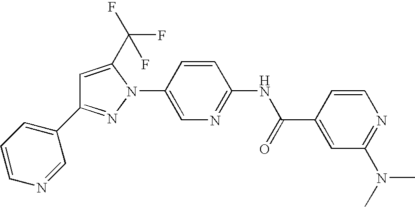

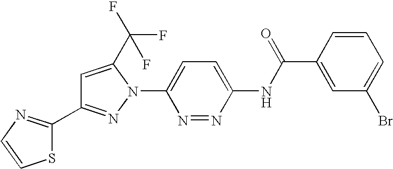

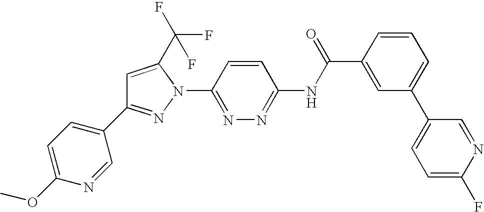

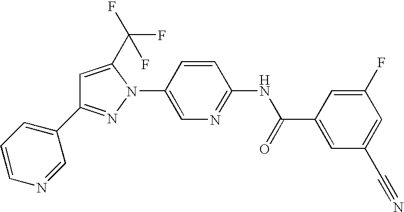

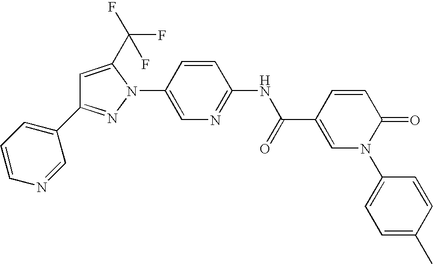

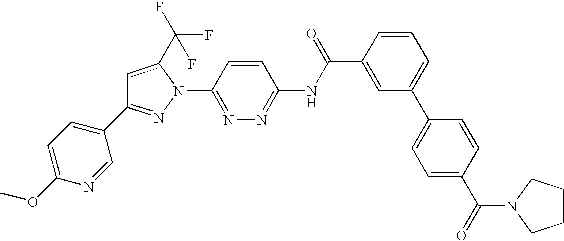

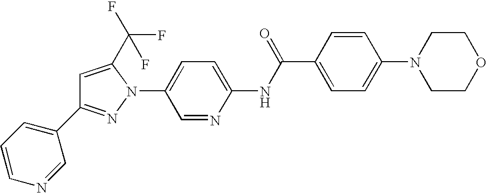

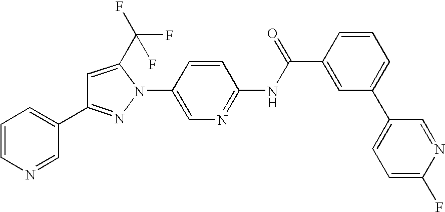

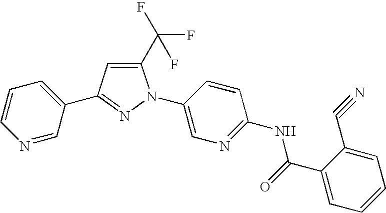

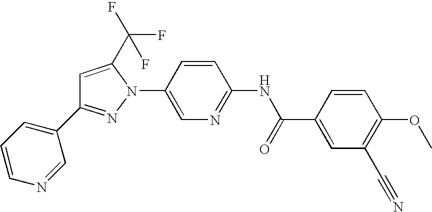

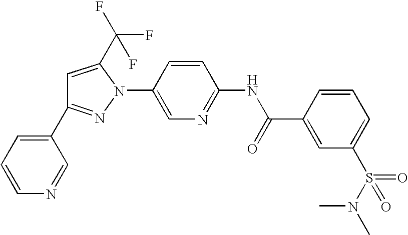

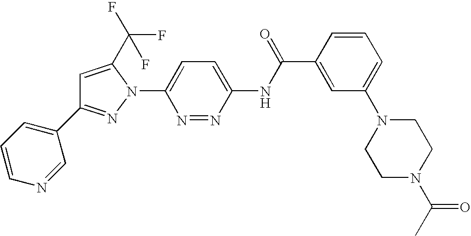

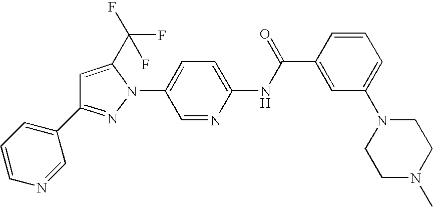

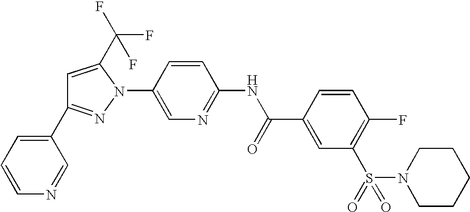

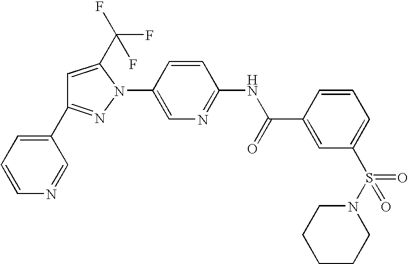

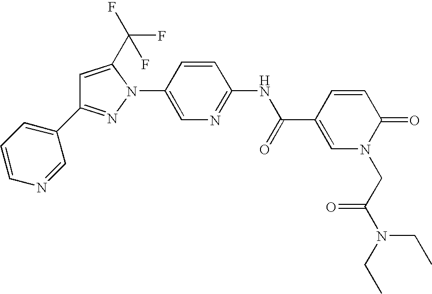

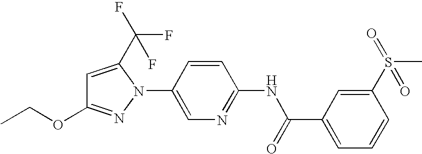

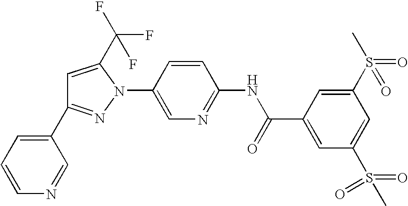

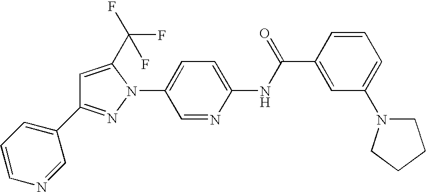

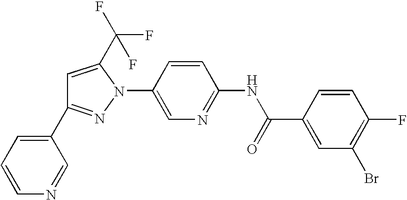

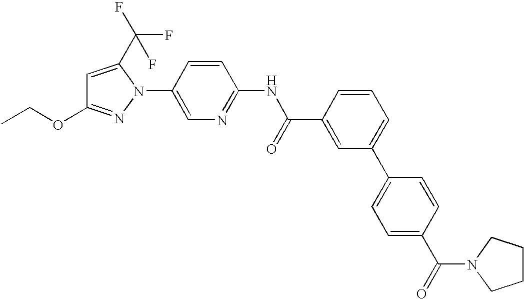

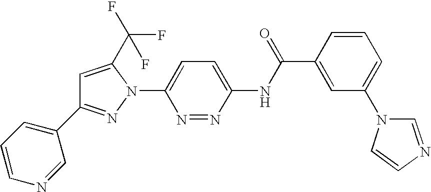

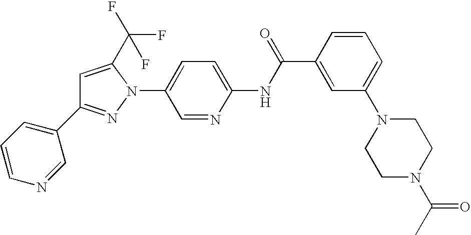

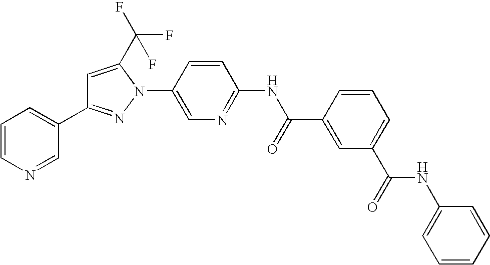

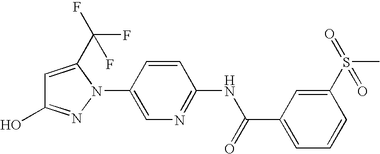

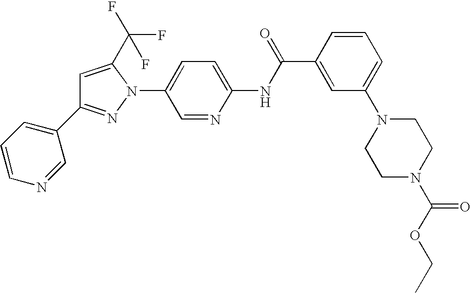

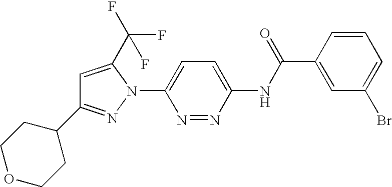

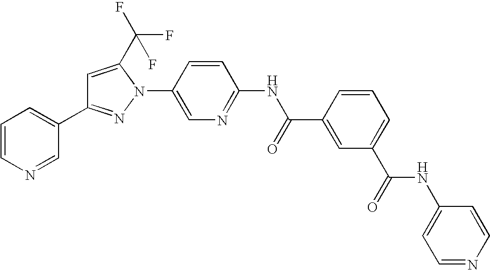

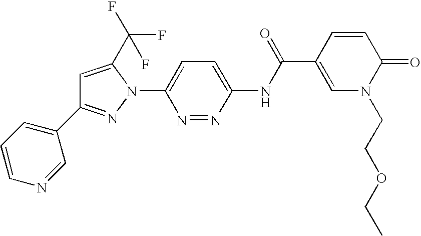

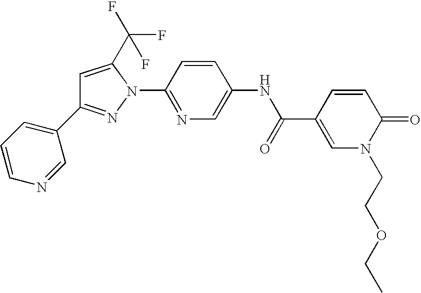

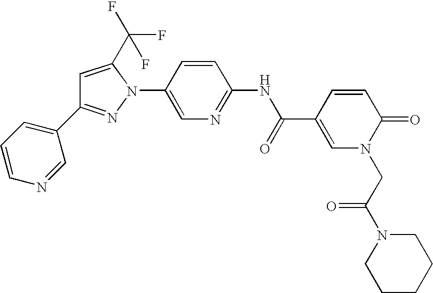

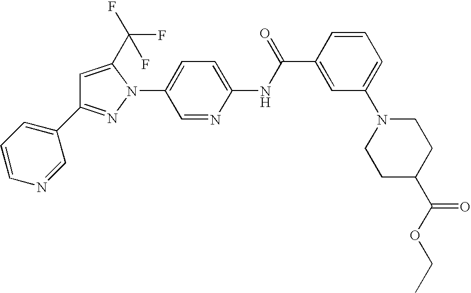

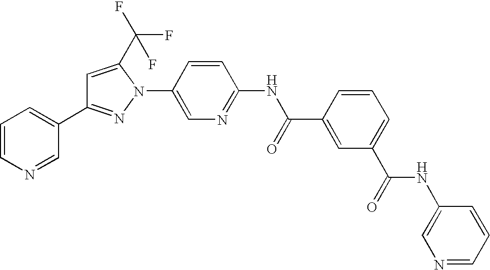

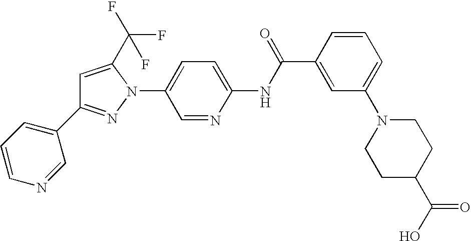

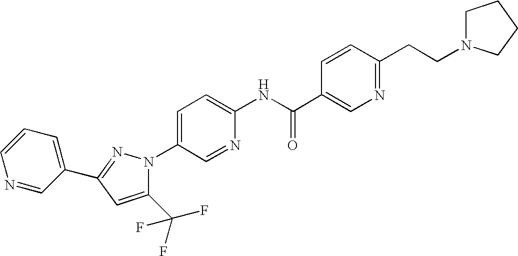

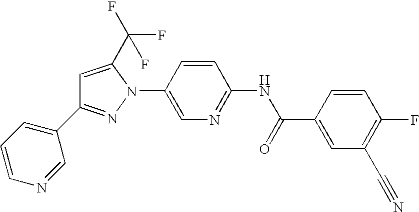

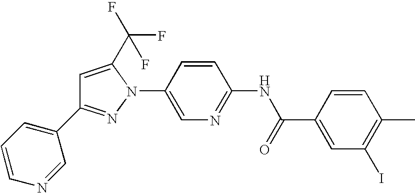

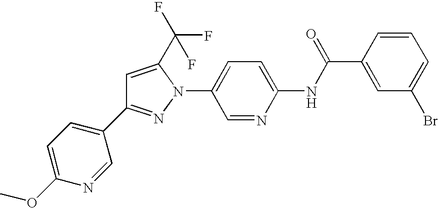

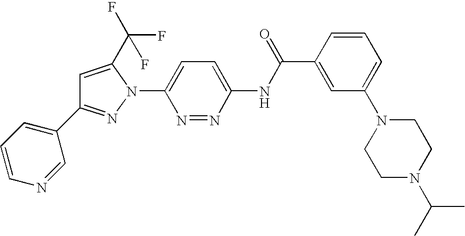

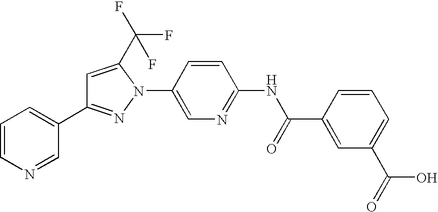

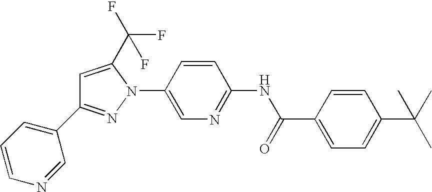

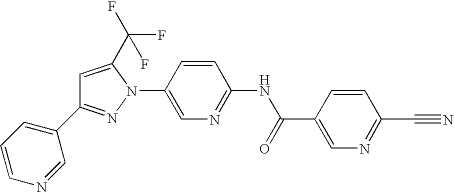

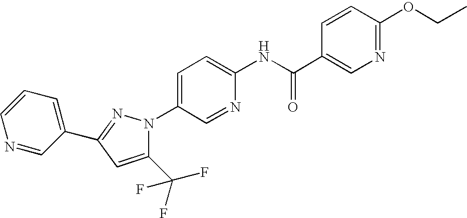

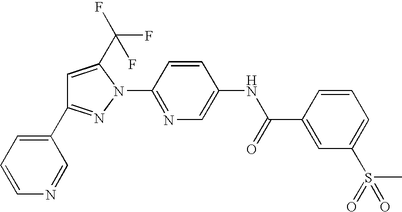

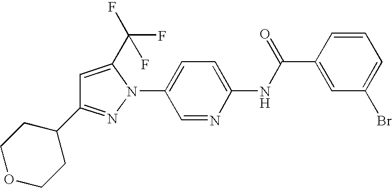

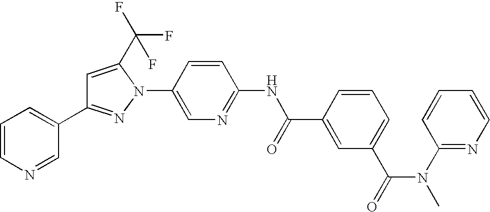

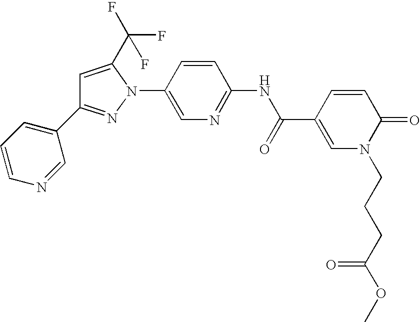

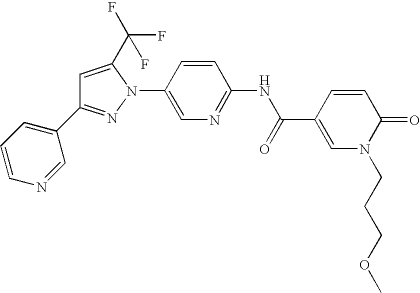

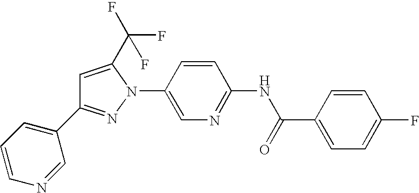

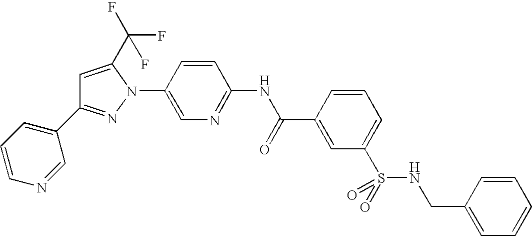

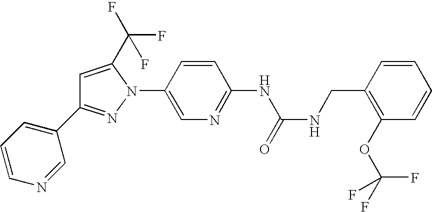

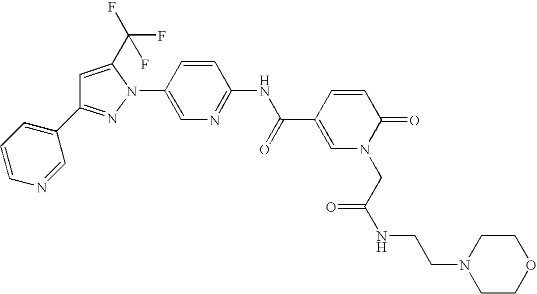

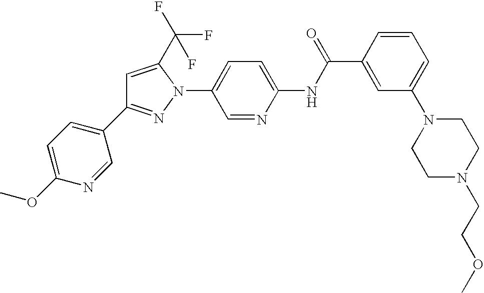

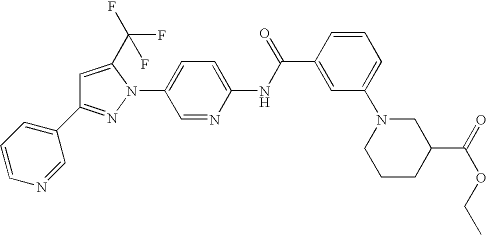

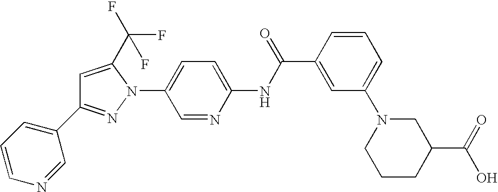

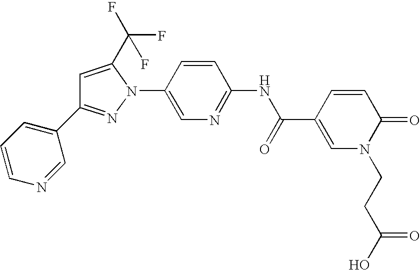

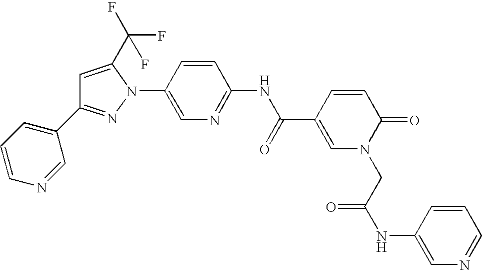

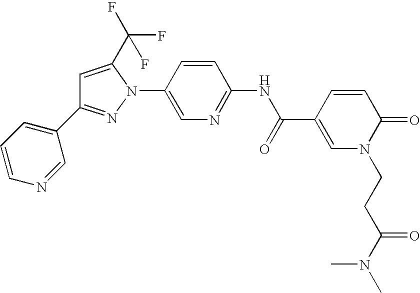

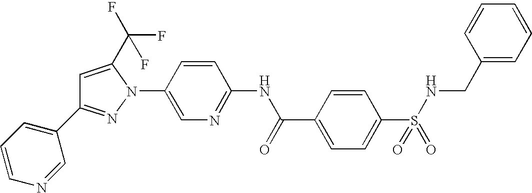

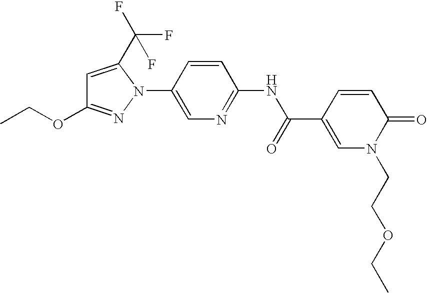

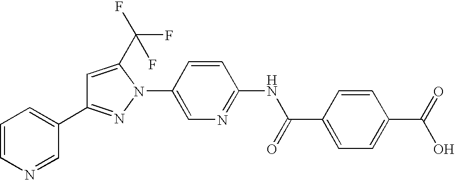

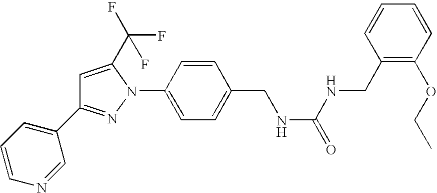

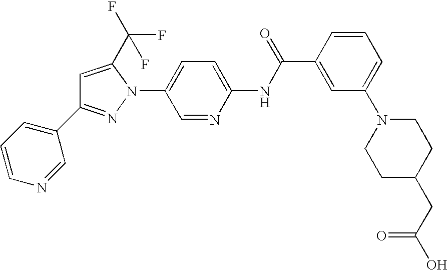

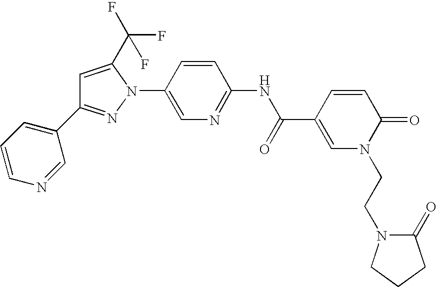

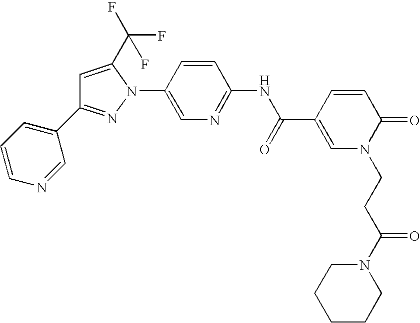

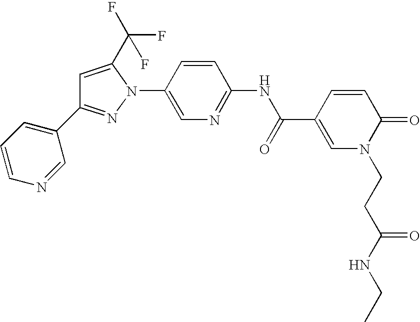

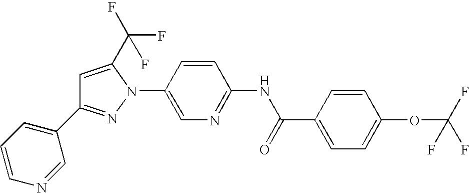

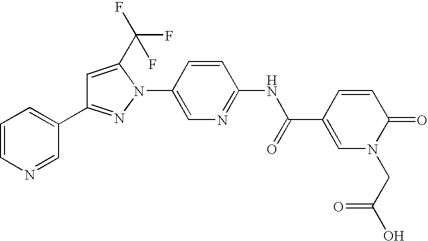

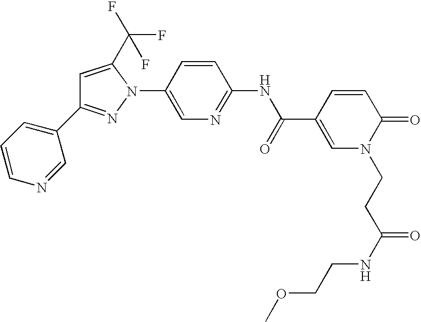

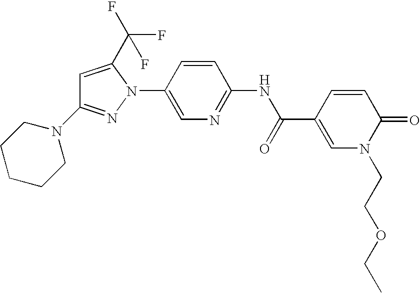

or the pharmaceutically acceptable salts thereof.

In all the compounds disclosed hereinabove in this application, in the event the nomenclature is in conflict with the structure, it shall be understood that the compound is defined by the structure.

The invention includes the use of any compounds of described above containing one or more asymmetric carbon atoms may occur as racemates and racemic mixtures, single enantiomers, diastereomeric mixtures and individual diastereomers. All such isomeric forms of these compounds are expressly included in the present invention. Each stereogenic carbon may be in the R or S configuration, or a combination of configurations.

Some of the compounds of the invention can exist in more than one tautomeric form. The invention includes methods using all such tautomers.

All terms as used herein in this specification, unless otherwise stated, shall be understood in their ordinary meaning as known in the art. For example, C₁₋₄alkoxy includes the organic radical C₁₋₄alkyl with a terminal oxygen, such as methoxy, ethoxy, propoxy, butoxy.

All organic radicals: alkyl, alkenyl and alkynyl groups, or such groups which are incorporated in other radicals such as acyl and alkoxy, shall be understood as being branched or unbranched where structurally possible and unless otherwise specified, and may be partially or fully halogenated.

The term “lower” referred to above and hereinafter in connection with organic radicals or compounds respectively defines such as branched or unbranched with up to and including 7, preferably up to and including 4 and advantageously one or two carbon atoms.

A cyclic group shall be understood to mean carbocycle, heterocycle or heteroaryl, each may be partially or fully halogenated.

An acyl group is a radical defined as —C(O)—R, where R is an organic radical or a cyclic group. Acyl represents, for example, carbocyclic or heterocyclic aroyl, cycloalkylcarbonyl, (oxa or thia)-cycloalkylcarbonyl, lower alkanoyl, (lower alkoxy, hydroxy or acyloxy)-lower alkanoyl, (mono- or di-carbocyclic or heterocyclic)-(lower alkanoyl or lower alkoxy-, hydroxy- or acyloxy-substituted lower alkanoyl), or biaroyl.

Carbocycles include hydrocarbon rings containing from three to fourteen carbon atoms. These carbocycles may be either aromatic either aromatic or non-aromatic ring systems. The non-aromatic ring systems may be mono- or polyunsaturated, monocyclic, bicyclic or tricyclic and may be bridged. Preferred carbocycles include but are not limited to cyclopropyl, cyclobutyl, cyclopentyl, cyclopentenyl, cyclohexyl, cyclohexenyl, cycloheptanyl, cycloheptenyl, phenyl, benzyl, indanyl, indenyl, benzocyclobutanyl, dihydronaphthyl, tetrahydronaphthyl, naphthyl, decahydronaphthyl, benzocycloheptanyl, adamantyl, norbornyl, fluorene, and benzocycloheptenyl. Certain terms for cycloalkyl such as cyclobutanyl and cyclobutyl shall be used interchangeably.

The term “heterocycle” refers to a stable nonaromatic 4-8 membered (but preferably, 5 or 6 membered) monocyclic or nonaromatic 8-11 membered bicyclic heterocycle radical which may be either saturated or unsaturated. Each heterocycle consists of carbon atoms and one or more, preferably from 1 to 4 heteroatoms chosen from nitrogen, oxygen and sulfur. The heterocycle may be attached by any atom of the cycle, which results in the creation of a stable structure. Unless otherwise stated, heterocycles include but are not limited to, for example pyrrolidinyl, pyrrolinyl, morpholinyl, thiomorpholinyl, thiomorpholinyl sulfoxide, thiomorpholinyl sulfone, dioxalanyl, piperidinyl, piperazinyl, tetrahydrofuranyl, tetrahydropyranyl, tetrahydrofuranyl, 1,3-dioxolanone, 1,3-dioxanone, 1,4-dioxanyl, piperidinonyl, tetrahydropyrimidonyl, pentamethylene sulfide, pentamethylene sulfoxide, pentamethylene sulfone, tetramethylene sulfide, tetramethylene sulfoxide and tetramethylene sulfone.

The term “heteroaryl” shall be understood to mean an aromatic 5-8 membered monocyclic or 8-11 membered bicyclic ring containing 1-4 heteroatoms such as N, O and S. Unless otherwise stated, such heteroaryls include aziridinyl, thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl, pyranyl, quinoxalinyl, indolyl, benzimidazolyl, benzoxazolyl, benzothiazolyl, benzothienyl, quinolinyl, quinazolinyl, naphthyridinyl, indazolyl, triazolyl, pyrazolo[3,4-b]pyrimidinyl, purinyl, pyrrolo[2,3-b]pyridinyl, pyrazolo[3,4-b]pyridinyl, tubercidinyl, oxazo[4,5-b]pyridinyl, imidazo[4,5-b]pyridinyl and

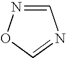

The term “heteroatom” as used herein shall be understood to mean atoms other than carbon such as oxygen, nitrogen, sulfur and phosphorous.

As used herein, “nitrogen” and “sulfur” include any oxidized form of nitrogen and sulfur and the quaternized form of any basic nitrogen. All heteroatoms in open chain or cyclic radicals include all oxidized forms.

In all alkyl groups or carbon chains one or more carbon atoms can be optionally replaced by heteroatoms: O, S or N, it shall be understood that if N is not substituted then it is NH, it shall also be understood that the heteroatoms may replace either terminal carbon atoms or internal carbon atoms within a branched or unbranched carbon chain. Such groups can be substituted as herein above described by groups such as oxo to result in definitions such as but not limited to: alkoxycarbonyl, acyl, amido and thioxo.

The term “aryl” as used herein shall be understood to mean aromatic carbocycle or heteroaryl as defined herein. Each aryl or heteroaryl unless otherwise specified includes it's partially or fully hydrogenated derivative and/or is partially or fully halogenated. For example, quinolinyl may include decahydroquinolinyl and tetrahydroquinolinyl, naphthyl may include it's hydrogenated derivatives such as tetrahydranaphthyl. Other partially or fully hydrogenated derivatives of the aryl and heteroaryl compounds described herein will be apparent to one of ordinary skill in the art.

The term “halogen” as used in the present specification shall be understood to mean bromine, chlorine, fluorine or iodine, preferably fluorine. The definitions “partially or fully halogenated”; partially or fully fluorinated; “substituted by one or more halogen atoms”, includes for example, mono, di or tri halo derivatives on one or more carbon atoms. For alkyl, a nonlimiting example would be —CH₂CHF₂, —CF₃ etc.

The compounds of the invention are only those which are contemplated to be ‘chemically stable’ as will be appreciated by those skilled in the art. For example, a compound which would have a ‘dangling valency’, or a ‘carbanion’ are not compounds contemplated by the inventive methods disclosed herein.

The invention includes pharmaceutically acceptable derivatives of compounds of the invention. A “pharmaceutically acceptable derivative” refers to any pharmaceutically acceptable salt or ester, or any other compound which, upon administration to a patient, is capable of providing (directly or indirectly) a compound useful for the invention, or a pharmacologically active metabolite or pharmacologically active residue thereof. A pharmacologically active metabolite shall be understood to mean any compound of the invention capable of being metabolized enzymatically or chemically. This includes, for example, hydroxylated or oxidized derivative compounds of the invention.

Pharmaceutically acceptable salts include those derived from pharmaceutically acceptable inorganic and organic acids and bases. Examples of suitable acids include hydrochloric, hydrobromic, sulfuric, nitric, perchloric, fumaric, maleic, phosphoric, glycolic, lactic, salicylic, succinic, toluene-p-sulfuric, tartaric, acetic, citric, methanesulfonic, formic, benzoic, malonic, naphthalene-2-sulfuric and benzenesulfonic acids. Other acids, such as oxalic acid, while not themselves pharmaceutically acceptable, may be employed in the preparation of salts useful as intermediates in obtaining the compounds and their pharmaceutically acceptable acid addition salts. Salts derived from appropriate bases include alkali metal (e.g., sodium), alkaline earth metal (e.g., magnesium), ammonium and N—(C₁-C₄ alkyl)₄⁺ salts.

In addition, within the scope of the invention is use of prodrugs of compounds of the invention. Prodrugs include those compounds that, upon simple chemical transformation, are modified to produce compounds of the invention. Simple chemical transformations include hydrolysis, oxidation and reduction. Specifically, when a prodrug is administered to a patient, the prodrug may be transformed into a compound disclosed hereinabove, thereby imparting the desired pharmacological effect.

The compounds described herein are either commercially available or can be made by methods and any necessary intermediates well known in the art.

In order that this invention be more fully understood, the following examples are set forth. These examples are for the purpose of illustrating preferred embodiments of this invention, and are not to be construed as limiting the scope of the invention in any way.

The examples which follow are illustrative and, as recognized by one skilled in the art, particular reagents or conditions could be modified as needed for individual compounds without undue experimentation. Starting materials used in the scheme below are either commercially available or easily prepared from commercially available materials by those skilled in the art.

### General Synthetic Methods

The invention also provides processes for making compounds of Formula (I), (II) and (Ia-Ih). In all schemes, unless specified otherwise, R₂, R₃, X₁, X₂ and G in the formulas below shall have the meaning of R₂, R₃, X₁, X₂ and G in Formula (I), (II) and (Ia-Ih) of the invention described herein above.

Optimum reaction conditions and reaction times may vary depending on the particular reactants used. Unless otherwise specified, solvents, temperatures, pressures, and other reaction conditions may be readily selected by one of ordinary skill in the art. Specific procedures are provided in the Synthetic Examples section. Typically, reaction progress may be monitored by thin layer chromatography (TLC), if desired, and intermediates and products may be purified by chromatography on silica gel and/or by recrystallization. The appropriately substituted starting materials and intermediates used in the preparation of compounds of the invention are either commercially available or readily prepared by methods known in the literature to those skilled in the art, and are illustrated in the synthetic examples below.

Compounds of Formula (I), (II) and (Ia-If) may be synthesized by the method illustrated in Scheme 1

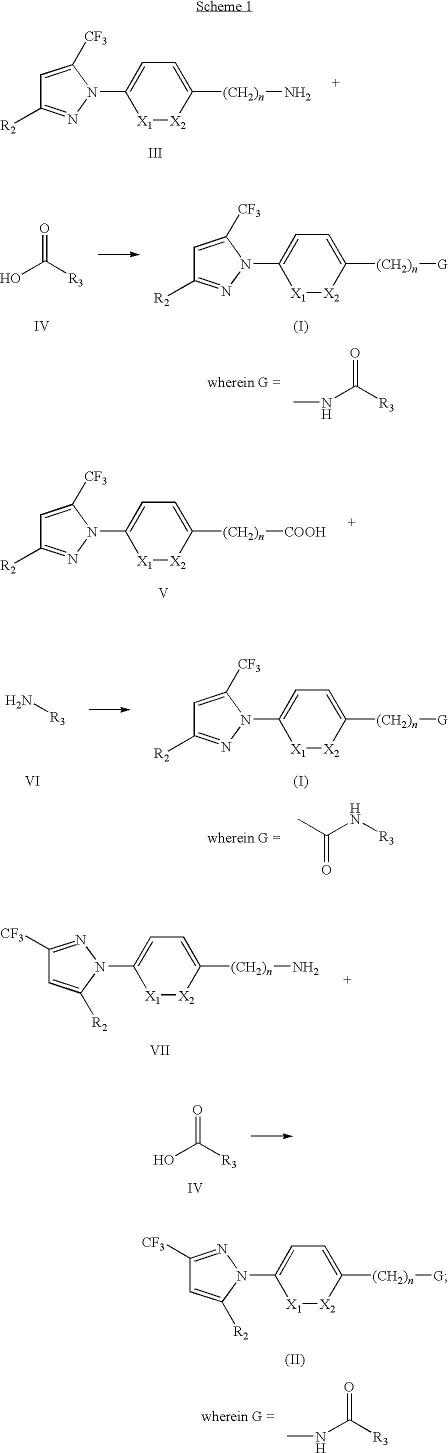

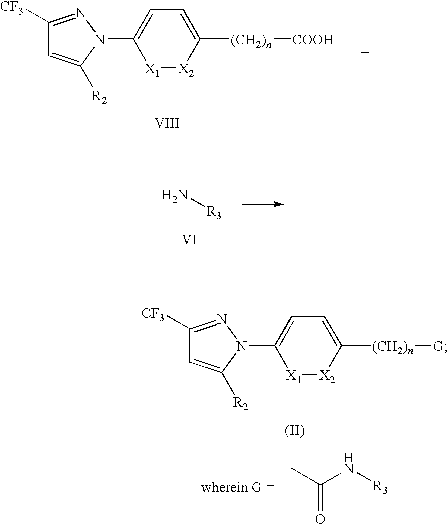

As illustrated in scheme 1, amide coupling of an amine (III or VII) with a carboxylic acid (IV) provides the desired compound of formula (I) or (II) wherein G is —NHCOR₃. Standard peptide coupling reactions known in the art (see for example M. Bodanszky, 1984, The Practice of Peptide Synthesis, Springer-Verlag) may be employed in these syntheses. An example of suitable coupling conditions is treatment of a solution of the carboxylic acid in a suitable solvent such as DMF with EDC, HOBT, and a base such as diisopropylethylamine, followed by the desired amine. Similarly, amide coupling of a carboxylic acid (V or VIII) with an amine (VI) provides the desired compound of formula (I) or (II) wherein G is —CONHR₃.

Alternatively, reaction of the carboxylic acid with reagents such as oxalyl chloride provides the corresponding acid chloride. Reaction of the acid chloride with the desired amine in a suitable solvent provides the compound of formula (I) or (II).

Further modification of the initial product of formula (I) or (II) by methods known in the art and illustrated in the Examples below, may be used to prepare additional compounds of this invention.

The intermediate amines of formula (III) and (VII) may be synthesized by the method outlined in scheme 2.

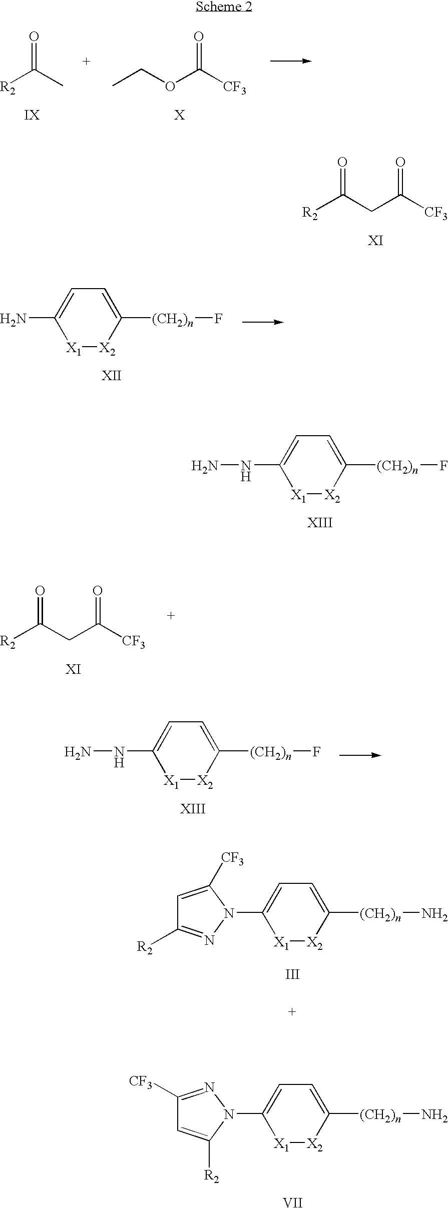

As shown in scheme 2, reaction of a ketone (IX) with ethyl trifluoroacetate (X) in the presence of a suitable base, in a suitable solvent provides a diketone (XI). Reaction of diketone (XI) with a hydrazine of formula (XIII), in a suitable solvent, followed by reaction with ammonium hydroxide provides the desired intermediates (III and (VII).

The hydrazine (XIII) may be either commercially available or may be prepared from the corresponding amine (XII) by using standard literature procedure.

Intermediate carboxylic acids of formula (V) and (VIII) may be synthesized by the method shown in scheme 3

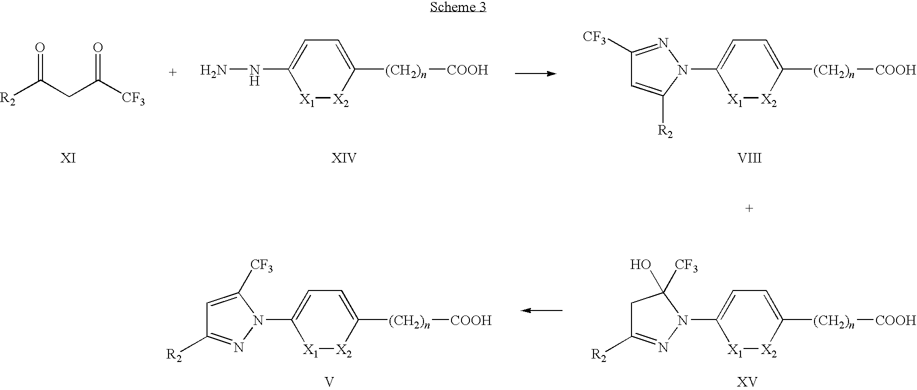

As illustrated in scheme 3, reaction of diketone (XI) with a hydrazine of formula (XIV), in a suitable solvent, provides the desired intermediate (VIII) and the trifluoromethyl alcohol (XV). Dehydration of alcohol (XV) with a suitable reagent, at a suitable temperature, provides the desired intermediate (V).

The hydrazine (XIV) may be either commercially available or may be prepared from the corresponding amine by using standard literature procedure.

Compounds of formula Ih may be prepared by the method shown in scheme 4

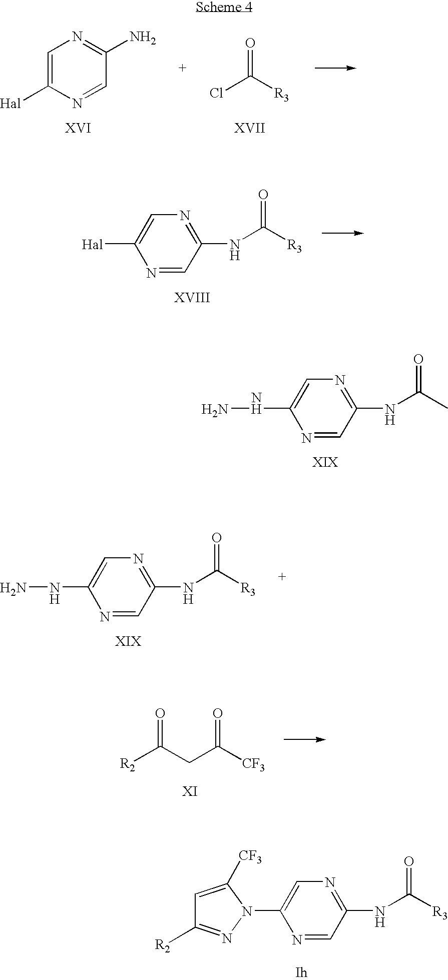

As illustrated in scheme 4, amide coupling of a halogenated pyrazine amine (XVI), wherein Hal is chloro, bromo or iodo, with a carboxylic acid chloride (XVII) in a suitable solvent, in the presence of a suitable base, provides the coupled intermediate of formula (XVIII) wherein G is —NHCOR₃. Reaction of the intermediate (XVIII) with hydrazine at a suitable temperature provides a hydrazine of formula (XIX). Reaction of the hydrazine (XIX) with diketone (XI) followed by dehydration provides a compound of formula (Ih)

Compounds of formula (Ig) may be made by the synthetic scheme 5, shown below.

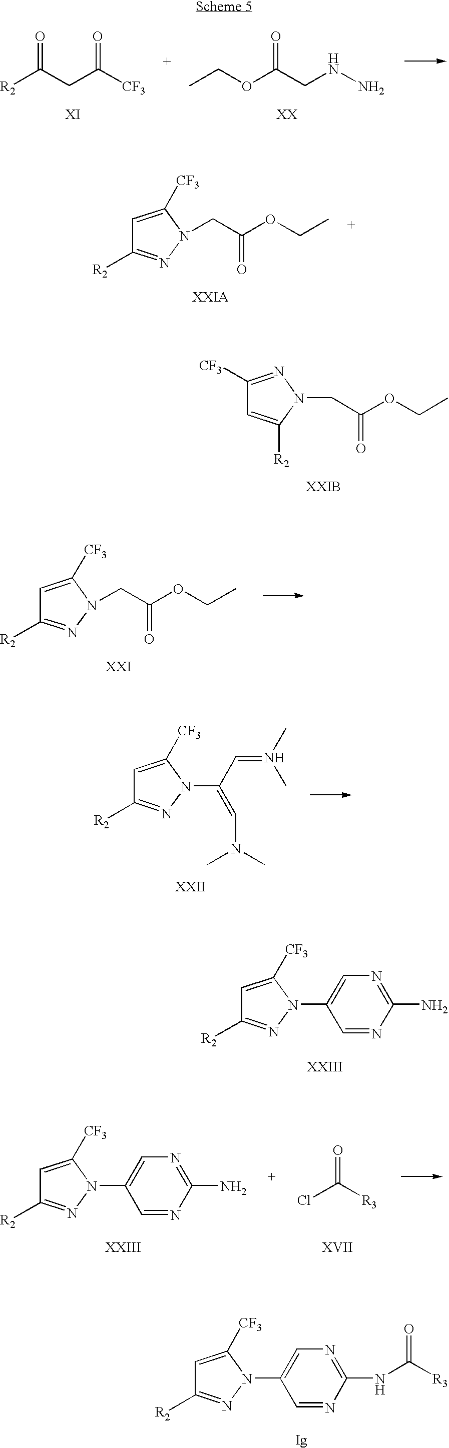

As illustrated in scheme 5, reaction of the diketone (XI) with ethyl hydrazinoacetate in a suitable solvent provides the two pyrazole regioisomers (XXIA and XXIB). Reaction of the pyrazole of formula (XXIA) with phosphoryl chloride in dimethylformamide in a suitable solvent such as dimethylformamide, at a suitable temperature, provides an intermediate of formula (XXII). Heating the diamino intermediate (XXII) in a suitable solvent in the presence of a suitable base provides the amino pyrimidine of formula (XXIII). Amide coupling of the amino pyrimidine (XXIII), with a carboxylic acid chloride (XVII) in a suitable solvent, in the presence of a suitable base, provides a compound of formula (Ig) wherein G is —NHCOR₃.

### Synthetic Methods

### Experimental Examples

### Example 1

### 3-Cyano-5-fluoro-N-[5-(3-pyridin-3-yl-5-trifluoromethyl-pyrazol-1-yl)-pyridin-2-yl]-benzamide

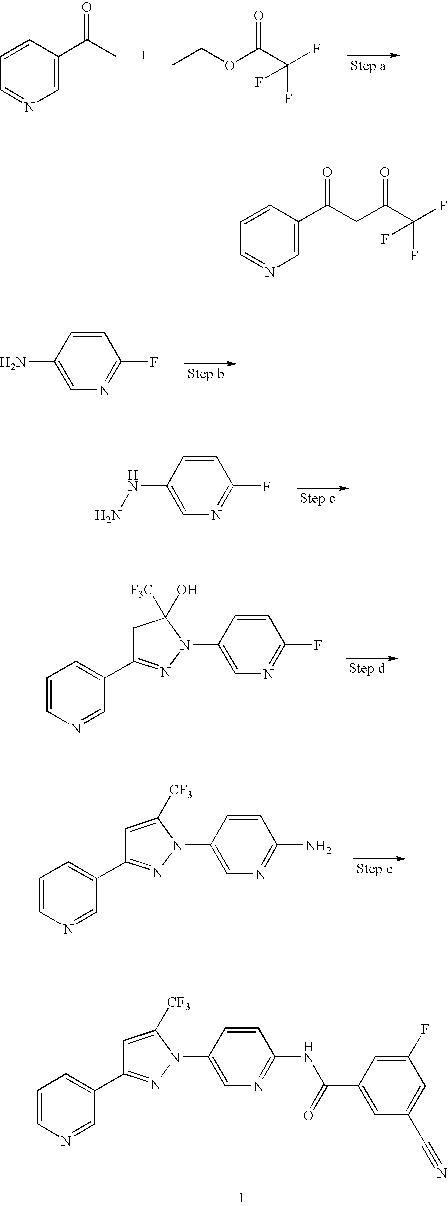

### Step a

Sodium (4.6 g; 0.20 mol) is added to a solution of methanol (50 mL) at 0° C. and allowed to warm to room temperature and then stirred for 1 h. The solvent is removed under vacuo, ether (250 mL) is added to the sodium methoxide formed, and the mixture is cooled to 0° C., before the slow addition of ethyl trifluoroacetate (24 mL, 0.20 mol) and 3-acetylpyridine (22 mL, 0.20 mol). The reaction mixture is heated at reflux for 2 hours, allowed to cool to room temperature, diluted with water (100 mL) and washed with ether (3×). The aqueous layer is acidified to pH 5 using acetic acid and the solid is filtered, rinsed with ice cold ether (200 mL) to give 4,4,4-trifluoro-1-pyridin-3-yl-butane-1,3-dione (35.7 g, 82%).

### Step b

Sodium nitrite (1.38 g, 0.020 mol) in water (4.0 mL) is added dropwise to a stirred solution of 5-amino-2-fluoropyridine (1.5 g; 0.013 mol) in 6 M aqueous HCl (5 mL) at −20° C. The reaction mixture is allowed to warm to 10° C. and stirred for 30 min before being cooled back to −20° C. Tin (II) chloride (6 g, 0.027 mol) is added to the reaction mixture and allowed to warm to 10° C. then stirred for a further 60 min. The reaction mixture is basified with 1 M aqueous potassium hydroxide, the solid is filtered off, and the aqueous layer is extracted with ethyl acetate (75 mL, 3×). The organic layers are combined, dried over sodium sulfate and concentrated under vacuo to give crude (6-fluoro-pyridin-3-yl)-hydrazine (1.12 g, 68%).

### Step c

A solution of (6-fluoro-pyridin-3-yl)-hydrazine (900 mg, 7.1 mmol), acetic acid (3.0 mL) in ethanol (25 mL) is added to a stirred solution of 4,4,4-trifluoro-1-pyridin-3-yl-butane-1,3-dione (1.54 g; 7.1 mmol) in ethanol (25 mL). The reaction mixture is heated at reflux for 16 hours, allowed to cool to room temperature, and concentrated under vacuo. The complex is purified by chromatography to give 2-(6-fluoro-pyridin-3-yl)-5-pyridin-3-yl-3-trifluoromethyl-3,4-dihydro-2H-pyrazol-3-ol (960 mg, 42%).

### Step d

A solution of 2-(6-fluoro-pyridin-3-yl)-5-pyridin-3-yl-3-trifluoromethyl-3,4-dihydro-2H-pyrazol-3-ol (326 mg, 1.0 mmol), in concentrated ammonium hydroxide (1.5 mL) and dioxane (1.5 mL) is heated at 125° C. under microwave conditions for 2 hours. The reaction mixture is allowed to cool to room temperature and concentrated under vacuo. The mixture is purified by chromatography to give 5-(3-pyridine-3-yl-5-trifluoromethyl-pyrazol-1-yl)pyridin-2-ylamine (177 mg, 58%).

### Step e

A solution of 3-cyano-5-fluorobenzoic acid (110 mg, 0.66 mmol) in thionyl chloride (2.5 mL) is refluxed for 1 hour and then concentrated under vacuo. Pyridine (2.5 mL) is then added to the preformed acid chloride followed by the addition of 5-(3-pyridine-3-yl-5-trifluoromethyl-pyrazol-1-yl)pyridin-2-ylamine (100 mg, 0.33 mmol) in pyridine (2.5 mL). The reaction mixture is stirred for 1 hours diluted with ethyl acetate, washed with water (25 mL, 3×), dried over sodium sulfate and concentrated under vacuo. The product is recrystallized with ethyl acetate/hexanes to give the title compound (85 mg, 57%). LC-MS (M⁺+1): 452.92.

The following compounds are prepared according to example 1 by replacing the carboxylic acid in step e with commercially available starting materials.

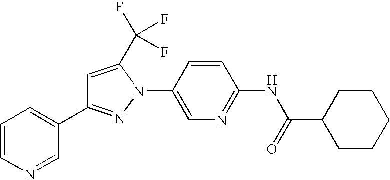

Cyclohexanecarboxylic acid [5-(3-pyridin-3-yl-5-trifluoromethyl-pyrazol-1-yl)-pyridin-2-yl]-amide; LC-MS (M¹+1): 415.96.

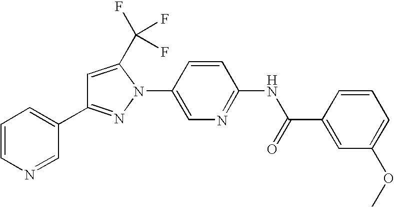

3-Methoxy-N-[5-(3-pyridin-3-yl-5-trifluoromethyl-pyrazol-1-yl)-pyridin-2-yl]-benzamide; LC-MS (M⁺+1): 439.96.

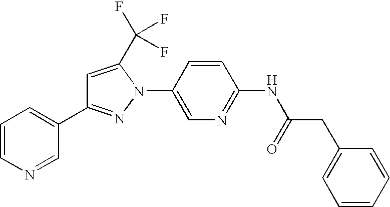

2-Phenyl-N-[5-(3-pyridin-3-yl-5-trifluoromethyl-pyrazol-1-yl)-pyridin-2-yl]-acetamide; LC-MS (M⁺+1): 424.34.

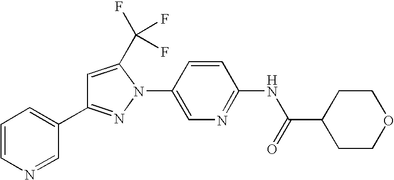

Tetrahydro-pyran-4-carboxylic acid [5-(3-pyridin-3-yl-5-trifluoromethyl-pyrazol-1-yl)-pyridin-2-yl]-amide; LC-MS (M⁺+1): 418.45.

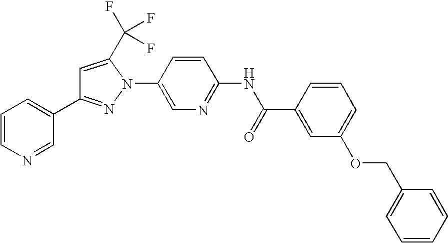

3-Benzyloxy-N-[5-(3-pyridin-3-yl-5-trifluoromethyl-pyrazol-1-yl)-pyridin-2-yl]-benzamide; LC-MS (M⁺+1): 516.05).

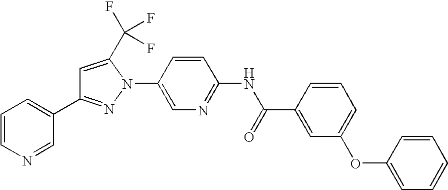

3-Phenoxy-N-[5-(3-pyridin-3-yl-5-trifluoromethyl-pyrazol-1-yl)-pyridin-2-yl]-benzamide; LC-MS (M⁺+1): 502.41.

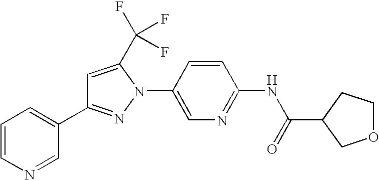

Tetrahydro-furan-3-carboxylic acid [5-(3-pyridin-3-yl-5-trifluoromethyl-pyrazol-1-yl)-pyridin-2-yl]-amide; LC-MS (M⁺+1): 404.34.

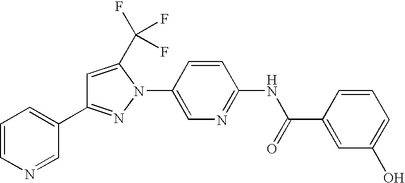

3-Hydroxy-N-[5-(3-pyridin-3-yl-5-trifluoromethyl-pyrazol-1-yl)-pyridin-2-yl]-benzamide; LC-MS (M⁺+1): 426.40.

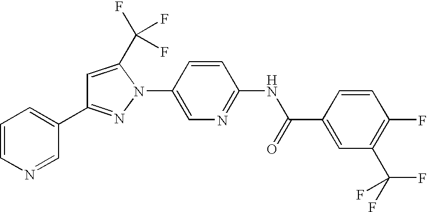

4-Fluoro-N-[5-(3-pyridin-3-yl-5-trifluoromethyl-pyrazol-1-yl)-pyridin-2-yl]-3-trifluoromethyl-benzamide; LC-MS (M⁺+1): 495.97.

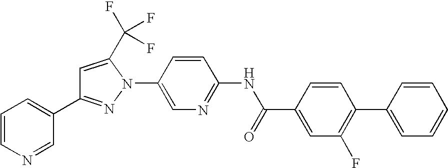

2-Fluoro-biphenyl-4-carboxylic acid [5-(3-pyridin-3-yl-5-trifluoromethyl-pyrazol-1-yl)-pyridin-2-yl]-amide; LC-MS (M⁺+1): 504.26.

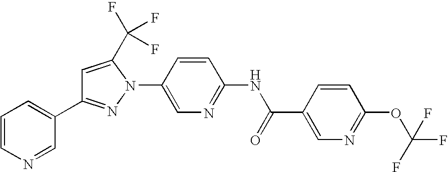

N-[5-(3-Pyridin-3-yl-5-trifluoromethyl-pyrazol-1-yl)-pyridin-2-yl]-6-trifluoromethoxy-nicotinamide; LC-MS (M⁺+1): 508.94.

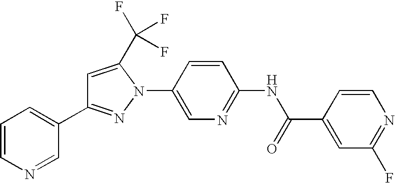

2-Fluoro-N-[5-(3-pyridin-3-yl-5-trifluoromethyl-pyrazol-1-yl)-pyridin-2-yl]-isonicotinamide; LC-MS (M⁺+1): 428.94.

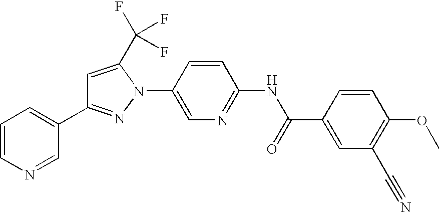

3-Cyano-4-methoxy-N-[5-(3-pyridin-3-yl-5-trifluoromethyl-pyrazol-1-yl)-pyridin-2-yl]-benzamide; LC-MS (M⁺+1): 464.99.

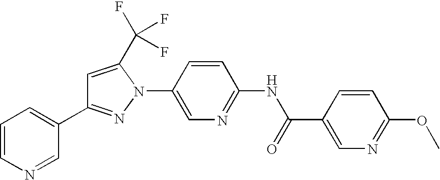

6-Methoxy-N-[5-(3-pyridin-3-yl-5-trifluoromethyl-pyrazol-1-yl)-pyridin-2-yl]-nicotinamide; LC-MS (M⁺+1): 441.01.

### Example 2

### 3-Cyano-N-[5-(3-pyridin-3-yl-5-trifluoromethyl-pyrazol-1-yl)-pyridin-2-yl]-benzamide

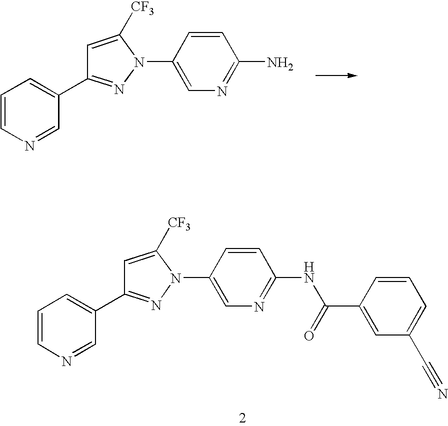

A solution of 5-(3-pyridine-3-yl-5-trifluoromethyl-pyrazol-1-yl)pyridin-2-ylamine (100 mg, 0.33 mmol), prepared according to example 1,3-cyanobenzoyl chloride (150 mg, 0.91 mmol) in pyridine (2.5 mL) is heated at reflux for 2 hours. The reaction mixture is allowed to cool to room temperature, diluted with ethyl acetate, washed with water (25 mL, 3×), dried over sodium sulfate and concentrated under vacuo. The mixture is purified by chromatography to give the title compound (21 mg, 15%). LC-MS (M⁺+1): 435.40.

The following compounds are prepared according to example 2 by replacing the acyl chloride with commercially available starting materials.

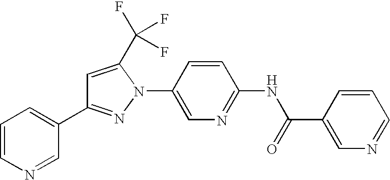

N-[5-(3-Pyridin-3-yl-5-trifluoromethyl-pyrazol-1-yl)-pyridin-2-yl]-nicotinamide; LC-MS (M⁺+1): 411.55.

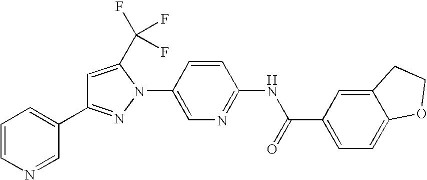

2,3-Dihydro-benzofuran-5-carboxylic acid [5-(3-pyridin-3-yl-5-trifluoromethyl-pyrazol-1-yl)-pyridin-2-yl]-amide; LC-MS (M⁺+1): 452.02.

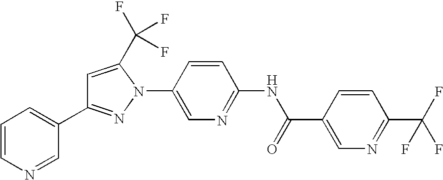

N-[5-(3-Pyridin-3-yl-5-trifluoromethyl-pyrazol-1-yl)-pyridin-2-yl]-6-trifluoromethyl-nicotinamide; LC-MS (M⁺+1): 479.03.

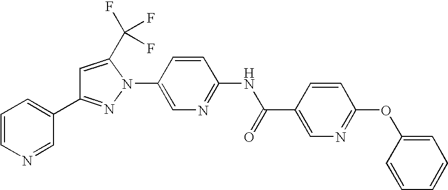

6-Phenoxy-N-[5-(3-pyridin-3-yl-5-trifluoromethyl-pyrazol-1-yl)-pyridin-2-yl]-nicotinamide; LC-MS (M⁺+1): 503.39.

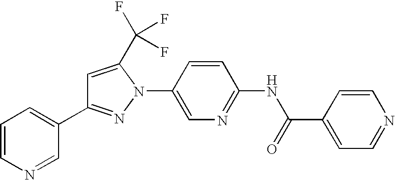

N-[5-(3-Pyridin-3-yl-5-trifluoromethyl-pyrazol-1-yl)-pyridin-2-yl]-isonicotinamide; LC-MS (M⁺+1): 411.04.

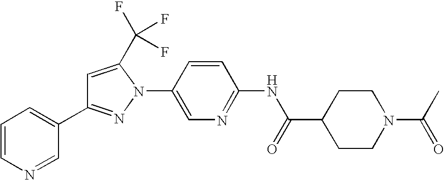

1-Acetyl-piperidine-4-carboxylic acid. [5-(3-pyridin-3-yl-5-trifluoromethyl-pyrazol-1-yl)-pyridin-2-yl]-amide; LC-MS (M⁺+1): 459.44.

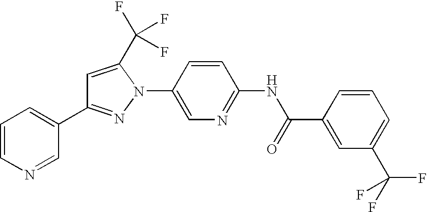

N-[5-(3-Pyridin-3-yl-5-trifluoromethyl-pyrazol-1-yl)-pyridin-2-yl]-3-trifluoromethyl-benzamide; LC-MS (M⁺+1): 478.36.

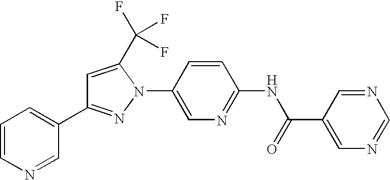

Pyrimidine-5-carboxylic acid [5-(3-pyridin-3-yl-5-trifluoromethyl-pyrazol-1-yl)-pyridin-2-yl]-amide; LC-MS (M⁺+1): 412.41.

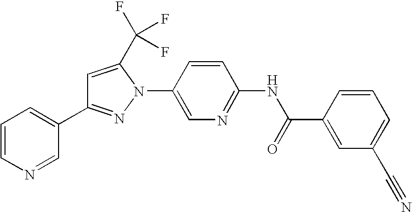

3-Cyano-N-[5-(3-pyridin-3-yl-5-trifluoromethyl-pyrazol-1-yl)-pyridin-2-yl]-benzamide; LC-MS (M⁺+1): 435.40.

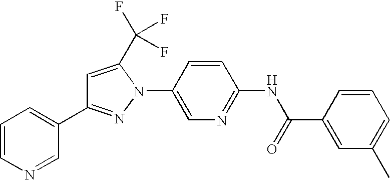

3-Methyl-N-[5-(3-pyridin-3-yl-5-trifluoromethyl-pyrazol-1-yl)-pyridin-2-yl]-benzamide, LC-MS (M⁺+1): 424.04.

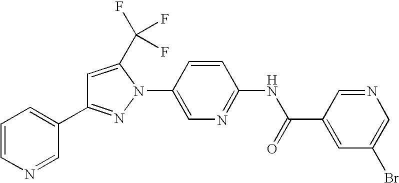

5-Bromo-N-[5-(3 pyridin-3-yl-5-trifluoromethyl-pyrazol-1-yl)-pyridin-2-yl]-nicotinamide; LC-MS (M⁺+1): 491.32.

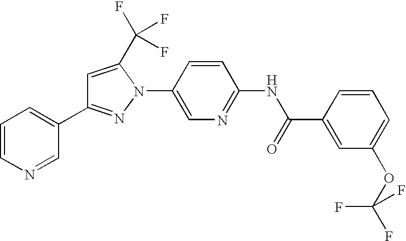

N-[5-(3-Pyridin-3-yl-5-trifluoromethyl-pyrazol-1-yl)-pyridin-2-yl]-3-trifluoromethoxy-benzamide; LC-MS (M⁺+1): 494.36.

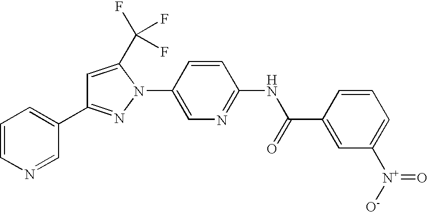

3-Nitro-N-[5-(3-pyridin-3-yl-5-trifluoromethyl-pyrazol-1-yl)-pyridin-2-yl]-benzamide; LC-MS (M⁺+1): 455.40.

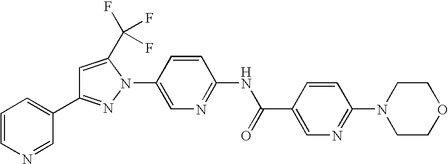

6-Morpholin-4-yl-N-[5-(3-pyridin-3-yl-5-trifluoromethyl-pyrazol-1-yl)-pyridin-2-yl]-nicotinamide; LC-MS (M⁺+1): 496.41.

### Example 3

### 1-(2-Methoxy-ethyl)-6-oxo-1,6-dihydro-pyridine-3-carboxylic Acid [5-(3-pyridin-3-yl-5-trifluoromethyl-pyrazol-1-yl)-pyridin-2-yl]-amide

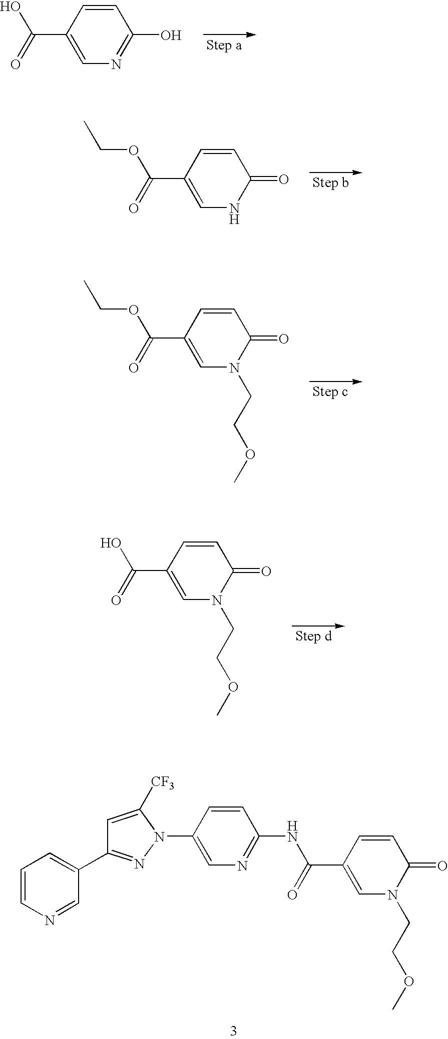

### Step a

A solution of 6-hydroxynicotinic acid (15.0 g, 107.83 mmol) in thionyl chloride (20 mL) is heated at reflux for 1 hour. The reaction mixture is allowed to cool room temperature, concentrated under vacuo, treated with ethanol (100 mL), and filtered to give desired solid. The solid is washed with ether (200 mL, 3×), and dried to give 6-oxo-1,6-dihydro-pyridine-3-carboxylic acid ethyl ester (17.3 g, 96%).

### Step b

A solution of 6-oxo-1,6-dihydro-pyridine-3-carboxylic acid ethyl ester (1 g, 5.98 mmol), 2-bromoethyl methyl ether (0.85 mL; 9.00 mmol), potassium hydroxide (1.17 g; 20.85 mmol) in ethanol (150 mL) is heated at reflux for 16 hours. The reaction is allowed to cool to room temperature, and concentrated under vacuo. The crude product is diluted with ethyl acetate, washed with water (50 mL, 3×), dried over sodium sulfate and concentrated under vacuo to give 1-(2-methoxy-ethyl)-6-oxo-1,6-dihydro-pyridine-3-carboxylic acid ethyl ester (370 mg, 27%).

### Step c

A solution of 1-(2-methoxy-ethyl)-6-oxo-1,6-dihydro-pyridine-3-carboxylic acid ethyl ester (370 mg, 1.64 mmol), lithium hydroxide (140 mg, 3.34 mmol) in dioxane (3 mL) and water (3 mL) is stirred at room temperature for 1 hour. The dioxane is removed under vacuo, the aqueous layer is washed with water (50 mL, 2×), and then acidified to pH 5 using 1 M aqueous HCl and extracted with ethyl acetate (50 mL, 3×). The ethyl acetate layers are combined, dried over sodium sulfate and concentrated under vacuo to give 1-(2-methoxy-ethyl)-6-oxo-1,6-dihydro-pyridine-3-carboxylic acid (280 mg, 86%).

### Step d

A solution of 1-(2-methoxy-ethyl)-6-oxo-1,6-dihydro-pyridine-3-carboxylic acid (280 mg, 1.42 mmol) in thionyl chloride (5.0 mL) is heated at reflux for 1 hour and then concentrated under vacuo. Pyridine (2.5 mL) is added to the preformed acid chloride followed by the addition of a solution of 5-(3-pyridine-3-yl-5-trifluoromethyl-pyrazol-1-yl)pyridin-2-ylamine (220 mg, 0.72 mmol), prepared according to example 1, in pyridine (2.5 mL). The reaction mixture is stirred for 1 hour, diluted with ethyl acetate, washed with water (50 mL, 3×), dried over sodium sulfate and concentrated under vacuo. The mixture is purified by chromatography to give the title compound (259 mg, 74%). LC-MS (M⁺+1): 485.04.

The following compounds are prepared according to example 3 by replacing the alkyl halide in step b with commercially available starting materials.

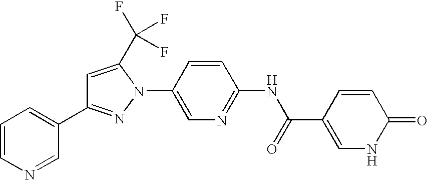

6-Oxo-1,6-dihydro-pyridine-3-carboxylic acid [5-(3-pyridin-3-yl-5-trifluoromethyl-pyrazol-1-yl)-pyridin-2-yl]-amide; LC-MS (M⁺+1): 427.39.

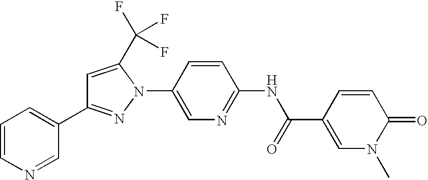

1-Methyl-6-oxo-1,6-dihydro-pyridine-3-carboxylic acid [5-(3-pyridin-3-yl-5-trifluoromethyl-pyrazol-1-yl)-pyridin-2-yl]-amide; LC-MS (M⁺+1): 440.97.

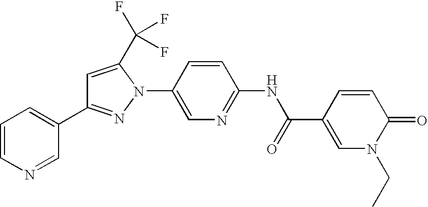

1-Ethyl-6-oxo-1,6-dihydro-pyridine-3-carboxylic acid [5-(3-pyridin-3-yl-5-trifluoromethyl-pyrazol-1-yl)-pyridin-2-yl]-amide; LC-MS (M⁺+1): 455.38.

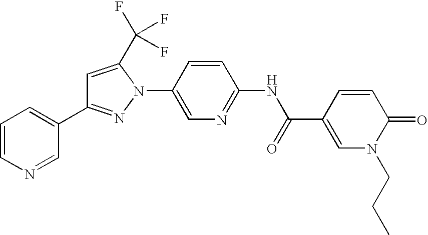

6-Oxo-1-propyl-1,6-dihydro-pyridine-3-carboxylic acid [5-(3-pyridin-3-yl-5-trifluoromethyl-pyrazol-1-yl)-pyridin-2-yl]-amide; LC-MS (M⁺+1): 469.35.

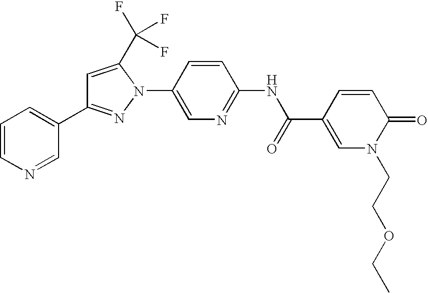

1-(2-Ethoxy-ethyl)-6-oxo-1,6-dihydro-pyridine-3-carboxylic acid [5-(3-pyridin-3-yl-5-trifluoromethyl-pyrazol-1-yl)-pyridin-2-yl]-amide; LC-MS (M⁺+1): 499.02.

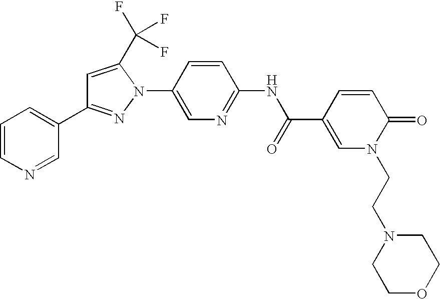

1-(2-Morpholin-4-yl-ethyl)-6-oxo-1,6-dihydro-pyridine-3-carboxylic acid [5-(3-pyridin-3-yl-5-trifluoromethyl-pyrazol-1-yl)-pyridin-2-yl]-amide; LC-MS (M⁺+1): 540.84.

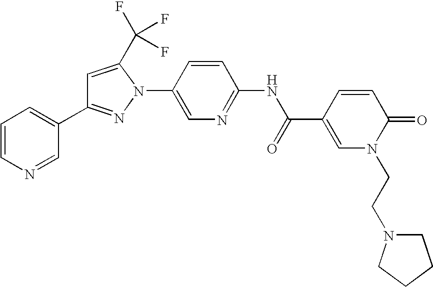

6-Oxo-1-(2-pyrrolidin-1-yl-ethyl)-1,6-dihydro-pyridine-3-carboxylic acid [5-(3-pyridin-3-yl-5-trifluoromethyl-pyrazol-1-yl)-pyridin-2-yl]-amide; LC-MS (M⁺+1): 524.89.

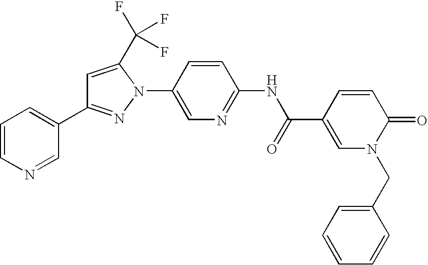

1-Benzyl-6-oxo-1,6-dihydro-pyridine-3-carboxylic acid [5-(3-pyridin-3-yl-5-trifluoromethyl-pyrazol-1-yl)-pyridin-2-yl]-amide; LC-MS (M⁺+1): 517.29.

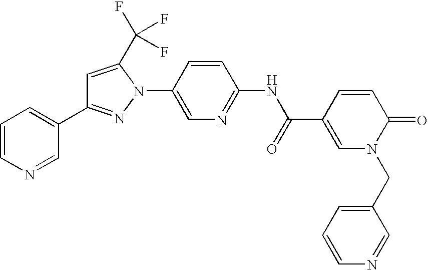

6-Oxo-1-pyridin-3-ylmethyl-1,6-dihydro-pyridine-3-carboxylic acid [5-(3-pyridin-3-yl-5-trifluoromethyl-pyrazol-1-yl)-pyridin-2-yl]-amide; LC-MS (M⁺+1): 518.35.

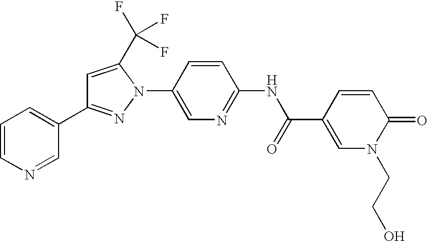

1-(2-Hydroxy-ethyl)-6-oxo-1,6-dihydro-pyridine-3-carboxylic acid [5-(3-pyridin-3-yl-5-trifluoromethyl-pyrazol-1-yl)-pyridin-2-yl]-amide; LC-MS (M⁺+1): 471.27.

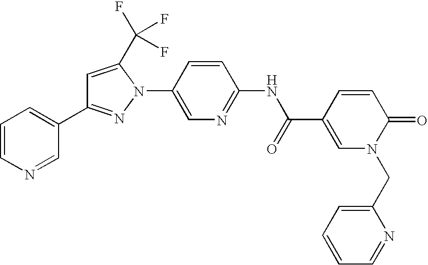

6-Oxo-1-pyridin-2-ylmethyl-1,6-dihydro-pyridine-3-carboxylic acid [5-(3-pyridin-3-yl-5-trifluoromethyl-pyrazol-1-yl)-pyridin-2-yl]-amide; LC-MS (M⁺+1): 517.90.

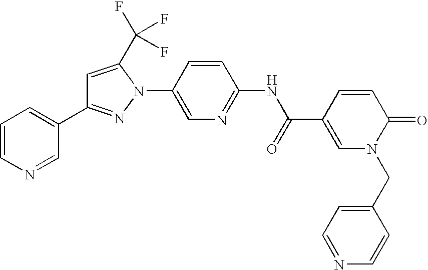

6-Oxo-1-pyridin-4-ylmethyl-1,6-dihydro-pyridine-3-carboxylic acid. [5-(3-pyridin-3-yl-5-trifluoromethyl-pyrazol-1-yl)-pyridin 2-yl]-amide; LC-MS (M⁺+1): 517.92.

### Example 4

### 1-(2-Morpholin-4-yl-2-oxo-ethyl)-6-oxo-1,6-dihydro-pyridine-3-carboxylic Acid [5-(3-pyridin-3-yl-5-trifluoromethyl-pyrazol-1-yl)-pyridin-2-yl]-amide

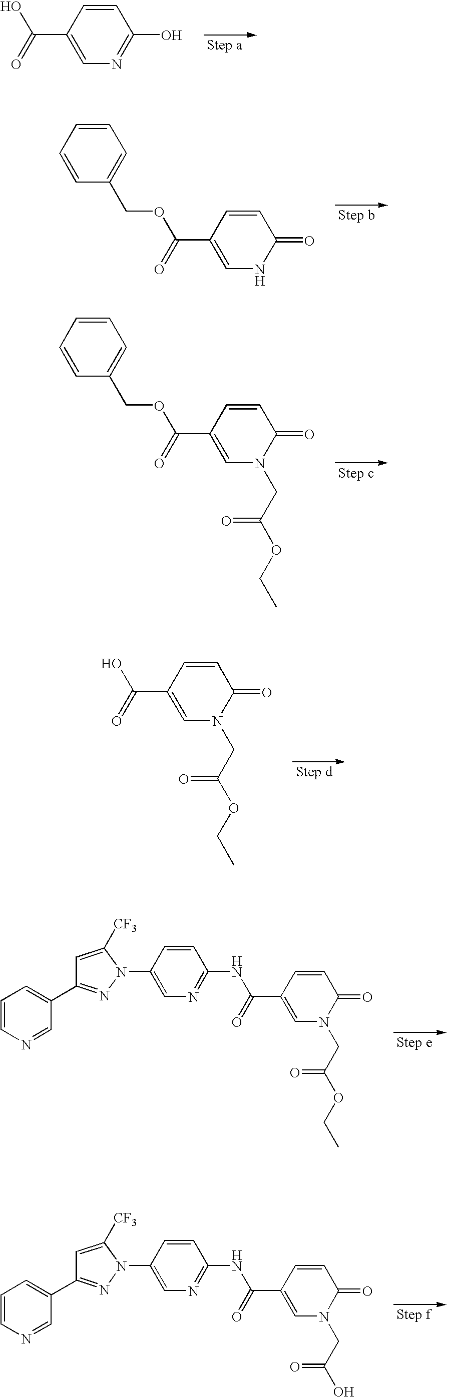

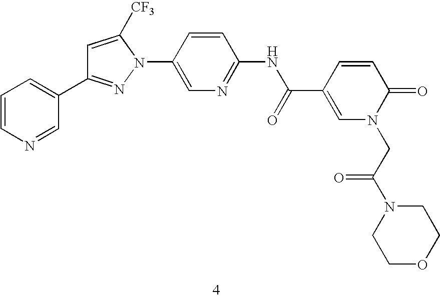

Step a

A solution of 6-hydroxynicotinic acid (17.0 g, 122.2 mmol) in thionyl chloride (20 mL) is heated at reflux for 1 hour. The reaction mixture is allowed to cool to room temperature, concentrated under vacuo, treated with benzyl alcohol (25 mL), triturated with ethyl acetate, filtered and dried to give 6-oxo-1,6-dihydro-pyridine-3-carboxylic acid benzyl ester (17.9 g, 64%).

### Step b

Sodium hydride (700 mg of a 60% dispersion in mineral oil; 17.50 mmol) is added slowly to a stirred solution of 6-oxo-1,6-dihydro-pyridine-3-carboxylic acid benzyl ester (2 g, 8.7 mmol) in tetrahydrofuran (25 mL) at 0° C. The reaction mixture is allowed to warm to room temperature, stirred for a further 30 min, followed by the addition of ethyl bromoacetate (1.45 mL, 13.08 mmol) and then stirred for an additional 4 hours. The reaction mixture is diluted with ethyl acetate, washed with water (100 mL, 3×), dried over sodium sulfate and concentrated under vacuo. The mixture is purified by chromatography to give 1-ethoxycarbonylmethyl-6-oxo-1,6-dihydro-pyridine-3-carboxylic acid benzyl ester (1.33 g, 48%).

### Step c

A solution of 1-ethoxycarbonylmethyl-6-oxo-1,6-dihydro-pyridine-3-carboxylic acid benzyl ester (1.33 g, 4.22 mmol), Palladium on carbon (260 mg, 10% wt (dry basis) on activated carbon, wet degussa type) in ethanol (25 mL) is stirred under an hydrogen atmosphere at room temperature for 4 hours. The reaction mixture is filtered through Celite to give 1-ethoxycarbonylmethyl-6-oxo-1,6-dihydro-pyridine-3-carboxylic acid (900 mg, 95%).

### Step d

A solution of 1-ethoxycarbonylmethyl-6-oxo-1,6-dihydro-pyridine-3-carboxylic acid (900 mg, 4.0 mmol) in oxalyl chloride (5.0 mL) and dimethylformamide (1 drop) is stirred at room temperature for 1 hour and then concentrated under vacuo. Pyridine (5 mL) is then added to the preformed acid chloride followed by the addition of a solution of 5-(3-pyridine-3-yl-5-trifluoromethyl-pyrazol-1-yl)pyridin-2-ylamine (610 mg; 2.0 mmol), prepared according to example 1, in pyridine (5 mL). The reaction mixture is stirred for 1 hour, diluted with ethyl acetate, washed with water (50 mL, 3×), dried over sodium sulfate and concentrated under vacuo. The mixture is purified by chromatography to give {2-oxo-5-[5-(3-pyridin-3-yl-5-trifluoromethyl-pyrazol-1-yl)-pyridin-2-ylcarbamoyl]-2H-pyridin-1-yl}-acetic acid ethyl ester (494 mg, 48%). LC-MS (M⁺+1): 513.15.

### Step e and f

A solution of {2-oxo-5-[5-(3-pyridin-3-yl-5-trifluoromethyl-pyrazol-1-yl)-pyridin-2-ylcarbamoyl]-2H-pyridin-1-yl}-acetic acid ethyl ester (40 mg, 0.08 mmol), lithium hydroxide monohydrate (10 mg; 0.24 mmol) in dioxane (2.5 mL) and water (2.5 mL) is stirred at room temperature for 1 hour. The reaction mixture is acidified to pH 6 using 4 M HCl in dioxane and then concentrated under vacuo to give {2-oxo-5-[5-(3-pyridin-3-yl-5-trifluoromethyl-pyrazol-1-yl)-pyridin-2-ylcarbamoyl]-2H-pyridin-1-yl}-acetic acid. To the above acid is added morpholine (35 μL; 0.401 mmol), 1-hydroxybenzotriazole (55 mg; 0.39 mmol) and dimethylformamide (5.0 mL) and this mixture is stirred for 15 min before the addition of 1-[3-(dimethylamino)propyl]-3-ethylcarbodiimide hydrochloride (75 mg, 0.39 mmol). The reaction mixture is stirred for 16 hours at room temperature and then concentrated under vacuo. The reaction mixture is diluted with ethyl acetate, washed with water (10 mL, 3×), dried over sodium sulfate and concentrated under vacuo. The mixture is purified by chromatography to give the title compound (14 mg, 10%). LC-MS (M⁺+1): 554.28.

The following compounds are prepared according to example 4 by replacing the amine in step f with commercially available starting materials.

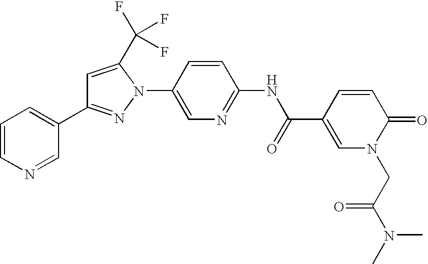

1-Dimethylcarbamoylmethyl-6-oxo-1,6-dihydro-pyridine-3-carboxylic acid [5-(3-pyridin-3-yl-5-trifluoromethyl-pyrazol-1-yl)-pyridin-2-yl]-amide; LC-MS (M⁺+1): 512.03 (M+H⁺).

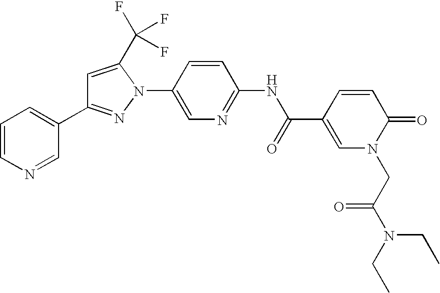

1-Diethylcarbamoylmethyl-6-oxo-1,6-dihydro-pyridine-3-carboxylic acid [5-(3-pyridin-3-yl-5-trifluoromethyl-pyrazol-1-yl)-pyridin-2-yl]-amide; LC-MS (M⁺+1): 540.05.

### Example 5

### 6-Oxo-1-p-tolyl-1,6-dihydro-pyridine-3-carboxylic Acid [5-(3-pyridin-3-yl-5-trifluoromethyl-pyrazol-1-yl)-pyridin-2-yl]-amide

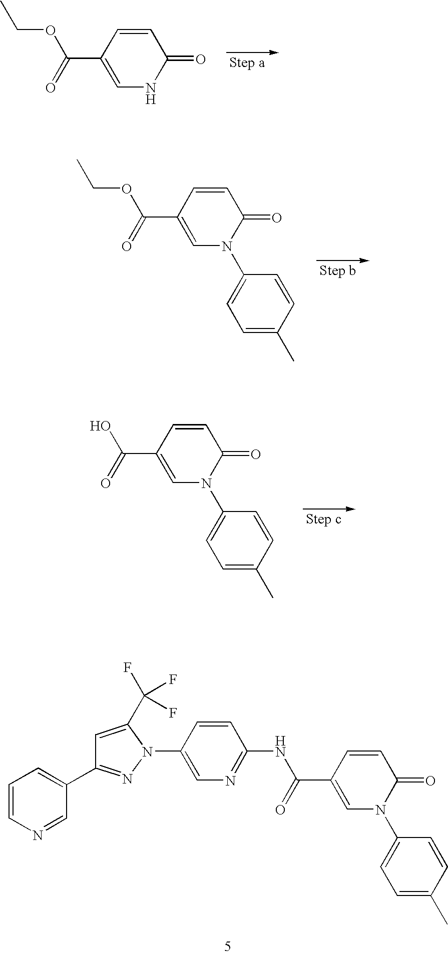

### Step a

A mixture of 6-oxo-1,6-dihydro-pyridine-3-carboxylic acid ethyl ester (335 mg, 2.00 mmol), prepared according to example 3,4-iodo-toluene (525 mg, 2.41 mmol), Copper (I) iodide (80 mg, 0.42 mmol), N,N′-dimethylethylenediamine (90 μL, 0.85 mmol), potassium phosphate (850 mg, 4.00 mmol) in dioxane (3.0 mL) is heated under a nitrogen atmosphere at 110° C. in a sealed tube for 16 hours. The reaction mixture is allowed to cool to room temperature, diluted with ethyl acetate, washed with water (50 mL, 3×), dried over sodium sulfate and concentrated under vacuo. The mixture is purified by chromatography to give 6-oxo-1-p-tolyl-1,6-dihydro-pyridine-3-carboxylic acid ethyl ester (85 mg; 16.5%).

### Step b & c

A solution of 6-oxo-1-p-tolyl-1,6-dihydro-pyridine-3-carboxylic acid ethyl ester (85 mg, 0.33 mmol), lithium hydroxide (30 mg, 0.72 mmol) in dioxane (2.5 mL) and water (2.5 mL) is stirred at room temperature for 1 hour. The reaction mixture is acidified to pH 5 using 4 M HCl in dioxane, and concentrated under vacuo to give 6-oxo-1-p-tolyl-1,6-dihydro-pyridine-3-carboxylic acid.

A solution of the above residue in thionyl chloride (5.0 mL) is heated at reflux for 1 hour and then concentrated under vacuo. Pyridine (2.5 mL) is then added to the preformed acid chloride followed by the addition of a solution of 5-(3-pyridine-3-yl-5-trifluoromethyl-pyrazol-1-yl)pyridin-2-ylamine (70 mg, 0.23 mmol), prepared according to example 1, in pyridine (2.5 mL). The reaction mixture is stirred for 1 hour, diluted with ethyl acetate, washed with water (25 mL, 3×), dried over sodium sulfate and concentrated under vacuo. The mixture is purified by chromatography to give the title compound (19 mg, 16%). LC-MS (M⁺+1): 517.01.

### Example 6

### 2-Dimethylamino-N-[5-(3-pyridin-3-yl-5-trifluoromethyl-pyrazol-1-yl)-pyridin-2-yl]-isonicotinamide

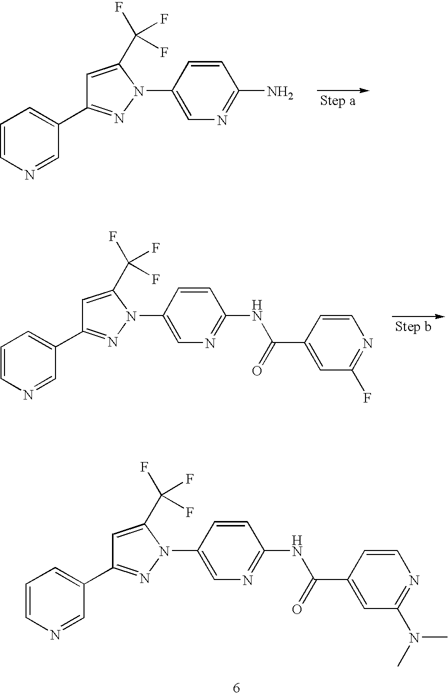

### Step a

A solution of 2-fluoronicotinic acid (200 mg, 1.42 mmol) in thionyl chloride (5.0 mL) is heated at reflux for 1 hour and then concentrated under vacuo. Pyridine (2.5 mL) is then added to the preformed acid chloride followed by a solution of 5-(3-pyridine-3-yl-5-trifluoromethyl-pyrazol-1-yl)pyridin-2-ylamine (200 mg, 0.66 mmol), prepared according to example 1, in pyridine (2.5 mL). The reaction mixture is stirred for 1 hour, diluted with ethyl acetate, washed with water (25 mL, 3×), dried over sodium sulfate and then concentrated under vacuo. The mixture is purified by chromatography to give 2-fluoro-N-[5-(3-pyridin-3-yl-5-trifluoromethyl-pyrazol-1-yl)-pyridin-2-yl]-isonicotinamide (100 mg, 41%). LC-MS (M⁺+1): 428.94.

### Step b

A solution of 2-fluoro-N-[5-(3-pyridin-3-yl-5-trifluoromethyl-pyrazol-1-yl)-pyridin-2-yl]-isonicotinamide (45 mg, 0.11 mmol), dimethylamine hydrochloride (20 mg, 0.25 mmol), triethylamine (35 μL, 0.25 mmol) in dimethylformamide (2.0 mL) is heated at 100° C. for 48 hours. The reaction mixture is concentrated under vacuo and the crude product is purified by chromatography to give the title compound. LC-MS (M⁺+1): 453.97.

The following compounds are prepared according to example 6 by replacing the amine in step b with commercially available starting materials.

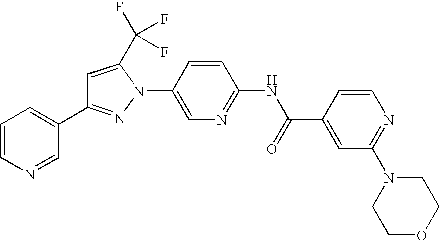

2-Morpholin-4-yl-N-[5-(3-pyridin-3-yl-5-trifluoromethyl-pyrazol-1-yl)-pyridin-2-yl]-isonicotinamide; LC-MS (M⁺+1): 496.98.

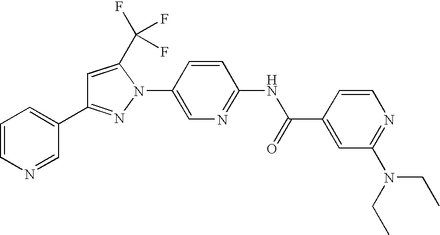

2-Diethylamino-N-[5-(3-pyridin-3-yl-5-trifluoromethyl-pyrazol-1-yl)-pyridin-2-yl]-isonicotinamide; LC-MS (M⁺+1): 482.05.

### Example 7

### 1-Methyl-2-oxo-1,2-dihydro-pyridine-4-carboxylic Acid [5-(3-pyridin-3-yl-5-trifluoromethyl-pyrazol-1-yl)-pyridin-2-yl]-amide

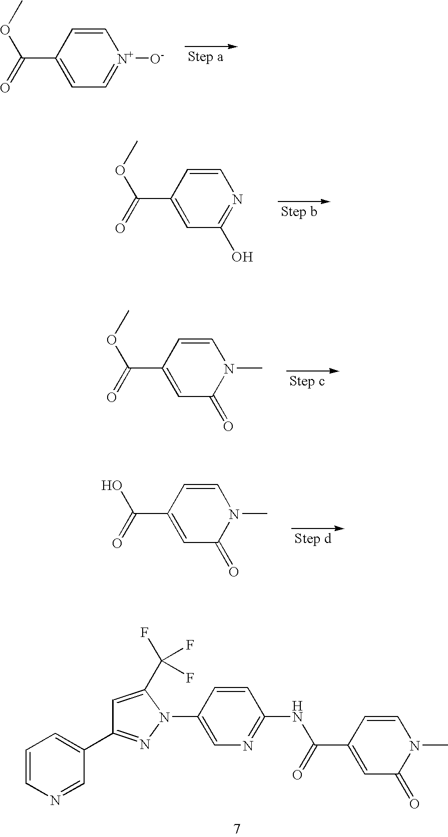

### Step a

A solution of methyl isonicotinate-N-oxide (3 g, 19.59 mmol) in acetic anhydride (30 mL) is heated at reflux for 16 hours. The reaction mixture is allowed to cool to room temperature, concentrated under vacuo, diluted with methanol and with celite added, the mixture is heated at reflux for 30 min. The reaction mixture is filtered, reduced under vacuo and triturated with methanol to give 2-hydroxy-isonicotinic acid methyl ester (400 mg, 13%).

### Step b

Sodium hydride (80 mg of a 60% dispersion in mineral oil, 3.3 mmol) is added to a solution of 2-hydroxy-isonicotinic acid methyl ester (200 mg, 1.31 mmol) in dimethylformamide (2.5 mL) at room temperature and stirred for 30 min. Methyl iodide (175 mg, 2.81 mmol) is added to the reaction mixture and stirred for 16 hours at room temperature. The reaction mixture is diluted with ethyl acetate, washed with water (25 mL, 3×), dried over sodium sulfate and concentrated under vacuo to give 1-methyl-2-oxo-1,2-dihydro-pyridine-4-carboxylic acid methyl ester (100 mg, 46%).

### Step c & d

A solution of 1-methyl-2-oxo-1,2-dihydro-pyridine-4-carboxylic acid methyl ester (100 mg, 0.60 mmol), lithium hydroxide monohydrate (50 mg, 1.19 mmol) in dioxane (2.5 mL) and water (2.5 mL) is stirred at room temperature for 2 hours. The reaction mixture is acidified to pH 6 using 4 M HCl in dioxane and then concentrated under vacuo to give 1-methyl-2-oxo-1,2-dihydro-pyridine-4-carboxylic acid.

The above acid is heated at reflux in thionyl chloride (5.0 mL) for 1 hour and then concentrated under vacuo. Pyridine (2.5 mL) is then added to the preformed acid chloride followed by a solution of 5-(3-pyridine-3-yl-5-trifluoromethyl-pyrazol-1-yl)pyridin-2-ylamine (90 mg; 0.30 mmol), prepared according to example 1, in pyridine (2.5 mL). The reaction mixture is stirred for 1 hour, diluted with ethyl acetate, washed with water (25 mL, 3×), dried over sodium sulfate and concentrated under vacuo. The mixture is purified by chromatography to give the title compound (18 mg, 14%). LC-MS (M⁺+1): 441.01.

The following compounds are prepared according to example 7 by replacing the alkyl halide in step b with commercially available starting materials.

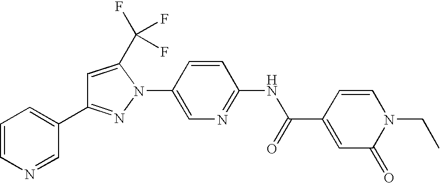

1-Ethyl-2-oxo-1,2-dihydro-pyridine-4-carboxylic acid [5-(3-pyridin-3-yl-5-trifluoromethyl-pyrazol-1-yl)-pyridin-2-yl]-amide; LC-MS (M⁺+1): 454.97.

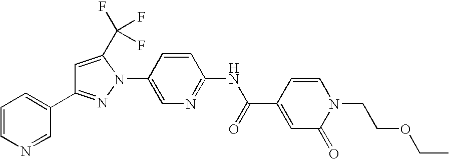

1-(2-Ethoxy-ethyl)-2-oxo-1,2-dihydro-pyridine-4-carboxylic acid [5-(3-pyridin-3-yl-5-trifluoromethyl-pyrazol-1-yl)-pyridin-2-yl]-amide; LC-MS (M⁺+1): 498.97 (M+H⁺).

### Example 8

### N-[5-(3-Pyridin-3-yl-5-trifluoromethyl-pyrazol-1-yl)-pyridin-2-yl]-3-pyrimidin-5-yl-benzamide

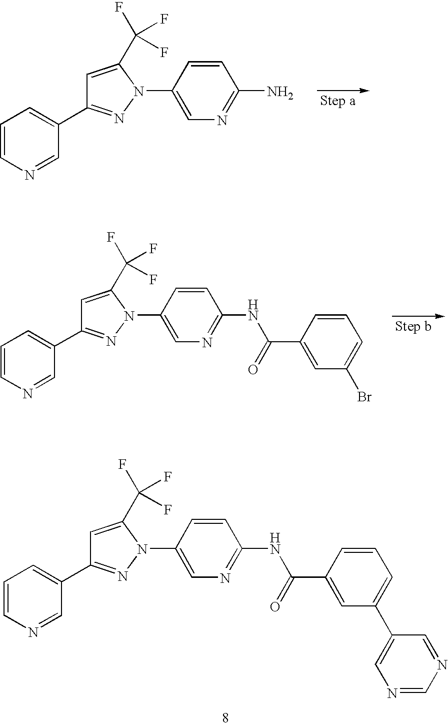

### Step a

A solution of 5-(3-pyridine-3-yl-5-trifluoromethyl-pyrazol-1-yl)pyridin-2-ylamine (140 mg, 0.46 mmol), prepared according to example 1,3-bromobenzoyl chloride (150 μL, 1.12 mmol) in pyridine (5.0 mL) is heated at reflux for 2 hours. The reaction mixture is allowed to cool to room temperature, concentrated under vacuo, and the mixture is purified by chromatography to give 3-bromo-N-[5-(3-pyridin-3-yl-5-trifluoromethyl-pyrazol-1-yl)-pyridin-2-yl]-benzamide (116 mg, 52%). LC-MS (M⁺+1): 490.27.

### Step b

A solution of 3-bromo-N-[5-(3-pyridin-3-yl-5-trifluoromethyl-pyrazol-1-yl)-pyridin-2-yl]-benzamide (100 mg, 0.21 mmol), 5-pyrimidylboronic acid (50 mg, 0.40 mmol), tetrakis(triphenylphosphine)palladium(0) (24 mg; 0.02 mmol), cesium carbonate (66 mg, 0.20 mmol) in dimethylformamide (4 mL) is heated at 100° C. under microwave conditions for 10 min. The reaction mixture is diluted with ethyl acetate, washed with water (25 mL, 3×), dried over sodium sulfate and concentrated under vacuo. The crude mixture is purified by chromatography to give the title compound (12 mg, 12%). LC-MS (M⁺+1): 489.88.

The following compound is prepared according to example 8 by replacing the alkyl halide in step b with commercially available starting material.

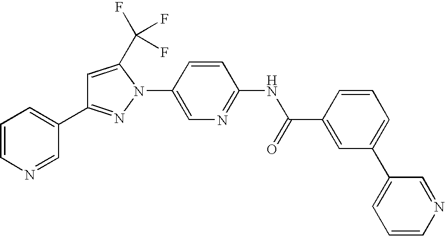

3-Pyridin-3-yl-N-[5-(3-pyridin-3-yl-5-trifluoromethyl-pyrazol-1-yl)-pyridin-2-yl]-benzamide; LC-MS (M⁺+1): 487.38.

### Example 9

### 1-(2,4-Dichloro-benzyl)-3-[5-(3-pyridin-3-yl-5-trifluoromethyl-pyrazol-1-yl)-pyridin-2-yl]-urea

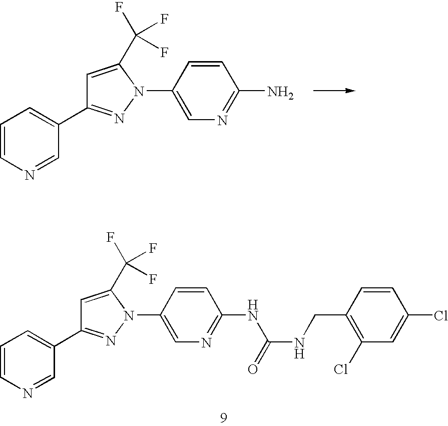

A solution of 5-(3-pyridine-3-yl-5-trifluoromethyl-pyrazol-1-yl)pyridin-2-ylamine (50 mg, 0.16 mmol), prepared according to example 1, 2,4-dichlorobenzylisocyanate (25 μL, 0.17 mmol) in acetonitrile (2.5 mL) is stirred at room temperature for 16 hours. The reaction mixture is filtered to give a crude solid product which is purified by chromatography to give the title compound (33 mg, 40%. LC-MS (M⁺+1): 507.27.

The following compounds are prepared according to example 9 by replacing the isocyanate with commercially available starting materials.

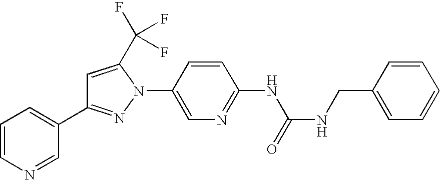

1-Benzyl-3-[5-(3-pyridin-3-yl-5-trifluoromethyl-pyrazol-1-yl)-pyridin-2-yl]-urea; LC-MS (M⁺+1): 439.01.

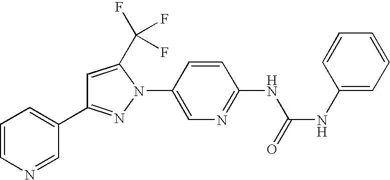

1-Phenyl-3-[5-(3-pyridin-3-yl-5-trifluoromethyl-pyrazol-1-yl)-pyridin-2-yl]-urea; LC-MS (M⁺+1): 424.95.

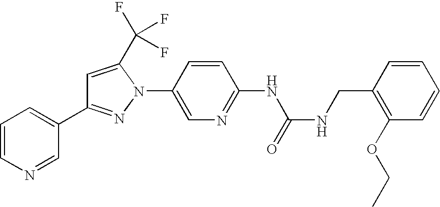

1-(2-Ethoxy-benzyl)-3-[5-(3-pyridin-3-yl-5-trifluoromethyl-pyrazol-1-yl)-pyridin-2-yl]-urea; LC-MS (M⁺+1): 482.98.

### Example 10

### Cyclohexanecarboxylic Acid [5-(5-pyridin-3-yl-3-trifluoromethyl-pyrazol-1-yl)-pyridin-2-yl]-amide

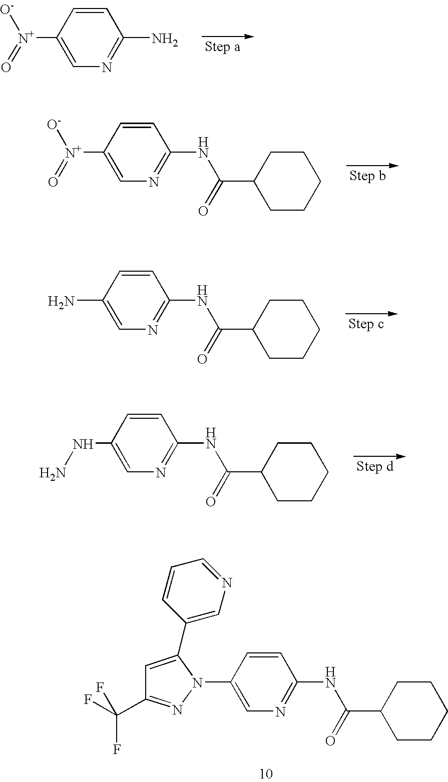

### Step a

A solution of 2-amino-5-nitropyridine (1.5 g, 10.78 mmol), triethylamine (1.6 mL, 11.43 mmol), cyclohexanecarbonyl chloride (1.44 mL, 10.76 mmol) in tetrahydrofuran (25 mL) is stirred at room temperature for 2 hours. The reaction mixture is concentrated under vacuo, diluted with ethyl acetate, washed with 1 M aqueous HCl (75 mL, 3×), dried over sodium sulfate and concentrated under vacuo. The crude product is purified by chromatography to give cyclohexanecarboxylic acid (5-nitro-pyridin-2-yl)-amide (1 g, 37%).

### Step b

A solution of cyclohexanecarboxylic acid (5-nitro-pyridin-2-yl)-amide (1 g, 4.01 mmol), palladium on carbon (150 mg, 10% wt (dry basis) on activated carbon, wet degussa type) in methanol (25 mL) is stirred under an hydrogen atmosphere at room temperature for 16 hours. The reaction mixture is filtered to give cyclohexanecarboxylic acid (5-amino-pyridin-2-yl)-amide (0.9 g, 99%).

### Step c

Sodium nitrite (95 mg, 1.0 mmol) is added slowly to a solution of cyclohexanecarboxylic acid (5-amino-pyridin-2-yl)-amide (200 mg, 0.912 mmol) in 6 M aqueous HCl (5 mL) at 0° C. and is stirred for 45 min. Tin(II)chloride (412 mg, 1.826 mmol) is added and the reaction mixture is allowed to warm to room temperature slowly while stirring for 16 hours. The reaction mixture is basified with 40% aqueous potassium hydroxide, extracted with ethyl acetate (25 mL, 3×), and the organic layers are combined, dried over sodium sulfate and concentrated under vacuo to give cyclohexanecarboxylic acid (5-hydrazino-pyridin-2-yl)-amide (150 mg, 70%).

### Step d

A solution of 4,4,4-trifluoro-1-pyridin-3-yl-butane-1,3-dione (120 mg, 0.55 mmol), prepared according to example 1, cyclohexanecarboxylic acid (5-hydrazino-pyridin-2-yl)-amide (150 mg, 0.64 mmol), acetic acid (1 mL) in ethanol (10 mL) is heated at reflux for 2 hours, allowed to cool to room temperature, and concentrated under vacuo. The mixture is purified by chromatography to give the title compound (60 mg, 26%). LC-MS (M⁺+1): 432.04.

The following compounds are prepared according to example 10 by replacing the acyl chloride in step a with commercially available starting materials.

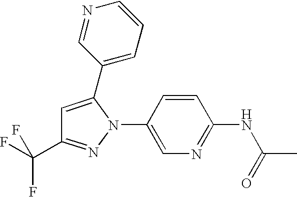

N-[5-(5-Pyridin-3-yl-3-trifluoromethyl-pyrazol-1-yl)-pyridin-2-yl]-acetamide; LC-MS (M⁺+1): 348.41.

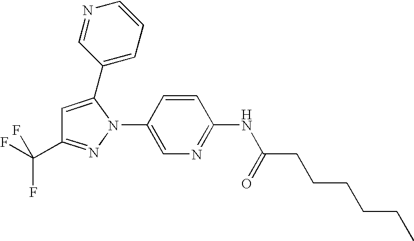

Heptanoic acid [5-(5-pyridin-3-yl-3-trifluoromethyl-pyrazol-1-yl)-pyridin-2-yl]-amide LC-MS (M⁺+1): 418.41.

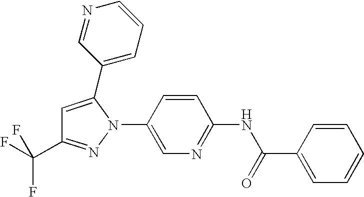

N-[5-(5-Pyridin-3-yl-3-trifluoromethyl-pyrazol-1-yl)-pyridin-2-yl]-benzamide; LC-MS (M⁺+1): 409.90.

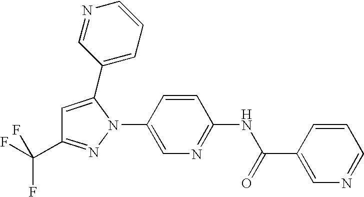

N-[5-(5-Pyridin-3-yl-3-trifluoromethyl-pyrazol-1-yl)-pyridin-2-yl]-nicotinamide; LC-MS (M⁺+1): 411.35.

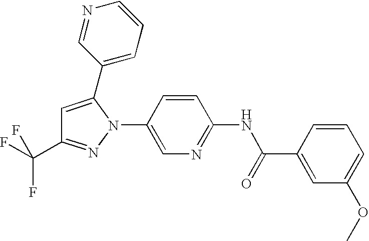

3-Methoxy-N-[5-(5-pyridin-3-yl-3-trifluoromethyl-pyrazol-1-yl)-pyridin-2-yl]-benzamide; LC-MS (M⁺+1): 440.33.

### Example 11

### N-Pyridin-4-yl-4-(3-pyridin-3-yl-5-trifluoromethyl-pyrazol-1-yl)-benzamide

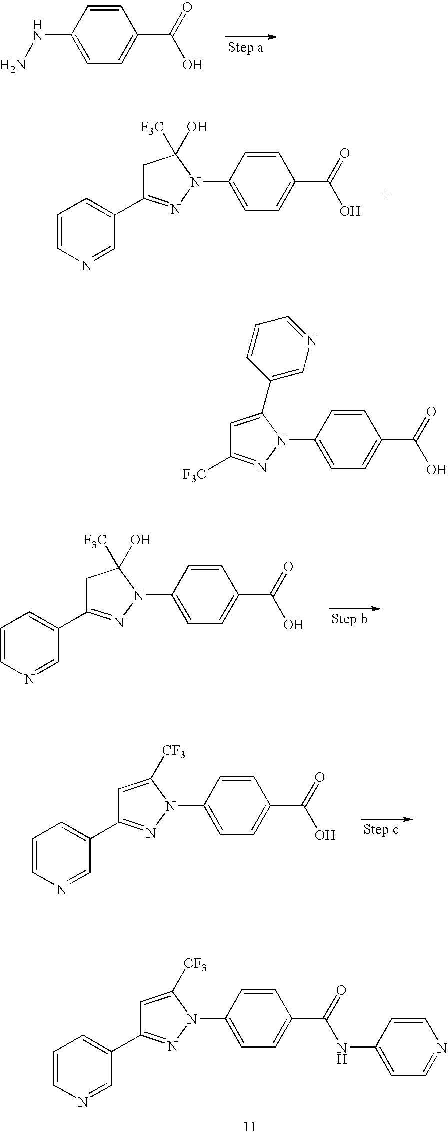

### Step a

A solution of 4,4,4-trifluoro-1-pyridin-3-yl-butane-1,3-dione (5.0 g, 23.03 mmol), prepared according to example 1,4-hydrazinobenzoic acid (3.5 g, 23.00 mmol) is heated at reflux in ethanol (250 mL) for 16 hours. The reaction mixture is allowed to cool to room temperature, and the solid is filtered to give 4-(5-pyridin-3-yl-3-trifluoromethyl-pyrazol-1-yl)-benzoic acid (2.44 g, 30%). The mother liquor is concentrated under vacuo, then triturated with ethyl acetate and filtered to give 4-(5-phenyl-3-trifluoromethyl-pyrazol-1-yl)-benzoic acid (1.56 g, 20%).

### Step b

A solution of 4-(5-hydroxy-3-phenyl-5-trifluoromethyl-4,5-dihydro-pyrazol-1-yl)-benzoic acid (2.0 g, 5.69 mmol) in acetic acid (25 mL) is heated at reflux for 16 hours. The reaction mixture is concentrated under vacuo, triturated with ethyl acetate and filtered to give 4-(3-pyridin-3-yl-5-trifluoromethyl-pyrazol-1-yl)-benzoic acid (0.93 g, 49%).

### Step c

4-Aminopyridine (35 mg, 0.37 mmol) is added to a stirred solution of 4-(3-pyridin-3-yl-5-trifluoromethyl-pyrazol-1-yl)-benzoic acid (100 mg, 0.3 mmol), 1-[3-(dimethylamino) propyl]-3-ethylcarbodiimide hydrochloride (150 mg, 0.77 mmol), triethylamine (0.1 mL, 0.71 mmol) in dichloromethane (5.0 mL) at room temperature and the reaction is stirred for 16 hours. The reaction mixture is diluted with dichloromethane, washed with saturated aqueous sodium bicarbonate solution (25 mL, 3×), dried over sodium sulfate and concentrated under vacuo. The mixture is purified by chromatography to give the title compound (43 mg, 35%). LC-MS (M⁺+1): 409.98.

The following compounds are prepared according to example 11 by replacing the amine in step c with commercially available starting materials.

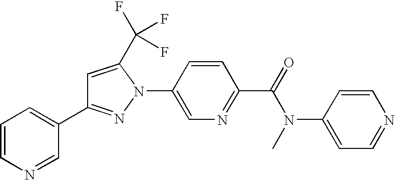

N-Methyl-N-pyridin-4-yl-4-(3-pyridin-3-yl-5-trifluoromethyl-pyrazol-1-yl)-benzamide; LC-MS (M⁺+1): 424.40.

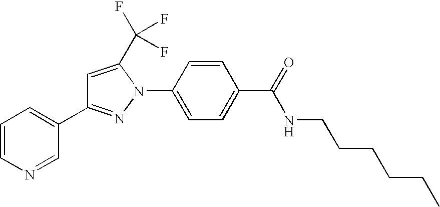

N-Hexyl-4-(3-pyridin-3-yl-5-trifluoromethyl-pyrazol-1-yl)-benzamide; LC-MS (M⁺+1): 417.46.

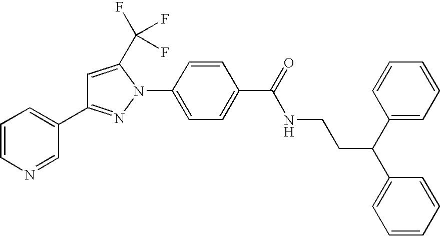

N-(3,3-Diphenyl-propyl)-4-(3-pyridin-3-yl-5-trifluoromethyl-pyrazol-1-yl)-benzamide; LC-MS (M⁺+1): 527.45.

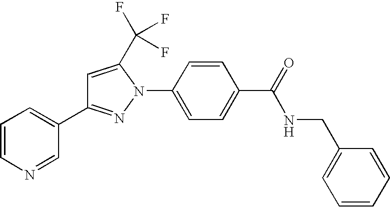

N-Benzyl-4-(3-pyridin-3-yl-5-trifluoromethyl-pyrazol-1-yl)-benzamide; LC-MS (M⁺+1): 423.45.

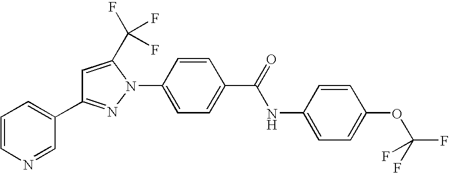

4-(3-Pyridin-3-yl-5-trifluoromethyl-pyrazol-1-yl)-N-(4-trifluoromethoxy-phenyl)-benzamide; LC-MS (M⁺+1): 493.36 (M+H⁺).

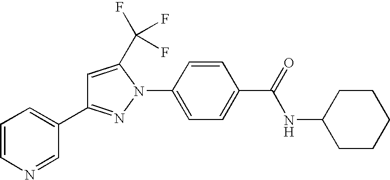

N-Cyclohexyl-4-(3-pyridin-3-yl-5-trifluoromethyl-pyrazol-1-yl)-benzamide; LC-MS (M⁺+1): 415.47.

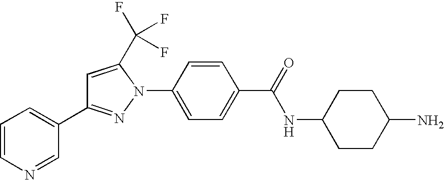

N-(4-Amino-cyclohexyl)-4-(3-pyridin-3-yl-5-trifluoroethyl-pyrazol-1-yl)-benzamide LC-MS (M⁺+1): 431.39.

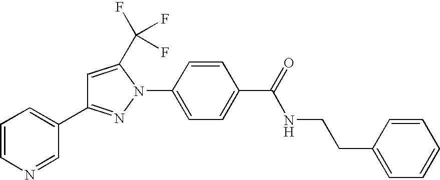

N-Phenethyl-4-(3-pyridin-3-yl-5-trifluoromethyl-pyrazol-1-yl)-benzamide; LC-MS (M⁺+1): 437.69.

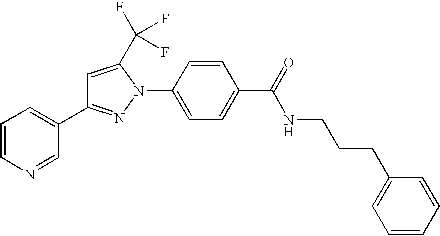

N-(3-Phenyl-propyl)-4-(3-pyridin-3-yl-5-trifluoromethyl-pyrazol-1-yl)-benzamide; LC-MS (M⁺+1): 451.52.

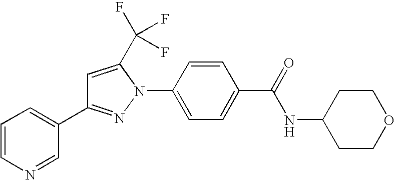

4-(3-Pyridin-3-yl-5-trifluoromethyl-pyrazol-1-yl)-N-(tetrahydro-pyran-4-yl)-benzamide; LC-MS (M⁺+1): 417.41.

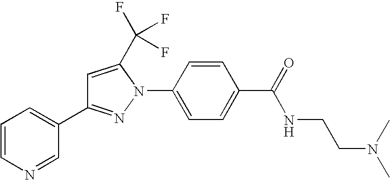

N-(2-Dimethylamino-ethyl)-4-(3-pyridin-3-yl-5-trifluoromethyl-pyrazol-1-yl)-benzamide; LC-MS (M⁺+1): 404.34.

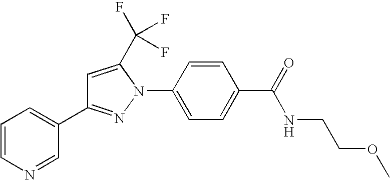

N-(2-Methoxy-ethyl)-4-(3-pyridin-3-yl-5-trifluoromethyl-pyrazol-1-yl)-benzamide; LC-MS (M⁺+1): 391.37.

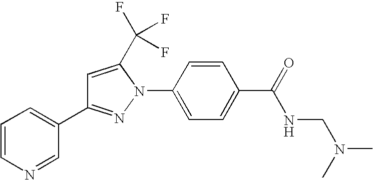

N-Dimethylaminomethyl-4-(3-pyridin-3-yl-5-trifluoromethyl-pyrazol-1-yl)-benzamide; LC-MS (M⁺+1): 389.38.

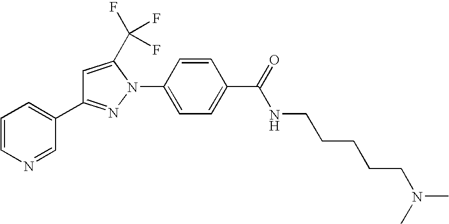

N-(5-Dimethylamino-pentyl)-4-(3-pyridin-3-yl-5-trifluoromethyl-pyrazol-1-yl)-benzamide; LC-MS (M⁺+1): 445.98.

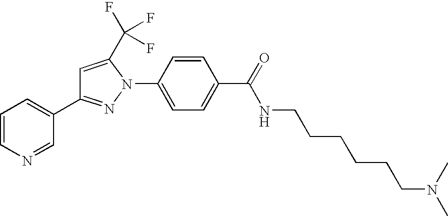

N-(6-Dimethylamino-hexyl)-4-(3-pyridin-3-yl-5-trifluoromethyl-pyrazol-1-yl)-benzamide; LC-MS (M⁺+1): 459.99.

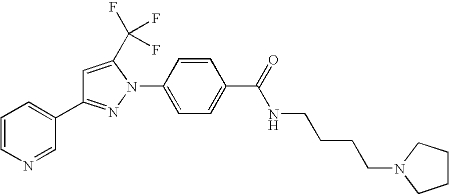

4-(3-Pyridin-3-yl-5-trifluoromethyl-pyrazol-1-yl)-N-(4-pyrrolidin-1-yl-butyl)-benzamide; LC-MS (M⁺+1): 457.94.

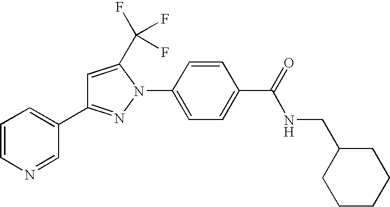

N-Cyclohexylmethyl-4-(3-pyridin-3-yl-5-trifluoromethyl-pyrazol-1-yl)-benzamide; LC-MS (M⁺+1): 429.40.

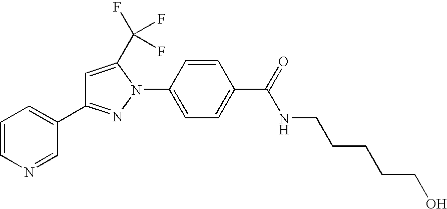

N-(5-Hydroxy-pentyl)-4-(3-pyridin-3-yl-5-trifluoromethyl-pyrazol-1-yl)-benzamide; LC-MS (M⁺+1): 419.38.

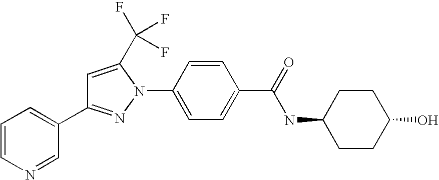

N-(4-Hydroxy-cyclohexyl)-4-(3-pyridin-3-yl-5-trifluoromethyl-pyrazol-1-yl)-benzamide; LC-MS (M⁺+1): 431.39.

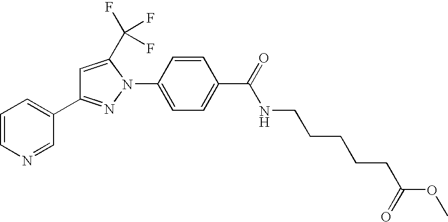

6-[4-(3-Pyridin-3-yl-5-trifluoromethyl-pyrazol-1-yl)-benzoylamino]-hexanoic acid methyl ester; LC-MS (M⁺+1): 461.43.

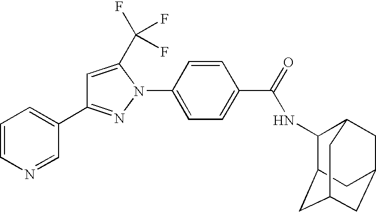

N-Adamantan-2-yl-4-(3-pyridin-3-yl-5-trifluoromethyl-pyrazol-1-yl)-benzamide; LC-MS (M⁺+1): 466.98.

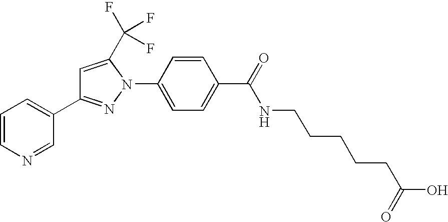

6-[4-(3-Pyridin-3-yl-5-trifluoromethyl-pyrazol-1-yl)-benzoylamino]-hexanoic acid; LC-MS (M⁺+1): 446.94.

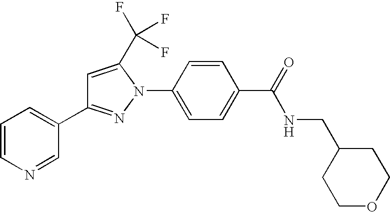

4-(3-Pyridin-3-yl-5-trifluoromethyl-pyrazol-1-yl)-N-(tetrahydro-pyran-4-ylmethyl)-benzamide, LC-MS (M⁺+1): 430.94.

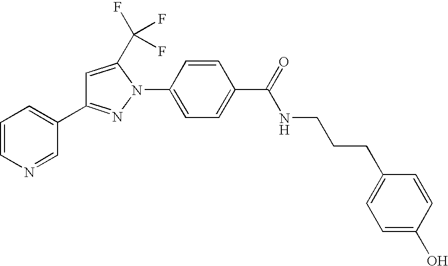

N-[3-(4-Hydroxy-phenyl)-propyl]-4-(3-pyridin-3-yl-5-trifluoromethyl-pyrazol-1-yl)-benzamide; LC-MS (M⁺+1): 466.94.

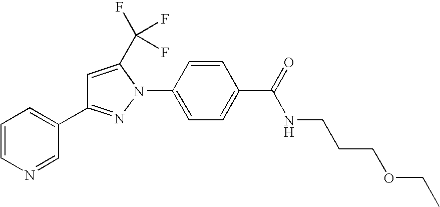

N-(3-Ethoxy-propyl)-4-(3-pyridin-3-yl-5-trifluoromethyl-pyrazol-1-yl)-benzamide; LC-MS (M⁺+1): 419.40.

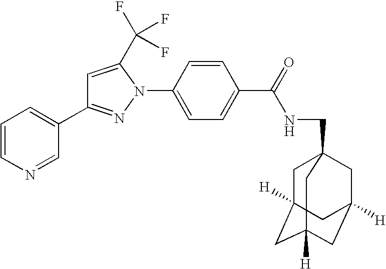

N-Adamantan-1-Ylmethyl-4-(3-pyridin-3-yl-5-trifluoromethyl-pyrazol-1-yl)-benzamide; LC-MS (M⁺+1): 481.42.

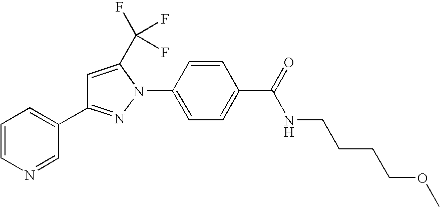

N-(4-Methoxy-butyl)-4-(3-pyridin-3-yl-5-trifluoromethyl-pyrazol-1-yl)-benzamide; LC-MS (M⁺+1): 420.40.

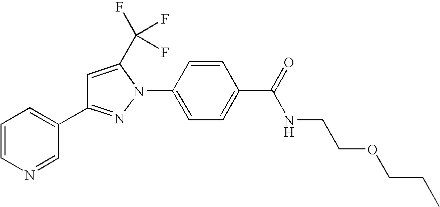

N-(2-Propoxy-ethyl)-4-(3-pyridin-3-yl-5-trifluoromethyl-pyrazol-1-yl)-benzamide; LC-MS (M⁺+1): 418.98.

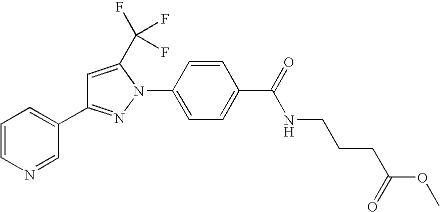

4-[4-(3-Pyridin-3-yl-5-trifluoromethyl-pyrazol-1-yl)-benzoylamino]-butyric acid methyl ester; LC-MS (M¹+1): 432.97.

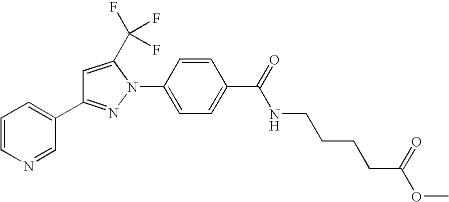

5-[4-(3-Pyridin-3-yl-5-trifluoromethyl-pyrazol-1-yl)-benzoylamino]-pentanoic acid methyl ester; LC-MS (M⁺+1): 446.97.

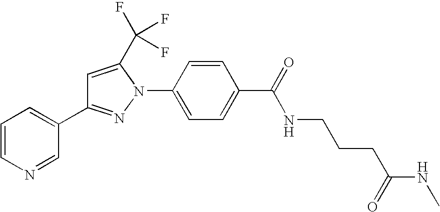

N-(3-Methylcarbamoyl-propyl)-4-(3-pyridin-3-yl-5-trifluoromethyl-pyrazol-1-yl)-benzamide; LC-MS (M⁺+1): 431.97.

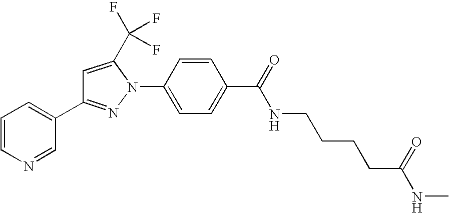

N-(4-Methylcarbamoyl-butyl)-4-(3-pyridin-3-yl-5-trifluoromethyl-pyrazol-1-yl)-benzamide; LC-MS (M⁺+1): 446.00.

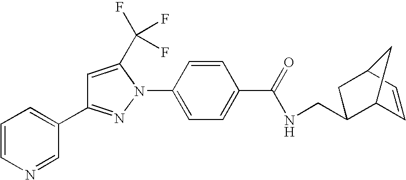

N-Bicyclo[2.2.1]hept-5-en-2-ylmethyl-4-(3-pyridin-3-yl-5-trifluoromethyl-pyrazol-1-yl)-benzamide; LC-MS (M⁺+1): 439.36

### Example 12

### N-Pyridin-4-yl-4-(5-pyridin-3-yl-3-trifluoromethyl-pyrazol-1-yl)-benzamide

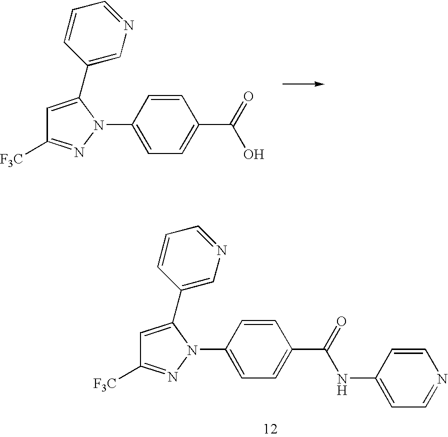

4-Aminopyridine (28 mg, 0.30 mmol) is added to a stirred solution of 4-(5-Pyridin-3-yl-3-trifluoromethyl-pyrazol-1-yl)-benzoic acid (80 mg, 0.24 mmol), prepared according to example 11, 1-[3-(dimethylamino) propyl]-3-ethylcarbodiimide hydrochloride (116 mg, 0.60 mmol), dimethylaminopyridine (1 mg) in dichloromethane (2.5 mL) at room temperature and the reaction is stirred for 16 hours. The reaction mixture is diluted with dichloromethane, washed with saturated aqueous sodium bicarbonate solution (25 mL, 3×), dried over sodium sulfate and concentrated under vacuo. The mixture is purified by chromatography to give the title compound (24 mg, 24%). LC-MS (M⁺+1): 410.43.

The following compounds are prepared according to example 12 by replacing the amine with commercially available starting materials.

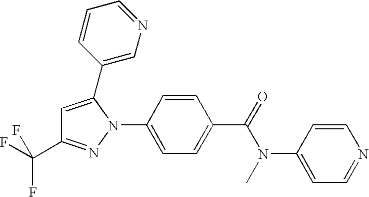

N-Methyl-N-pyridin-4-yl-4-(5-pyridin-3-yl-3-trifluoromethyl-pyrazol-1-yl)-benzamide; LC-MS (M⁺+1): 424.40.

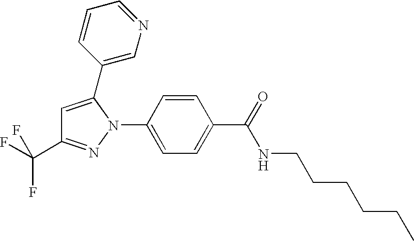

N-Hexyl-4-(5-pyridin-3-yl-3-trifluoromethyl-pyrazol-1-yl)-benzamide; LC-MS (M⁺+1): 417.46.

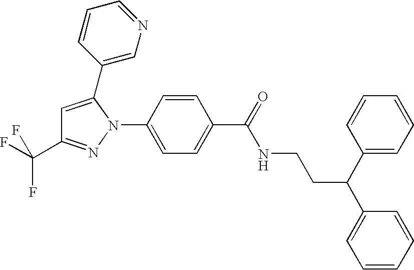

N-(3,3-Diphenyl-propyl)-4-(5-pyridin-3-yl-3-trifluoromethyl-pyrazol-1-yl)-benzamide; LC-MS (M⁺+1): 527.47.

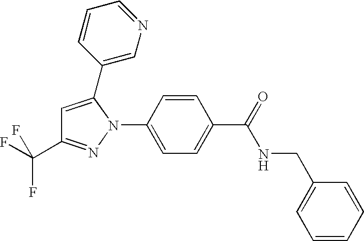

N-Benzyl-4-(5-pyridin-3-yl-3-trifluoromethyl-pyrazol-1-yl)-benzamide; LC-MS (M⁺+1): 423.43.

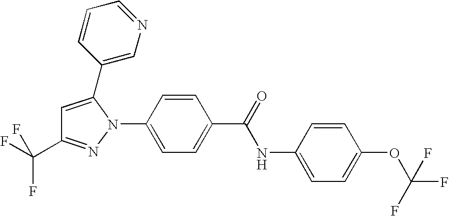

4-(5-Pyridin-3-yl-3-trifluoromethyl-pyrazol-1-yl)-N-(4-trifluoromethoxy-phenyl)-benzamide; LC-MS (M⁺+1): 493.38.

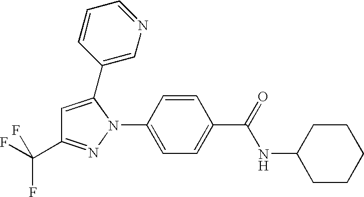

N-Cyclohexyl-4-(5-pyridin-3-yl-3-trifluoromethyl-pyrazol-1-yl)-benzamide; LC-MS (M⁺+1): 415.46.

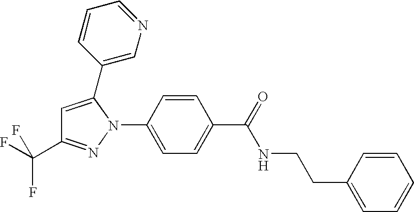

N-Phenethyl-4-(5-pyridin-3-yl-3-trifluoromethyl-pyrazol-1-yl)-benzamide; LC-MS (M⁺+1): 436.95

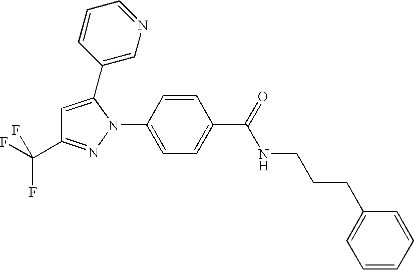

N-(3-Phenyl-propyl)-4-(5-pyridin-3-yl-3-trifluoromethyl-pyrazol-1-yl)-benzamide; LC-MS (M⁺+1): 451.55.

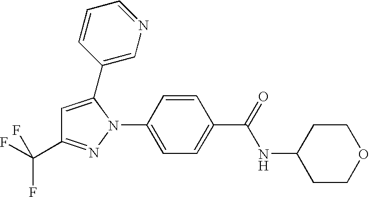

4-(5-Pyridin-3-yl-3-trifluoromethyl-pyrazol-1-yl)-N-(tetrahydro-pyran-4-yl)-benzamide LC-MS (M⁺+1): 417.41.

### Example 13

### N-[4-(3-Pyridin-3-yl-5-trifluoromethyl-pyrazol-1-yl)-benzyl]-nicotinamide

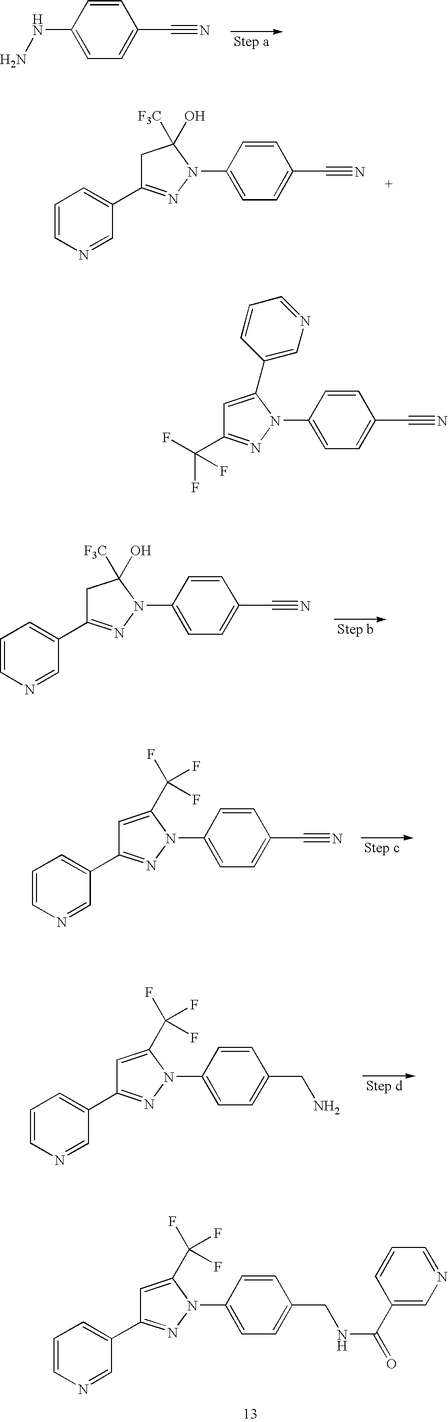

### Step a

A solution of 4-cyanophenylhydrazine (4.2 g, 24.9 mmol), acetic acid (3.0 mL) in ethanol (25 mL) is added to a stirred solution of 4,4,4-trifluoro-1-pyridin-3-yl-butane-1,3-dione (5.4 g, 24.9 mmol), prepared according to example 1, in ethanol (25 mL). The reaction mixture is heated at reflux for 16 hours, allowed to cool to room temperature, and the solid is filtered to give 4-(5-hydroxy-3-pyridin-3-yl-5-trifluoromethyl-4,5-dihydro-pyrazol-1-yl)-benzonitrile (1.5 g, 18%). The mother liquor is concentrated under vacuo, then triturated with ethyl acetate and filtered to give 4-(5-pyridin-3-yl-3-trifluoromethyl-pyrazol-1-yl)-benzonitrile (5.48 g, 70%).

### Step b

A solution of 4-(5-hydroxy-3-pyridin-3-yl-5-trifluoromethyl-4,5-dihydro-pyrazol-1-yl)-benzonitrile (620 mg, 1.87 mmol) in acetic acid (25 mL) is heated at reflux for 16 hours. The reaction mixture is concentrated under vacuo, diluted with ethyl acetate, washed with water (3×), dried over sodium sulfate and concentrated under vacuo to give 4-(3-pyridin-3-yl-5-trifluoromethyl-pyrazol-1-yl)-benzonitrile (356 mg, 60%).

### Step c

Lithium aluminum hydride (130 mg, 3.42 mmol) is added to a solution of 4-(3-pyridin-3-yl-5-trifluoromethyl-pyrazol-1-yl)-benzonitrile (356 mg; 1.13 mmol) in ether (5 mL) at 0° C. and is allowed to stir for 4 hours. The reaction mixture is quenched by the dropwise addition of water (130 μL), then 15% aqueous sodium hydroxide solution (130 μL) and water (390 μL), and is then stirred at room temperature for 15 min. The reaction mixture is filtered through celite, dried over sodium sulfate, and concentrated under vacuo to give 4-(3-pyridin-3-yl-5-trifluoromethyl-pyrazol-1-yl)-benzylamine (250 mg, 70%).

### Step d

A solution of 4-(3-pyridin-3-yl-5-trifluoromethyl-pyrazol-1-yl)-benzylamine (50 mg, 0.16 mmol), nicotinoyl chloride hydrochloride (50 mg, 0.35 mmol), triethylamine (100 μL, 0.72 mmol) in dichloromethane (2.5 mL) is stirred at room temperature for 4 hours. The reaction mixture is diluted with ethyl acetate, washed with water (25 mL, 3×), dried over sodium sulfate and concentrated under vacuo. The mixture is purified by chromatography to give the title compound (15 mg, 23%). LC-MS (M⁺+1): 423.35.

The following compounds are prepared according to example 13 by replacing the acyl chloride in step d with commercially available starting materials.

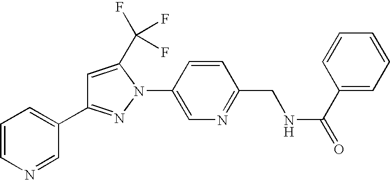

N-[5-(3-Pyridin-3-yl-5-trifluoromethyl-pyrazol-1-yl)-pyridin-2-ylmethyl]-benzamide; LC-MS (M⁺+1): 423.36.

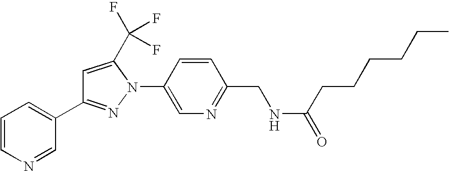

Heptanoic acid [5-(3-pyridin-3-yl-5-trifluoromethyl-pyrazol-1-yl)-pyridin-2-ylmethyl]-amide; LC-MS (M⁺+1): 431.47.

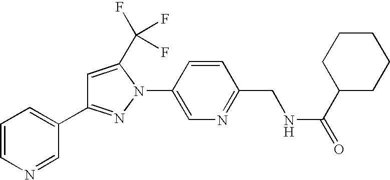

Cyclohexanecarboxylic acid [5-(3-pyridin-3-yl-5-trifluoromethyl-pyrazol-1-yl)-pyridin-2-ylmethyl]-amide; LC-MS (M⁺+1): 429.07.

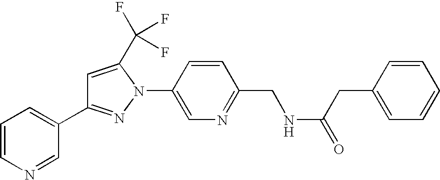

2-Phenyl-N-[5-(3-pyridin-3-yl-5-trifluoromethyl-pyrazol-1-yl)-pyridin-2-ylmethyl]-acetamide; LC-MS (M⁺+1): 437.36.

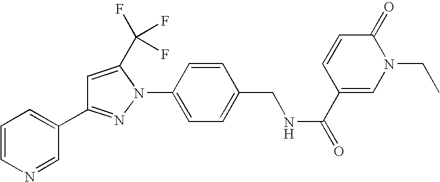

1-Ethyl-6-oxo-1,6-dihydro-pyridine-3-carboxylic acid 4-(3-pyridin-3-yl-5-trifluoromethyl-pyrazol-1-yl)-benzylamide; LC-MS (M⁺+1): 468.18.

### Example 14

### 1-Benzyl-3-[4-(3-pyridin-3-yl-5-trifluoromethyl-pyrazol-1-yl)-benzyl]-urea

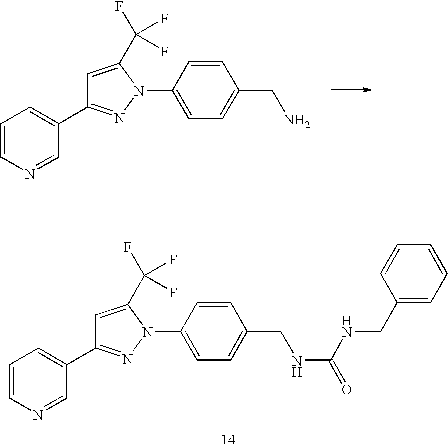

A solution of N-[4-(3-pyridin-3-yl-5-trifluoromethyl-pyrazol-1-yl)-benzyl]-nicotinamide 100 mg, 0.31 mmol), prepared according to example 13, benzylisocyanate (45 μL, 0.032 mmol) in acetonitrile (2.5 mL) is stirred at room temperature for 16 hours. The reaction mixture is filtered, and concentrated under vacuo. The mixture is purified by chromatography to give the title compound (26 mg, 18%). LC-MS (M⁺+1): 452.02.

The following compounds are prepared according to example 14 by replacing the isocyanate with commercially available starting materials.

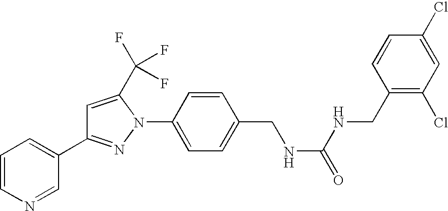

1-(2,4-Dichloro-benzyl)-3-[4-(3-pyridin-3-yl-5-trifluoromethyl-pyrazol-1-yl)-benzyl]-urea; LC-MS (M⁺+1): 519.96.

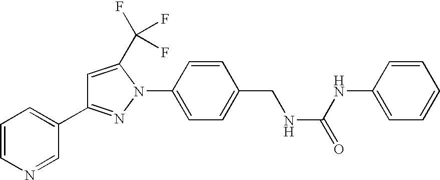

1-Phenyl-3-[4-(3-pyridin-3-yl-5-trifluoromethyl-pyrazol-1-yl)-benzyl]-urea; LC-MS (M⁺+1): 438.17.

### Example 15

### N-[4-(5-Pyridin-3-yl-3-trifluoromethyl-pyrazol-1-yl)-benzyl]-benzamide

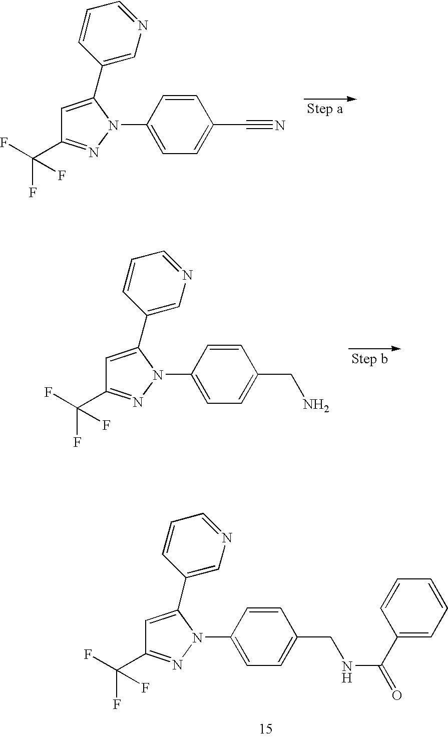

### Step a

Lithium aluminum hydride (50 mg, 1.32 mmol) is added to a solution of 4-(5-pyridin-3-yl-3-trifluoromethyl-pyrazol-1-yl)-benzonitrile (200 mg, 0.64 mmol), prepared according to example 13 in ether (5 mL) and at 0° C. and allowed to stir for 4 hours. The reaction mixture is quenched by the dropwise addition of 1 M aqueous sodium hydroxide solution (100 μL) and then stirred for 15 min. The reaction mixture is filtered through Celite, dried over sodium sulfate and concentrated under vacuo to give 4-(5-pyridin-3-yl-3-trifluoromethyl-pyrazol-1-yl)-benzylamine (100 mg, 49%).

### Step b

A solution of 4-(5-pyridin-3-yl-3-trifluoromethyl-pyrazol-1-yl)-benzylamine (50 mg, 0.16 mmol), benzoyl chloride (25 μL, 0.22 mmol), triethylamine (100 μL, 0.72 mmol) in dichloromethane (2.5 mL) is stirred at room temperature for 4 hours. The reaction mixture is diluted with ethyl acetate, washed with water (25 mL, 3×), dried over sodium sulfate and concentrated under vacuo. The crude mixture is purified by chromatography to give the title compound (32 mg, 48%). LC-MS (M⁺+1): 423.35.

The following compound is prepared according to example 15 by replacing the acyl chloride in step b with commercially available starting materials.

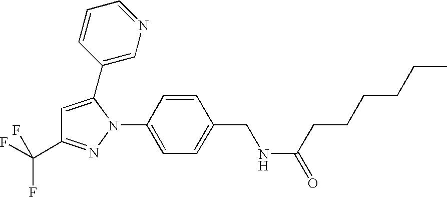

Heptanoic acid 4-(5-pyridin-3-yl-3-trifluoromethyl-pyrazol-1-yl)-benzylamide; LC-MS (M⁺+1): 431.43.

### Example 16

### 3-Methanesulfonyl-N-[5-(3-pyridin-3-yl-5-trifluoromethyl-pyrazol-1-yl)-pyridin-2-yl]-benzamide

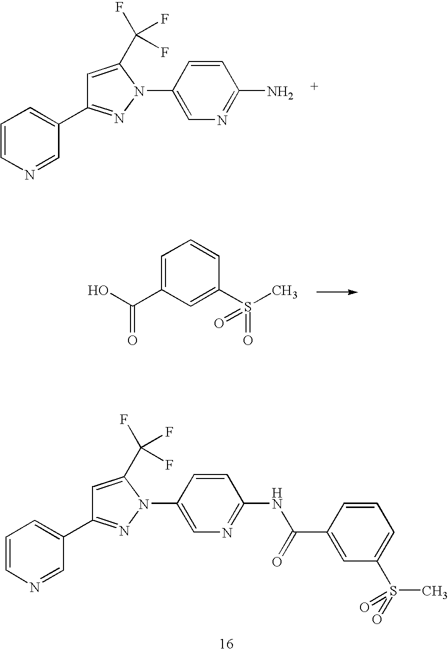

5-(3-Pyridin-3-yl-5-trifluoromethyl-pyrazol-1-yl)-pyridin-2-ylamine is prepared according to example 1. To the solution of 3-methanesulfonyl-benzoic acid (98.4 mg, 0.49 mmol) in tetrahydrofuran (2 mL) at room temperature is added oxalyl chloride (0.09 mL, 0.98 mmol) and 1 drop of dimethylformamide. The reaction mixture is stirred for 1 hour, and then the solvent and excess reagent are removed under vacuum. The residue is further dried under high vacuum for 1 hour after which pyridine (1.0 mL) is added to the residue. 5-(3-Pyridin-3-yl-5-trifluoromethyl-pyrazol-1-yl)-pyridin-2-ylamine (75 mg, 0.25 mmol) in pyridine (1.0 mL) is added to the solution and the mixture is stirred for 1 hour at room temperature. Water (15 mL) is added and the mixture is extracted with ethyl acetate (15 mL, 3×). The organic layers are combined, dried over sodium sulfate and concentrated under vacuum. The residue is purified by chromatography to give the title compound (108 mg, 90%). LC-MS (M⁺+1): 487.98.

The following compounds are prepared according to example 16 by replacing the carboxylic acid with commercially available starting materials.

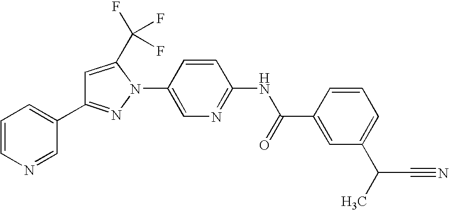

3-(Cyano-methyl-methyl)-N-[5-(3-pyridin-3-yl-5-trifluoromethyl-pyrazol-1-yl)-pyridin-2-yl]-benzamide; LC-MS (M⁺+1): 462.99.

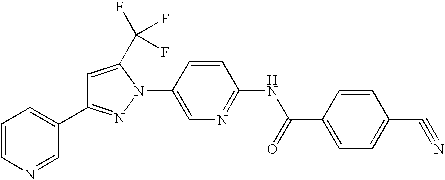

4-Cyano-N-[5-(3-pyridin-3-yl-5-trifluoromethyl-pyrazol-1-yl)-pyridin-2-yl]-benzamide; LC-MS (M⁺+1): 435.05

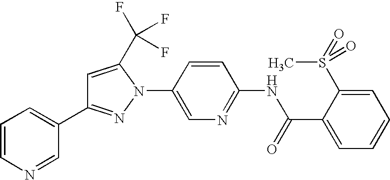

2-Methanesulfonyl-N-[5-(3-pyridin-3-yl-5-trifluoromethyl-pyrazol-1-yl)-pyridin-2-yl]-benzamide; LC-MS (M⁺+1): 487.91.

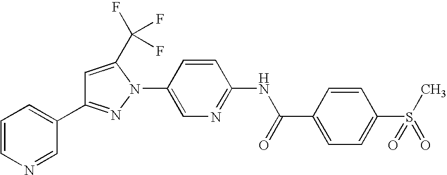

4-Methanesulfonyl-N-[5-(3-pyridin-3-yl-5-trifluoromethyl-pyrazol-1-yl)-pyridin-2-yl]-benzamide; LC-MS (M⁺+1): 488.06.

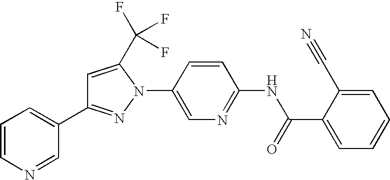

2-Cyano-N-[5-(3-pyridin-3-yl-5-trifluoromethyl-pyrazol-1-yl)-pyridin-2-yl]-benzamide; LC-MS (M⁺+1): 435.04.

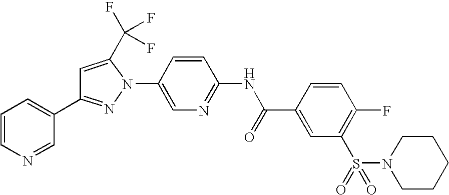

4-Fluoro-3-(piperidine-1-sulfonyl)-N-[5-(3-pyridin-3-yl-5-trifluoromethyl-pyrazol-1-yl)-pyridin-2-yl]-benzamide; LC-MS (M⁺+1): 575.03.

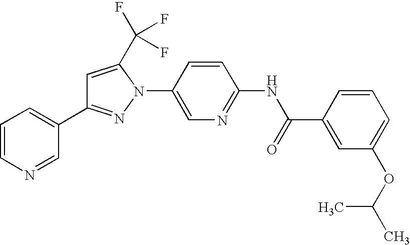

3-Isopropoxy-N-[5-(1-pyridin-3-yl-5-trifluoromethyl-pyrazol-1-yl)-pyridin-2-yl]-benzamide; LC-MS (M⁺+1): 468.11.

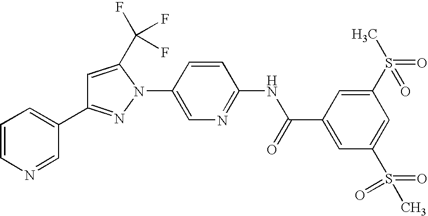

3,5-Bis-methanesulfonyl-N-[5-(3-pyridin-3-yl-5-trifluoromethyl-pyrazol-1-yl)-pyridin-2-yl]-benzamide; LC-MS (M⁺+1): 566.06.

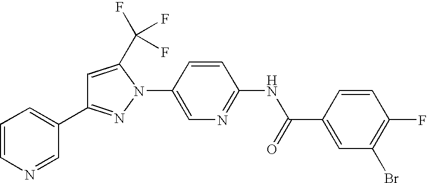

3-Bromo-4-fluoro-N-[5-(3-pyridin-3-yl-5-trifluoromethyl-pyrazol-1-yl)-pyridin-2-yl]-benzamide; LC-MS (M⁺): 506.32.

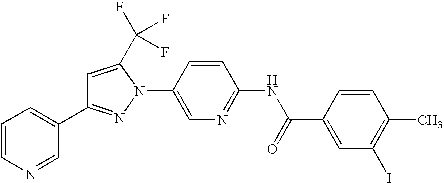

3-Iodo-4-methyl-N-[5-(3-pyridin-3-yl-5-trifluoromethyl-pyrazol-1-yl)-pyridin-2-yl]-benzamide; LC-MS (M⁺+1): 550.01.

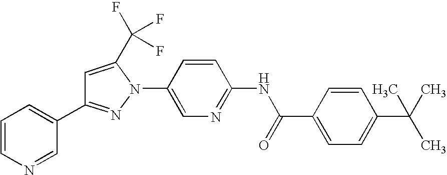

4-tert-Butyl-N-[5-(3-pyridin-3-yl-5-trifluoromethyl-pyrazol-1-yl)-pyridin-2-yl]-benzamide; LC-MS (M⁺+1): 466.16.

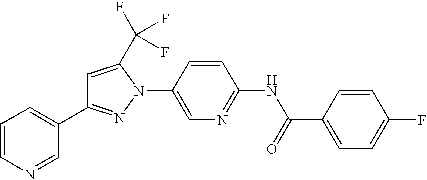

4-Fluoro-N-[5-(3-pyridin-3-yl-5-trifluoromethyl-pyrazol-1-yl)-pyridin-2-yl]-benzamide; LC-MS (M⁺+1): 428.07.

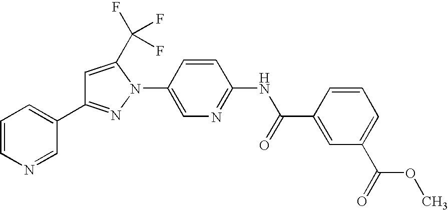

N-[5-(3-Pyridin-3-yl-5-trifluoromethyl-pyrazol-1-yl)-pyridin-2-yl]-isophthalamic acid methyl ester; LC-MS (M⁺+1): 468.09.

### Example 17

### Biphenyl-3,4′-dicarboxylic Acid 4′-methylamide 3-{[5-(3-pyridin-3-yl-5-trifluoromethyl-pyrazol-1-yl)-pyridin-2-yl]-amide}

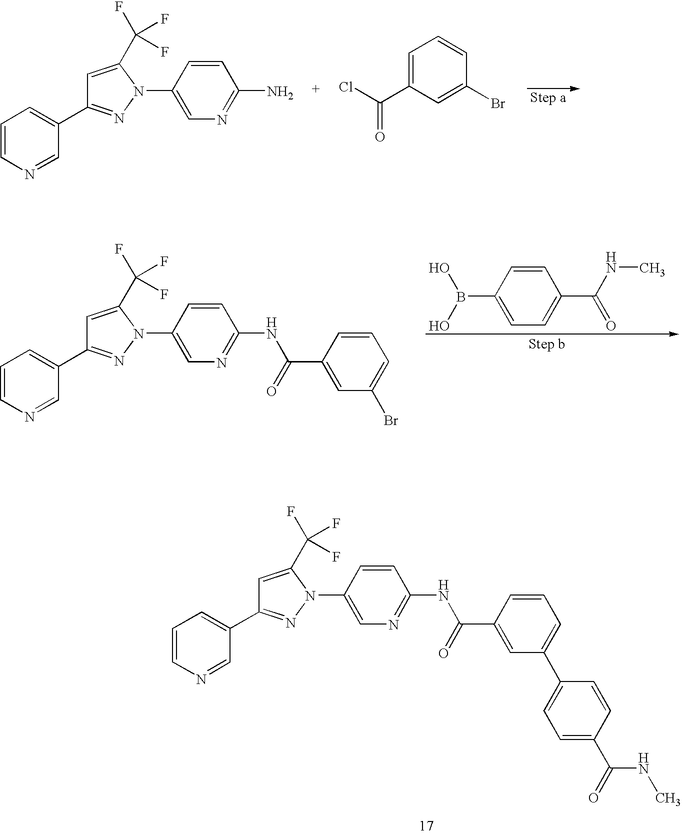

### Step a

5-(3-Pyridin-3-yl-5-trifluoromethyl-pyrazol-1-yl)-pyridin-2-ylamine is prepared according to example 1. To the solution of 5-(3-Pyridin-3-yl-5-trifluoromethyl-pyrazol-1-yl)-pyridin-2-ylamine (200 mg, 0.66 mmol) in pyridine (2.0 mL) at room temperature is added 3-bromo-benzoyl chloride (0.22 mL, 1.64 mmol) and the mixture is stirred for 1 hour. Then water (20 mL) is added and a pale yellow solid forms. The solid is filtered, washed with water, and then re-dissolved in tetrahydrofuran (20 mL). Hydrazine (0.2 mL of a 1 M solution in tetrahydrofuran) is added and the mixture is stirred for 15 min before all the solvent is removed under vacuum to give 320 mg of 3-Bromo-N-[5-(3-pyridin-3-yl-5-trifluoromethyl-pyrazol-1-yl)-pyridin-2-yl]-benzamide with 85% purity. This solid is used in the next step without further purification.

### Step b

3-Bromo-N-[5-(3-pyridin-3-yl-5-trifluoromethyl-pyrazol-1-yl)-pyridin-2-yl]-benzamide (100 mg 0.18 mmol), 4-(N-methylaminocarbonyl)phenylboronic acid (63 mg 0.35 mmol), tetrakis(triphenylphosphine)palladium(0) (31 mg, 0.03 mmol) and cesium carbonate (57 mg, 0.18 mmol) are placed in a microwave reaction tube and dimethylformamide (2 mL) is added. The mixture is microwaved at 120° C. for 2.5 hours. Water (7 mL) is added and the mixture is extracted with ethyl acetate (5 mL, 3×). The organic layers are combined, washed with water (5 mL, 3×) and dried over sodium sulfate to give crude product. The residue is purified by chromatography to give the title compound (47 mg, 50%). LC-MS (M⁺+1): 543.03.

The following compounds are prepared according to example 17 by replacing the boronic acid in step b with commercially available starting materials.

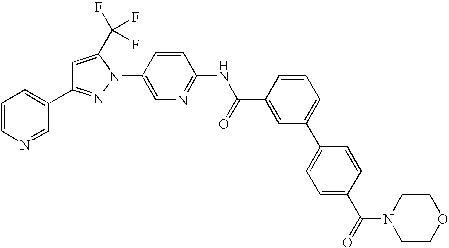

4′-(Morpholine-4-carbonyl)-biphenyl-3-carboxylic acid [5-(3-pyridin-3-yl-5-trifluoromethyl-pyrazol-1-yl)-pyridin-2-yl]-amide; LC-MS (M⁺+1): 599.08.

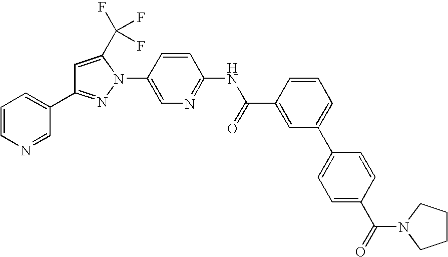

4′-(Pyrrolidine-1-carbonyl)-biphenyl-3-carboxylic acid [5-(3-pyridin-3-yl-5-trifluoromethyl-pyrazol-1-yl)-pyridin-2-yl]-amide LC-MS (M⁺+1): 583.02.

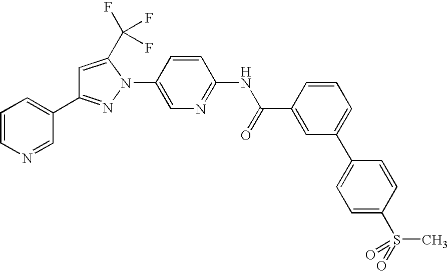

4′-Methanesulfonyl-biphenyl-3-carboxylic acid [5-(3-pyridin-3-yl-5-trifluoromethyl-pyrazol-1-yl)-pyridin-2-yl]-amide, LC-MS (M⁺+1): 564.03.

### Example 18

### N-Methyl-N′-[5-(3-pyridin-3-yl-5-trifluoromethyl-pyrazol-1-yl)-pyridin-2-yl]-isophthalamide

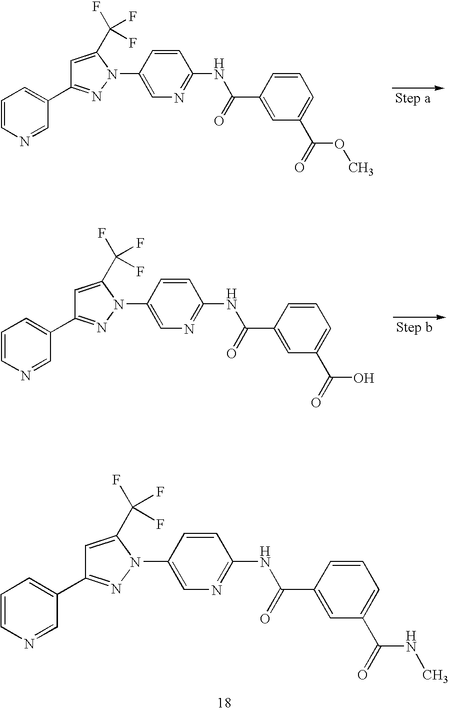

### Step a

N-[5-(3-Pyridin-3-yl-5-trifluoromethyl-pyrazol-1-yl)-pyridin-2-yl]-isophthalamic acid methyl ester is prepared according to example 16. N-[5-(3-Pyridin-3-yl-5-trifluoromethyl-pyrazol-1-yl)-pyridin-2-yl]-isophthalamic acid methyl ester (1.5 g, 3.2 mmol) is dissolved in dioxane (60 mL) and lithium hydroxide monohydrate (269 mg, 6.4 mmol) in water (10 mL) is added. The mixture is stirred for 6.5 hours at room temperature. Then 1 M aqueous HCl (8.0 mL) is added and a white solid forms. Water (200 mL) is added and the mixture is extracted with ethyl acetate (200 mL, 3×). The organic layers are combined and dried over sodium sulfate. Removal of the solvent under vacuum affords N-[5-(3-pyridin-3-yl-5-trifluoromethyl-pyrazol-1-yl)-pyridin-2-yl]-isophthalamic acid (1.4 g, 98%) which is used in the next step without further purification.

### Step b

To the suspension of N-[5-(3-pyridin-3-yl-5-trifluoromethyl-pyrazol-1-yl)-pyridin-2-yl]-isophthalamic acid (800 mg, 1.77 mmol) in tetrahydrofuran (25 mL) at room temperature are added methylamine (4.4 mL of a 2 M in tetrahydrofuran, 8.8 mmol) and 1-hydroxybenzotriazole (1.15 g, 8.5 mmol). 1-(3-Dimethylaminopropyl)-3-ethylcarbodiimide (1.44 g, 7.5 mmol) is then added in small portions and the reaction mixture is stirred for 1 hour. Saturated aqueous sodium bicarbonate solution (20 mL) and water (20 mL) are added and the mixture is extracted with ethyl acetate (60 mL, 3×). The organic layers are combined and dried over sodium sulfate to give the crude product. Purification by re-crystallization and chromatography affords the title compound (682 mg, 83%). LC-MS (M⁺+1): 466.99.

The following compounds are prepared according to example 18 by replacing the amine in step b with commercially available starting materials.

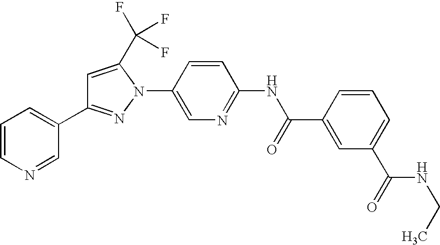

N-Ethyl-N′-[5-(3-pyridin-3-yl-5-trifluoromethyl-pyrazol-1-yl)-pyridin-2-yl]-isophthalamide; LC-MS (M⁺+1): 481.08.

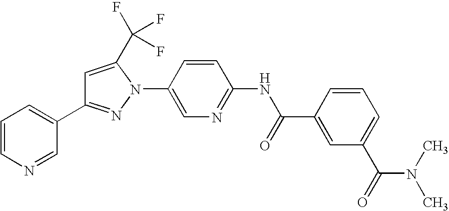

N,N-Dimethyl-N′-[5-(3-pyridin-3-yl-5-trifluoromethyl-pyrazol-1-yl)-pyridin-2-yl]-isophthalamide; LC-MS (M⁺+1): 481.11.

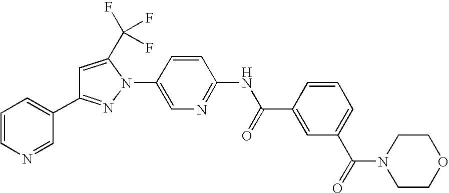

3-(Morpholine-4-carbonyl)-N-[5-(3-pyridin-3-yl-5-trifluoromethyl-pyrazol-1-yl)-pyridin-2-yl]-benzamide; LC-MS (M⁺+1): 523.16.

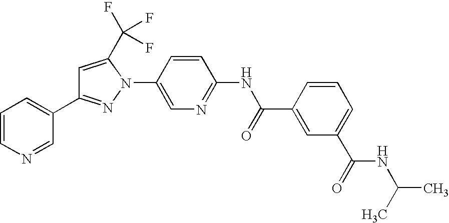

N-Isopropyl-N′-[5-(3-pyridin-3-yl-5-trifluoromethyl-pyrazol-1-yl)-pyridin-2-yl]-isophthalamide; LC-MS (M⁺+1): 495.16.

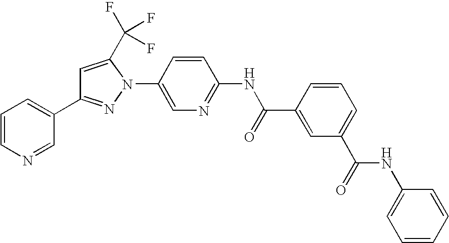

N-Phenyl-N′-[5-(3-pyridin-3-yl-5-trifluoromethyl-pyrazol-1-yl)-pyridin-2-yl]-isophthalamide; LC-MS (M⁺+1): 529.16.

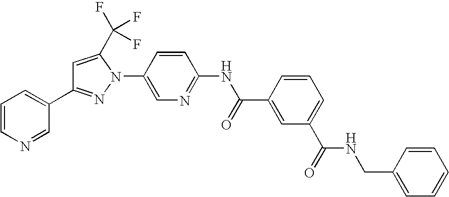

N-Benzyl-N′-[5-(3-pyridin-3-yl-5-trifluoromethyl-pyrazol-1-yl)-pyridin-2-yl]-isophthalamide; LC-MS (M⁺+1): 543.18.

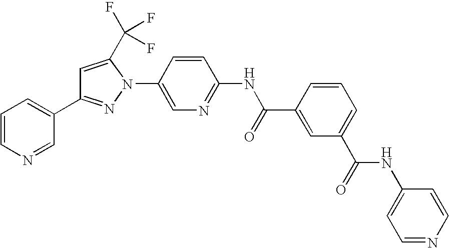

N-Pyridin-4-yl-N′-[5-(3-pyridin-3-yl-5-trifluoromethyl-pyrazol-1-yl)-pyridin-2-yl]-isophthalamide; LC-MS (M⁺+1): 530.15.

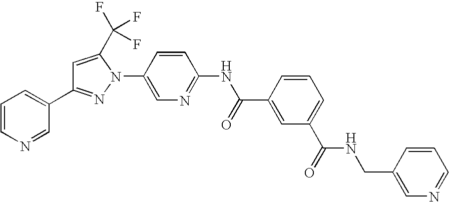

N-Pyridin-3-ylmethyl-N′-[5-(3-pyridin-3-yl-5-trifluoromethyl-pyrazol-1-yl)-pyridin-2-yl]-isophthalamide; LC-MS (M⁺+1): 544.16.

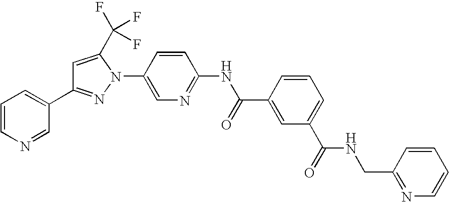

N-Pyridin-2-ylmethyl-N′-[5-(3-pyridin-3-yl-5-trifluoromethyl-pyrazol-1-yl)-pyridin-2-yl]-isophthalamide; LC-MS (M⁺+1): 544.16.

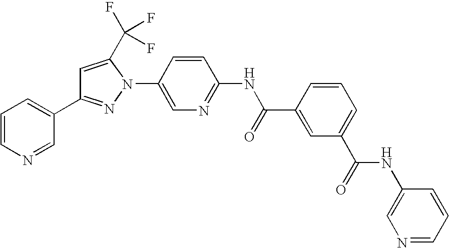

N-Pyridin-3-yl-N′-[5-(3-pyridin-3-yl-5-trifluoromethyl-pyrazol-1-yl)-pyridin-2-yl]-isophthalamide; LC-MS (M⁺+1): 530.13.

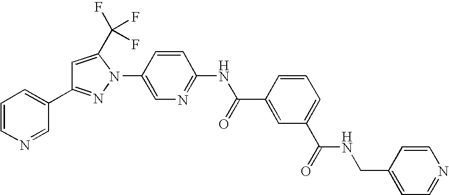

N-Pyridin-4-ylmethyl-N′-[5-(3-pyridin-3-yl-5-trifluoromethyl-pyrazol-1-yl)-pyridin-2-yl]-isophthalamide; LC-MS (M⁺+1): 544.16.

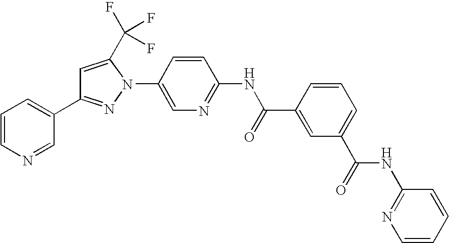

N-Pyridin-2-yl-N′-[5-(3-pyridin-3-yl-5-trifluoromethyl-pyrazol-1-yl)-pyridin-2-yl]-isophthalamide; LC-MS (M⁺+1): 530.13.

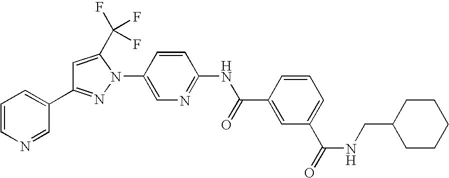

N-Cyclohexylmethyl-N′-[5-(3-pyridin-3-yl-5-trifluoromethyl-pyrazol-1-yl)-pyridin-2-yl]-isophthalamide; LC-MS (M⁺+1): 549.24.

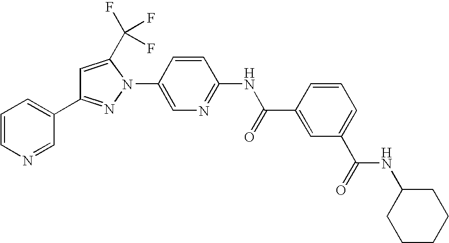

N-Cyclohexyl-N′-[5-(3-pyridin-3-yl-5-trifluoromethyl-pyrazol-1-yl)-pyridin-2-yl]-isophthalamide; LC-MS (M⁺+1): 535.23.

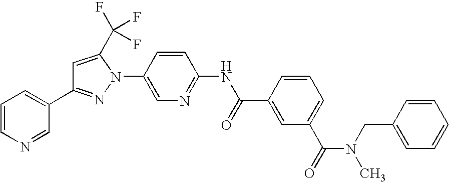

N-Benzyl-N-methyl-N′-[5-(3-pyridin-3-yl-5-trifluoromethyl-pyrazol-1-yl)-pyridin-2-yl]-isophthalamide; LC-MS (M⁺+1): 557.21.

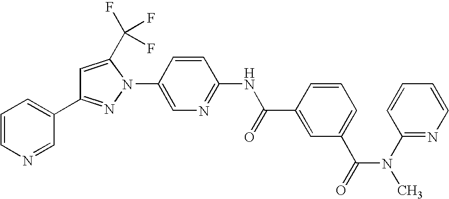

N-Methyl-N-pyridin-2-yl-N-[5-(3-pyridin-3-yl-5-trifluoromethyl-pyrazol-1-yl)-pyridin-2-yl]-isophthalamide; LC-MS (M⁺+1): 544.16.

### Example 19

### 3-Methylsulfamoyl-N-[5-(3-pyridin-3-yl-5-trifluoromethyl-pyrazol-1-yl)-pyridin-2-yl]-benzamide

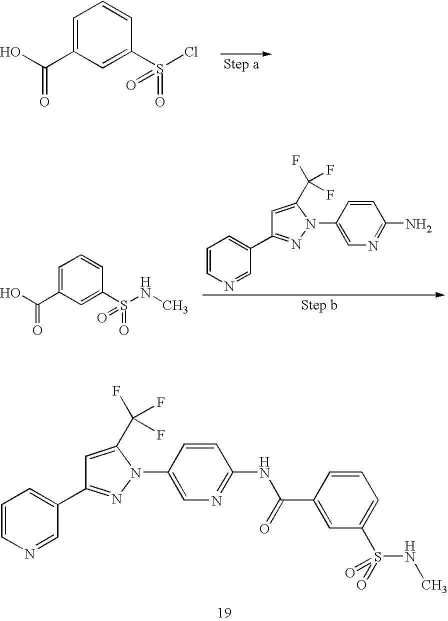

### Step a

Methylamine (1.1 mL of 2 M solution in tetrahydrofuran, 2.2 mmol) and triethylamine (0.6 mL, 4.3 mmol) are added to dichloromethane (10 mL) at 0° C. Then 3-chlorosulfonyl-benzoic acid (500 mg, 2.2 mmol) is added in small portions. The mixture is stirred for 30 minutes at 0° C. before 1 M aqueous HCl (8 mL) is added. The mixture is extracted with ethyl acetate (25 mL, 3×). The organic layers are combined and dried over sodium sulfate. Removal of solvent under vacuum affords 3-methylsulfamoyl-benzoic acid (480 mg) with 92% purity (2.1 mmol, 95%). This material is used in the next step without further purification.

### Step b

5-(3-Pyridin-3-yl-5-trifluoromethyl-pyrazol-1-yl)-pyridin-2-ylamine is prepared according to example 1. To the solution of 3-methylsulfamoyl-benzoic acid (250 mg, 1.07 mmol) in tetrahydrofuran (10 mL) at room temperature is added oxalyl chloride (0.19 mL, 2.14 mmol) and 0.1 mL of dimethylformamide. The reaction mixture is stirred for 1 hour, and then the solvent and excess reagent are removed under vacuum. The residue is further dried under high vacuum for 1 hour after which pyridine (2.5 mL), THF (0.5 mL) and dimethylformamide (1.0 mL) are added to the residue. 5-(3-Pyridin-3-yl-5-trifluoromethyl-pyrazol-1-yl)-pyridin-2-ylamine (80 mg, 0.26 mmol) in pyridine (2.5 mL) is added to the solution and the mixture is stirred for 1 hour at room temperature. Water (15 mL) is added and the mixture is extracted with ethyl acetate (15 mL, 3×). The organic layers are combined, dried over sodium sulfate and concentrated under vacuum. The residue is purified by chromatography to give the title compound (88 mg, 67%). LC-MS (M⁺+1): 502.94.

The following compounds are prepared according to example 19 by replacing the amine in step a with commercially available starting materials.

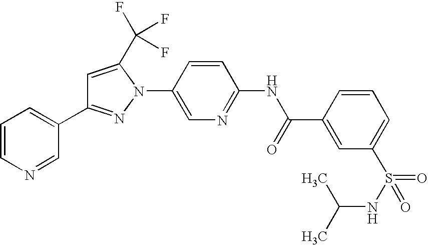

3-Isopropylsulfamoyl-N-[5-(3-pyridin-3-yl-5-trifluoromethyl-pyrazol-1-yl)-pyridin-2-yl]-benzamide; LC-MS (M⁺+1): 530.98.

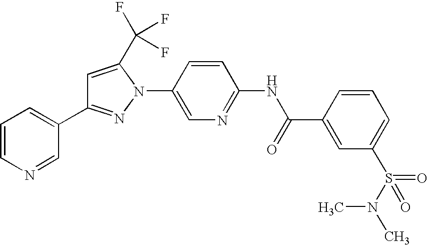

3-Dimethylsulfamoyl-N-[5-(3-pyridin-3-yl-5-trifluoromethyl-pyrazol-1-yl)-pyridin-2-yl]-benzamide; LC-MS (M⁺+1): 516.97.

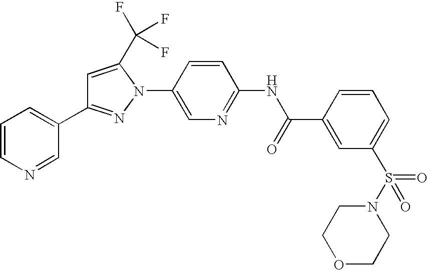

3-(Morpholine-4-sulfonyl)-N-[5-(3-pyridin-3-yl-5-trifluoromethyl-pyrazol-1-yl)-pyridin-2-yl]-benzamide; LC-MS (M⁺+1): 559.01.

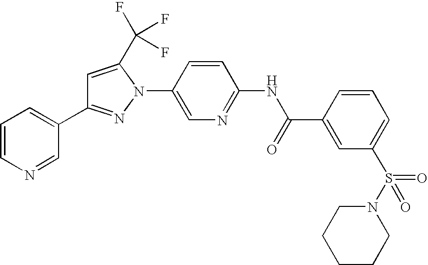

3-(Piperidine-1-sulfonyl)-N-[5-(3-pyridin-3-yl-5-trifluoromethyl-pyrazol-1-yl)-pyridin-2-yl]-benzamide; LC-MS (M⁺+1): 557.06.

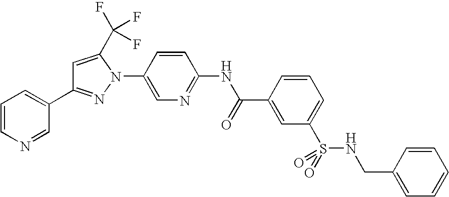

3-Benzylsulfamoyl-N-[5-(3-pyridin-3-yl-5-trifluoromethyl-pyrazol-1-yl)-pyridin-2-yl]-benzamide; LC-MS (M⁺+1): 579.18.

### Example 20

### N-Methyl-N′-[5-(3-pyridin-3-yl-5-trifluoromethyl-pyrazol-1-yl)-pyridin-2-yl]-terephthalamide

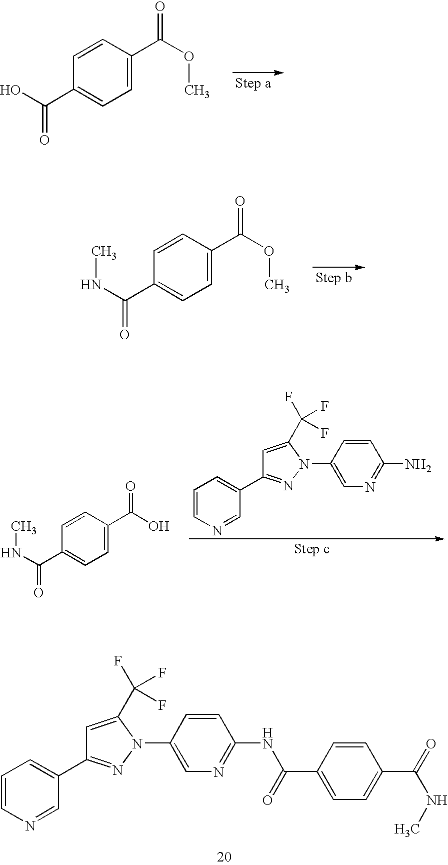

### Step a

To a solution of terephthalic acid monomethyl ester (500 mg, 2.8 mmol) in toluene (2.5 mL) at room temperature are added dimethylformamide (10 drops) and thionyl chloride (0.41 mL, (5.6 mmol). The mixture is heated up to 92° C. for 1 hour. It is then cooled to room temperature and the solvent and excess thionyl chloride are removed under vacuum. The residue is dried for 1 hour under high vacuum and pyridine (1 mL) and dimethylformamide (1 mL) are added to dissolve the residue. Methylamine (1.4 mL of a 2 M solution in tetrahydrofuran, 2.8 mmol) is then added to the reaction and the mixture is stirred for 45 min. After the reaction is complete, water (5 mL) is added and the mixture is extracted with ethyl acetate (5 mL, 3×). The organic layers are combined and dried over sodium sulfate. Removal of the solvent under vacuum affords N-Methyl-terephthalamic acid methyl ester (380 mg, 71%) which is used in the next step without further purification.

### Step b

Lithium hydroxide monohydrate (120 mg, 2.9 mmol), dissolved in water (3 mL), is added to a solution of N-methyl-terephthalamic acid methyl ester (380 mg, 2.0 mmol) in dioxane (11 mL) at room temperature. The mixture is stirred for 16 hours, and aqueous 1 M HCl (6 mL) is added and the mixture is extracted with ethyl acetate (20 mL, 3×). The organic layers are combined and dried over sodium sulfate. Removal of the solvent under vacuum affords N-methyl-terephthalamic acid with 95% purity (334 mg, 90%).

### Step c

5-(3-Pyridin-3-yl-5-trifluoromethyl-pyrazol-1-yl)-pyridin-2-ylamine is prepared according to example 1. To the solution of N-methyl-terephthalamic acid (104 mg, 0.58 mmol) in tetrahydrofuran (5.0 mL) at room temperature is added oxalyl chloride (0.10 mL, 1.15 mmol) and 0.1 mL of dimethylformamide. The reaction mixture is stirred for 1 hour, and then the solvent and excess reagent are removed under vacuum. The residue is further dried under high vacuum for 1 hour after which pyridine (2.0 mL) and dimethylformamide (1.0 mL) are added to the residue. 5-(3-Pyridin-3-yl-5-trifluoromethyl-pyrazol-1-yl)-pyridin-2-ylamine (75 mg, 0.25 mmol) in pyridine (2.0 mL) is added to the solution and the mixture is stirred for 1 hour at room temperature. Water (5 mL) is added and the mixture is extracted with ethyl acetate (20 mL, 3×). The organic layers are combined, dried over sodium sulfate and concentrated under vacuum. The residue is purified by chromatography and re-crystallization to give the title compound (21.8 mg, 19%). LC-MS (M⁺+1): 467.13.

### Example 21

### 3-(Propane-2-sulfonyl)-N-[5-(3-pyridin-3-yl-5-trifluoromethyl-pyrazol-1-yl)-pyridin-2-yl]-benzamide

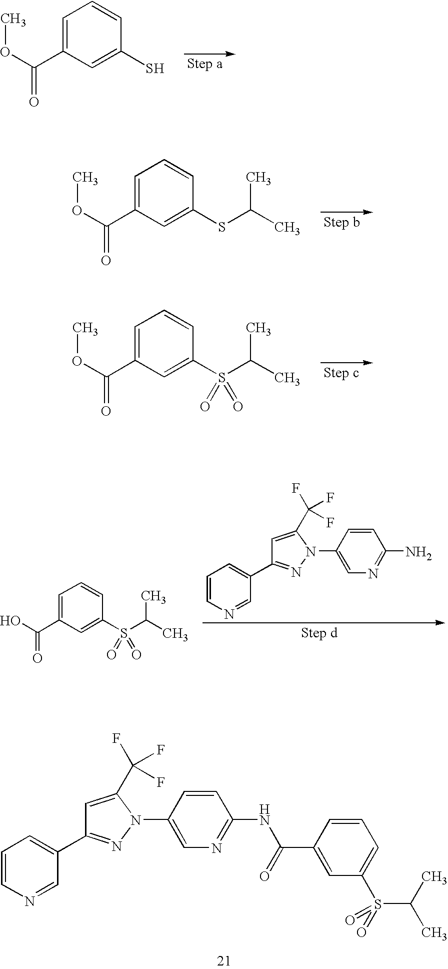

### Step a

To a solution of 3-mercaptobenzoic acid methyl ester (637 mg, 3.8 mmol) and 2-iodo-propane (0.57 mL, 5.7 mmol) in tetrahydrofuran (5.0 mL) at room temperature is added sodium hydride (303 mg of a 60% dispersion in mineral oil, 7.6 mmol) in small portions over 5 min. The mixture is stirred for 1 hour at room temperature, and then aqueous 1 M HCl (15 mL) is added. The mixture is extracted with ethyl acetate (30 mL, 3×) and the organic layers are combined and dried over sodium sulfate. Removal of the solvent under vacuum affords the crude 3-isopropylsulfanyl-benzoic acid methyl ester (798 mg, 3.8 mmol) which is used in the next step without further purification.

### Step b

To a solution of crude 3-isopropylsulfanyl-benzoic acid methyl ester (798 mg, 3.8 mmol) in dichloromethane at room temperature is added 3-chloroperoxybenzoic acid (2.0 g, 8.1 mmol) in small portions over 10 min. The mixture is stirred for 1 hour and aqueous saturated sodium bicarbonate solution (40 mL) is added. The aqueous phase is then extracted with ethyl acetate (60 mL, 3×), the organic layers are combined, and washed with aqueous saturated sodium bicarbonate solution (10 mL, 2×) and water (10 mL). The organic layer is then dried over sodium sulfate and removal of the solvent under vacuum affords crude 3-(propane-2-sulfonyl)-benzoic acid methyl ester which is used in the next step without further purification.

### Step c

Lithium hydroxide monohydrate (100 mg, 2.4 mmol), dissolved in water (2.0 mL), is added to the solution of crude 3-(propane-2-sulfonyl)-benzoic acid methyl ester (290 mg, 1.20 mmol) in dioxane (3.0 mL). The mixture is stirred for 1 hour and aqueous 1 M HCl (5 mL) is added. The aqueous phase is then extracted with ethyl acetate (20 mL, 3×) and the organic layers are combined and dried over sodium sulfate to give crude 3-(propane-2-sulfonyl)-benzoic acid (241 mg, 1.06 mmol) which is pure enough for the next step.

### Step d

5-(3-Pyridin-3-yl-5-trifluoromethyl-pyrazol-1-yl)-pyridin-2-ylamine is prepared according to example 1. To the solution of 3-(propane-2-sulfonyl)-benzoic acid (240 mg, 1.05 mmol) in tetrahydrofuran (5.0 mL) at room temperature are added oxalyl chloride (0.18 mL, 2.10 mmol) and 0.2 mL of dimethylformamide. The reaction mixture is stirred for 1 hour, and then the solvent and excess reagent are removed under vacuum. The residue is further dried under high vacuum for 1 hour after which pyridine (2.0 mL) is added to the residue. 5-(3-Pyridin-3-yl-5-trifluoromethyl-pyrazol-1-yl)-pyridin-2-ylamine (75 mg, 0.25 mmol) in pyridine (2.0 mL) is added to the suspension and the mixture is stirred for 1 hour at room temperature. Water (10 mL) is added and the mixture is extracted with ethyl acetate (20 mL, 3×). The organic layers are combined, dried over sodium sulfate and concentrated under vacuum. The residue is purified by chromatography to give the title compound (240 mg, 89%). LC-MS (M⁺+1): 516.17.

### Example 22

### Cyclohexanecarboxylic acid [6-(3-pyridin-3-yl-5-trifluoromethyl-pyrazol-1-yl)-pyridin-3-yl]-amide

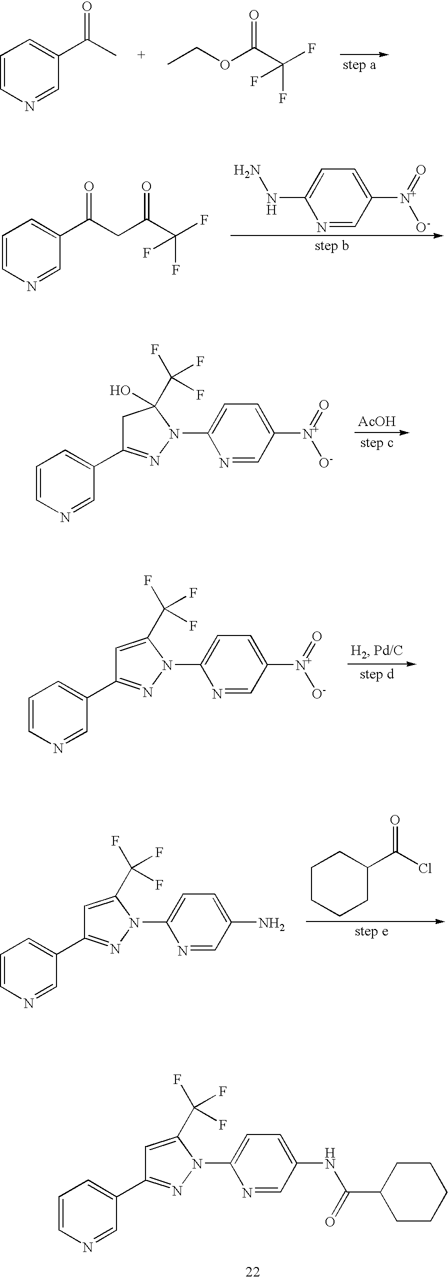

### Step a

To a solution of ethyl trifluoroacetate (24 mL, 0.202 mol) in ether (250 mL) are added sodium methoxide (10.9 g, 0.202 mol) and 3-acetylpyridine (22 mL, 0.202 mol) at room temperature. The solution is heated at reflux for 2 hours. The reaction is cooled to room temperature and water (100 mL) is added. The resulting mixture is extracted with ether (100 mL) and the aqueous layer is collected. The aqueous solution is acidified to pH 4 with acetic acid. The precipitate is collected by filtration and dried under vacuum. The resulting product, 4,4,4-trifluoro-1-pyridin-3-yl-butane-1,3-dione (32.8 g, 75.5%), is used without further purification.

### Step b

A solution of 4,4,4-trifluoro-1-pyridin-3-yl-butane-1,3-dione (651 mg, 3 mmol) and 5-nitro-2-hydrazino-pyridine (462 mg, 3 mmol) in anhydrous ethanol (15 mL) is heated in a sealed flask at 50° C. for 24 hours. The precipitate is filtered off and washed with ether. The resulting product, 2-(5-Nitro-pyridin-2-yl)-5-pyridin-3-yl-3-trifluoromethyl-3,4-dihydro-2H-pyrazol-3-ol (465 mg, 44%) is used without further purification.

### Step c

2-(5-Nitro-pyridin-2-yl)-5-pyridin-3-yl-3-trifluoromethyl-3,4-dihydro-2H-pyrazol-3-ol (465 mg, 1.32 mmol) is dissolved in acetic acid (3 mL) and the solution is microwaved at 200° C. for 2 hours. The solution is evaporated and the resulting product, 5-Nitro-2-(3-pyridin-3-yl-5-trifluoromethyl-pyrazol-1-yl)-pyridine (440 mg, 100%) is used without further purification.

### Step d

5-Nitro-2-(3-pyridin-3-yl-5-trifluoromethyl-pyrazol-1-yl)-pyridine (150 mg, 0.45 mmol) is dissolved in ethanol (15 mL) with gentle heating. Catalytic amounts of 10% palladium on carbon are added and the solution is hydrogenated under balloon pressure overnight. The mixture is filtered through Celite and evaporated. The resulting product, 6-(3-Pyridin-3-yl-5-trifluoromethyl-pyrazol-1-yl)-pyridin-3-ylamine (125 mg, 91%) is used without further purification.

### Step e

6-(3-Pyridin-3-yl-5-trifluoromethyl-pyrazol-1-yl)-pyridin-3-ylamine (20 mg, 0.065 mmol) is dissolved in tetrahydrofuran (2 mL) and N,N-diisopropylethylamine (35 μL, 0.2 mmol) and cyclohexanecarbonyl chloride (14 μL, 0.1 mmol) is added successively. The solution is stirred for 30 min after which it is diluted with saturated aqueous ammonium chloride solution. The aqueous phase is extracted with dichloromethane and the combined organic phases are dried over magnesium sulfate. The solvent is evaporated and the residue is purified by chromatography to afford the title compound (8 mg, 30%). LC-MS (M⁺+1): 416.40.

The following compounds are prepared according to example 22 by replacing the acyl chloride in step e with commercially available starting materials.

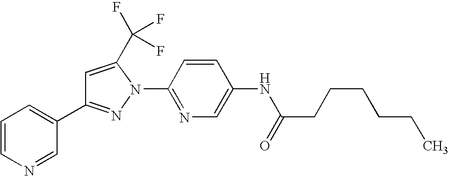

Heptanoic acid [6-(3-pyridin-3-yl-5-trifluoromethyl-pyrazol-1-yl)-pyridin-3-yl]-amide; LC-MS (M⁺+1): 418.39.

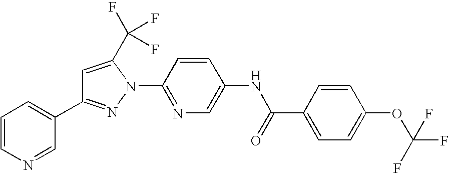

N-[6-(3-Pyridin-3-yl-5-trifluoromethyl-pyrazol-1-yl)-pyridin-3-yl]-4-trifluoromethoxy-benzamide; LC-MS (M⁺+1): 494.29.

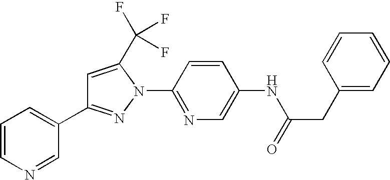

2-Phenyl-N-[6-(3-pyridin-3-yl-5-trifluoromethyl-pyrazol-1-yl)-pyridin-3-yl]-acetamide; LC-MS (M⁺+1): 424.36.

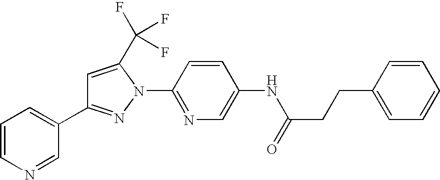

3-Phenyl-N-[6-(3-pyridin-3-yl-5-trifluoromethyl-pyrazol-1-yl)-pyridin-3-yl]-propionamide; LC-MS (M⁺+1): 438.39.

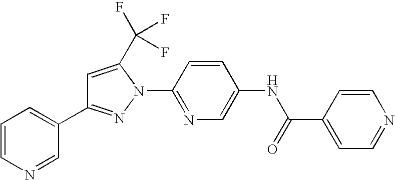

N-[6-(3-Pyridin-3-yl-5-trifluoromethyl-pyrazol-1-yl)-pyridin-3-yl]-isonicotinamide; LC-MS (M⁺+1): 411.34.

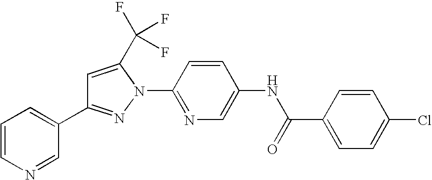

4-Chloro-N-[6-(3-pyridin-3-yl-5-trifluoromethyl-pyrazol-1-yl)-pyridin-3-yl]-benzamide; LC-MS (M⁺+1): 444.30.

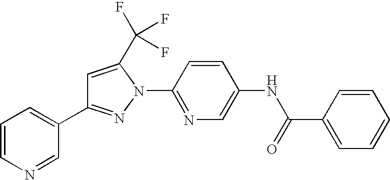

N-[6-(3-Pyridin-3-yl-5-trifluoromethyl-pyrazol-1-yl)-pyridin-3-yl]-benzamide; LC-MS (M⁺+1): 410.33.

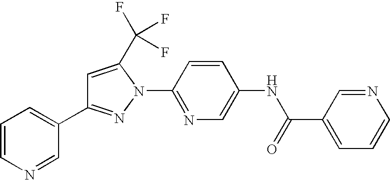

N-[6-(3-Pyridin-3-yl-5-trifluoromethyl-pyrazol-1-yl)-pyridin-3-yl]-nicotinamide; LC-MS (M⁺+1): 411.35.

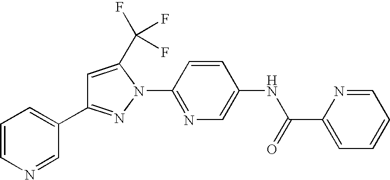

Pyridine-2-carboxylic acid [6-(3-pyridin-3-yl-5-trifluoromethyl-pyrazol-1-yl)-pyridin-3-yl]-amide; LC-MS (M⁺+1): 411.34.

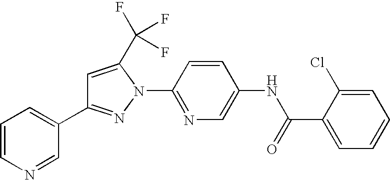

2-Chloro-N-[6-(3-pyridin-3-yl-5-trifluoromethyl-pyrazol-1-yl)-pyridin-3-yl]-benzamide; LC-MS (M⁺+1): 444.28

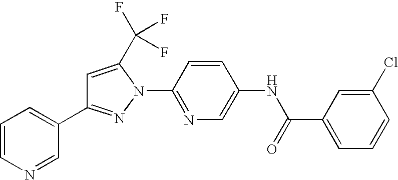

3-Chloro-N-[6-(3-pyridin-3-yl-5-trifluoromethyl-pyrazol-1-yl)-pyridin-3-yl]-benzamide; LC-MS (M⁺+1): 444.29.

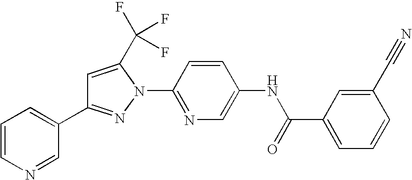

3-Cyano-N-[6-(3-pyridin-3-yl-5-trifluoromethyl-pyrazol-1-yl)-pyridin-3-yl]-benzamide; LC-MS (M⁺+1): 435.31.

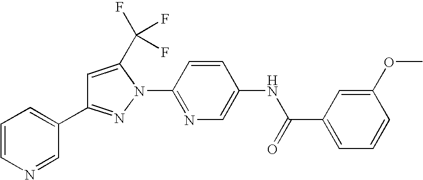

3-Methoxy-N-[6-(3-pyridin-3-yl-5-trifluoromethyl-pyrazol-1-yl)-pyridin-3-yl]-benzamide; LC-MS (M⁺+1): 440.34.

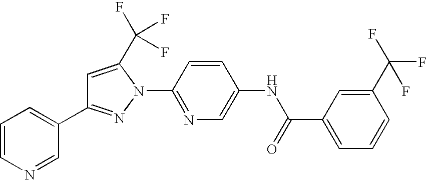

N-[6-(3-Pyridin-3-yl-5-trifluoromethyl-pyrazol-1-yl)-pyridin-3-yl]-3-trifluoromethyl-benzamide; LC-MS (M⁺+1): 478.29.

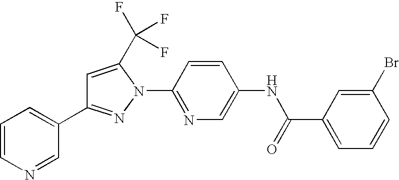

3-Bromo-N-[6-(3-pyridin-3-yl-5-trifluoromethyl-pyrazol-1-yl)-pyridin-3-yl]-benzamide; LC-MS (M⁺+1): 489.20.

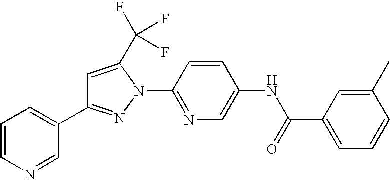

3-Methyl-N-[6-(3-pyridin-3-yl-5-trifluoromethyl-pyrazol-1-yl)-pyridin-3-yl]-benzamide; LC-MS (M⁺+1): 424.35.

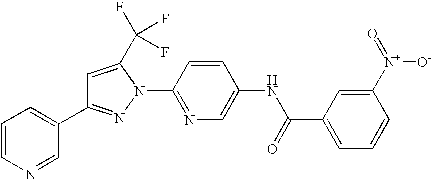

3-Nitro-N-[6-(3-pyridin-3-yl-5-trifluoromethyl-pyrazol-1-yl)-pyridin-3-yl]-benzamide; LC-MS (M⁺+1): 455.30.

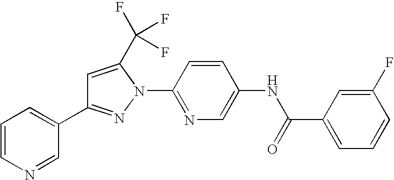

3-Fluoro-N-[6-(3-pyridin-3-yl-5-trifluoromethyl-pyrazol-1-yl)-pyridin-3-yl]-benzamide; LC-MS (M⁺+1): 428.37.

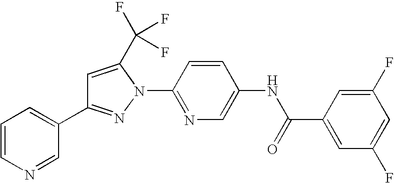

3,5-Difluoro-N-[6-(3-pyridin-3-yl-5-trifluoromethyl-pyrazol-1-yl)-pyridin-3-yl]-benzamide; LC-MS (M¹+1): 446.33.

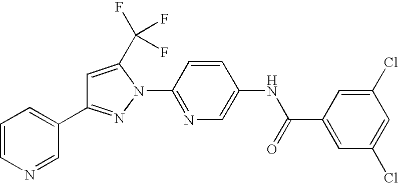

3,5-Dichloro-N-[6-(3-pyridin-3-yl-5-trifluoromethyl-pyrazol-1-yl)-pyridin-3-yl]-benzamide; LC-MS (M++e): 478.23.

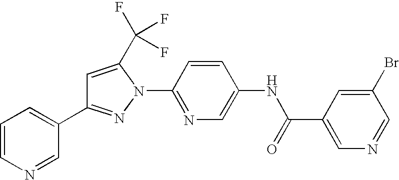

5-Bromo-N-[6-(3-pyridin-3-yl-5-trifluoromethyl-pyrazol-1-yl)-pyridin-3-yl]-nicotinamide; LC-MS (M⁺+1): 489.26.

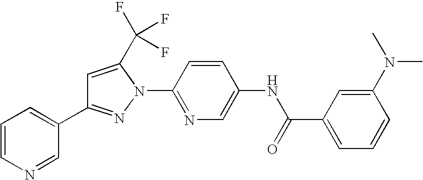

3-Dimethylamino-N-[6-(3-pyridin-3-yl-5-trifluoromethyl-pyrazol-1-yl)-pyridin-3-yl]-benzamide; LC-MS (M⁺+1): 453.38.

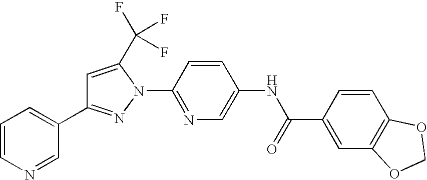

Benzo[1,3]dioxole-5-carboxylic acid [6-(3-pyridin-3-yl-5-trifluoromethyl-pyrazol-1-yl)-pyridin-3-yl]-amide; LC-MS (M⁺+1): 454.32.

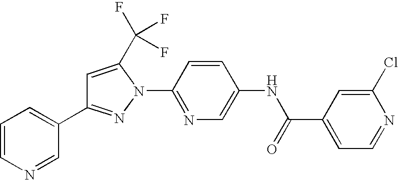

2-Chloro-N-[6-(3-pyridin-3-yl-5-trifluoromethyl-pyrazol-1-yl)-pyridin-3-yl]-isonicotinamide; LC-MS (M⁺+1): 445.31.

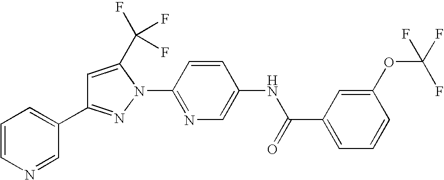

N-[6-(3-Pyridin-3-yl-5-trifluoromethyl-pyrazol-1-yl)-pyridin-3-yl]-3-trifluoromethoxy-benzamide; LC-MS (M⁺+1): 494.33.

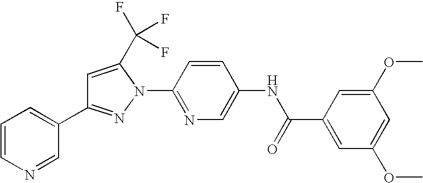

3,5-Dimethoxy-N-[6-(3-pyridin-3-yl-5-trifluoromethyl-pyrazol-1-yl)-pyridin-3-yl]-benzamide; LC-MS (M⁺+1): 470.35.

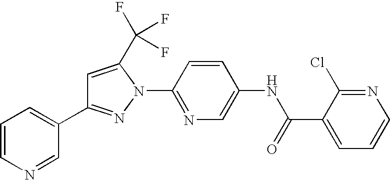

2-Chloro-N-[6-(3-pyridin-3-yl-5-trifluoromethyl-pyrazol-1-yl)-pyridin-3-yl]-nicotinamide; LC-MS (M⁺+1): 445.30.

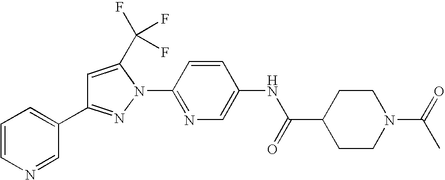

1-Acetyl-piperidine-4-carboxylic acid [6-(3-pyridin-3-yl-5-trifluoromethyl-pyrazol-1-yl)-pyridin-3-yl]-amide; LC-MS (M⁺+1): 459.40.

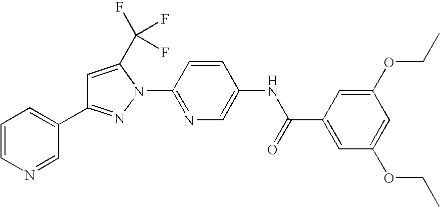

3,5-Diethoxy-N-[6-(3-pyridin-3-yl-5-trifluoromethyl-pyrazol-1-yl)-pyridin-3-yl]-benzamide; LC-MS (M⁺+1): 498.38.

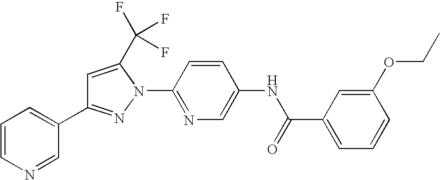

3-Ethoxy-N-[6-(3-pyridin-3-yl-5-trifluoromethyl-pyrazol-1-yl)-pyridin-3-yl]-benzamide; LC-MS (M⁺+1): 454.36.

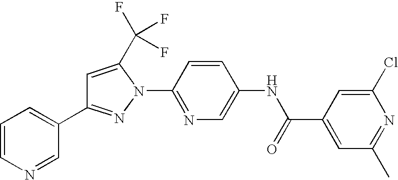

2-Chloro-6-methyl-N-[6-(3-pyridin-3-yl-5-trifluoromethyl-pyrazol-1-yl)-pyridin-3-yl]-isonicotinamide; LC-MS (M⁺+1): 459.27.

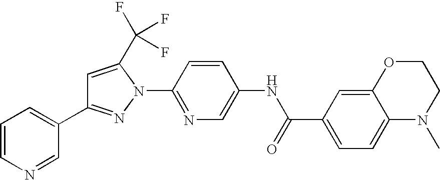

4-Methyl-3,4-dihydro-2H-benzo[1,4]oxazine-7-carboxylic acid [6-(3-pyridin-3-yl-5-trifluoromethyl-pyrazol-1-yl)-pyridin-3-yl]-amide; LC-MS (M⁺+1): 481.36.

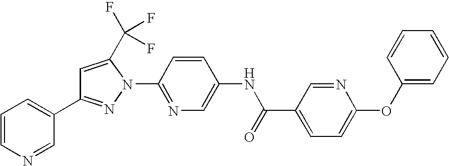

6-Phenoxy-N-[6-(3-pyridin-3-yl-5-trifluoromethyl-pyrazol-1-yl)-pyridin-3-yl]-nicotinamide; LC-MS (M¹+1): 503.05.

### Example 23

### 3-Amino-N-[6-(3-pyridin-3-yl-5-trifluoromethyl-pyrazol-1-yl)-pyridin-3-yl]-benzamide

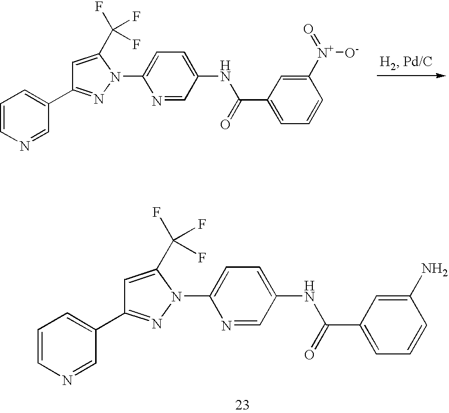

3-Nitro-N-[6-(3-pyridin-3-yl-5-trifluoromethyl-pyrazol-1-yl)-pyridin-3-yl]-benzamide (42 mg, 0.09 mmol), prepared according to example 22, is dissolved in ethanol (5 mL) with gentle heating. Catalytic amounts of 10% palladium on carbon are added and the solution is hydrogenated under balloon pressure for four hours. The mixture is filtered through Celite and evaporated. The residue is purified by chromatography to afford the title compound (16 mg, 43%). LC-MS (M⁺+1): 425.33.

### Example 24

### Tetrahydro-pyran-4-carboxylic Acid [6-(3-pyridin-3-yl-5-trifluoromethyl-pyrazol-1-yl)-pyridin-3-yl]-amide

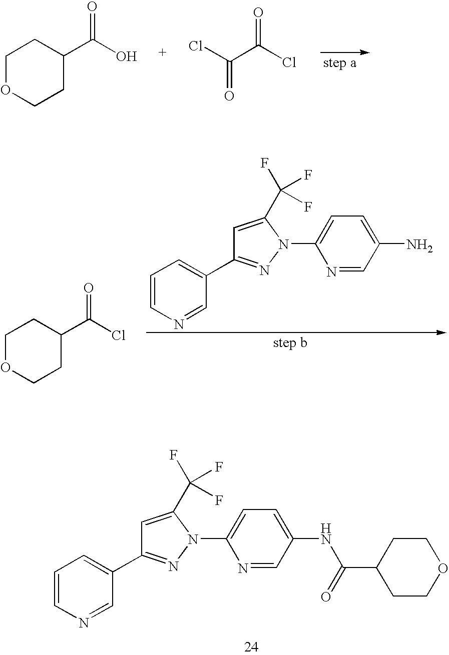

### Step a

Tetrahydro-pyran-4-carboxylic acid (175 mg, 1.3 mmol) is dissolved in dichloromethane (7 mL) and oxalyl chloride (0.23 mL, 2.6 mmol), and catalytic amounts of dimethylformamide are added. The solution is stirred for one hour after which all volatiles are evaporated. The residue is dried under high vacuum for three hours and the resulting product, tetrahydro-pyran-4-carbonyl chloride (192 mg, 100%), is used without further purification.

### Step b

6-(3-Pyridin-3-yl-5-trifluoromethyl-pyrazol-1-yl)-pyridin-3-ylamine (20 mg, 0.065 mmol), prepared according to step d in example 22, is dissolved in tetrahydrofuran (2 mL) and N,N-diisopropylethylamine (35 μL, 0.2 mmol), and tetrahydro-pyran-4-carbonyl chloride (16 mg, 0.1 mmol) are added successively. The solution is stirred for 30 min after which it is diluted with saturated aqueous ammonium chloride solution. The aqueous phase is extracted with dichloromethane and the combined organic phases are dried over magnesium sulfate. The solvent is evaporated and the residue is purified by chromatography to afford the title compound (12 mg, 44%). LC-MS (M⁺+1): 418.37.

The following compounds are prepared according to example 24 by replacing the carboxylic acid in step a with commercially available starting materials.

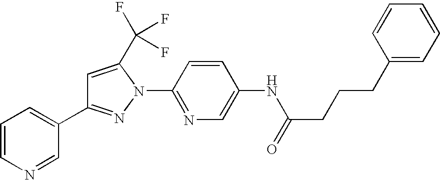

4-Phenyl-N-[6-(3-pyridin-3-yl-5-trifluoromethyl-pyrazol-1-yl)-pyridin-3-yl]-butyramide; LC-MS (M⁺+1): 452.36.

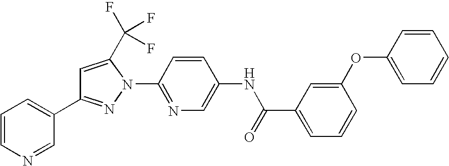

3-Phenoxy-N-[6-(3-pyridin-3-yl-5-trifluoromethyl-pyrazol-1-yl)-pyridin-3-yl]-benzamide; LC-MS (M⁺+1): 502.42.

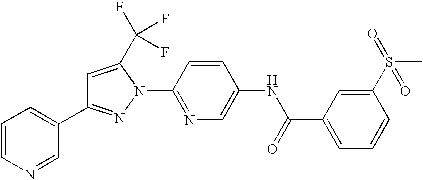

3-Methanesulfonyl-N-[6-(3-pyridin-3-yl-5-trifluoromethyl-pyrazol-1-yl)-pyridin-3-yl]-benzamide; LC-MS (M⁺+1): 488.02

The following compound is prepared according to example 24 by replacing the carboxylic acid in step a with 4,4-diphenyl-butyric acid, prepared according to Chem. Pharm. Bull. 1990, 38(6), 1570.

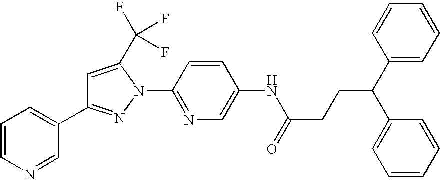

4,4-Diphenyl-N-[6-(3-pyridin-3-yl-5-trifluoromethyl-pyrazol-1-yl)-pyridin-3-yl]-butyramide; LC-MS (M⁺+1): 528.40.

### Example 25

### 2-(3-Morpholin-4-yl-propylamino)-N-[6-(3-pyridin-3-yl-5-trifluoromethyl-pyrazol-1-yl)-pyridin-3-yl]-isonicotinamide

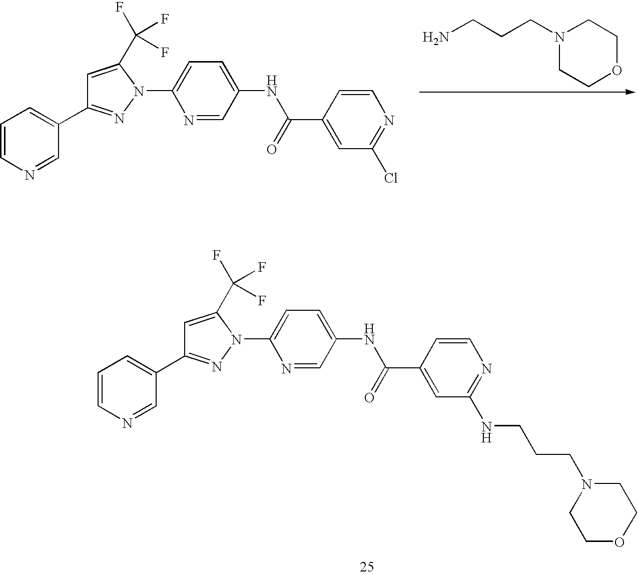

2-Chloro-N-[6-(3-pyridin-3-yl-5-trifluoromethyl-pyrazol-1-yl)-pyridin-3-yl]-isonicotinamide (40 mg, 0.09 mmol), prepared according to example 22, is dissolved in tetrahydrofuran (3 mL), 3-Morpholin-4-yl-propylamine (128 mg, 0.9 mmol) is added, and the solution is microwaved for 14 hours at 150° C. The mixture is diluted with saturated sodium bicarbonate solution and the aqueous phase is extracted with dichloromethane. The combined phases are washed with brine, and dried over magnesium sulfate. The solvent is evaporated and the residue is purified by chromatography to afford the title compound (27 mg, 54%). LC-MS (M¹+1): 553.02.

The following compound is prepared according to example 25 by replacing the amine with commercially available starting material.

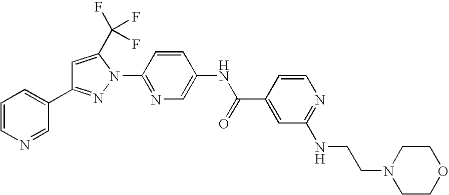

2-(2-Morpholin-4-yl-ethylamino)-N-[6-(3-pyridin-3-yl-5-trifluoromethyl-pyrazol-1-yl)-pyridin-3-yl]-isonicotinamide; LC-MS (M⁺+1): 539.33.

### Example 26

### 2-(2-Morpholin-4-yl-ethylamino)-N-[6-(3-pyridin-3-yl-5-trifluoromethyl-pyrazol-1-yl)-pyridin-3-yl]-nicotinamide

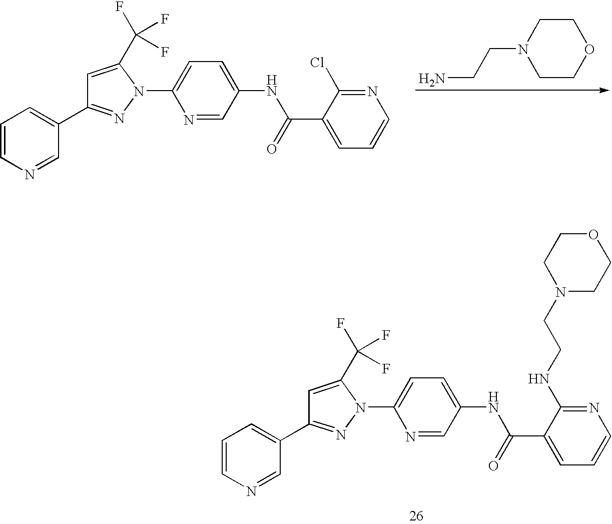

2-Chloro-N-[6-(3-pyridin-3-yl-5-trifluoromethyl-pyrazol-1-yl)-pyridin-3-yl]-nicotinamide (19 mg, 0.04 mmol), prepared according to example 22, is dissolved in dimethylformamide (2 mL), 4-(2-aminoethyl)morpholine is added (28 μL, 0.2 mmol), and the solution is microwaved at 120° C. for 1 hour. The mixture is diluted with saturated aqueous sodium bicarbonate solution and extracted with ethyl acetate. The organic extracts are washed with water, brine, and dried over magnesium sulfate. The solvent is evaporated and the residue is purified by chromatography to afford the title compound (7 mg, 39%). LC-MS (M⁺+1): 538.95.

The following compound is prepared according to example 26 by replacing the amine with commercially available starting material.

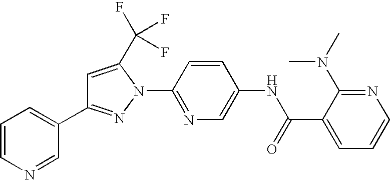

2-Dimethylamino-N-[6-(3-pyridin-3-yl-5-trifluoromethyl-pyrazol-1-yl)-pyridin-3-yl]-nicotinamide; LC-MS (M⁺+1): 453.88.

### Example 27

### 3-Propoxy-N-[6-(3-pyridin-3-yl-5-trifluoromethyl-pyrazol-1-yl)-pyridin-3-yl]-benzamide

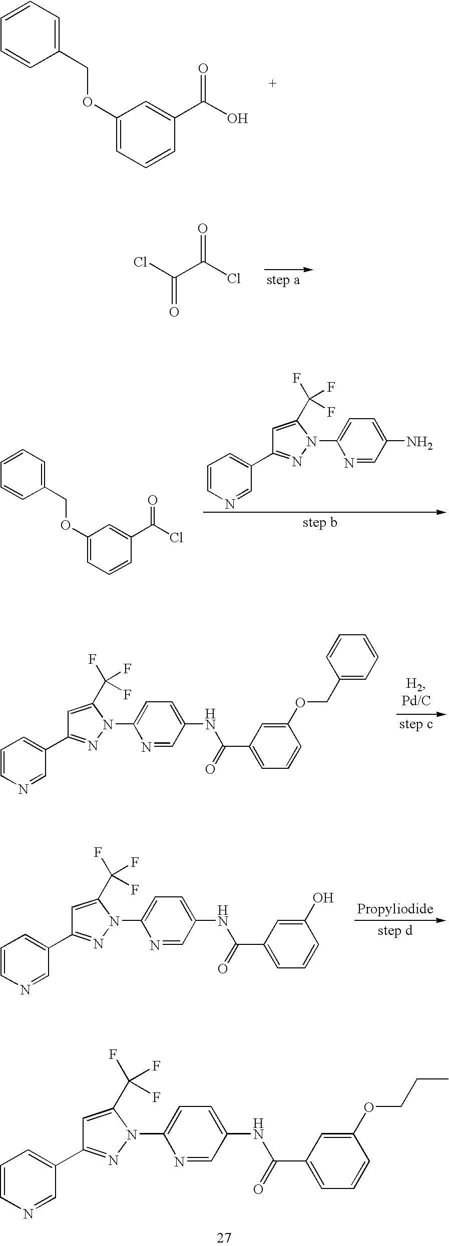

### Step a

3-Benzyloxyl-benzoic acid (460 mg, 2 mmol), prepared according to J. Chem. Soc. 1943, 430, is dissolved in dichloromethane (10 mL) and oxalyl chloride (1.5 mL of a 2 M solution in dichloromethane, 3 mmol), and catalytic amounts of dimethylformamide are added. The solution is stirred for 1 hour after which all volatiles are evaporated. The residue is dried under high vacuum for three hours and the resulting product, 3-benzyloxyl-benzoyl chloride (490 mg, 100%), is used without further purification.

### Step b

6-(3-Pyridin-3-yl-5-trifluoromethyl-pyrazol-1-yl)-pyridin-3-ylamine (215 mg, 0.7 mmol), prepared according to step d in example 22, is dissolved in tetrahydrofuran (10 mL) and N,N-diisopropylethylamine (370 μL, 2.1 mmol), and 3-benzyloxyl-benzoyl chloride (200 μL, 0.84 mmol) is added successively. The solution is stirred for 30 min after which it is diluted with saturated aqueous ammonium chloride solution. The aqueous phase is extracted with dichloromethane and the combined organic phases are dried over magnesium sulfate. The solvent is evaporated and the residue is purified by chromatography to afford 3-benzyloxy-N-[6-(3-pyridin-3-yl-5-trifluoromethyl-pyrazol-1-yl)-pyridin-3-yl]-benzamide (253 mg, 70%).

### Step c

3-Benzyloxy-N-[6-(3-pyridin-3-yl-5-trifluoromethyl-pyrazol-1-yl)-pyridin-3-yl]-benzamide (240 mg, 0.47 mmol) is dissolved in ethanol (10 mL), catalytic amounts of 10% palladium on carbon are added and the solution is hydrogenated under a balloon atmosphere overnight. The mixture is filtered through Celite, the solvent is evaporated, and the residue is purified by chromatography to afford 3-hydroxy-N-[6-(3-pyridin-3-yl-5-trifluoromethyl-pyrazol-1-yl)-pyridin-3-yl]-benzamide (185 mg, 92%).

### Step d

3-Hydroxy-N-[6-(3-pyridin-3-yl-5-trifluoromethyl-pyrazol-1-yl)-pyridin-3-yl]-benzamide (20 mg, 0.05 mmol), propyl iodide (5 μL, 0.05 mmol), and potassium carbonate (15 mg, 0.11 mmol) are placed in a sealable tube and dissolved in acetonitrile (3 mL). The tube is heated at 70° C. overnight. The solution is poured into saturated aqueous ammonium chloride solution and the aqueous phase is extracted with dichloromethane. The extract is dried over magnesium sulfate, filtered, and evaporated. The residue is purified by chromatography to afford the title compound (13 mg, 59%). LC-MS (M⁺+1): 468.38.

The following compounds are prepared according to example 27 by replacing the alkyl halide in step d with commercially available starting materials.

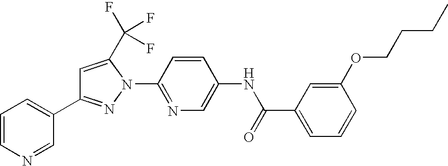

3-Butoxy-N-[6-(3-pyridin-3-yl-5-trifluoromethyl-pyrazol-1-yl)-pyridin-3-yl]-benzamide; LC-MS (M⁺+1): 482.40.

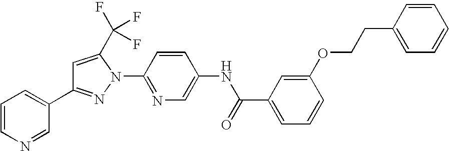

3-Phenethyloxy-N-[6-(3-pyridin-3-yl-5-trifluoromethyl-pyrazol-1-yl)-pyridin-3-yl]-benzamide; LC-MS (M⁺+1): 530.36.

### Example 28

### 3-[2-(2-Oxo-pyrrolidin-1-yl)-ethoxy]-N-[6-(3-pyridin-3-yl-5-trifluoromethyl-pyrazol-1-yl)-pyridin-3-yl]-benzamide

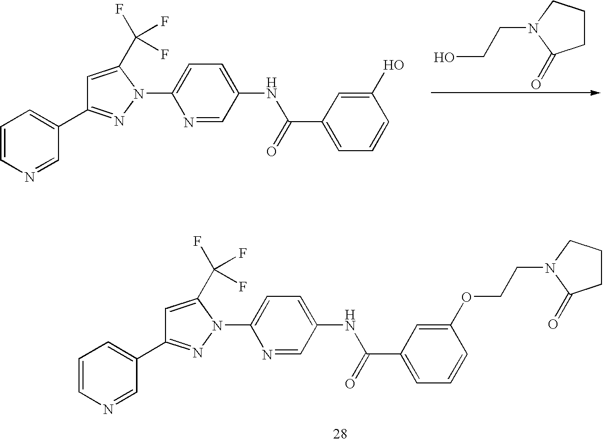

3-Hydroxy-N-[6-(3-pyridin-3-yl-5-trifluoromethyl-pyrazol-1-yl)-pyridin-3-yl]-benzamide (40 mg, 0.09 mmol), prepared according to step c in example 27, triphenylphosphine (94 mg, 0.36 mmol), and 1-(2-hydroxyethyl)-2-pyrrolidinone (52 μL, 0.45 mmol) is dissolved in tetrahydrofuran (3 mL) and diethyl azodicarboxylate (57 μL, 0.36 mmol) is added at room temperature. The solution is stirred for 4 hours and then evaporated. The residue is purified by chromatography to afford the title compound (43 mg, 89%). LC-MS (M⁺+1): 537.11.

The following compounds are prepared according to example 28 by replacing the alcohol with commercially available starting materials.

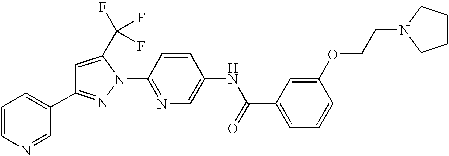

N-[6-(3-Pyridin-3-yl-5-trifluoromethyl-pyrazol-1-yl)-pyridin-3-yl]-3-(2-pyrrolidin-1-yl-ethoxy)-benzamide; LC-MS (M⁺+1): 523.17.

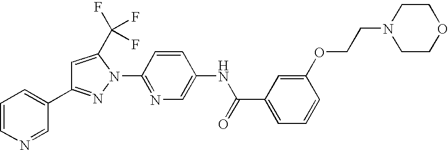

3-(2-Morpholin-4-yl-ethoxy)-N-[6-(3-pyridin-3-yl-5-trifluoromethyl-pyrazol-1-yl)-pyridin-3-yl]-benzamide; LC-MS (M⁺+1): 539.14.

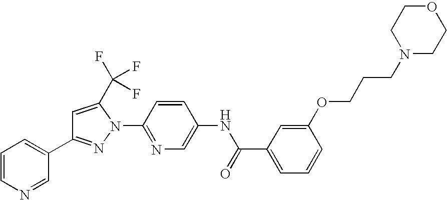

3-(3-Morpholin-4-yl-propoxy)-N-[6-(3-pyridin-3-yl-5-trifluoromethyl-pyrazol-1-yl)-pyridin-3-yl]-benzamide; LC-MS (M⁺+1): 553.14.

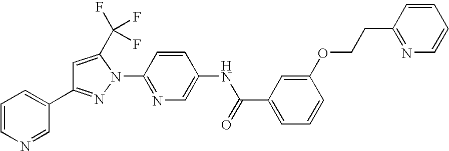

3-(2-Pyridin-2-yl-ethoxy)-N-[6-(3-pyridin-3-yl-5-trifluoromethyl-pyrazol-1-yl)-pyridin-3-yl]-benzamide; LC-MS (M⁺+1): 531.42.

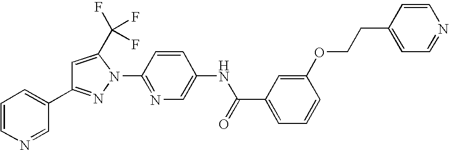

3-(2-Pyridin-4-yl-ethoxy)-N-[6-(3-pyridin-3-yl-5-trifluoromethyl-pyrazol-1-yl)-pyridin-3-yl]-benzamide; LC-MS (M⁺+1): 531.40.

### Example 29

### N-[6-(3-Pyridin-3-yl-5-trifluoromethyl-pyrazol-1-yl)-pyridin-3-yl]-3-pyrimidin-5-yl-benzamide

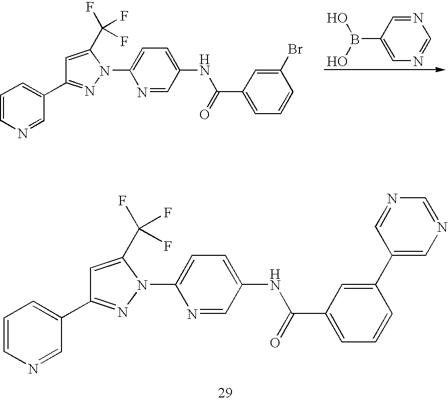

To a solution of 3-Bromo-N-[6-(3-pyridin-3-yl-5-trifluoromethyl-pyrazol-1-yl)-pyridin-3-yl]-benzamide (72 mg, 0.15 mmol), prepared according to example 22, in dimethylformamide (5 mL) is added 3-pyrimidine boronic acid (37 mg, 0.30 mmol), tetrakis(triphenylphosphine)palladium(0) (17 mg, 0.015 mmol), and cesium carbonate (49 mg, 0.15 mmol). The solution is heated to 100° C. in a microwave reactor for 10 minutes. The brown solution is cooled down, diluted with water, and extracted with ethyl acetate. The combined organic layers are washed with water (20 mL, 4×), brine, dried over magnesium sulfate, and then evaporated. The residue is purified by chromatography to afford the title compound (29 mg, 40%). LC-MS (M⁺+1): 488.36.

### Example 30

### 1-(2-Methoxy-ethyl)-6-oxo-1,6-dihydro-pyridine-3-carboxylic Acid [6-(3-pyridin-3-yl-5-trifluoromethyl-pyrazol-1-yl)-pyridin-3-yl]-amide

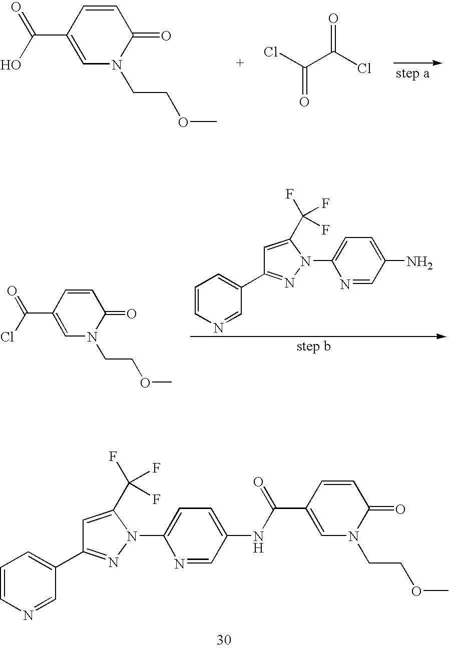

### Step a

1-(2-Methoxy-ethyl)-6-oxo-1,6-dihydro-pyridine-3-carboxylic acid (485 mg, 2.46 mmol), prepared according to step c in example 3, is dissolved in tetrahydrofuran (30 mL) and oxalyl chloride (0.43 mL, 4.9 mmol), and catalytic amounts of dimethylformamide are added. The solution is stirred for 1 hour after which all volatiles are evaporated. The residue is dried under high vacuum for three hours and the resulting product, 1-(2-Methoxy-ethyl)-6-oxo-1,6-dihydro-pyridine-3-carbonyl chloride (530 mg, 100%), is used without further purification.

### Step b

Crude 1-(2-methoxy-ethyl)-6-oxo-1,6-dihydro-pyridine-3-carbonyl chloride (530 mg, 2.46 mmol) is dissolved in pyridine (30 mL), 6-(3-Pyridin-3-yl-5-trifluoromethyl-pyrazol-1-yl)-pyridin-3-ylamine (500 mg, 1.64 mmol), prepared according to step d in example 22, is added and the solution is stirred for 60 min. The solution is evaporated, dried under high vacuum, and the residue is partitioned between ethyl acetate and water. The organic phase is extracted with saturated aqueous sodium bicarbonate solution and saturated aqueous ammonium chloride solution and all the aqueous layers are combined and back-extracted with ethyl acetate. The organic layers are combined, dried over magnesium sulfate, filtered, and evaporated. The residue is purified by chromatography to afford the title compound (565 mg, 71%). LC-MS (M⁺+1): 485.26.

The following compound is prepared according to example 30 by replacing the carboxylic acid in step a with 1-(2-Ethoxy-ethyl)-6-oxo-1,6-dihydro-pyridine-3-carboxylic acid, prepared according to example 3.

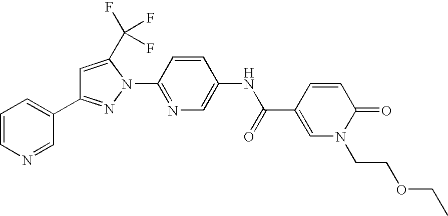

1-(2-Ethoxy-ethyl)-6-oxo-1,6-dihydro-pyridine-3-carboxylic acid [6-(3-pyridin-3-yl-5-trifluoromethyl-pyrazol-1-yl)-pyridin-3-yl]-amide; LC-MS (M⁺+1): 499.22.

### Example 31

### 1-(2-Hydroxy-ethyl)-6-oxo-1,6-dihydro-pyridine-3-carboxylic Acid [6-(3-pyridin-3-yl-5-trifluoromethyl-pyrazol-1-yl)-pyridin-3-yl]-amide

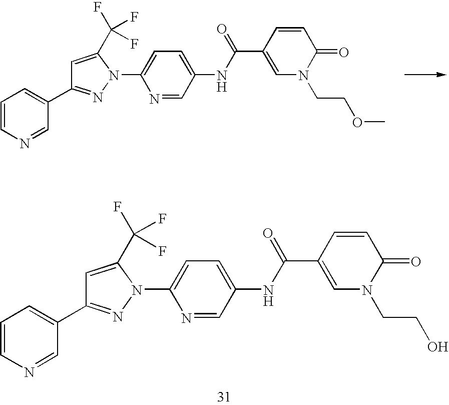

1-(2-Methoxy-ethyl)-6-oxo-1,6-dihydro-pyridine-3-carboxylic acid [6-(3-pyridin-3-yl-5-trifluoromethyl-pyrazol-1-yl)-pyridin-3-yl]-amide (100 mg, 0.2 mmol), prepared according to example 30, was dissolved in dichloromethane (5 mL) and boron tribromide (0.4 mL of a 1 M solution in dichloromethane, 0.4 mmol) is added dropwise at −78° C. The solution is allowed to warm up to room temperature over the course of 1 hour. The reaction is quenched by the addition of methanol (2 mL), diluted with saturated sodium bicarbonate solution and extracted with dichloromethane. The organic layers are combined, dried over MgSO₄, filtered, and evaporated. The residue is purified by chromatography to afford the title compound (68 mg, 70%). LC-MS (M⁺+1): 471.03.

### Example 32

### 1-Dimethylcarbamoylmethyl-6-oxo-1,6-dihydro-pyridine-3-carboxylic Acid [6-(3-pyridin-3-yl-5-trifluoromethyl-pyrazol-1-yl)-pyridin-3-yl]-amide

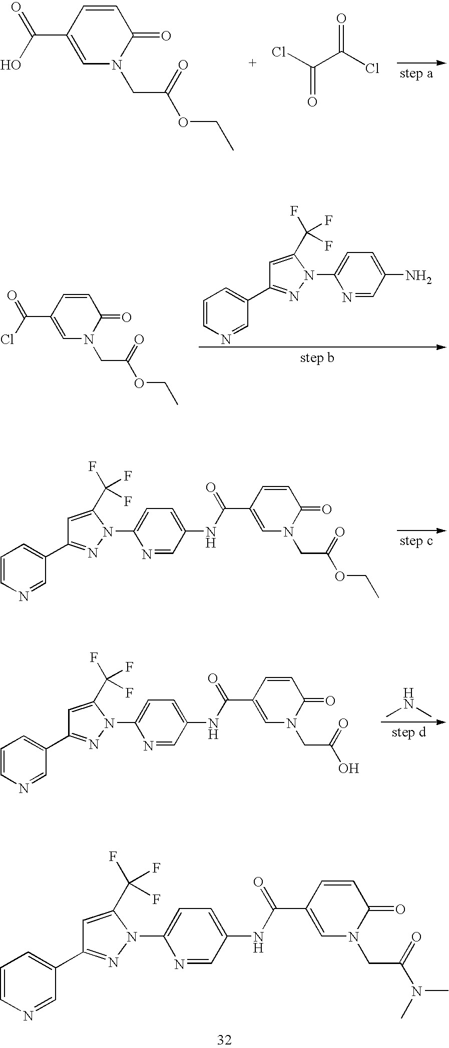

### Step a

1-Ethoxycarbonylmethyl-6-oxo-1,6-dihydro-pyridine-3-carboxylic acid (554 mg, 2.46 mmol), prepared according to step c in example 4, is dissolved in tetrahydrofuran (30 mL) and oxalyl chloride (0.43 mL, 4.9 mmol), and catalytic amounts of dimethylformamide are added. The solution is stirred for 1 hour after which all volatiles are evaporated. The residue is dried under high vacuum for three hours and the resulting product, 1-(2-Methoxy-ethyl)-6-oxo-1,6-dihydro-pyridine-3-carbonyl chloride (598 mg, 100%), is used without further purification.

### Step b

1-Ethoxycarbonylmethyl-6-oxo-1,6-dihydro-pyridine-3-carbonyl chloride (598 mg, 2.46 mmol) is dissolved in pyridine (30 mL), 6-(3-Pyridin-3-yl-5-trifluoromethyl-pyrazol-1-yl)-pyridin-3-ylamine (500 mg, 1.64 mmol), prepared according to step d in example 22, is added and the solution is stirred for 60 min. The solution is evaporated, dried under high vacuum, and the residue is partitioned between ethyl acetate and water. The organic phase is extracted with saturated aqueous sodium bicarbonate solution and saturated aqueous ammonium chloride solution and all the aqueous layers are combined and back-extracted with ethyl acetate. The organic layers are combined, dried over magnesium sulfate, filtered, and evaporated. The residue is purified by chromatography to afford {2-oxo-5-[6-(3-pyridin-3-yl-5-trifluoromethyl-pyrazol-1-yl)-pyridin-3-ylcarbamoyl]-2H-pyridin-1-yl}-acetic acid ethyl ester (521 mg, 62%).

### Step c

{2-Oxo-5-[6-(3-pyridin-3-yl-5-trifluoromethyl-pyrazol-1-yl)-pyridin-3-ylcarbamoyl]-2H-pyridin-1-yl}-acetic acid ethyl ester (50 mg, 0.1 mmol) is dissolved in dioxane (2.5 mL) and LiOH (5 mg, 0.2 mmol), dissolved in water (2.5 mL), is added. The solution is stirred for 2 hours after which it was acidified (˜pH 2) with aqueous 1 M HCl. The precipitate is filtered off and the aqueous solution is extracted with ethyl acetate. The extracts are washed with water and brine, dried over magnesium sulfate, and evaporated. The residue is combined with the precipitate to afford {2-oxo-5-[6-(3-pyridin-3-yl-5-trifluoromethyl-pyrazol-1-yl)-pyridin-3-ylcarbamoyl]-2H-pyridin-1-yl}-acetic acid (25 mg, 51%), which was used without ether purification.

### Step d

{2-Oxo-5-[6-(3-pyridin-3-yl-5-trifluoromethyl-pyrazol-1-yl)-pyridin-3-ylcarbamoyl]-2H-pyridin-1-yl}-acetic acid (57 mg, 0.12 mmol), 1-[3-(dimethylamino)propyl]-2-ethylcarbodiimide hydrochloride (113 mg, 0.59 mmol), and 1-hydroxybenzotriazole hydrate (79 mg, 0.59 mmol) are dissolved in tetrahydrofuran, dimethylamine (0.3 mL of a 2 M solution in tetrahydrofuran, 0.6 mmol) is added and the suspension is vigorously stirred overnight. Methanol is added to dissolve the solids and the solution is then diluted with brine. The aqueous phase is extracted with dichloromethane several times, and the combined organic phases are dried over magnesium sulfate, filtered, and evaporated. The residue is purified by chromatography to afford the title compound (38 mg, 63%). LC-MS (M⁺+1): 512.28.

### Example 33

### 1-(2-Ethoxy-ethyl)-6-oxo-1,6-dihydro-pyridine-3-carboxylic Acid [5-(3-ethoxy-5-trifluoromethyl-pyrazol-1-yl)-pyridin-2-yl]-amide

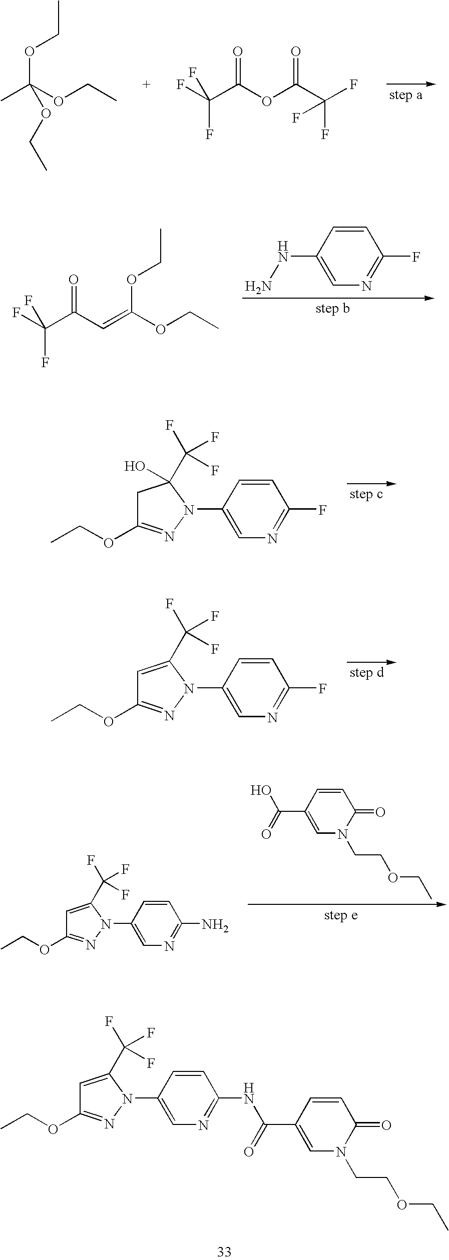

### Step a

To a stirred solution of ethyl orthoacetate (5.7 mL, 30 mmol) and pyridine (5.6 mL, 69 mmol) in chloroform (30 mL) is added dropwise, with ice cooling, trifluoroacetic anhydride (8.7 mL, 61 mmol) and the mixture is stirred at room temperature for 1 hour. The solution is quickly washed with ice-cold aqueous 10% sodium carbonate solution and water, and dried over sodium sulfate. The solvent and pyridine are evaporated and the residue is dried under high vacuum to afford 4,4-diethoxy-1,1,1-trifluoro-but-3-en-2-one (5.7 g, 90%).

### Step b

4,4-Diethoxy-1,1,1-trifluoro-but-3-en-2-one (2.1 g, 10 mmol) and 2-fluoro-5-hydrazino-pyridine (1.4 g, 11 mmol), prepared according to step b in example 1, are dissolved in ethanol/water (40 mL, 9/1) and the solution is microwaved for 30 min at 100° C. The solution is cooled to room temperature, evaporated, and the residue is re-dissolved in ethyl acetate. The organic solution is dried over magnesium sulfate, filtered, and evaporated. The residue is purified by chromatography to afford 5-ethoxy-2-(6-fluoro-pyridin-3-yl)-3-trifluoromethyl-3,4-dihydro-2H-pyrazol-3-ol (1.35 g, 46%).

### Step c

5-Ethoxy-2-(6-fluoro-pyridin-3-yl)-3-trifluoromethyl-3,4-dihydro-2H-pyrazol-3-ol (1.35 g, 4.6 mmol) is dissolved in acetic acid (30 mL) and the solution is microwaved for 45 min at 100° C. The solution is evaporated and the residue is filtered through a plug of silica gel to afford 5-(3-3thoxy-5-trifluoromethyl-pyrazol-1-yl)-2-fluoro-pyridine (640 mg, 50%).

### Step d

5-(3-Ethoxy-5-trifluoromethyl-pyrazol-1-yl)-2-fluoro-pyridine (1 g, 3.6 mmol) is dissolved in dioxane (4 mL) and an equal amount of concentrated ammonium hydroxide is added. The solution is microwaved at 130° C. for 120 min, after which the solution is evaporated. The residue is re-dissolved in dichloromethane, magnesium sulfate is added and the solution is filtered and evaporated. The residue is purified by chromatography to afford 5-(3-ethoxy-5-trifluoromethyl-pyrazol-1-yl)-pyridin-2-ylamine (710 mg, 72%)

### Step e

1-(2-Ethoxy-ethyl)-6-oxo-1,6-dihydro-pyridine-3-carboxylic acid (1.4 g, 6.8 mmol), prepared according to step c in example 3, is dissolved in tetrahydrofuran (20 mL) and oxalyl chloride (1.2 mL, 13.7 mmol), and catalytic amounts of dimethylformamide are added. The solution is stirred for 1 hour after which all volatiles are evaporated. The residue is dried under high vacuum for three hours. The acid chloride is re-dissolved in pyridine (20 mL), 5-(3-ethoxy-5-trifluoromethyl-pyrazol-1-yl)-pyridin-2-ylamine (620 mg, 2.3 mmol) is added, and the solution is stirred for 1 hour. The solution is evaporated and the residue is partitioned between ethyl acetate and saturated sodium bicarbonate solution. The organic phase is extracted with saturated aqueous sodium bicarbonate solution and saturated ammonium chloride solution. The solvent is dried over magnesium sulfate, filtered, and evaporated. The residue is purified by chromatography to afford the title compound (800 mg, 75%). LC-MS (M⁺+1): 466.08.

The following compounds are prepared according to example 33 by replacing the carboxylic acid in step e with commercially available starting materials.

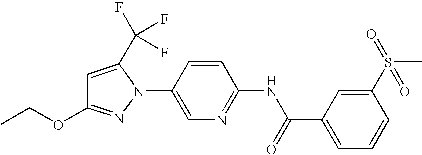

N-[5-(3-Ethoxy-5-trifluoromethyl-pyrazol-1-yl)-pyridin-2-yl]-3-methanesulfonyl-benzamide; LC-MS (M⁺+1): 455.25.

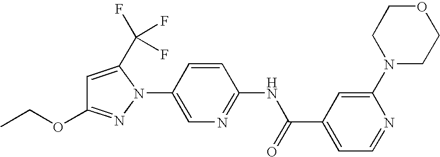

N-[5-(3-Ethoxy-5-trifluoromethyl-pyrazol-1-yl)-pyridin-2-yl]-2-morpholin-4-yl-isonicotinamide; LC-MS (M⁺+1): 462.29.

The following compound is prepared according to example 33 by replacing the carboxylic acid in step e with 3-Morpholin-4-yl-benzoic acid prepared according to step b in example 57.

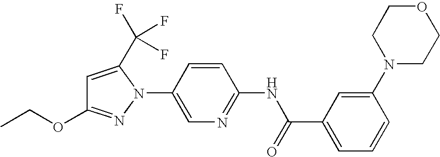

N-[5-(3-Ethoxy-5-trifluoromethyl-pyrazol-1-yl)-pyridin-2-yl]-3-morpholin-4-yl-benzamide; LC-MS (M⁺+1): 462.29.

### Example 34

### 3-Cyano-N-[6-(3-ethoxy-5-trifluoromethyl-pyrazol-1-yl)-pyridin-3-yl]-benzamide

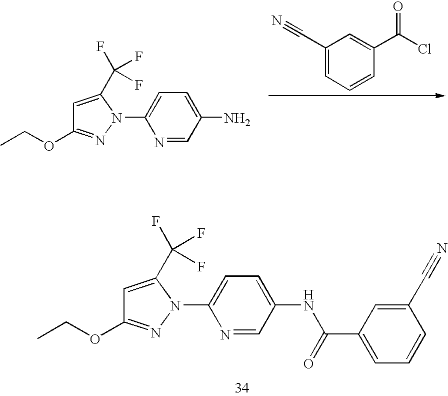

5-(3-Ethoxy-5-trifluoromethyl-pyrazol-1-yl)-pyridin-2-ylamine (40 mg, 0.15 mmol), prepared according to step d in example 33, is dissolved in pyridine (4 mL), 3-cyanobenzoyl chloride (36 mg, 0.22 mmol) is added, and the solution is stirred for 1 hour. The solution is evaporated and the residue is partitioned between ethyl acetate and saturated sodium bicarbonate solution. The organic phase is extracted with saturated aqueous sodium bicarbonate solution and saturated aqueous ammonium chloride solution. The solvent is dried over magnesium sulfate, filtered, and evaporated. The residue is purified by chromatography to afford the title compound (33 mg, 56%). LC-MS (M⁺+1): 402.37

The following compound is prepared according to example 34 by replacing the acyl chloride with commercially available starting material.

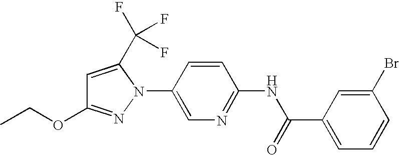

3-Bromo-N-[5-(3-ethoxy-5-trifluoromethyl-pyrazol-1-yl)-pyridin-2-yl]-benzamide; LC-MS (M⁺+1): 457.13

### Example 35

### 4′-(Pyrrolidine-1-carbonyl)-biphenyl-3-carboxylic Acid [5-(3-ethoxy-5-trifluoromethyl-pyrazol-1-yl)-pyridin-2-yl]-amide

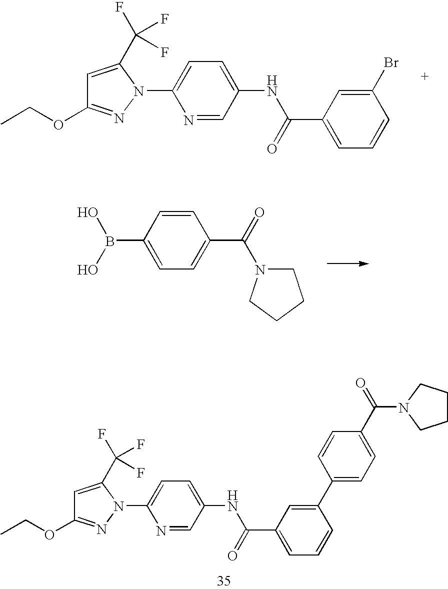

To a solution of 3-Bromo-N-[5-(3-ethoxy-5-trifluoromethyl-pyrazol-1-yl)-pyridin-2-yl]-benzamide (55 mg, 0.12 mmol), prepared according example 34, in dimethylformamide (3 mL) is added 4-(Pyrrolidine-1-carbonyl)-phenyl boronic acid (53 mg, 0.24 mmol), tetrakistriphenylphosphine palladium(0) (14 mg, 0.012 mmol), and cesium carbonate (39 mg, 0.12 mmol). The solution is heated to 140° C. in a microwave reactor for 2 hours. The brown solution is cooled down, diluted with water, and extracted with ethyl acetate. The combined organic layers are washed with water, brine, dried over magnesium sulfate, and then evaporated. The residue is purified by chromatography to afford the title compound (31 mg, 47%). LC-MS (M⁺+1): 550.32.

### Example 36

### N-Pyridin-4-yl-6-(3-pyridin-3-yl-5-trifluoromethyl-pyrazol-1-yl)-nicotinamide

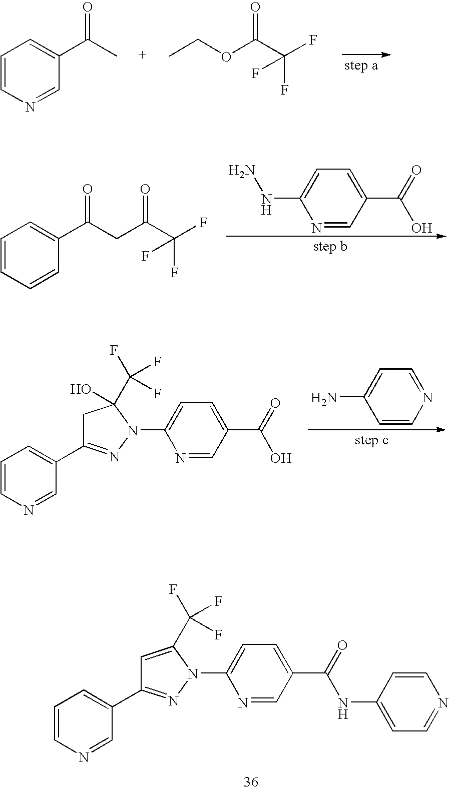

### Step a

To a solution of ethyl trifluoroacetate (24 mL, 0.202 mol) in ether (250 mL) are added sodium methoxide (10.9 g, 0.202 mol) and 3-acetylpyridine (22 mL, 0.202 mol) at room temperature. The solution is heated at reflux for 2 hours. The reaction is cooled to room temperature and water (100 mL) is added. The resulting mixture is extracted with ether (100 mL) and the aqueous layer is collected. The aqueous solution is acidified to pH 4 with 0.2 M acetic acid. The solid that precipitates out from the solution is collected by means of filtration and is dried under vacuum. The resulting product, 4,4,4-trifluoro-1-pyridin-3-yl-butane-1,3-dione (32.8 g, 75.5%), is used in the next step of the synthesis without further purification.

### Step b

To a solution of 4,4,4-trifluoro-1-pyridin-3-yl-butane-1,3-dione (142 mg, 0.653 mmol) in ethanol (5 mL) are added 6-hydrazinonicotinic acid (100 mg, 0.653 mmol) and aqueous 12 M HCl (0.1 mL) at room temperature. The solution is heated up to 80° C. for 6 hours. The solution is cooled to room temperature and concentrated under reduced pressure. The solid residue is then dissolved in boiling ethanol (3 mL). Ethyl acetate (20 mL) is added to the solution and the solid that precipitates out of the solution is collected by means of filtration. The solid is washed with cold ethanol (5 mL) and then dried under vacuum. The resulting product, 6-(5-hydroxy-3-pyridin-3-yl-5-trifluoromethyl-4,5-dihydro-pyrazol-1-yl)-nicotinic acid (210 mg, 91.3%), is used in the next step of the synthesis without further purification.

### Step c

To a solution of 6-(5-hydroxy-3-pyridin-3-yl-5-trifluoromethyl-4,5-dihydro-pyrazol-1-yl)-nicotinic acid (30 mg, 0.085 mmol) in dimethylformamide (5 mL) are added 4-amino-pyridine (17 mg, 0.180 mmol), 1-(3-dimethylaminopropyl)-3-ethylcarbodiimide hydrochloride (52 mg, 0.270 mmol), 1-hydroxybenzotriazole hydrate (24 mg, 0.180 mmol) and diisopropylethyl amine (0.032 mL, 0.180 mmol) at room temperature. The solution is stirred at the same temperature for 24 hours. Saturated aqueous sodium bicarbonate solution (5 mL) is added and the solution mixture is extracted with ethylacetate (20 mL). The organic layer is dried with magnesium sulfate and filtered. The filtrate is concentrated under reduced pressure. The residue is purified by chromatography to afford the title compound (19.2 mg, 52%). LC-MS (M⁺+1): 411.36

The following compounds are prepared according to example 36 by replacing the amine in step c with commercially available starting materials.

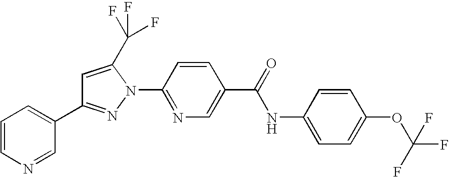

6-(3-Pyridin-3-yl-5-trifluoromethyl-pyrazol-1-yl)-N-(4-trifluoromethoxy-phenyl)-nicotinamide; LC-MS (M⁺+1): 493.88.

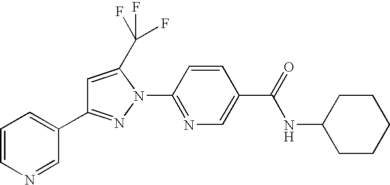

N-Cyclohexyl-6-(3-pyridin-3-yl-5-trifluoromethyl-pyrazol-1-yl)-nicotinamide; LC-MS (M⁺+1): 416.39.

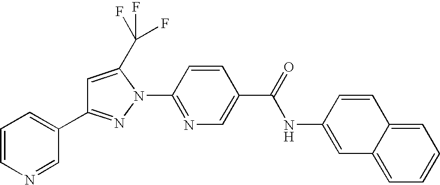

N-Naphthalen-2-yl-6-(3-pyridin-3-yl-5-trifluoromethyl-pyrazol-1-yl)-nicotinamide; LC-MS (M⁺+1): 460.35.

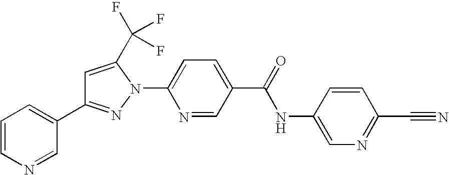

N-(6-Cyano-pyridin-3-yl)-6-(3-pyridin-3-yl-5-trifluoromethyl-pyrazol-1-yl)-nicotinamide; LC-MS (M⁺+1): 436.34.

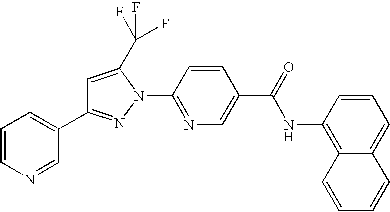

N-Naphthalen-1-yl-6-(3-pyridin-3-yl-5-trifluoromethyl-pyrazol-1-yl)-nicotinamide; LC-MS (M⁺+1): 460.38.

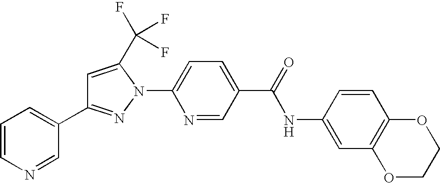

N-(2,3-Dihydro-benzo[1,4]dioxin-6-yl)-6-(3-pyridin-3-yl-5-trifluoromethyl-pyrazol-1-yl)-nicotinamide; LC-MS (M⁺+1): 468.33.

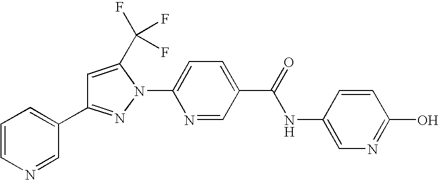

N-(6-Hydroxy-pyridin-3-yl)-6-(3-pyridin-3-yl-5-trifluoromethyl-pyrazol-1-yl)-nicotinamide; LC-MS (M⁺+1): 427.32.

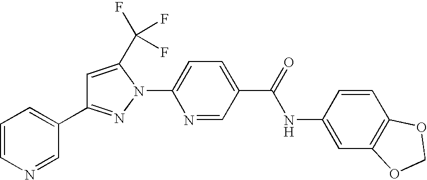

N-Benzo[1,3]dioxol-5-yl-6-(3-pyridin-3-yl-5-trifluoromethyl-pyrazol-1-yl)-nicotinamide; LC-MS (M⁺+1): 454.34.

### Example 37

### N-(3,3-Diphenyl-propyl)-6-(3-pyridin-3-yl-5-trifluoromethyl-pyrazol-1-yl)-nicotinamide

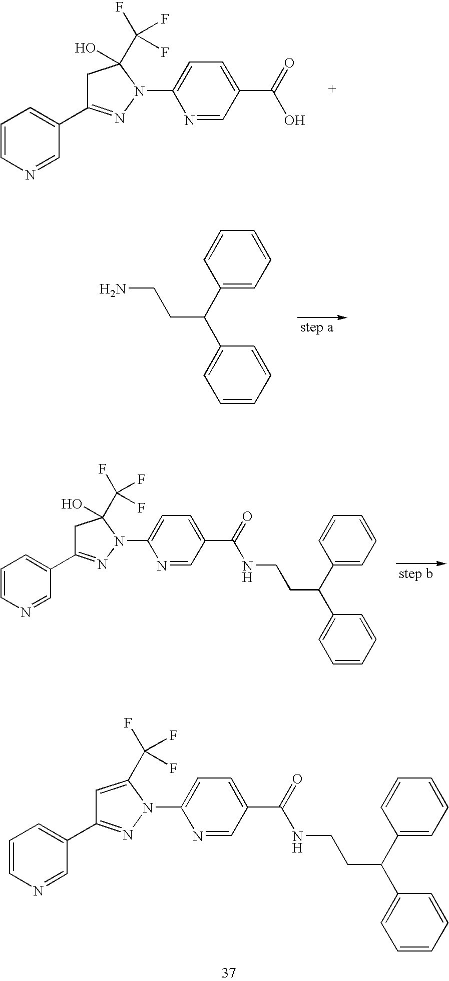

### Step a

6-(5-Hydroxy-3-pyridin-3-yl-5-trifluoromethyl-4,5-dihydro-pyrazol-1-yl)-nicotinic acid is prepared according to step b in example 36. To a solution of 6-(5-hydroxy-3-pyridin-3-yl-5-trifluoromethyl-4,5-dihydro-pyrazol-1-yl)-nicotinic acid (70 mg, 0.199 mmol) in dimethylformamide (5 mL) are added N,N-diphenylpropyl amine (55 μL, 0.398 mmol), 1-(3-dimethylaminopropyl)-3-ethylcarbodiimide (114 mg, 0.59 mmol), 1-hydroxybenzo-triazole hydrate (81 mg, 0.59 mmol) and diisopropylethyl amine (0.1 mL, 0.59 mmol) at room temperature. The solution is stirred at the same temperature for 24 hours. Saturated aqueous sodium bicarbonate solution (5 mL) is added and the solution mixture is extracted with ethyl acetate (20 mL). The organic layer is dried with magnesium sulfate and filtered. The filtrate is concentrated under reduced pressure. The residue is purified by chromatography to afford N-(3,3-diphenyl-propyl)-6-(5-hydroxy-3-pyridin-3-yl-5-trifluoromethyl-4,5-dihydro-pyrazol-1-yl)-nicotinamide (54 mg, 49%).

### Step b

N-(3,3-Diphenyl-propyl)-6-(5-hydroxy-3-pyridin-3-yl-5-trifluoromethyl-4,5-dihydro-pyrazol-1-yl)-nicotinamide (60 mg, 0.110 mmol) is dissolved in acetic acid (3 mL) at room temperature. The solution is heated to 120° C. for 6 hours. The resulting solution is cooled to room temperature and is extracted with ethyl acetate (10 mL, 3×) and water (15 mL). The combined organic layer is dried with magnesium sulfate and filtered. The filtrate is concentrated under reduced pressure and the residue is purified by chromatography to afford the title compound (58 mg, 67%). LC-MS (M⁺+1): 528.37.

The following compounds are prepared according to example 37 by replacing the amine in step a with commercially available starting materials.

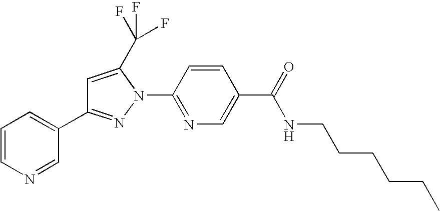

N-Hexyl-6-(3-pyridin-3-yl-5-trifluoromethyl-pyrazol-1-yl)-nicotinamide; LC-MS (M⁺+1): 418.45.

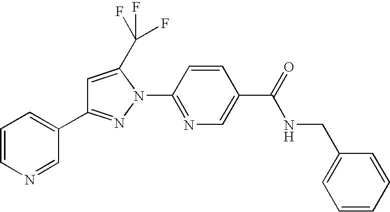

N-Benzyl-6-(3-pyridin-3-yl-5-difluoromethyl-pyrazol-1-yl)-nicotinamide; LC-MS (M⁺+1): 424.36.

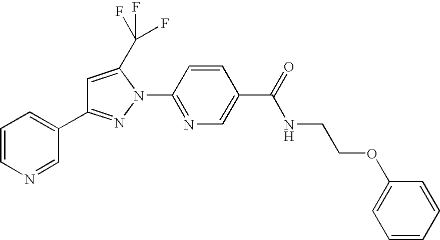

N-(2-Phenoxy-ethyl)-6-(3-pyridin-3-yl-5-trifluoromethyl-pyrazol-1-yl)-nicotinamide; LC-MS (M⁺+1): 454.36.

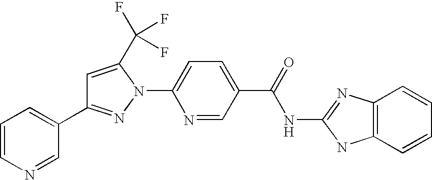

N-(1H-Benzoimidazol-2-yl)-6-(3-pyridin-3-yl-5-trifluoromethyl-pyrazol-1-yl)-nicotinamide; LC-MS (M⁺): 449.87.

### Example 38

### N-[4-(2-Piperidin-1-yl-ethoxy)-naphthalen-1-yl]-6-(3-pyridin-3-yl-5-trifluoromethyl-pyrazol-1-yl)-nicotinamide

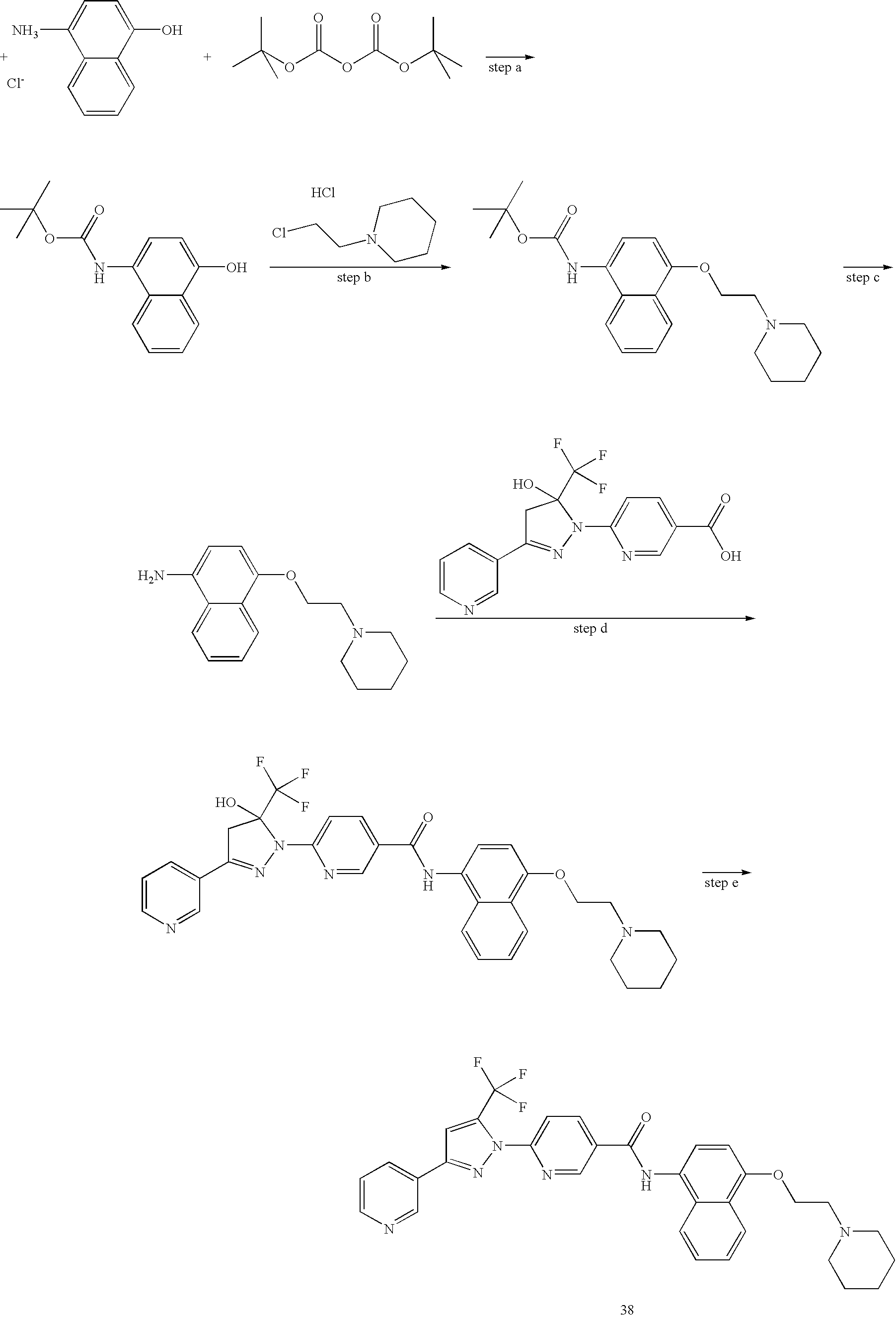

### Step a

To a solution of aminonaphthol (89 g, 0.458 mol) in tetrahydrofuran (700 mL) is added triethylamine (70.4 mL, 0.504 mol) at room temperature. The solution is stirred at the same temperature for 30 minutes and a solution of di-t-butyl dicarbonate (103 g, 0.472 mol) in tetrahydrofuran (100 mL) is added. The resulting solution is heated up to 65° C. for 5 hours. The solution is cooled to room temperature and the solid that precipitates out from solution is removed by means of filtration. The filtrate is concentrated under reduced pressure and the residue is dried under vacuum to afford (4-hydroxy-naphthalen-1-yl)-carbamic acid tert-butyl ester (118.8 g, 47.5%).

### Step b

To a solution of (4-hydroxy-naphthalen-1-yl)-carbamic acid tert-butyl ester (100 mg, 0.386 mmol) in dimethylformamide (10 mL) are added 1-(2-chloroethyl)piperidine monochloride (136 mg, 0.736 mmol) and potassium carbonate (160 mg, 1.158 mmol) at room temperature. The solution is stirred at the same temperature for 12 hours. The solution is extracted with ethyl acetate (20 mL, 3×) and water (30 mL) and the combined organic layers are dried over magnesium sulfate and filtered. The filtrate is concentrated under reduced pressure and the residue, [4-(2-piperidin-1-yl-ethoxy)-naphthalen-1-yl]-carbamic acid tert-butyl ester (143 mg, 70%), is used in the next step of the synthesis without further purification.

### Step c

To a solution of [4-(2-piperidin-1-yl-ethoxy)-naphthalen-1-yl]-carbamic acid tert-butyl ester (100 mg, 0.270 mmol) in ethanol (10 mL) is added aqueous 12 M HCl (0.5 mL) at room temperature. The solution is heated up to 50° C. for 12 hours and then cooled to room temperature. The solution is concentrated under reduced pressure and the residue is purified by chromatography to afford 4-(2-piperidin-1-yl-ethoxy)-naphthalen-1-ylamine (73 mg, 70%).

### Step d

6-(5-Hydroxy-3-pyridin-3-yl-5-trifluoromethyl-4,5-dihydro-pyrazol-1-yl)-nicotinic acid is prepared according step b prepare example 36. To a solution of 6-(5-hydroxy-3-pyridin-3-yl-5-trifluoromethyl-4,5-dihydro-pyrazol-1-yl)-nicotinic acid (50 mg, 0.142 mmol) in dimethylformamide (10 mL) are added 4-(2-piperidin-1-yl-ethoxy)-naphthalen-1-ylamine (58 mg, 0.213 mmol), 1-(3-dimethylaminopropyl)-3-ethylcarbodiimide (83 mg, 0.426 mmol), 1-hydroxybenzo-triazole hydrate (38 mg, 0.284 mmol) and diisopropyl-ethyl amino (0.049 mL, 0.284 mmol) at room temperature. The solution is stirred at the same temperature for 24 hours. Saturated aqueous sodium bicarbonate solution (5 mL) is added and the solution mixture is extracted with ethyl acetate (20 mL). The organic layer is dried over magnesium sulfate and filtered. The filtrate is concentrated under reduced pressure and the residue is purified by chromatography to afford 6-(5-Hydroxy-3-pyridin-3-yl-5-trifluoromethyl-4,5-dihydro-pyrazol-1-yl)-[4-(2-morpholin-4-yl-ethoxy)-naphthalen-1-yl]-nicotinamide (65 mg, 76%).

### Step e

6-(5-Hydroxy-3-pyridin-3-yl-5-trifluoromethyl-4,5-dihydro-pyrazol-1-yl)-N-[4-(2-morpholin-4-yl-ethoxy)-naphthalen-1-yl]-nicotinamide (50 mg, 0.083 mmol) is dissolved in acetic acid (5 mL) at room temperature. The solution is heated to 120° C. for 6 hours. The resulting solution is cooled to room temperature and is extracted with ethyl acetate (10 mL, 3×) and water (15 mL). The combined organic layer is dried with magnesium sulfate and filtered. The filtrate is concentrated under reduced pressure and the residue is purified by chromatography to afford the title compound (8 mg, 16%). LC-MS (M⁺+1): 586.95.

The following compound is prepared according to example 38 by replacing the amine in step a with commercially available starting material.

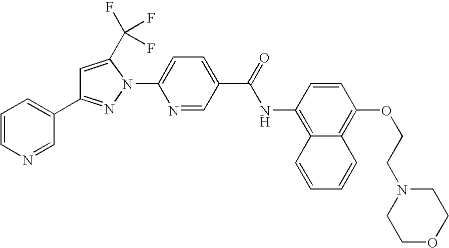

N-[4-(2-Morpholin-4-yl-ethoxy)-naphthalen-1-yl]-6-(3-pyridin-3-yl-5-trifluoromethyl-pyrazol-1-yl)-nicotinamide; LC-MS (M⁺+1): 589.4

### Example 39

### N-Hexyl-6-(5-hydroxy-3-pyridin-3-yl-5-trifluoromethyl-4,5-dihydro-pyrazol-1-yl)-nicotinamide

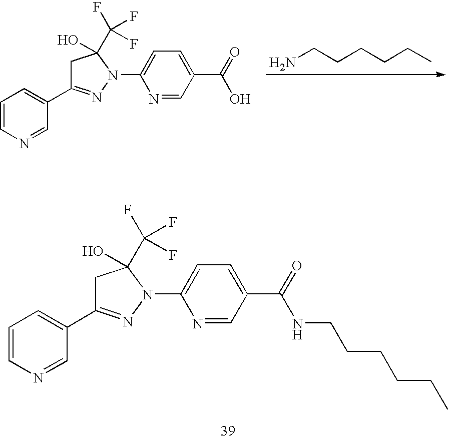

6-(5-Hydroxy-3-pyridin-3-yl-5-trifluoromethyl-4,5-dihydro-pyrazol-1-yl)-nicotinic acid is prepared according to step b in example 36. To a solution of 6-(5-hydroxy-3-pyridin-3-yl-5-trifluoromethyl-4,5-dihydro-pyrazol-1-yl)-nicotinic acid (70 mg, 0.199 mmol) in dimethylformamide (5 mL) are added N-hexylamine (0.053 mL, 0.398 mmol), 1-(3-dimethylaminopropyl)-3-ethylcarbodiimide (114 mg, 0.59 mmol), 1-hydroxybenzo-triazole hydrate (81 mg, 0.59 mmol), and diisopropylethyl amine (0.1 mL, 0.59 mmol) at room temperature. The solution is stirred at the same temperature for 24 hours. Saturated aqueous sodium bicarbonate solution (5 mL) is added and the solution mixture is extracted with ethyl acetate (20 mL). The organic layer is dried over magnesium sulfate and filtered. The filtrate is concentrated under reduced pressure and the residue is purified by chromatography to afford N-hexyl-6-(5-hydroxy-3-pyridin-3-yl-5-trifluoromethyl-4,5-dihydro-pyrazol-1-yl)-nicotinamide (60 mg, 69%). LC-MS (M⁺): 435.97.

The following compounds are prepared according to example 39 by replacing the amine with commercially available starting materials.

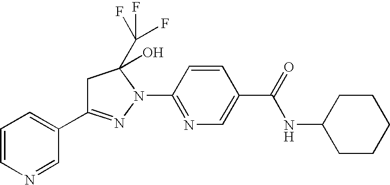

N-Cyclohexyl-6-(5-hydroxy-3-pyridin-3-yl-5-trifluoromethyl-4,5-dihydro-pyrazol-1-yl)-nicotinamide; LC-MS (M⁺): 433.98.

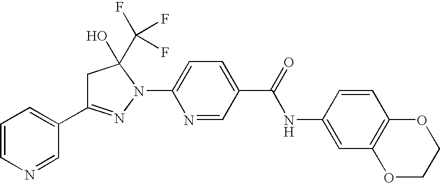

N-(2,3-Dihydro-benzo[1,4]dioxin-6-yl)-6-(5-hydroxy-3-pyridin-3-yl-5-trifluoromethyl-4,5-dihydro-pyrazol-1-yl)-nicotinamide; LC-MS (M⁺+1): 486.39.

### Example 40

### 6-(3-Pyridin-3-yl-5-trifluoromethyl-pyrazol-1-yl)-2H-pyridazin-3-one

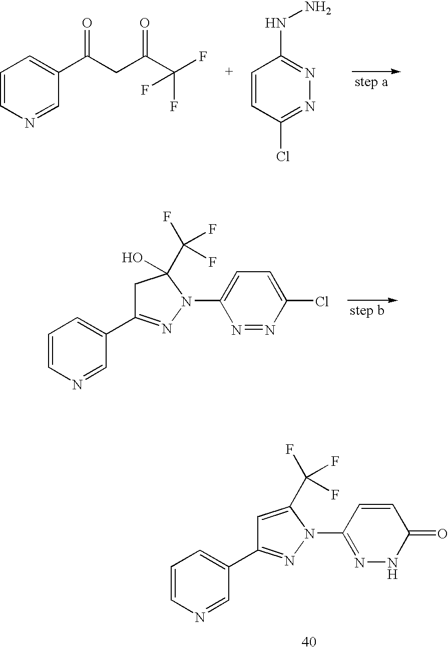

### Step a

4,4,4-Trifluoro-1-pyridin-3-yl-butane-1,3-dione is prepared according to step a in example 35. 4,4,4-Trifluoro-1-pyridin-3-yl-butane-1,3-dione (883 mg, 4 mmol) is dissolved in ethanol (10 mL) and 3-chloro-6-hydrazinopyridazine (145 mg, 3.4 mmol), 12 M aqueous HCl (0.5 mL) are added at room temperature. The solution is heated up to 80° C. for 6 hours and is then cooled to room temperature. The solution is concentrated under reduced pressure and the residue is dissolved in boiling ethanol (5 mL). Ethyl acetate (30 mL) is added to the solution and the precipitate is collected by means of filtration. The solid is washed with cold ethanol (1 mL) and dried under vacuum. The resulting product, 2-(6-chloro-pyridazin-3-yl)-5-pyridin-3-yl-3-trifluoromethyl-3,4-dihydro-2H-pyrazol-3-ol (650 mg, 56%), is used in the next step of the synthesis without further purification.

### Step b

2-(6-Chloro-pyridazin-3-yl)-5-pyridin-3-yl-3-trifluoromethyl-3,4-dihydro-2H-pyrazol-3-ol (150 mg, 0.44 mmol) is dissolved in acetic acid (10 mL) at room temperature. The solution is heated to 120° C. for 6 hours. The resulting solution is cooled to room temperature and extracted with ethyl acetate (20 mL, 3×) and water (30 mL). The combined organic layer is dried with magnesium sulfate and filtered. The filtrate is concentrated under reduced pressure and the residue is purified by chromatography to afford the title compound (134 mg, 69%). LC-MS (M⁺): 307.96.

### Example 41

### 5-(3-Pyridin-3-yl-5-trifluoromethyl-pyrazol-1-yl)-pyridine-2-carboxylic Acid pyridin-4-ylamide

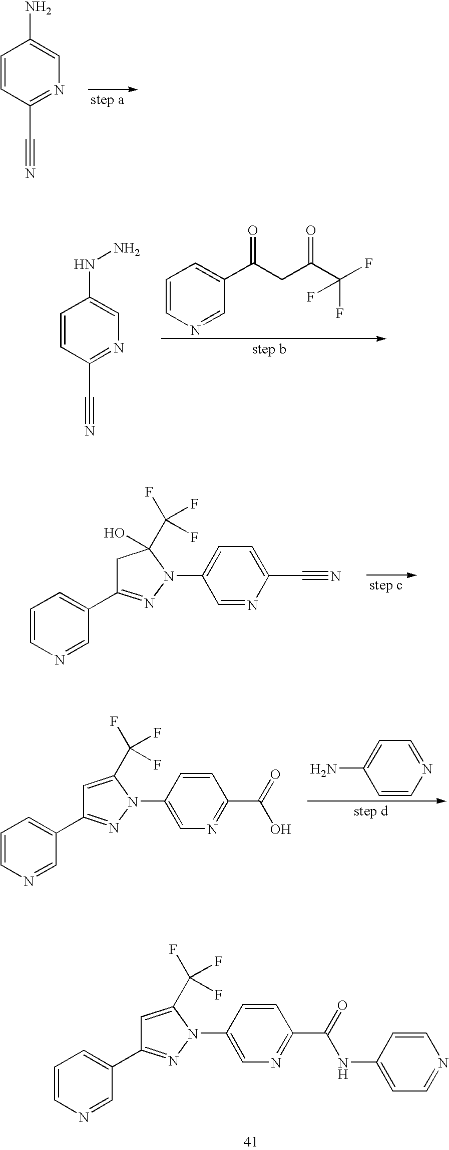

### Step a

To a solution of 5-amino-2-cyanopyridine (200 mg, 1.6 mmol) in 6 M aqueous HCl (10 mL) is added sodium nitrite (116 mg, 1.6 mmol) at 0° C. The solution is stirred at the same temperature for 1 hour. Tin(II)chloride hydrate (742 mg, 3.2 mmol) is added and the solution is allowed to warm to room temperature. The solution is kept in an ice bath and potassium hydroxide is added to neutralize the solution to pH 7. The solid that precipitates out from the solution is collected by filtration and is washed with cold ethanol (5 mL). The resulting product, 5-hydrazino-pyridine-2-carbonitrile (216 mg, 83%), is dried under vacuum and used in the next step of the synthesis without further purification.

### Step b

4,4,4-Trifluoro-1-pyridin-3-yl-butane-1,3-dione is prepared according to step a in example 35. 4,4,4-Trifluoro-1-pyridin-3-yl-butane-1,3-dione (122 mg, 0.56 mmol) is dissolved in ethanol (5 mL) and 5-hydrazino-pyridine-2-carbonitrile (50 mg, 3.4 mmol) in 12 M aqueous HCl (1 mL) are added at room temperature. The solution is heated up to 80° C. for 6 hours and is then cooled to room temperature. The solution is concentrated under reduced pressure and the residue is dissolved in boiling ethanol (5 mL). ethyl acetate (30 mL) is added to the solution and the solid that precipitates out of solution is collected by means of filtration. The solid is washed with cold ethanol (1 mL) and dried under vacuum. The resulting product, 5-(5-hydroxy-3-pyridin-3-yl-5-trifluoromethyl-4,5-dihydro-pyrazol-1-yl)-pyridine-2-carbonitrile (52 mg, 42%), is used in the next step of the synthesis without further purification.

### Step c

To a solution of 5-(5-Hydroxy-3-pyridin-3-yl-5-trifluoromethyl-4,5-dihydro-pyrazol-1-yl)-pyridine-2-carbonitrile (50 mg, 0.150 mmol) in ethanol (5 mL) is added potassium hydroxide (84 mg, 1.5 mmol). The solution is heated in a sealed tube at 80° C. for 6 hours and then cooled to room temperature. The solution is acidified to pH 2 with 12 M aqueous HCl and concentrated under reduced pressure. The residual solid is collected by filtration and is washed with cold ether (5 mL). The product, 5-(3-pyridin-3-yl-5-trifluoromethyl-pyrazol-1-yl)-pyridine-2-carboxylic acid (50 mg, 80%), is used in the next step of the synthesis without further purification.

### Step d

To a solution of 5-(3-pyridin-3-yl-5-trifluoromethyl-pyrazol-1-yl)-pyridine-2-carboxylic acid (50 mg, 0.15 mmol) in dimethylformamide (5 mL) are added 4-aminopyridine (21 mg, 0.225 mmol), 1-(3-dimethylaminopropyl)-3-ethylcarbodiimide (86 mg, 0.45 mmol), 1-hydroxybenzo-triazole hydrate (20 mg, 0.15 mmol) and diisopropylethyl amine (0.05 mL, 0.3 mmol) at room temperature. The solution is stirred at the same temperature for 24 hours. Saturated aqueous sodium bicarbonate solution (10 mL) is added and the solution is extracted with ethyl acetate (20 mL, 3×). The combined organic layers are dried over magnesium sulfate and filtered. The filtrate is concentrated under reduced pressure and the residue is purified by chromatography to afford the title compound (18 mg, 29%). LC-MS (M⁺+1): 411.34.

The following compounds are prepared according to example 41 by replacing the amine in step d with commercially available starting materials.

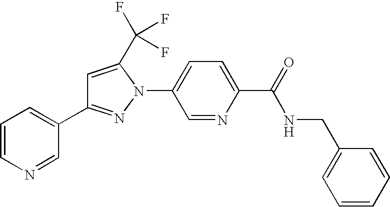

5-(3-Pyridin-3-yl-5-trifluoromethyl-pyrazol-1-yl)-pyridine-2-carboxylic acid benzylamide; LC-MS (M⁺+1): 424.34.

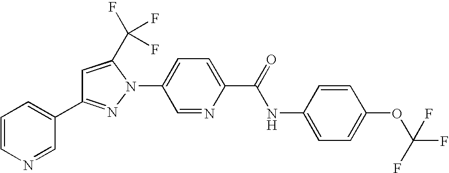

5-(3-Pyridin-3-yl-5-trifluoromethyl-pyrazol-1-yl)-pyridine-2-carboxylic acid (4-trifluoromethoxy-phenyl)-amide; LC-MS (M⁺+1): 494.28

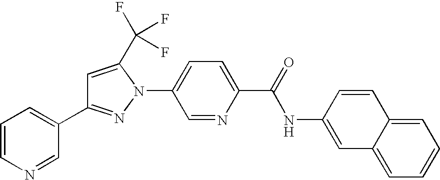

5-(3-Pyridin-3-yl-5-trifluoromethyl-pyrazol-1-yl)-pyridine-2-carboxylic acid naphthalen-2-ylamide; LC-MS (M⁺+1): 460.34

### Example 42

### Cyclohexanecarboxylic Acid [5-(3-pyridin-3-yl-5-trifluoromethyl-pyrazol-1-yl)-pyrazin-2-yl]-amide

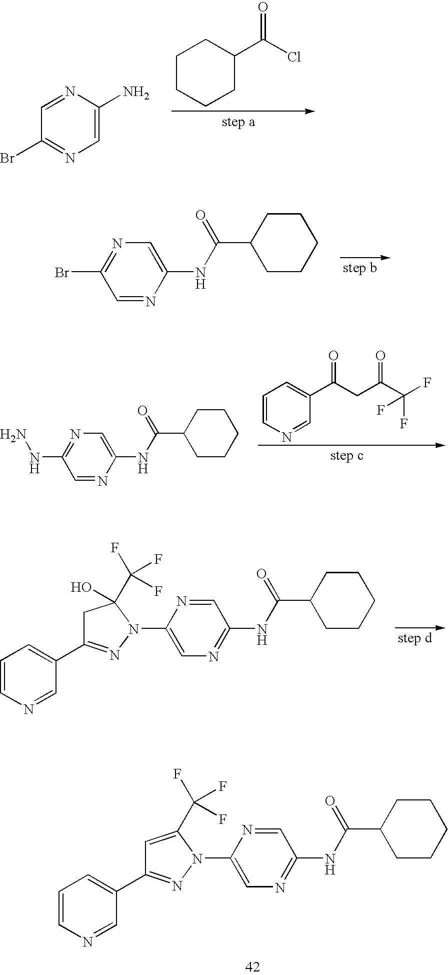

### Step a

To a solution of 2-amino-5-bromopyrazine (200 mg, 1.15 mmol) in tetrahydrofuran (10 mL) are added diisopropylethyl amine (0.4 mL, 2.29 mmol) and cyclohexanecarbonyl chloride (0.18 mL, 1.38 mmol) at 0° C. The solution is stirred at the same temperature for 1 hour. Saturated aqueous ammonium chloride solution (10 mL) is added and the solution is extracted with ethyl acetate (20 mL, 3×). The combined organic layers are dried over magnesium sulfate and filtered. The filtrate is concentrated under reduced pressure and the residue is purified by chromatography to afford cyclohexanecarboxylic acid (5-bromo-pyrazin-2-yl)-amide (300 mg, 92%).

### Step b

To a solution of cyclohexanecarboxylic acid (5-bromo-pyrazin-2-yl)-amide (20 mg, 0.07 mmol) in ethanol (5 mL) is added hydrazine (0.01 mL, 0.35 mmol) at room temperature. The solution is heated to 120° C. in a microwave reactor for 3 hours. The solution is cooled to room temperature and concentrated under reduced pressure. The residue is purified by chromatography to afford cyclohexanecarboxylic acid (5-hydrazino-pyrazin-2-yl)-amide (10 mg, 61%).

### Step c

4,4,4-Trifluoro-1-pyridin-3-yl-butane-1,3-dione is prepared according to step a in example 35. 4,4,4-Trifluoro-1-pyridin-3-yl-butane-1,3-dione (36 mg, 0.17 mmol) is dissolved in ethanol (5 mL) and cyclohexanecarboxylic acid (5-hydrazino-pyrazin-2-yl)-amide (20 mg, 0.085 mmol), 12 M aqueous HCl (1 mL) are added at room temperature. The solution is heated up to 50° C. for 1 hour and then cooled to room temperature. The solution is concentrated under reduced pressure and the residue is purified by chromatography to afford cyclohexanecarboxylic acid [5-(5-hydroxy-3-pyridin-3-yl-5-trifluoromethyl-4,5-dihydro-pyrazol-1-yl)-pyrazin-2-yl]-amide (20 mg, 54%).

### Step d

Cyclohexanecarboxylic acid [5-(5-hydroxy-3-pyridin-3-yl-5-trifluoromethyl-4,5-dihydro-pyrazol-1-yl)-pyrazin-2-yl]-amide (50 mg, 0.115 mmol) is dissolved in acetic acid (5 mL) at room temperature. The solution is heated to 120° C. for 1 hour. The resulting solution is cooled to room temperature and is extracted with ethyl acetate (10 mL, 3×) and water (15 mL). The combined organic layer is dried with magnesium sulfate and filtered. The filtrate is concentrated under reduced pressure and the residue is purified by chromatography to afford the title compound (13 mg, 27%). LC-MS (M⁺+1): 417.36.

The following compounds are prepared according to example 42 by replacing the acyl chloride in step a with commercially available starting materials.

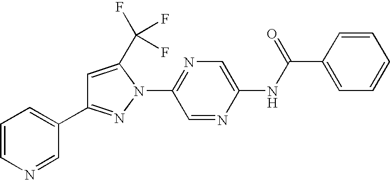

N-[5-(3-Pyridin-3-yl-5-trifluoromethyl-pyrazol-1-yl)-pyrazin-2-yl]-benzamid2; LC-MS (M⁺+1): 411.33.

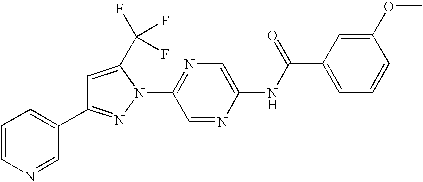

3-Methoxy-N-[5-(3-pyridin-3-yl-5-trifluoromethyl-pyrazol-1-yl)-pyrazin-2-yl]-benzamide; LC-MS (M⁺): 440.88.

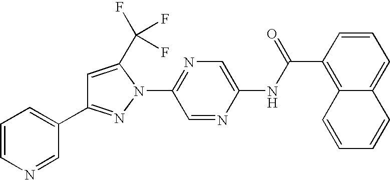

Naphthalene-1-carboxylic acid [5-(3-pyridin-3-yl-5-trifluoromethyl-pyrazol-1-yl)-pyrazin-2-yl]-amide; LC-MS (M⁺): 460.89.

### Example 43

### N-[5-(5-Pyridin-3-yl-3-trifluoromethyl-pyrazol-1-yl)-pyrimidin-2-yl]-benzamide

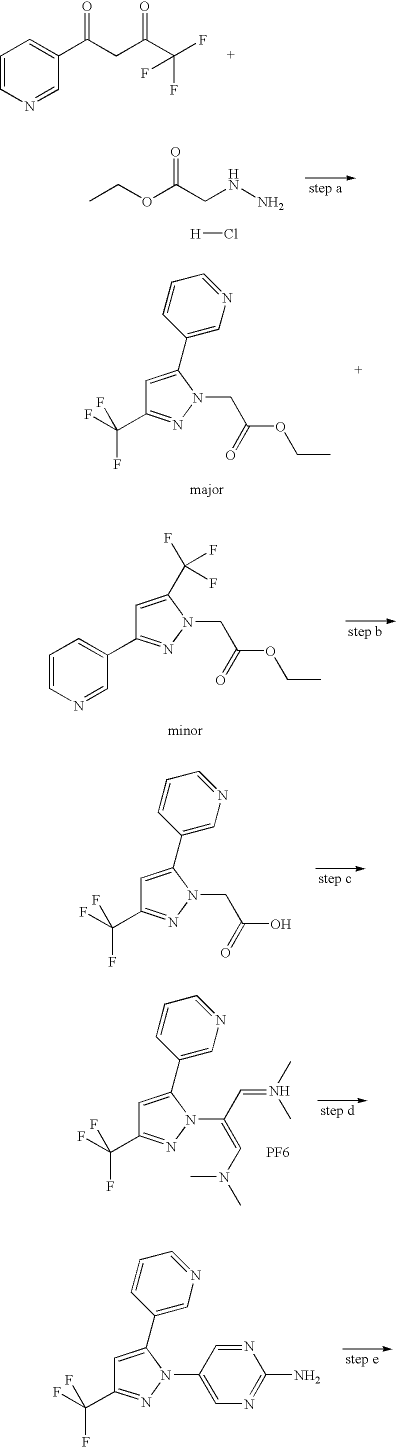

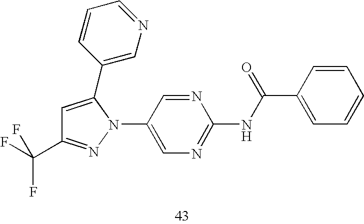

### Step a

4,4,4-Trifluoro-1-pyridin-3-yl-butane-1,3-dione is prepared according to step a in example 35. To a solution of 4,4,4-trifluoro-1-pyridin-3-yl-butane-1,3-dione (200 mg, 0.92 mmol) in ethanol (5 mL) are added ethyl hydrazinoacetate hydrochloride (220 mg, 1.82 mmol) and 12 M aqueous HCl (0.5 mL). The solution is heated to 80° C. for 2 hours. The solution is cooled to room temperature and concentrated under reduced pressure. The residual solid is collected by mean of filtration and is washed with cold ethanol (20 mL). NMR studies reveal this solid to be the major regioisomer, 5-pyridin-3-yl-3-trifluoromethyl-pyrazol-1-yl)-acetic acid ethyl ester (160 mg, 58%). The filtrate was then concentrated under reduced pressure and the residue purified by chromatography to afford the minor regioisomer, 3-Pyridin-3-yl-5-trifluoromethyl-pyrazol-1-yl)-acetic acid ethyl ester (53 mg, 19%).

### Step b

To a solution of (5-pyridin-3-yl-3-trifluoromethyl-pyrazol-1-yl)-acetic acid ethyl ester (200 mg, 0.67 mmol) in methanol (10 mL) are added lithium hydroxide (84 mg, 2 mmol) and water (2 mL) at room temperature. The solution is stirred at the same temperature for 30 minutes. The solution is acidified to pH 2 with 12 M aqueous HCl in an ice bath. The resulting solution is concentrated under reduced pressure and the residual solution is diluted with water (10 mL) and extracted ethyl acetate (20 mL, 3×). The combined organic layers are dried over magnesium sulfate and filtered. The filtrate is concentrated under reduced pressure and the residue is dried under vacuum. The resulting product, (5-pyridin-3-yl-3-trifluoromethyl-pyrazol-1-yl)-acetic acid (100 mg, 55%) is used in the next step of the synthesis without further purification.

### Step c

Phosphoryl chloride (0.35 mmL 3.7 mmol) is added dropwise to a dimethylformamide (2 mL) at 0° C. under nitrogen atmosphere. The solution is stirred at the same temperature for 15 minutes. A solution of (5-pyridin-3-yl-3-trifluoromethyl-pyrazol-1-yl)-acetic acid (500 mg, 1.8 mmol) in dimethylformamide (2 mL) is added to the above solution. The solution is heated up to 105° C. for 3 hours and then cooled to room temperature. The resulting reddish brown oil is poured into a solution of sodium hexafluorophosphate (948 mg, 5.5 mmol) in ice water (10 mL). The solid that precipitates out of the solution is collected by filtration. The brown hydroscopic (Z)-N¹,N¹,N³,N³-Tetramethyl-2-(5-pyridin-3-yl-3-trifluoromethyl-pyrazol-1-yl)-propene-1,3-diamine hexafluorophosphate (325 mg, 52%) is used in the next step of the synthesis without further purification.

### Step d

To a solution of the above (Z)-N¹,N¹,N³,N³-Tetramethyl-2-(5-pyridin-3-yl-3-trifluoromethyl-pyrazol-1-yl)-propene-1,3-diamine hexafluorophosphate (200 mg, 0.41 mmol) in ethanol (10 mL) are added sodium hydride (83 mg, 2.1 mmol) and guanidine carbonate (90 mg, 0.49 mmol) at room temperature under nitrogen atmosphere. The solution is heated up to 80° C. for 6 hours. The solution is cooled to room temperature and then cooled in an ice bath. Water (10 mL) is added and the solution is extracted with ethyl acetate (20 mL, 3×). The combined organic layers are dried over magnesium sulfate and filtered. The filtrate is concentrated under reduced pressure and the residue is purified by chromatography to afford 5-(5-Pyridin-3-yl-3-trifluoromethyl-pyrazol-1-yl)-pyrimidin-2-ylamine (76 mg, 60%).

### Step e

To a solution of 5-(5-pyridin-3-yl-3-trifluoromethyl-pyrazol-1-yl)-pyrimidin-2-ylamine (20 mg, 0.065 mmol) in pyridine (5 mL) are added 4-N,N-dimethylaminopyridine (15.8 mg, 0.13 mmol) and benzoyl chloride (0.009 mL, 0.078 mmol) at room temperature. The solution is stirred at the same temperature for 24 hours. Saturated aqueous sodium bicarbonate solution (5 mL) is added and the solution mixture is diluted with water (10 mL) and extracted with ethyl acetate (20 mL, 3×). The combined organic layers are dried over magnesium sulfate, filtered, concentrated under reduced pressure and the residue is purified by chromatography to afford the title compound (19 mg, 73%). LC-MS (M⁺+1): 411.34.

### Example 44

### N-[5-(3-Pyridin-3-yl-5-trifluoromethyl-pyrazol-1-yl)-pyrimidin-2-yl]-benzamide

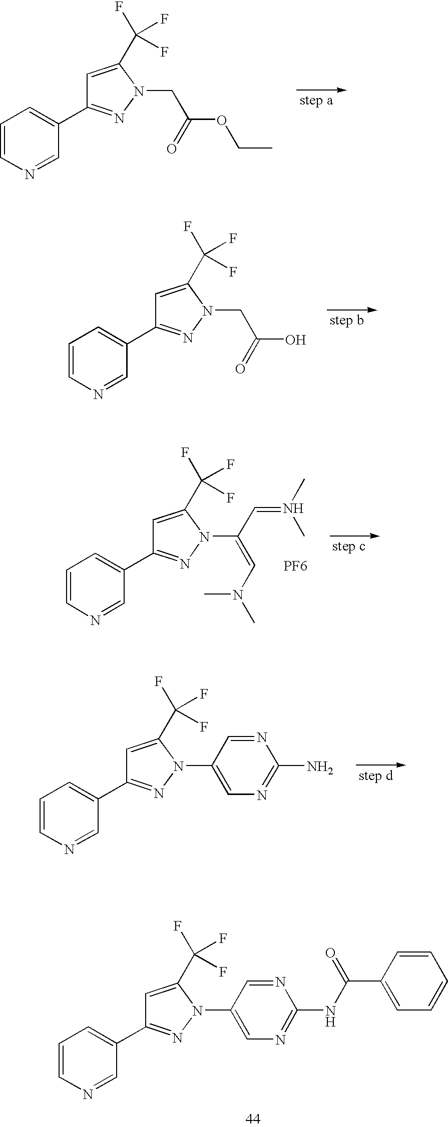

### Step a

(3-Pyridin-3-yl-5-trifluoromethyl-pyrazol-1-yl)-acetic acid ethyl ester is prepared according to step a in example 43. To a solution of (3-pyridin-3-yl-5-trifluoromethyl-pyrazol-1-yl)-acetic acid ethyl ester (50 mg, 0.17 mmol) in methanol (5 mL) are added lithium hydroxide (21 mg, 0.5 mmol) and water (1 mL) at room temperature. The solution is stirred at the same temperature for 30 minutes. The solution is acidified to pH 2 with 12 M aqueous HCl in an ice bath. The resulting solution is concentrated under reduced pressure and the residue is diluted with water (10 mL) and extracted with ethyl acetate (20 mL, 3×). The combined organic layers are dried with magnesium sulfate and then filtered. The filtrate is concentrated under reduced pressure and the solid is dried under vacuum. The resulting product, (3-pyridin-3-yl-5-trifluoromethyl-pyrazol-1-yl)-acetic acid (20 mg, 55%) is used in the next step of the synthesis without further purification.

### Step b

Phosphoryl chloride (0.18 mL 1.85 mmol) is added dropwise to dimethylformamide (2 mL) at 0° C. under nitrogen atmosphere. The solution is stirred at the same temperature for 15 minutes. A solution of (3-pyridin-3-yl-5-trifluoromethyl-pyrazol-1-yl)-acetic acid (250 mg, 0.9 mmol) in dimethylformamide (2 mL) is added to the above solution. The solution is heated up to 105° C. for 3 hours and then cooled to room temperature. The resulting reddish brown oil is poured into a solution of sodium hexafluorophosphate (474 mg, 2.8 mmol) in ice water (10 mL). The solid that precipitates out of the solution is collected by filtration. The brown hydroscopic (Z)-N¹,N¹,N³,N³-Tetramethyl-2-(3-pyridin-3-yl-5-trifluoromethyl-pyrazol-1-yl)-propene-1,3-diamine hexafluorophosphate (163 mg, 52%) is used in the next step of the synthesis without further purification.

### Step c

To a solution of the above (Z)-N¹,N¹,N³,N³-Tetramethyl-2-(3-pyridin-3-yl-5-trifluoromethyl-pyrazol-1-yl)-propene-1,3-diamine hexafluorophosphate (20 mg, 0.041 mmol) in ethanol (5 mL) are added sodium hydride (8 mg, 0.205 mmol) and guanidine carbonate (15 mg, 0.49 mmol) at room temperature under nitrogen atmosphere. The solution is heated up to 80° C. for 6 hours. The solution is cooled to room temperature and then put into an ice bath. Water (10 mL) is added and the solution is then extracted with ethyl acetate (20 mL, 3×). The combined organic layers are dried with magnesium sulfate and then filtered. The filtrate is concentrated under reduced pressure and the residue is purified by chromatography to afford 5-(3-Pyridin-3-yl-5-trifluoromethyl-pyrazol-1-yl)-pyrimidin-2-ylamine (9 mg, 71%).

### Step d

To a solution of 5-(3-pyridin-3-yl-5-trifluoromethyl-pyrazol-1-yl)-pyrimidin-2-ylamine (10 mg, 0.033 mmol) in pyridine (5 mL) are added 4-(dimethylamino)pyridine (8 mg, 0.066 mmol) and benzoyl chloride (0.006 mL, 0.05 mmol) at room temperature. The solution is stirred at the same temperature for 24 hours. Saturated aqueous sodium bicarbonate solution (5 mL) is added and the solution mixture is diluted with water (10 mL) and extracted with ethyl acetate (20 mL, 3×). The combined organic layers are dried over magnesium sulfate, filtered, concentrated under reduced pressure, and the residue is purified by chromatography to afford the title compound (6.5 mg, 48%) as a white solid LC-MS (M⁺+1): 411.02.

### Example 45

### 3-Bromo-N-[6-(3-pyridin-3-yl-5-trifluoromethyl-pyrazol-1-yl)-pyridazin-3-yl]-benzamide

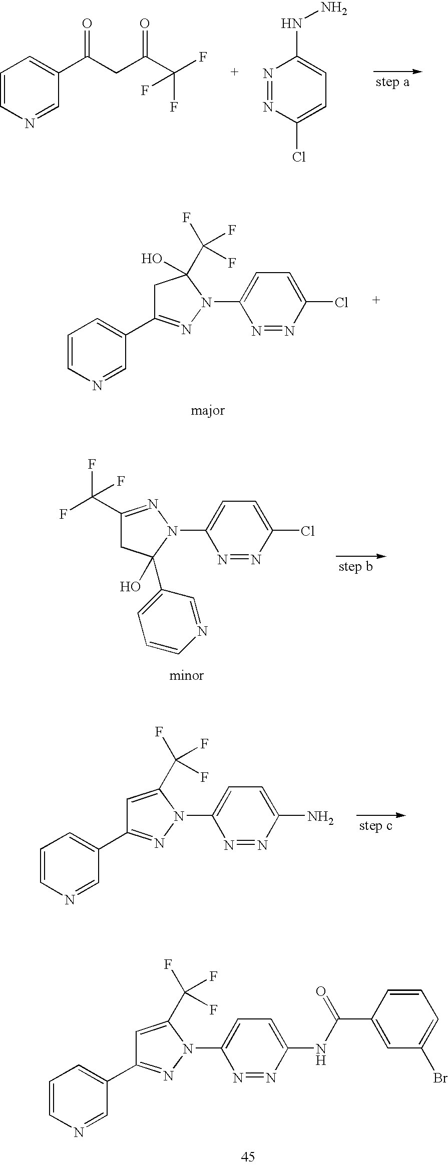

### Step a

4,4,4-Trifluoro-1-pyridin-3-yl-butane-1,3-dione is prepared according to step a in example 35. To a solution of 4,4,4-trifluoro-1-pyridin-3-yl-butane-1,3-dione (883 mg, 4.1 mmol) in ethanol (10 mL) are added 3-chloro-6-hydrazinopyridazine (500 mg, 3.4 mmol) and 12 M aqueous HCl (1 mL). The solution is heated up to 80° C. for 6 hours. The solution is cooled to room temperature and then concentrated under reduced pressure. The residue is dissolved in boiling ethanol (3 mL). Ethyl acetate (20 mL) is added and the solid that precipitates out of solution is collected by means of filtration. The solid is washed with cold ethanol (5 mL) and dried under vacuum. The resulting solid is confirmed by NMR to be the major product, 2-(6-chloro-pyridazin-3-yl)-5-pyridin-3-yl-3-trifluoromethyl-3,4-dihydro-2H-pyrazol-3-ol (650 mg, 56%). The filtrate is concentrated under reduced pressure and the residue is purified by chromatography to afford the minor product, 2-(6-chloro-pyridazin-3-yl)-3-pyridin-3-yl-5-trifluoromethyl-3,4-dihydro-2H-pyrazol-3-ol (450 mg, 39%).

### Step b

2-(6-Chloro-pyridazin-3-yl)-5-pyridin-3-yl-3-trifluoromethyl-3,4-dihydro-2H-pyrazol-3-ol (30 mg, 0.087 mmol) is dissolved in 7 M ammonia in methanol (5 mL) in a sealed tube. The solution is heated up to 110° C. for 8 hours in a microwave reactor. The solution is cooled to room temperature and then concentrated under reduced pressure. The residue is purified by chromatography to afford 6-(3-pyridin-3-yl-5-trifluoromethyl-pyrazol-1-yl)-pyridazin-3-ylamine (18 mg, 68%).

### Step c

To a solution of 6-(3-pyridin-3-yl-5-trifluoromethyl-pyrazol-1-yl)-pyridazin-3-ylamine (50 mg, 0.16 mmol) in tetrahydrofuran (5 mL) are added N,N-diisopropylethylamine (0.057 mL, 0.33 mmol) and 3-bromobenzoyl chloride (0.026 mL, 0.196 mmol) at 0° C. under nitrogen atmosphere. The solution is stirred at the same temperature for 1 hour. Saturated aqueous sodium bicarbonate solution is added and the solution is extracted with ethyl acetate (20 mL, 3×). The combined organic layers are dried over magnesium sulfate and then filtrated. The filtrate is concentrated under reduced pressure and the residue is purified by chromatography to afford the title compound (39 mg, 49%). LC-MS (M⁺+1): 491.21.

The following compounds are prepared according to example 45 by replacing the acyl chloride in step c with commercially available starting materials.

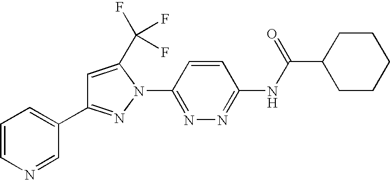

Cyclohexanecarboxylic acid [6-(3-pyridin-3-yl-5-trifluoromethyl-pyrazol-1-yl)-pyridazin-3-yl]-amide; LC-MS (M⁺+1): 417.36.

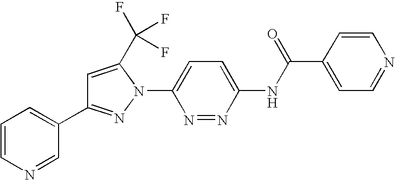

N-[6-(3-Pyridin-3-yl-5-trifluoromethyl-pyrazol-1-yl)-pyridazin-3-yl]-isonicotinamide; LC-MS (M⁺+1): 412.36.

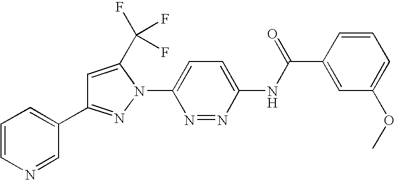

3-Methoxy-N-[6-(3-pyridin-3-yl-5-trifluoromethyl-pyrazol-1-yl)-pyridazin-3-yl]-benzamide; LC-MS (M⁺+1): 440.9.

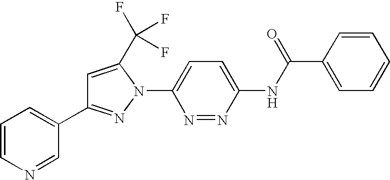

N-[6-(3-Pyridin-3-yl-5-trifluoromethyl-pyrazol-1-yl)-pyridazin-3-yl]-benzamide; LC-MS (M⁺+1): 411.35

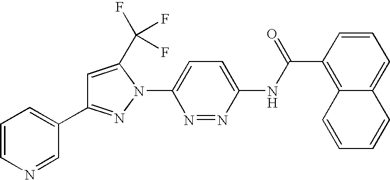

Naphthalene-1-carboxylic acid [6-(3-pyridin-3-yl-5-trifluoromethyl-pyrazol-1-yl)-pyridazin-3-yl]-amide; LC-MS (M⁺+1): 461.34.

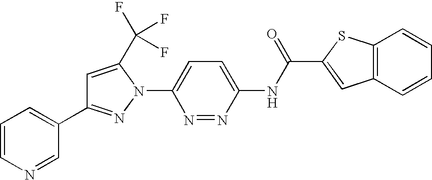

Benzo[b]thiophene-2-carboxylic acid [6-(3-pyridin-3-yl-5-trifluoromethyl-pyrazol-1-yl)-pyridazin-3-yl]-amide; LC-MS (M⁺+1): 467.3.

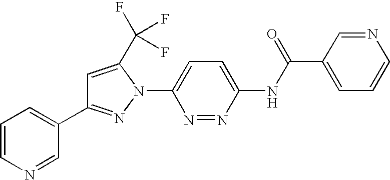

N-[6-(3-Pyridin-3-yl-5-trifluoromethyl-pyrazol-1-yl)-pyridazin-3-yl]-nicotinamide; LC-MS (M⁺+1): 412.33.

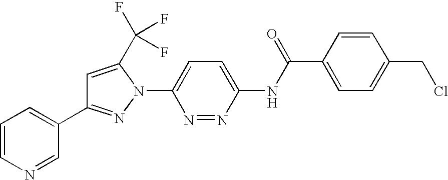

4-Chloromethyl-N-[6-(3-pyridin-3-yl-5-trifluoromethyl-pyrazol-1-yl)-pyridazin-3-yl]-benzamide; LC-MS (M⁺+1): 459.29.

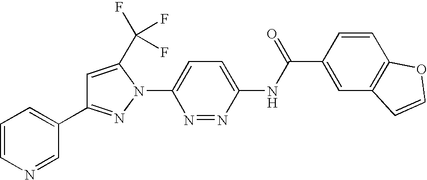

Benzofuran-5-carboxylic acid [6-(3-pyridin-3-yl-5-trifluoromethyl-pyrazol-1-yl)-pyridazin-3-yl]-amide; LC-MS (M⁺+1): 451.33.

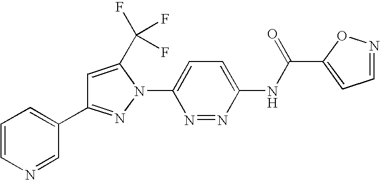

Isoxazole-5-carboxylic acid [6-(3-pyridin-3-yl-5-trifluoromethyl-pyrazol-1-yl)-pyridazin-3-yl]-amide; LC-MS (M⁺+1): 401.92.

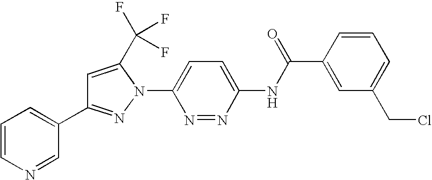

3-Chloromethyl-N-[6-(3-pyridin-3-yl-5-trifluoromethyl-pyrazol-1-yl)-pyridazin-3-yl]-benzamide; LC-MS (M⁺+1): 458.89.

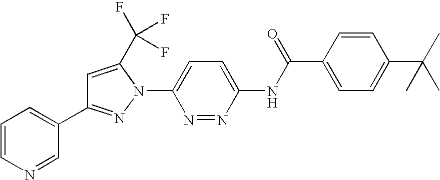

4-tert-Butyl-N-[6-(3-pyridin-3-yl-5-trifluoromethyl-pyrazol-1-yl)-pyridazin-3-yl]-benzamide; LC-MS (M⁺+1): 467.37.

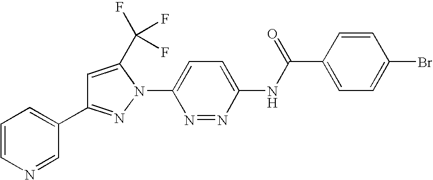

4-Bromo-N-[6-(3-pyridin-3-yl-5-trifluoromethyl-pyrazol-1-yl)-pyridazin-3-yl]-benzamide; LC-MS (M⁺+1): 489.96.

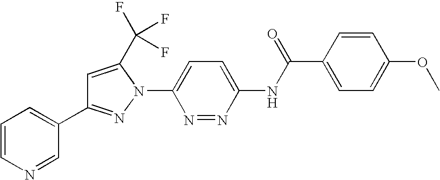

4-Methoxy-N-[6-(3-pyridin-3-yl-5-trifluoromethyl-pyrazol-1-yl)-pyridazin-3-yl]-benzamide; LC-MS (M⁺+1): 441.38.

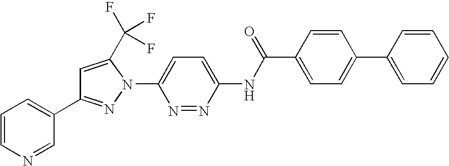

Biphenyl-4-carboxylic acid [6-(3-pyridin-3-yl-5-trifluoromethyl-pyrazol-1-yl)-pyridazin-3-yl]-amide; LC-MS (M⁺+1): 487.38.

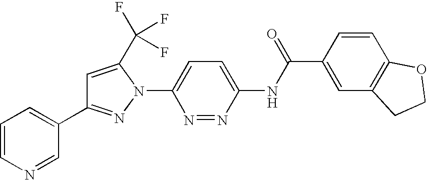

2,3-Dihydro-benzofuran-5-carboxylic acid [6-(3-pyridin-3-yl-5-trifluoromethyl-pyrazol-1-yl)-pyridazin-3-yl]-amide; LC-MS (M⁺+1): 453.35.

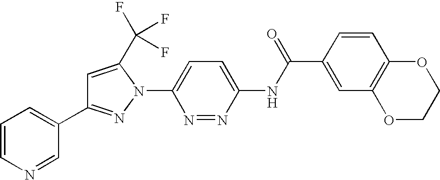

2,3-Dihydro-benzo[1,4]dioxine-6-carboxylic acid [6-(3-pyridin-3-yl-5-trifluoromethyl-pyrazol-1-yl)-pyridazin-3-yl]-amide; LC-MS (M⁺+1): 469.3.

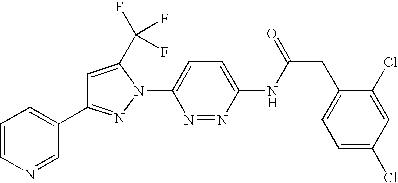

2-(2,4-Dichloro-phenyl)-N-[6-(3-pyridin-3-yl-5-trifluoromethyl-pyrazol-1-yl)-pyridazin-3-yl]-acetamide; LC-MS (M⁺+1): 495.94.

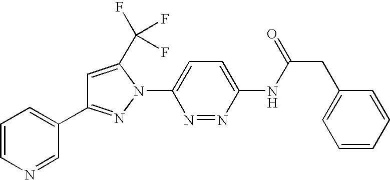

2-Phenyl-N-[6-(3-pyridin-3-yl-5-trifluoromethyl-pyrazol-1-yl)-pyridazin-3-yl]-acetamide; LC-MS (M⁺+1): 425.02.

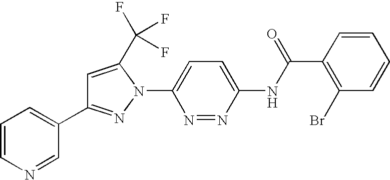

2-Bromo-N-[6-(3-pyridin-3-yl-5-trifluoromethyl-pyrazol-1-yl)-pyridazin-3-yl]-benzamide; LC-MS (M⁺+1): 489.27.

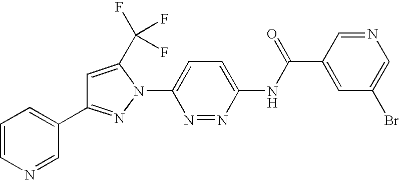

5-Bromo-N-[6-(3-pyridin-3-yl-5-trifluoromethyl-pyrazol-1-yl)-pyridazin-3-yl]-nicotinamide; LC-MS (M⁺+1): 490.29.

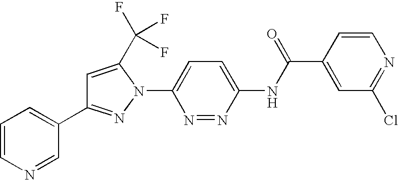

2-Chloro-N-[6-(3-pyridin-3-yl-5-trifluoromethyl-pyrazol-1-yl)-pyridazin-3-yl]-isonicotinamide; LC-MS (M⁺+1): 446.37.

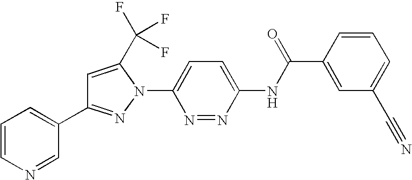

3-Cyano-N-[6-(3-pyridin-3-yl-5-trifluoromethyl-pyrazol-1-yl)-pyridazin-3-yl]-benzamide; LC-MS (M⁺+1): 436.35.

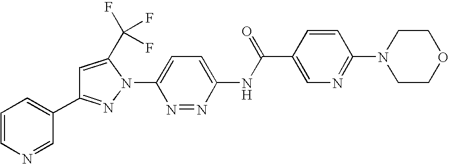

6-Morpholin-4-yl-N-[6-(3-pyridin-3-yl-5-trifluoromethyl-pyrazol-1-yl)-pyridazin-3-yl]-nicotinamide; LC-MS (M⁺+1): 497.32.

### Example 46

### Tetrahydro-pyran-4-carboxylic Acid [6-(3-pyridin-3-yl-5-trifluoromethyl-pyrazol-1-yl)-pyridazin-3-yl]-amide

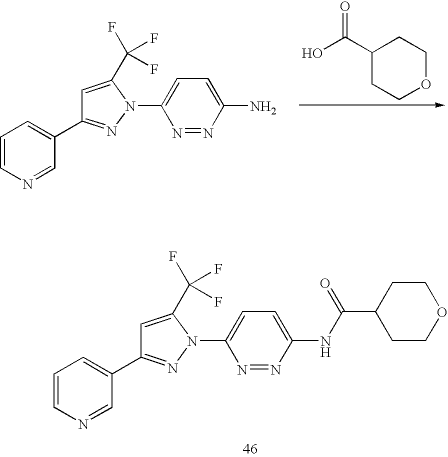

6-(3-Pyridin-3-yl-5-trifluoromethyl-pyrazol-1-yl)-pyridazin-3-ylamine is prepared according to step b in example 45. To a solution of 6-(3-pyridin-3-yl-5-trifluoromethyl-pyrazol-1-yl)-pyridazin-3-ylamine (30 mg, 0.098 mmol) in dimethylformamide (10 mL) are added tetrahydropyran-4-yl-carbonylic acid (19 mg, 0.147 mmol), 1-(3-dimethylaminopropyl)-3-ethylcarbodiimide (58 mg, 0.29 mmol), 1-hydroxybenzo-triazole hydrate (26 mg, 0.196 mmol) and N,N-diisopropylethylamine (0.034 mL, 0.196 mmol). The solution is stirred at room temperature for 24 hours. Saturated aqueous sodium bicarbonate solution (10 mL) is added and the solution is extracted with ethyl acetate (20 mL, 3×). The combined organic layers are dried over magnesium sulfate and then filtered. The filtrate is concentrated under reduced pressure. The residue is purified by chromatography to afford the title compound (5 mg, 12%). LC-MS (M⁺+1): 418.94.

### Example 47

### 4-Morpholin-4-yl-N-[6-(3-pyridin-3-yl-5-trifluoromethyl-pyrazol-1-yl)-pyridazin-3-yl]-benzamide

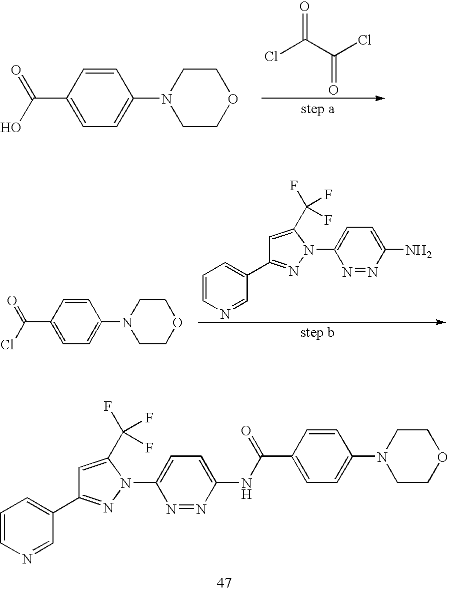

### Step a

To a suspension of 4-morpholinobenzoic acid (500 mg, 2.4 mmol) in dichloromethane (20 mL) is added oxalyl chloride (0.4 mL, 4.8 mmol) at room temperature under nitrogen atmosphere. The solution is heated at reflux for 3 hours. The solution is concentrated under reduced pressure and the residue, 4-morpholin-4-yl-benzoyl chloride (340 mg, 62%), is used in the next step of the synthesis without further purification.

### Step b

6-(3-Pyridin-3-yl-5-trifluoromethyl-pyrazol-1-yl)-pyridazin-3-ylamine is prepared according to step b in example 45. To a solution of 6-(3-pyridin-3-yl-5-trifluoromethyl-pyrazol-1-yl)-pyridazin-3-ylamine (200 mg, 0.65 mmol) in tetrahydrofuran (10 mL) are added N,N-diisopropylethylamine (0.23 mL, 1.0 mmol) and 4-morpholin-4-yl-benzoyl chloride (294 mg, 1.3 mmol) respectively at 0° C. under nitrogen atmosphere. The solution is stirred at the same temperature for 1 hour. Saturated aqueous sodium bicarbonate solution (10 mL) is added and the solution is extracted with ethyl acetate (20 mL, 3×). The combined organic layers are dried over magnesium sulfate and then filtered. The filtrate is concentrated under reduced pressure and the residue is purified by chromatography to afford the title compound (183 mg, 57%). LC-MS (M⁺+1): 496.31.

The following compounds are prepared according to example 47 by replacing the carboxylic acid with commercially available starting materials.

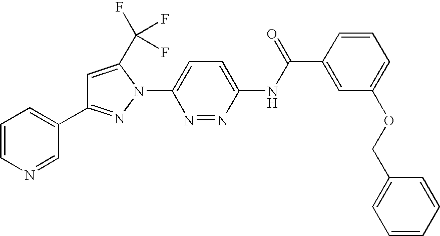

3-Benzyloxy-N-[6-(3-pyridin-3-yl-5-trifluoromethyl-pyrazol-1-yl)-pyridazin-3-yl]-benzamide; LC-MS (M⁺+1): 516.06.

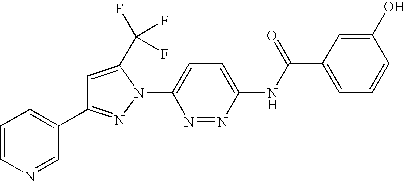

3-Hydroxy-N-[6-(3-pyridin-3-yl-5-trifluoromethyl-pyrazol-1-yl)-pyridazin-3-yl]-benzamide; LC-MS (M⁺+1): 427.03.

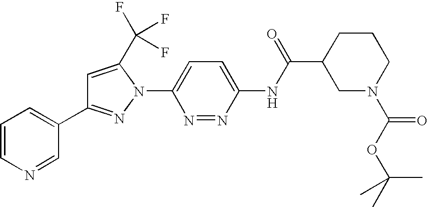

3-[6-(3-Pyridin-3-yl-5-trifluoromethyl-pyrazol-1-yl)-pyridazin-3-ylcarbamoyl]-piperidine-1-carboxylic acid tert-butyl ester; LC-MS (M⁺+1): 517.15.

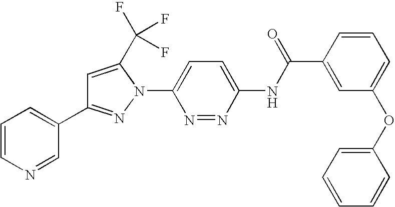

1-Phenoxy-N-[6-(3-pyridin-3-yl-5-trifluoromethyl-pyrazol-1-yl)-pyridazin-3-yl]-benzamide; LC-MS (M⁺+1): 503.38.

### Example 48

### Biphenyl-3,3′-dicarboxylic acid 3′-dimethylamide 3-{[6-(3-pyridin-3-yl-5-trifluoromethyl-pyrazol-1-yl)-pyridazin-3-yl]-amide}

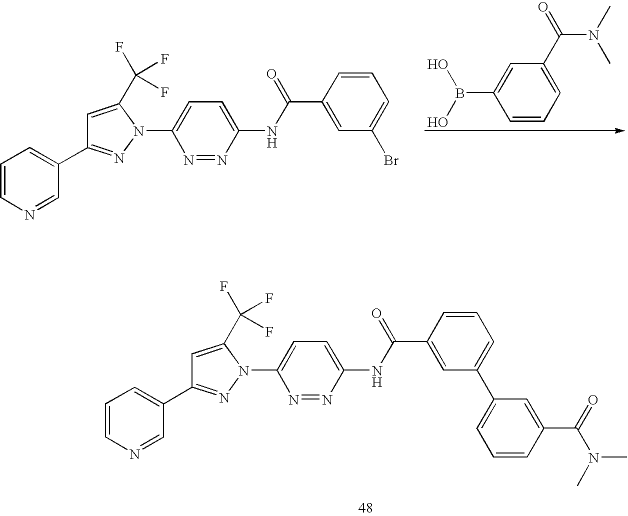

To a solution of 3-bromo-N-[6-(3-pyridin-3-yl-5-trifluoromethyl-pyrazol-1-yl)-pyridazin-3-yl]-benzamide (60 mg, 0.123 mmol), prepared according example 45, in dimethylformamide (5 mL) are added [3-(N,N-dimethylaminocarbonyl)phenyl]boronic acid (47 mg, 0.25 mmol), tetrakis(triphenylphosphine)palladium(0) (14 mg, 0.012 mmol) and cesium carbonate (40 mg, 0.123 mmol). The solution is heated to 100° C. for 10 minutes in a microwave reactor. The reddish brown solution is cooled to room temperature and 3-mercaptopropyl-functionalized silica gel (500 mg) is added to the solution. The resulting solution is stirred for 30 minutes and filtered. Water (20 mL) is added to the filtrate and the resulting mixture is extracted with ethyl acetate (20 mL, 3×). The combined organic layers are dried over magnesium sulfate and then filtered. The filtrate is concentrated under reduced pressure and the residue is purified by chromatography to afford the title compound (33 mg, 49%). LC-MS (M⁺+1): 558.69.

The following compounds are prepared according to example 48 by replacing the boronic acid with commercially available starting materials.

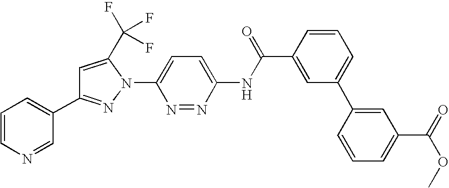

3′-[6-(3-Pyridin-3-yl-5-trifluoromethyl-pyrazol-1-yl)-pyridazin-3-ylcarbamoyl]-biphenyl-3-carboxylic acid methyl ester; LC-MS (M⁺+1): 545.37.

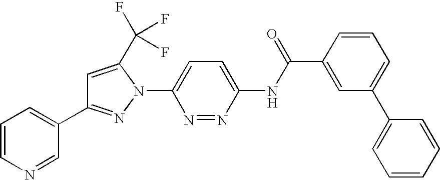

Biphenyl-3-carboxylic acid [6-(3-pyridin-3-yl-5-trifluoromethyl-pyrazol-1-yl)-pyridazin-3-yl]-amide; LC-MS (M⁺+1): 487.36.

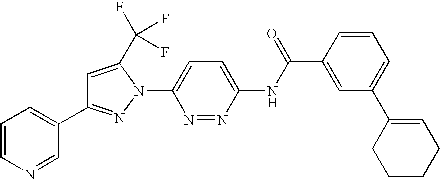

3-Cyclohex-1-enyl-N-[6-(3-pyridin-3-yl-5-trifluoromethyl-pyrazol-1-yl)-pyridazin-3-yl]-benzamide; LC-MS (M⁺+1): 491.41.

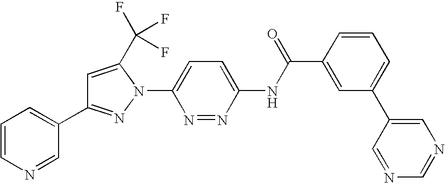

N-[6-(3-Pyridin-3-yl-5-trifluoromethyl-pyrazol-1-yl)-pyridazin-3-yl]-3-pyrimidin-5-yl-benzamide; LC-MS (M⁺+1): 489.05.

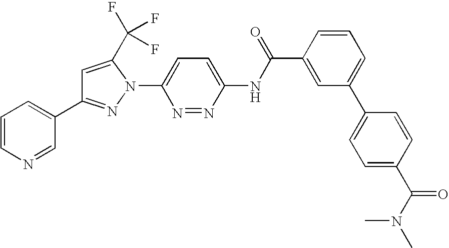

Biphenyl-3,4′-dicarboxylic acid 4′-dimethylamide 3-{[6-(3-pyridin-3-yl-5-trifluoromethyl-pyrazol-1-yl)-pyridazin-3-yl]-amide}; LC-MS (M⁺+1): 558.7.

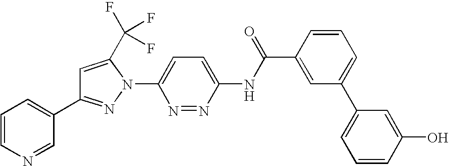

3′-Hydroxy-biphenyl-3-carboxylic acid [6-(3-pyridin-3-yl-5-trifluoromethyl-pyrazol-1-yl)-pyridazin-3-yl]-amide; LC-MS (M⁺+1): 503.1.

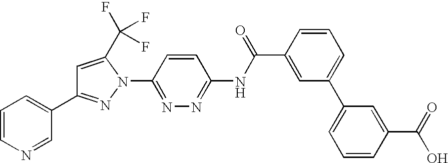

3′-[6-(3-Pyridin-3-yl-5-trifluoromethyl-pyrazol-1-yl)-pyridazin-3-ylcarbamoyl]-biphenyl-3-carboxylic acid; LC-MS (M⁺+1): 531.09.

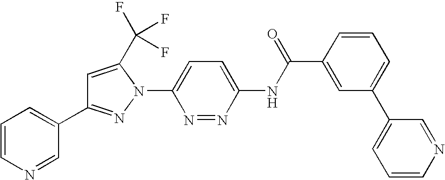

3-Pyridin-3-yl-N-[6-(3-pyridin-3-yl-5-trifluoromethyl-pyrazol-1-yl)-pyridazin-3-yl]-benzamide; LC-MS (M⁺+1): 488.4.

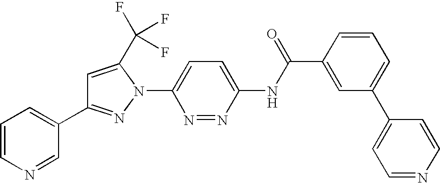

3-Pyridin-4-yl-N-[6-(3-pyridin-3-yl-5-trifluoromethyl-pyrazol-1-yl)-pyridazin-3-yl]-benzamide; LC-MS (M¹+1): 488.4.

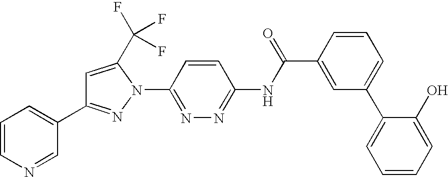

2′-Hydroxy-biphenyl-3-carboxylic acid [6-(3-pyridin-3-yl-5-trifluoromethyl-pyrazol-1-yl)-pyridazin-3-yl]-amide; LC-MS (M¹+1): 503.08.

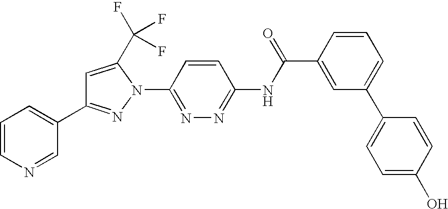

4′-Hydroxy-biphenyl-3-carboxylic acid [6-(3-pyridin-3-yl-5-trifluoromethyl-pyrazol-1-yl)-pyridazin-3-yl]-amide; LC-MS (M⁺+1): 503.08.

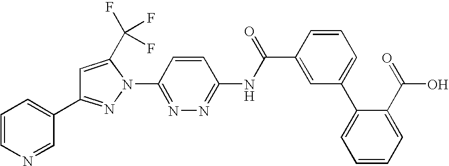

3′-[6-(3-Pyridin-3-yl-5-trifluoromethyl-pyrazol-1-yl)-pyridazin-3-ylcarbamoyl]-biphenyl-2-carboxylic acid; LC-MS (M⁺+1): 531.08.

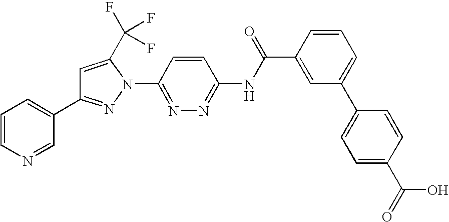

3′-[6-(3-Pyridin-3-yl-5-trifluoromethyl-pyrazol-1-yl)-pyridazin-3-ylcarbamoyl]-biphenyl-4-carboxylic acid; LC-MS (M⁺+1): 531.1.

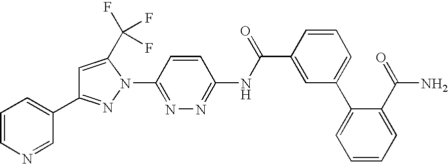

Biphenyl-2,3′-dicarboxylic acid 2-amide 3′-{[6-(3-pyridin-3-yl-5-trifluoromethyl-pyrazol-1-yl)-pyridazin-3-yl]-amide}; LC-MS (M⁺+1): 530.43.

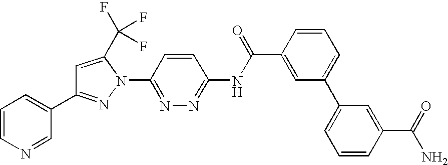

Biphenyl-3,3′-dicarboxylic acid 3′-amide 3-{[6-(3-pyridin-3-yl-5-trifluoromethyl-pyrazol-1-yl)-pyridazin-3-yl]-amide}; LC-MS (M⁺+1): 530.4.

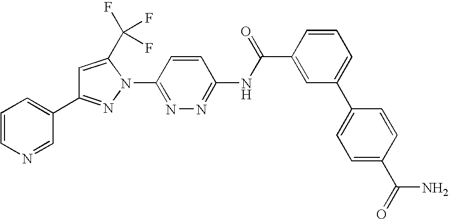

Biphenyl-3,4′-dicarboxylic acid 4′-amide 3-{[6-(3-pyridin-3-yl-5-trifluoromethyl-pyrazol-1-yl)-pyridazin-3-yl]-amide}; LC-MS (M⁺+1): 530.39.

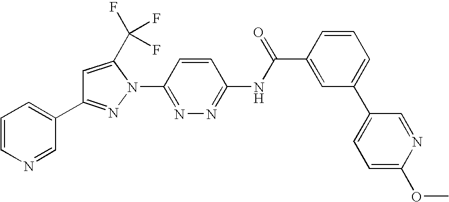

3-(6-Methoxy-pyridin-3-yl)-N-[6-(3-pyridin-3-yl-5-trifluoromethyl-pyrazol-1-yl)-pyridazin-3-yl]-benzamide; LC-MS (M⁺+1): 518.1.

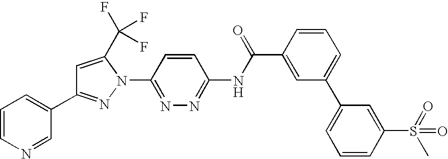

3′-Methanesulfonyl-biphenyl-3-carboxylic acid [6-(3-pyridin-3-yl-5-trifluoromethyl-pyrazol-1-yl)-pyridazin-3-yl]-amide LC-MS (M⁺+1): 565.05

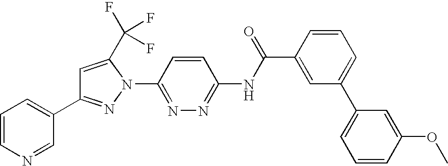

3′-Methoxy-biphenyl-3-carboxylic acid [6-(3-pyridin-3-yl-5-trifluoromethyl-pyrazol-1-yl)-pyridazin-3-yl]-amide; LC-MS (M⁺+1): 517.4.

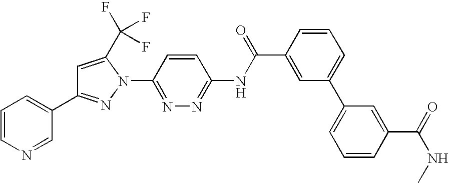

Biphenyl-3,3′-dicarboxylic acid 3′-methylamide 3-{[6-(3-pyridin-3-yl-5-trifluoromethyl-pyrazol-1-yl)-pyridazin-3-yl]-amide}; LC-MS (M⁺+1): 544.46.

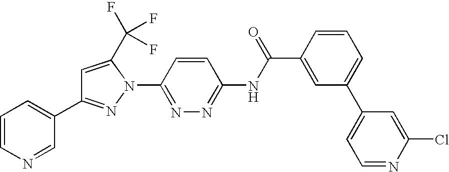

3-(2-Chloro-pyridin-4-yl)-N-[6-(3-pyridin-3-yl-5-trifluoromethyl-pyrazol-1-yl)-pyridazin-3-yl]-benzamide; LC-MS (M⁺): 521.06.

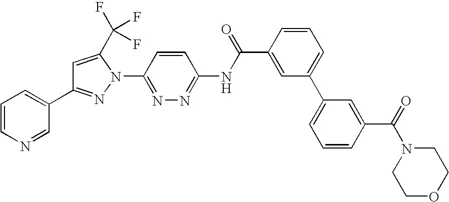

3′-(Morpholine-4-carbonyl)-biphenyl-3-carboxylic acid [6-(3-pyridin-3-yl-5-trifluoromethyl-pyrazol-1-yl)-pyridazin-3-yl]-amide; LC-MS (M¹+1): 600.57.

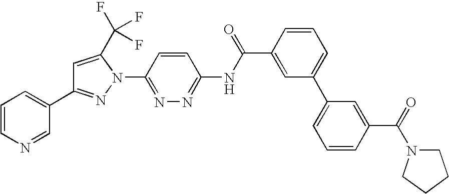

3′-(Pyrrolidine-1-carbonyl)-biphenyl-3-carboxylic acid [6-(3-pyridin-3-yl-5-trifluoromethyl-pyrazol-1-yl)-pyridazin-3-yl]-amide; LC-MS (M⁺+1): 584.66.

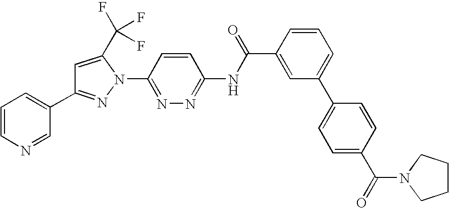

4′-(Pyrrolidine-1-carbonyl)-biphenyl-3-carboxylic acid [6-(3-pyridin-3-yl-5-trifluoromethyl-pyrazol-1-yl)-pyridazin-3-yl]-amide; LC-MS (M⁺+1): 584.64.

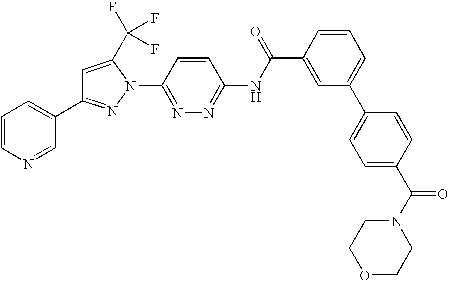

4′-(Morpholine-4-carbonyl)-biphenyl-3-carboxylic acid [6-(3-pyridin-3-yl-5-trifluoromethyl-pyrazol-1-yl)-pyridazin-3-yl]-amide; LC-MS (M⁺+1): 600.57.

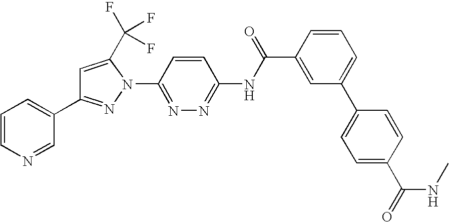

Biphenyl-3,4′-dicarboxylic acid 4′-methylamide 3-{[6-(3-pyridin-3-yl-5-trifluoromethyl-pyrazol-1-yl)-pyridazin-3-yl]-amide}; LC-MS (M⁺+1): 544.04.

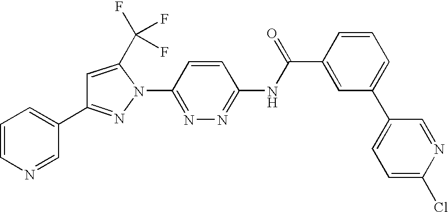

3-(6-Chloro-pyridin-3-yl)-N-[6-(3-pyridin-3-yl-5-trifluoromethyl-pyrazol-1-yl)-pyridazin-3-yl]-benzamide; LC-MS (M⁺+1): 522.25.

### Example 49

### Biphenyl-2-carboxylic Acid [6-(3-pyridin-3-yl-5-trifluoromethyl-pyrazol-1-yl)-pyridazin-3-yl]-amide

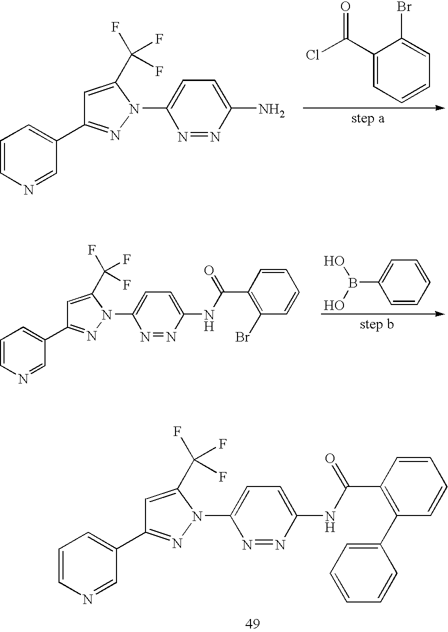

### Step a

6-(3-Pyridin-3-yl-5-trifluoromethyl-pyrazol-1-yl)-pyridazin-3-ylamine is prepared according to step b in example 45. To a solution of 6-(3-pyridin-3-yl-5-trifluoromethyl-pyrazol-1-yl)-pyridazin-3-ylamine (100 mg, 0.33 mmol) in tetrahydrofuran (10 mL) are added N,N-diisopropylethylamine (0.28 mL, 1.6 mmol) and 2-bromobenzoyl chloride (0.085 mL, 0.65 mmol) respectively at 0° C. under nitrogen atmosphere. The solution is stirred at the same temperature for 1 hour. Saturated aqueous sodium bicarbonate solution (10 mL) is added and the solution is extracted with ethyl acetate (20 mL, 3×). The combined organic layers are dried over magnesium sulfate and then filtered. The filtrate is concentrated under reduced pressure and the residue is purified by chromatography to afford. 2-Bromo-N-[6-(3-pyridin-3-yl-5-trifluoromethyl-pyrazol-1-yl)-pyridazin-3-yl]-benzamide (140 mg, 88%).

### Step b

To a solution of 2-bromo-N-[6-(3-pyridin-3-yl-5-trifluoromethyl-pyrazol-1-yl)-pyridazin-3-yl]-benzamide (50 mg, 0.102 mmol) in dimethylformamide (5 mL) are added phenylboronic acid (26 mg, 0.204 mmol), tetrakis(triphenylphosphine)palladium(0) (14 mg, 0.012 mmol) and cesium carbonate (33 mg, 0.102 mmol). The solution is heated to 100° C. for 10 minutes in a microwave reactor. The reddish brown solution is cooled to room temperature and 3-mercaptopropyl-functionalized silica gel (500 mg) is added to the solution. The resulting solution is stirred for 30 minutes and filtered. Water (20 mL) is added to the filtrate and the resulting mixture is extracted with ethyl acetate (20 mL, 3×). The combined organic layers are dried over magnesium sulfate and then filtered. The filtrate is concentrated under reduced pressure and the residue is purified by chromatography to afford the title compound (11 mg, 22%). LC-MS (M⁺+1): 487.41.

### Example 50

### N-[6-(3-Pyridin-3-yl-5-trifluoromethyl-pyrazol-1-yl)-pyridazin-3-yl]-5-pyrimidin-5-yl-nicotinamide

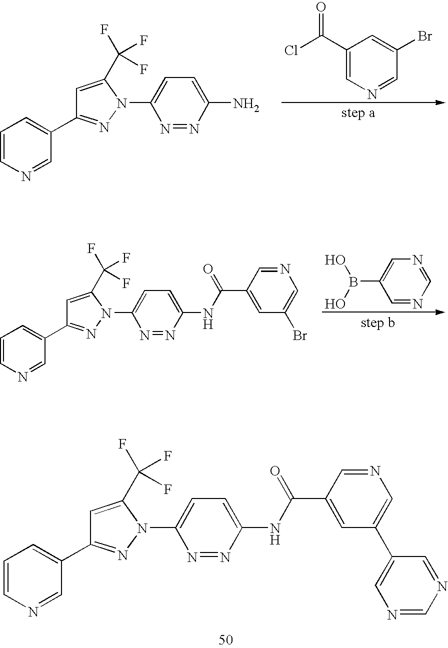

### Step a

6-(3-Pyridin-3-yl-5-trifluoromethyl-pyrazol-1-yl)-pyridazin-3-ylamine is prepared according to step b in example 45. To a solution of 6-(3-pyridin-3-yl-5-trifluoromethyl-pyrazol-1-yl)-pyridazin-3-ylamine (250 mg, 0.816 mmol) in tetrahydrofuran (10 mL) are added N,N-diisopropylethylamine (0.43 mL, 2.45 mmol) and 5-bromonicotinyl chloride (360 mg, 1.63 mmol) respectively at 0° C. under nitrogen atmosphere. The solution is stirred at the same temperature for 1 hour. Saturated aqueous sodium bicarbonate solution (10 mL) is added and the solution is extracted with ethyl acetate (20 mL, 3×). The combined organic layers are dried over magnesium sulfate and then filtered. The filtrate is concentrated under reduced pressure and the residue is purified by chromatography to afford 5-bromo-N-[6-(3-pyridin-3-yl-5-trifluoromethyl-pyrazol-1-yl)-pyridazin-3-yl]-nicotinamide (270 mg, 68%).

### Step b

To a solution of 5-bromo-N-[6-(3-pyridin-3-yl-5-trifluoromethyl-pyrazol-1-yl)-pyridazin-3-yl]-nicotinamide (60 mg, 0.122 mmol) in dimethylformamide (5 mL) are added 5-pyrimidinylboronic acid (30 mg, 0.24 mmol), tetrakis(triphenylphosphine)palladium(0) (14 mg, 0.012 mmol) and cesium carbonate (40 mg, 0.123 mmol). The solution is heated to 100° C. for 10 minutes in a microwave reactor. The reddish brown solution is cooled to room temperature and 3-mercaptopropyl-functionalized silica gel (500 mg) is added to the solution. The resulting solution is stirred for 30 minutes and then filtered. Water (20 mL) is added to the filtrate and the resulting mixture is extracted with ethyl acetate (20 mL, 3×). The combined organic layers are dried over magnesium sulfate and then filtered. The filtrate is concentrated under reduced pressure and the residue is purified by chromatography to afford the title compound (20 mg, 34%). LC-MS (M⁺+1): 490.44.

The following compounds are prepared according to example 50 by replacing the boronic acid in step b with commercially available starting materials.

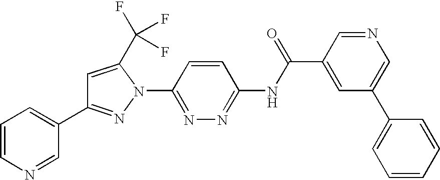

5-Phenyl-N-[6-(3-pyridin-3-yl-5-trifluoromethyl-pyrazol-1-yl)-pyridazin-3-yl]-nicotinamide; LC-MS (M⁺+1): 488.38.

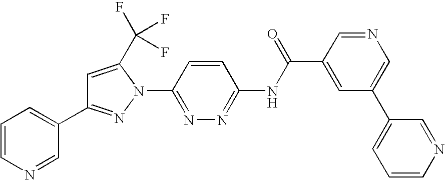

[3,3′]Bipyridinyl-5-carboxylic acid [6-(3-pyridin-3-yl-5-trifluoromethyl-pyrazol-1-yl)-pyridazin-3-yl]-amide; LC-MS (M⁺+1): 489.36.

### Example 51

### 3-Oxazol-5-yl-N-[6-(3-pyridin-3-yl-5-trifluoromethyl-pyrazol-1-yl)-pyridazin-3-yl]-benzamide

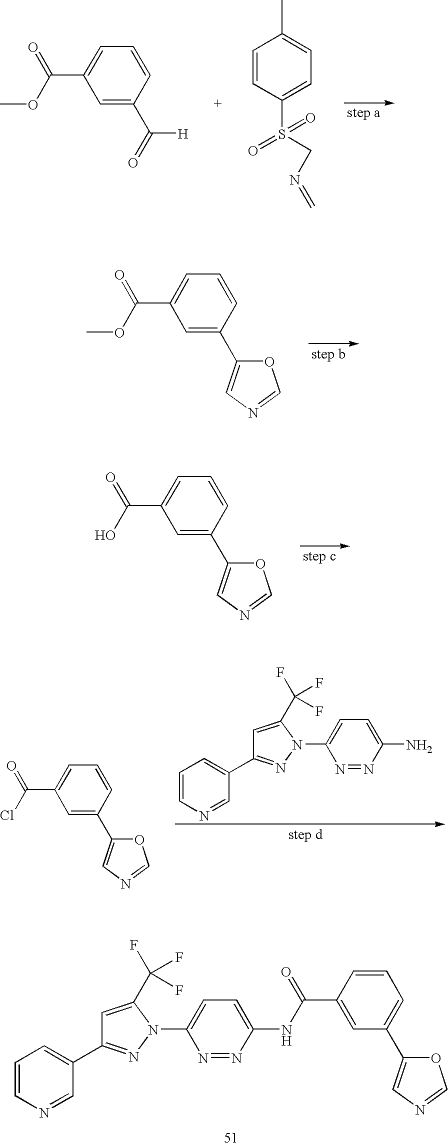

### Step a

To a solution of methyl-3-formylbenzoate (300 mg, 1.828 mmol) in methanol (20 mL) are added tosylmethyl isocyanide (541 mg, 2.7 mmol) and potassium carbonate (505 mg, 3.7 mmol) at room temperature. The solution mixture is heated at reflux for 3 hours. The solution is then cooled to room temperature and concentrated under reduced pressure. The residue, 3-oxazol-5-yl-benzoic acid methyl ester (200 mg, 54%), is used in the next step of the synthesis without further purification.

### Step b

To a solution of 3-oxazol-5-yl-benzoic acid methyl ester (300 mg, 1.48 mmol) in methanol (20 mL) and water (5 mL) is added lithium hydroxide (71 mg, 2.9 mmol) at room temperature. The solution is stirred at the same temperature for 24 hours. The solution is cooled in an ice bath and 12 M aqueous HCl is added to adjust the pH of the solution to 2. The solution is concentrated under reduced pressure and the remaining solid is collected by means of filtration. The solid is washed with cold methanol (5 mL) and dried under vacuum. The resulting product, 3-oxazol-5-yl-benzoic acid (164 mg, 59%), is used in the next step of the synthesis without further purification.

### Step c

To a solution of 3-oxazol-5-yl-benzoic acid (100 mg, 0.53 mmol) in dichlormethane (10 mL) is added oxalyl chloride (0.14 mL, 1.59 mmol) at room temperature under nitrogen atmosphere. The solution is heated at reflux for 3 hours and then cooled to room temperature. The solution is concentrated under reduced pressure and the residue, 3-oxazol-5-yl-benzoyl chloride (95 mg, 87%), is used in the next step of the synthesis without further purification.

### Step d

6-(3-Pyridin-3-yl-5-trifluoromethyl-pyrazol-1-yl)-pyridazin-3-ylamine is prepared according to step b in example 45. To a solution of 6-(3-pyridin-3-yl-5-trifluoromethyl-pyrazol-1-yl)-pyridazin-3-ylamine (100 mg, 0.327 mmol) in tetrahydrofuran (10 mL) are added N,N-diisopropylethylamine (0.28 mL, 1.63 mmol) and 3-Oxazol-5-yl-benzoyl chloride (136 mg, 0.65 mmol) respectively at 0° C. under nitrogen atmosphere. The solution is stirred at the same temperature for 1 hour. Saturated aqueous sodium bicarbonate solution (10 mL) is added and the solution is extracted with ethyl acetate (20 mL, 3×). The combined organic layers are dried over magnesium sulfate and then filtered. The filtrate is concentrated under reduced pressure and the residue is purified by chromatography to afford the title compound (45 mg, 29%). LC-MS (M⁺+1): 478.39.

### Example 52

### 2-Phenyl-N-[6-(3-pyridin-3-yl-5-trifluoromethyl-pyrazol-1-yl)-pyridazin-3-yl]-isonicotinamide

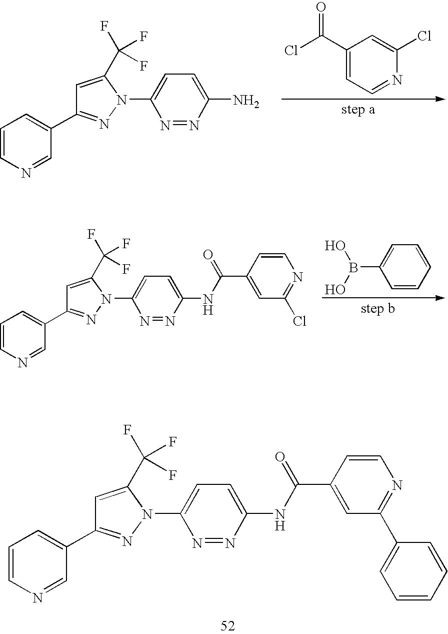

### Step a

6-(3-Pyridin-3-yl-5-trifluoromethyl-pyrazol-1-yl)-pyridazin-3-ylamine is prepared according to step b in example 45. To a solution of 6-(3-pyridin-3-yl-5-trifluoromethyl-pyrazol-1-yl)-pyridazin-3-ylamine (300 mg, 0.98 mmol) in tetrahydrofuran (10 mL) are added N,N-diisopropylethylamine (0.43 mL, 2.45 mmol) and 2-chloropyridine-4-carbonyl chloride (345 mg, 1.96 mmol) respectively at 0° C. under nitrogen atmosphere. The solution is stirred at the same temperature for 1 hour. Saturated aqueous sodium bicarbonate solution (10 mL) is added and the solution is extracted with ethyl acetate (20 mL, 3×). The combined organic layers are dried over magnesium sulfate and then filtered. The filtrate is concentrated under reduced pressure and the residue is purified by chromatography to afford 2-chloro-N-[6-(3-pyridin-3-yl-5-trifluoromethyl-pyrazol-1-yl)-pyridazin-3-yl]-isonicotinamide (350 mg, 80%).

### Step b

To a solution of 2-chloro-N-[6-(3-pyridin-3-yl-5-trifluoromethyl-pyrazol-1-yl)-pyridazin-3-yl]-isonicotinamide (60 mg, 0.135 mmol) in dimethylformamide (5 mL) are added phenylboronic acid (34 mg, 0.27 mmol), tetrakis(triphenylphosphine)palladium(0) (16 mg, 0.013 mmol) and cesium carbonate (44 mg, 0.135 mmol). The solution is heated to 100° C. for 10 minutes in a microwave reactor. The reddish brown solution is cooled to room temperature and 3-mercaptopropyl-functionalized silica gel (500 mg) is added to the solution. The resulting solution is stirred for 30 minutes and then filtered. Water (20 mL) is added to the filtrate and the resulting mixture is extracted with ethyl acetate (20 mL, 3×). The combined organic layers are dried over magnesium sulfate and then filtered. The filtrate is concentrated under reduced pressure and the residue is purified by chromatography to afford to afford the title compound (5 mg, 8%). LC-MS (M⁺+1): 488.38

The following compounds are prepared according to example 52 by replacing the boronic acid in step b with commercially available starting materials.

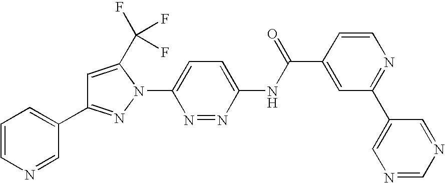

N-[6-(3-Pyridin-3-yl-5-trifluoromethyl-pyrazol-1-yl)-pyridazin-3-yl]-2-pyrimidin-5-yl-isonicotinamide; LC-MS (M⁺+1): 490.37

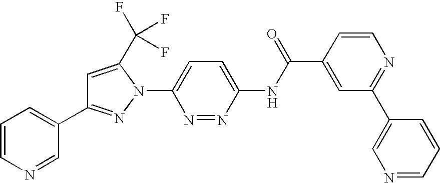

[2,3′]Bipyridinyl-4-carboxylic acid [6-(3-pyridin-3-yl-5-trifluoromethyl-pyrazol-1-yl)-pyridazin-3-yl]-amide; LC-MS (M⁺+1): 489.34.

### Example 53

### 3-Pyridin-2-yl-N-[6-(3-pyridin-3-yl-5-trifluoromethyl-pyrazol-1-yl)-pyridazin-3-yl]-benzamide

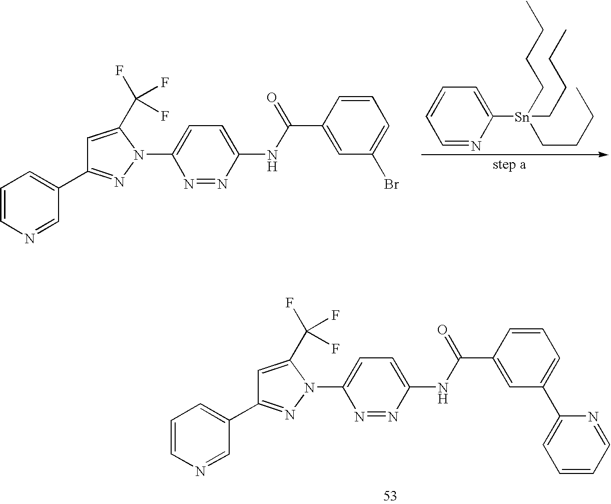

To a solution of 3-bromo-N-[6-(3-pyridin-3-yl-5-trifluoromethyl-pyrazol-1-yl)-pyridazin-3-yl]-benzamide (60 mg, 0.123 mmol), prepared according example 45, in dimethylformamide (5 mL) are added 2-tri-n-butylstannyl pyridine (90 mg, 0.25 mmol) and tetrakis(triphenylphosphine)palladium(0) (14 mg, 0.012 mmol). The solution is heated to 120° C. in a microwave reactor for 2 hours. The reddish brown solution is cooled to room temperature and 3-mercaptopropyl-functionalized silica gel (500 mg) is added to adsorb the palladium residue. The resulting pale yellow solution mixture is filtered. Water (20 mL) is added to the filtrate and the solution is extracted with ethyl acetate (20 mL, 3×). The combined organic layers are dried over magnesium sulfate and then filtered. The filtrate is concentrated under reduced pressure and the residue is purified by chromatography to afford the title compound (12 mg, 20%). LC-MS (M⁺+1): 488.34.

### Example 54

### N-[6-(3-Pyridin-3-yl-5-trifluoromethyl-pyrazol-1-yl)-pyridazin-3-yl]-3-(1H-tetrazol-5-yl)-benzamide

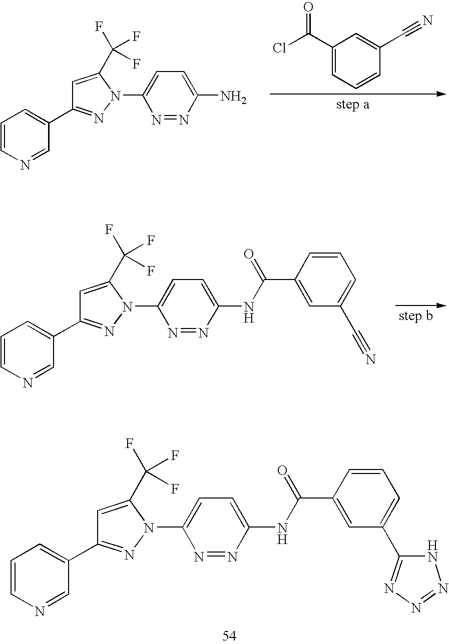

### Step a

6-(3-Pyridin-3-yl-5-trifluoromethyl-pyrazol-1-yl)-pyridazin-3-ylamine is prepared according to step b in example 45). To a solution of 6-(3-pyridin-3-yl-5-trifluoromethyl-pyrazol-1-yl)-pyridazin-3-ylamine (200 mg, 0.65 mmol) in etrahydrofuran (10 mL) are added N,N-diisopropylethylamine (0.34 mL, 1.96 mmol) and 3-cynaobenzoyl chloride (216 mg, 1.31 mmol) respectively at 0° C. under nitrogen atmosphere. The solution is stirred at the same temperature for 1 hour. Saturated aqueous sodium bicarbonate solution (10 mL) is added and the solution is extracted with ethyl acetate (20 mL, 3×). The combined organic layers are dried over magnesium sulfate and then filtered. The filtrate is concentrated under reduced pressure and the residue is purified by chromatography to afford 3-cyano-N-[6-(3-pyridin-3-yl-5-trifluoromethyl-pyrazol-1-yl)-pyridazin-3-yl]-benzamide (150 mg, 53%).

### Step b

To a solution of 3-cyano-N-[6-(3-pyridin-3-yl-5-trifluoromethyl-pyrazol-1-yl)-pyridazin-3-yl]-benzamide (50 mg, 0.12 mmol) in dimethylformamide (4 mL) are added azidotrimethylsilane (0.03 mL, 0.23 mmol) and dibutyltin oxide (87.5 mg, 0.35 mmol) at room temperature. The solution is heated up to 100° C. for 20 minutes in a microwave reactor. The solution is cooled to room temperature and water (20 mL) is added. The solution is extracted with ethyl acetate (20 mL, 3×). The combined organic layers are dried over magnesium sulfate and then filtered. The filtrate is concentrated under reduced pressure and the residue is purified by chromatography to afford the title compound (23 mg, 42%) as a white solid. LC-MS (M⁺+1): 479.03.

### Example 55

### 3-(2-Methyl-oxazol-5-yl)-N-[6-(3-pyridin-3-yl-5-trifluoromethyl-pyrazol-1-yl)-pyridazin-3-yl]-benzamide

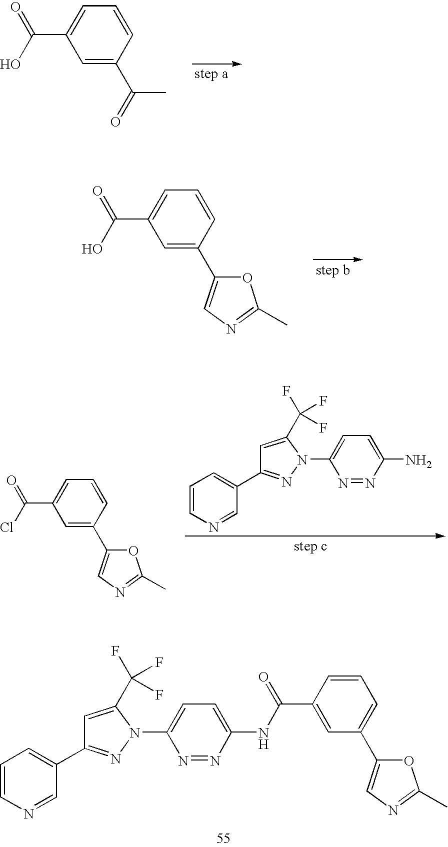

### Step a

To a stirring solution of thallium (III) acetate (2.3 g, 6.1 mmol) in acetonitrile (10 mL) is added trifluoromethansulfonic acid (0.81 mL, 9.1 mmol) at room temperature. The mixture is stirred at the same temperature for 10 minutes. A solution of 3-acetylbenzoic acid (500 mg, 3 mmol) in acetonitrile (5 mL) is added to the above mixture and the resulting solution is heated at reflux for 90 minutes. The solution is cooled to room temperature and concentrated under reduced pressure. The residue is extracted with ethyl acetate (20 mL, 3×) and water (10 mL). The combined organic layers are dried over magnesium sulfate and then filtered. The residue is purified by chromatography to afford 3-(2-Methyl-oxazol-5-yl)-benzoic acid (250 mg, 40%) as a white solid.

### Step b

To a solution of 3-(2-methyl-oxazol-5-yl)-benzoic acid (500 mg, 2.5 mmol) in dichloromethane (10 mL) is added oxalyl chloride (0.64 mL, 7.38 mmol) at room temperature under nitrogen atmosphere. The solution is heated at reflux for 3 hours and then cooled to room temperature. The solution is concentrated under reduced pressure and the residue, 3-(2-methyl-oxazol-5-yl)-benzoyl chloride (420 mg, 77%), is used in the next step of the synthesis without further purification.

### Step c

6-(3-Pyridin-3-yl-5-trifluoromethyl-pyrazol-1-yl)-pyridazin-3-ylamine is prepared according to step b in example 45. To a solution of 6-(3-pyridin-3-yl-5-trifluoromethyl-pyrazol-1-yl)-pyridazin-3-ylamine (50 mg, 0.163 mmol) in tetrahydrofuran (10 mL) are added N,N-diisopropylethylamine (0.14 mL, 0.816 mmol) and 3-(2-methyl-oxazol-5-yl)-benzoyl chloride (72 mg, 0.33 mmol) respectively at 0° C. under nitrogen atmosphere. The solution is stirred at the same temperature for 1 hour. Saturated aqueous sodium bicarbonate solution (10 mL) is added and the solution is extracted with ethyl acetate (20 mL, 3×). The combined organic layers are dried over magnesium sulfate and then filtered. The filtrate is concentrated under reduced pressure and the residue is purified by chromatography to afford the title compound (10 mg, 13%). LC-MS (M⁺+1): 492.34.

### Example 56

### 1-Ethyl-6-oxo-1,6-dihydro-pyridine-3-carboxylic Acid [6-(3-pyridin-3-yl-5-trifluoromethyl-pyrazol-1-yl)-pyridazin-3-yl]-amide and 6-Ethoxy-N-[6-(3-pyridin-3-yl-5-trifluoromethyl-pyrazol-1-yl)-pyridazin-3-yl]-nicotinamide

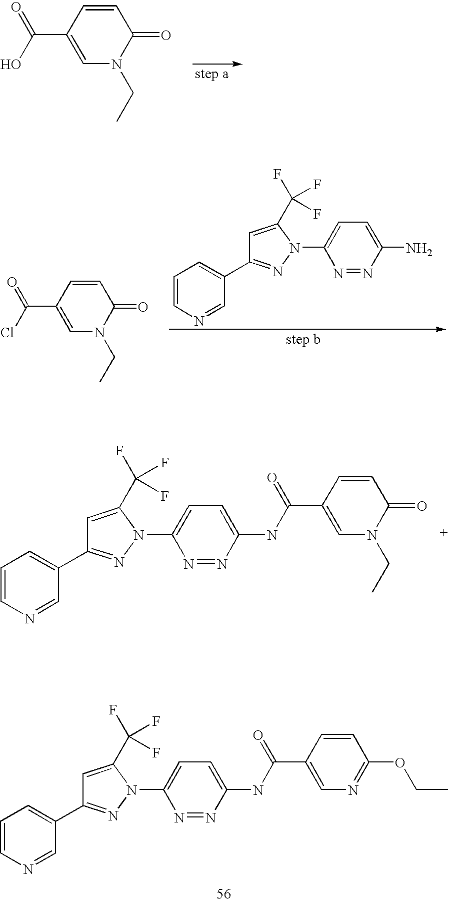

### Step a

To a solution of 1-ethyl-6-oxo-1,6-dihydro-pyridine-3-carboxylic acid (500 mg, 2.9 mmol), prepared according to step c example 3, in dichloromethane (20 mL) is added oxalyl chloride (0.5 mL, 5.9 mmol) at room temperature under nitrogen atmosphere. The solution is heated at reflux for 3 hours and is then cooled to room temperature. The solution is concentrated under reduced pressure and the residue, 1-ethyl-6-oxo-1,6-dihydro-pyridine-3-carbonyl chloride (354 mg, 64%), is used in the next step of the synthesis without further purification.

### Step b

6-(3-Pyridin-3-yl-5-trifluoromethyl-pyrazol-1-yl)-pyridazin-3-ylamine is prepared according to step b in example 45. To a solution of 6-(3-pyridin-3-yl-5-trifluoromethyl-pyrazol-1-yl)-pyridazin-3-ylamine (200 mg, 0.653 mmol) in tetrahydrofuran (10 mL) are added N,N-diisopropylethylamine (0.23 mL, 1.31 mmol) and 1-ethyl-6-oxo-1,6-dihydro-pyridine-3-carbonyl chloride (182 mg, 0.98 mmol) respectively at 0° C. under nitrogen atmosphere. The solution is stirred at the same temperature for 1 hour. Saturated aqueous sodium bicarbonate solution (10 mL) is added and the solution is extracted with ethyl acetate (20 mL, 3×). The combined organic layers are dried over magnesium sulfate and then filtered. The filtrate is concentrated under reduced pressure and the residue is purified by chromatography to afford 1-ethyl-6-oxo-1,6-dihydro-pyridine-3-carboxylic acid [6-(3-pyridin-3-yl-5-trifluoromethyl-pyrazol-1-yl)-pyridazin-3-yl]-amide (31 mg, 10%), LC-MS (M⁺+1): 456.80, and 6-ethoxy-N-[6-(3-pyridin-3-yl-5-trifluoromethyl-pyrazol-1-yl)-pyridazin-3-yl]-nicotinamide (16 mg, 5%) LC-MS (M⁺+1): 456.61.

### Example 57

### 3-(6-Fluoro-pyridin-3-yl)-N-[6-(3-pyridin-3-yl-5-trifluoromethyl-pyrazol-1-yl)-pyridazin-3-yl]-benzamide

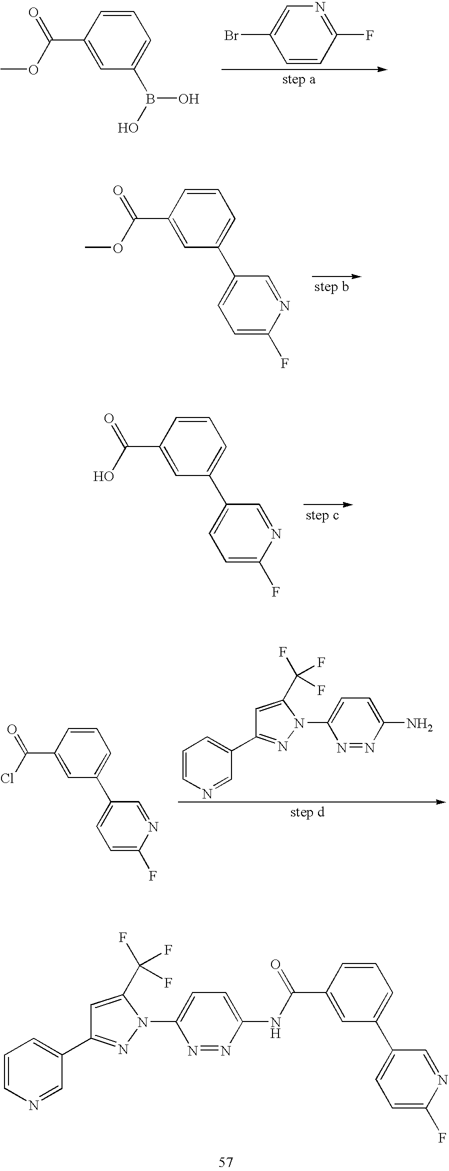

### Step a

To a solution of 3-methoxycarbonylphenylboronic acid (300 mg, 1.67 mmol) in dimethylformamide (5 mL) are added 5-bromo-2-fluoropyridine (0.34 mL, 3.3 mmol), tetrakis(triphenylphosphine)palladium(0) (192 mg, 0.17 mmol) and cesium carbonate (543 mg, 1.7 mmol). The solution is heated to 120° C. in a microwave reactor for 20 minutes. The reddish brown solution is cooled room temperature and 3-mercaptopropyl-functionalized silica gel (500 mg) is added to adsorb the palladium residue. The resulting pale yellow solution mixture is filtered. The filtrate is washed with water (10 mL) and then extracted with ethyl acetate (20 mL, 3×). The combined organic layers are dried over magnesium sulfate and then filtered. The filtrate is concentrated under reduced pressure and the residue is purified by chromatography to afford 3-(6-fluoro-pyridin-3-yl)-benzoic acid methyl ester (260 mg, 68%).

### Step b

To a solution of 3-(6-fluoro-pyridin-3-yl)-benzoic acid methyl ester (200 mg, 0.87 mmol) in 1,4-dioxane (20 mL) and water (5 mL) is added lithium hydroxide (41 mg, 1.7 mmol) at room temperature. The solution is stirred at room temperature for 4 hours. The solvent is removed under reduced pressure. The residue is acidified to pH 2 with 12 M aqueous HCl. The resulting solution was extracted with ethyl acetate (20 mL, 3×) and water (10 mL). The combined organic layers are dried over magnesium sulfate and then filtered. The filtrate is concentrated under reduced pressure and the residue is purified by chromatography to afford 3-(6-fluoro-pyridin-3-yl)-benzoic acid (150 mg, 80%).

### Step c

To a solution of 3-(6-fluoro-pyridin-3-yl)-benzoic acid (200 mg, 0.92 mmol) in dichloromethane (20 mL) is added oxalyl chloride (0.16 mL, 1.84 mmol) at room temperature under nitrogen atmosphere. The solution is heated at reflux for 3 hours and is then cooled to room temperature. The solution is concentrated under reduced pressure and the residue, 3-(6-fluoro-pyridin-3-yl)-benzoyl chloride (180 mg, 83%), is used in the next step of the synthesis without further purification.

### Step d

6-(3-Pyridin-3-yl-5-trifluoromethyl-pyrazol-1-yl)-pyridazin-3-ylamine is prepared according to step b in example 45. To a solution of 6-(3-pyridin-3-yl-5-trifluoromethyl-pyrazol-1-yl)-pyridazin-3-ylamine (200 mg, 0.653 mmol) in tetrahydrofuran (10 mL) are added N,N-diisopropylethylamine (0.23 mL, 1.31 mmol) and 3-(6-fluoro-pyridin-3-yl)-benzoyl chloride (307 mg, 1.31 mmol) respectively at 0° C. under nitrogen atmosphere. The solution is stirred at the same temperature for 1 hour. Saturated aqueous sodium bicarbonate solution (10 mL) is added and the solution is extracted with ethyl acetate (20 mL, 3×). The combined organic layers are dried over magnesium sulfate and then filtered. The filtrate is concentrated under reduced pressure and the residue is purified by chromatography to afford the title compound (95 mg, 29%). LC-MS (M⁺+1): 506.03.

The following compounds are prepared according to example 57 by replacing the 5-bromo-2-fluoropyridine in step a with commercially available starting materials.

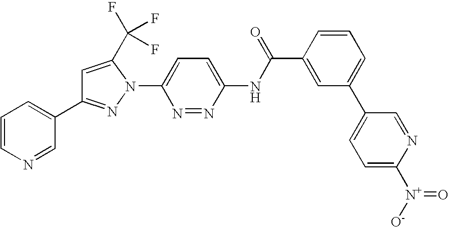

3-(6-Nitro-pyridin-3-yl)-N-[6-(3-pyridin-3-yl-5-trifluoromethyl-pyrazol-1-yl)-pyridazin-3-yl]-benzamide; LC-MS (M⁺+1): 533.26.

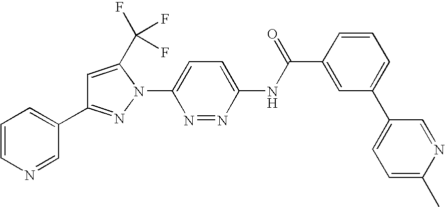

3-(6-Methyl-pyridin-3-yl)-N-[6-(3-pyridin-3-yl-5-trifluoromethyl-pyrazol-1-yl)-pyridazin-3-yl]-benzamide; LC-MS (M⁺+1): 502.28.

### Example 58

### 3-Morpholin-4-yl-N-[6-(3-pyridin-3-yl-5-trifluoromethyl-pyrazol-1-yl)-pyridazin-3-yl]-benzamide

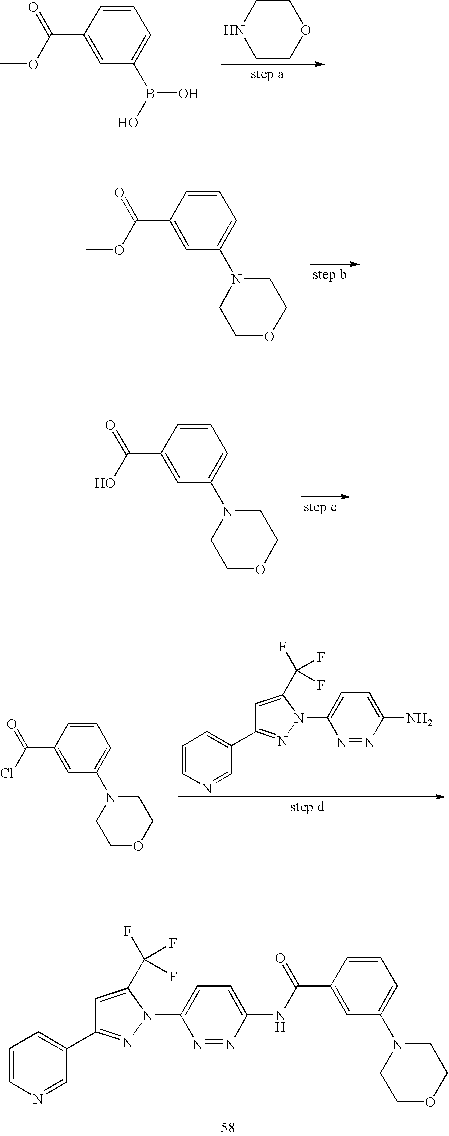

### Step a

To a solution of (3-methoxycarbonylphenyl)boronic acid (100 mg, 0.56 mmol) in toluene (5 mL) are added morpholine (0.1 mL, 1.1 mmol), copper (II) acetate (10 mg, 0.056 mmol) and myristic acid (25 mg, 0.11 mmol) at room temperature. The solution is stirred at the same temperature for 12 hours. The mixture is filtered and the filtrate is concentrated under reduced pressure. The residue is purified by chromatography to afford 3-morpholin-4-yl-benzoic acid methyl ester (90 mg, 73%).

### Step b

To a solution of 3-morpholin-4-yl-benzoic acid methyl ester (200 mg, 0.9 mmol) in 1,4-dioxane (20 mL) and water (5 mL) is added lithium hydroxide (43 mg, 1.8 mmol) at room temperature. The solution is stirred at room temperature for 4 hours. The solvent is removed under reduced pressure. The residue is acidified to pH 2 with 12 M aqueous HCl. The resulting solution was extracted with ethyl acetate (20 mL, 3×) and water (10 mL). The combined organic layers are dried over magnesium sulfate and then filtered. The filtrate is concentrated under reduced pressure and the residue is purified by chromatography to afford 3-morpholin-4-yl-benzoic acid (160 mg, 85%).

### Step c

To a solution of 3-morpholin-4-yl-benzoic acid (160 mg, 0.77 mmol) in dichloromethane (20 mL) is added oxalyl chloride (0.14 mL, 1.54 mmol) at room temperature under nitrogen atmosphere. The solution is heated at reflux for 3 hours and is then cooled to room temperature. The solution is concentrated under reduced pressure and the residue product, 3-morpholin-4-yl-benzoyl chloride (160 mg, 92%), is used in the next step of the synthesis without further purification.

### Step d

6-(3-Pyridin-3-yl-5-trifluoromethyl-pyrazol-1-yl)-pyridazin-3-ylamine is prepared according to step b in example 45. To a solution of 6-(3-pyridin-3-yl-5-trifluoromethyl-pyrazol-1-yl)-pyridazin-3-ylamine (200 mg, 0.653 mmol) in tetrahydrofuran (10 mL) are added N,N-diisopropylethylamine (0.23 mL, 1.31 mmol) and 3-morpholin-4-yl-benzoyl chloride (295 mg, 1.31 mmol) respectively at 0° C. under nitrogen atmosphere. The solution is stirred at the same temperature for 1 hour. Saturated aqueous sodium bicarbonate solution (10 mL) is added and the solution is extracted with ethyl acetate (20 mL, 3×). The combined organic layers are dried over magnesium sulfate and then filtered. The filtrate is concentrated under reduced pressure and the residue is purified by chromatography to afford the title compound (20 mg, 6%). LC-MS (M⁺+1): 496.01.

The following compounds are prepared according to example 58 by replacing morpholine in step a with commercially available starting materials.

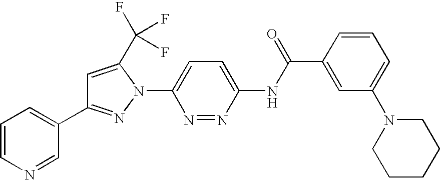

3-Piperidin-1-yl-N-[6-(3-pyridin-3-yl-5-trifluoromethyl-pyrazol-1-yl)-pyridazin-3-yl]-benzamide; LC-MS (M⁺+1): 494.32.

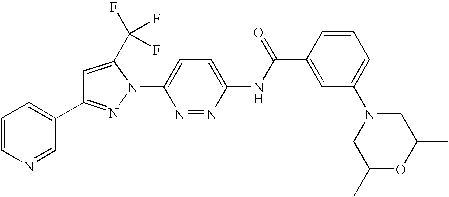

3-(2,6-Dimethyl-morpholin-4-yl)-N-[6-(3-pyridin-3-yl-5-trifluoromethyl-pyrazol-1-yl)-pyridazin-3-yl]-benzamide; LC-MS (M⁺+1): 524.31.

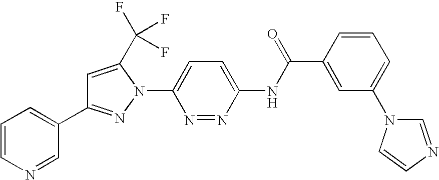

3-Imidazol-1-yl-N-[6-(3-pyridin-3-yl-5-trifluoromethyl-pyrazol-1-yl)-pyridazin-3-yl]-benzamide; LC-MS (M⁺+1): 477.24.

### Example 59

### 3-(6-Oxo-1,6-dihydro-pyridin-3-yl)-N-[6-(3-pyridin-3-yl-5-trifluoromethyl-pyrazol-1-yl)-pyridazin-3-yl]-benzamide

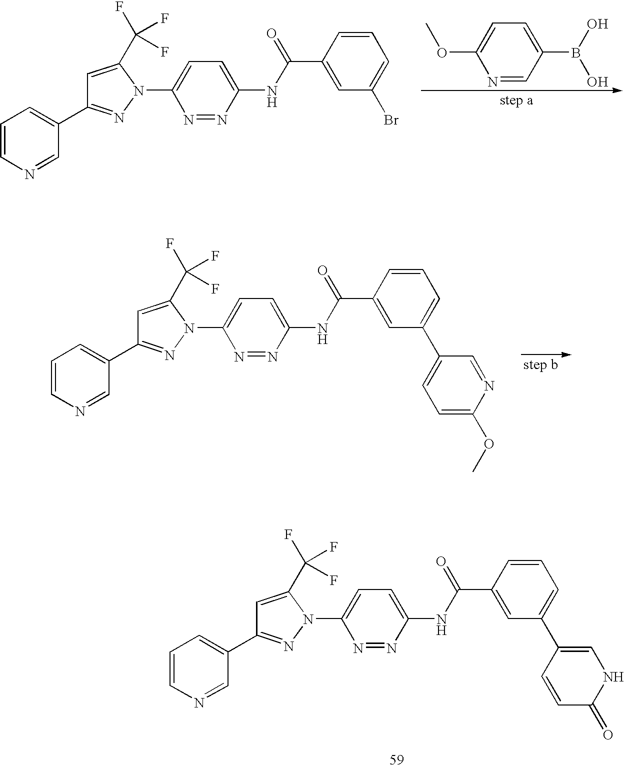

### Step a

To a solution of 3-bromo-N-[6-(3-pyridin-3-yl-5-trifluoromethyl-pyrazol-1-yl)-pyridazin-3-yl]-benzamide (60 mg, 0.123 mmol), prepared according to example 45, in dimethylformamide (5 mL) are added (2-methoxy-5-pyridinyl)boronic acid (38 mg, 0.25 mmol), tetrakis(triphenylphosphine)palladium(0) (14 mg, 0.012 mmol) and cesium carbonate (40 mg, 0.123 mmol). The solution is heated to 100° C. for 10 minutes in a microwave reactor. The reddish brown solution is cooled to room temperature and 3-mercaptopropyl-functionalized silica gel (500 mg) is added to the solution. The resulting solution is stirred for 30 minutes and is filtered. Water (20 mL) is added to the filtrate and the resulting mixture is extracted with ethyl acetate (20 mL, 3×). The combined organic layers are dried over magnesium sulfate and then filtered. The filtrate is concentrated under reduced pressure and the residue is purified by chromatography to afford 3-(6-methoxy-pyridin-3-yl)-N-[6-(3-pyridin-3-yl-5-trifluoromethyl-pyrazol-1-yl)-pyridazin-3-yl]-benzamide (15 mg, 24%).

### Step b

To a solution of sodium sulfide (30 mg, 0.39 mmol) in 1,3-dimethyl-2-imidazolinone (5 mL) is added trimethylsilyl chloride (0.05 mL, 0.39 mmol) at room temperature under nitrogen atmosphere. The solution is allowed to stir for 30 minutes at the same temperature prior to the addition of 3-(6-methoxy-pyridin-3-yl)-N-[6-(3-pyridin-3-yl-5-trifluoromethyl-pyrazol-1-yl)-pyridazin-3-yl]-benzamide (100 mg, 0.19 mmol). The solution mixture is sealed and heated to 150° C. in a microwave reactor for 10 minutes. The solution is cooled to room temperature, diluted with water (10 mL), and extracted with dichloromethane (20 mL, 3×). The combined organic layers are dried over magnesium sulfate and then filtered. The filtrate is concentrated under reduced pressure and the residue is purified by to afford the title compound (5 mg, 5%). LC-MS (M⁺+1): 503.94.

### Example 60

### 3-Bromo-N-{6-[3-(6-morpholin-4-yl-pyridin-3-yl)-5-trifluoromethyl-pyrazol-1-yl]-pyridazin-3-yl}-benzamide

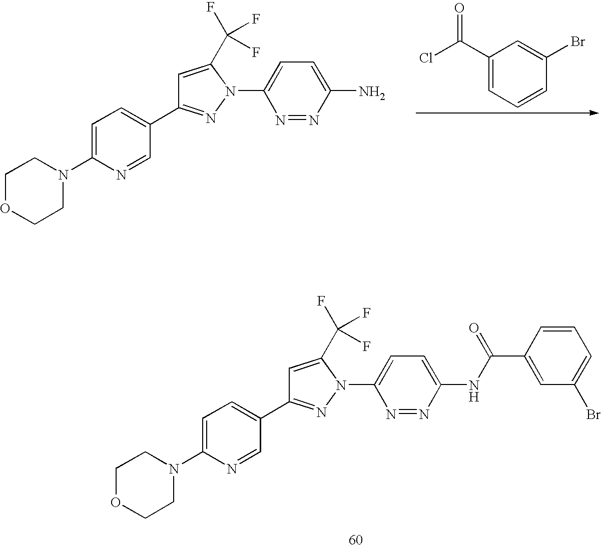

6-[3-(6-morpholin-4-yl-pyridin-3-yl)-5-trifluoromethyl-pyrazol-1-yl]-pyridazin-3-ylamine is prepared according to example 45 by replacing 3-acetylpyridine with 1-(6-morpholin-4-yl-pyridin-3-yl)-ethanone (prepared according to step a in example 36). To a solution of 6-[3-(6-morpholin-4-yl-pyridin-3-yl)-5-trifluoromethyl-pyrazol-1-yl]-pyridazin-3-ylamine (200 mg, 0.511 mmol) in tetrahydrofuran (20 mL) are added N,N-diisopropylethylamine (0.27 mL, 1.53 mmol) and 3-bromobenzoyl chloride (0.14 mL, 1 mmol) respectively at 0° C. under nitrogen atmosphere. The solution is stirred at the same temperature for 1 hour. Saturated aqueous sodium bicarbonate solution is added and the solution is extracted with ethyl acetate (20 mL, 3×). The combined organic layers are dried over magnesium sulfate and then filtrated. The filtrate is concentrated under reduced pressure and the residue is purified by chromatography to afford the title compound (150 mg, 51%). LC-MS (M⁺+1): 576.14.

The following compound is prepared according to example 60 by replacing the acyl chloride with commercially available starting material.

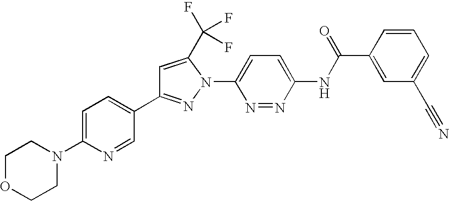

3-Cyano-N-{6-[3-(6-morpholin-4-yl-pyridin-3-yl)-5-trifluoromethyl-pyrazol-1-yl]-pyridazin-3-yl}-benzamide; LC-MS (M⁺+1): 521.3.

### Example 61

### 3-(6-Fluoro-pyridin-3-yl)-N-{6-[3-(6-morpholin-4-yl-pyridin-3-yl)-5-trifluoromethyl-pyrazol-1-yl]-pyridazin-3-yl}-benzamide

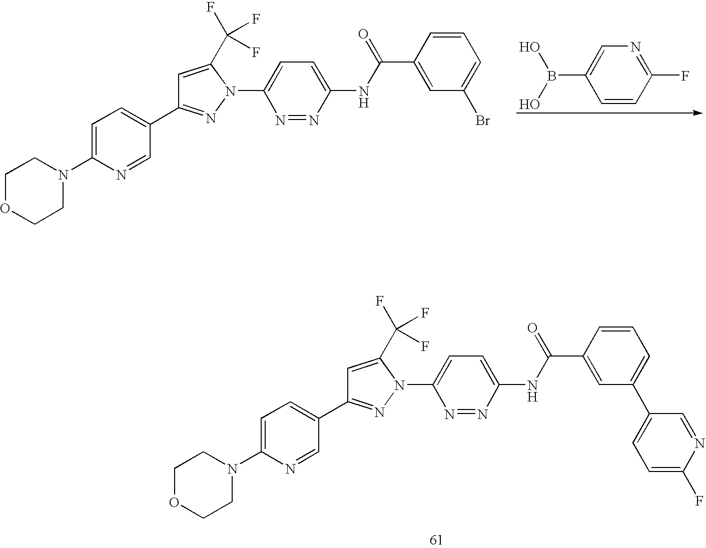

To a solution of 3-bromo-N-{6-[3-(6-morpholin-4-yl-pyridin-3-yl)-5-trifluoromethyl-pyrazol-1-yl]-pyridazin-3-yl}-benzamide (80 mg, 0.14 mmol), prepared according to example 59, in dimethylformamide (5 mL) are added 2-fluoropyridine-5-boronic acid (39 mg, 0.28 mmol), Tetrakis-(triphenylphosphine)palladium(0) (16 mg, 0.014 mmol) and cesium carbonate (45 mg, 0.14 mmol). The solution is heated to 100° C. for 10 minutes in a microwave reactor. The reddish brown solution is cooled to room temperature and 3-mercaptopropyl-functionalized silica gel (500 mg) is added to the solution. The resulting solution is stirred for 30 minutes and then filtered. Water (20 mL) is added to the filtrate and the resulting mixture is extracted with ethyl acetate (20 mL, 3×). The combined organic layers are dried over magnesium sulfate and then filtered. The filtrate is concentrated under reduced pressure and the residue is purified by chromatography to afford the title compound (52 mg, 63%). LC-MS (M⁺+1): 591.24

### Example 62

### 1-{3-[6-(3-Pyridin-3-yl-5-trifluoromethyl-pyrazol-1-yl)-pyridazin-3-ylcarbamoyl]-phenyl}-piperidine-4-carboxylic Acid Ethyl Ester

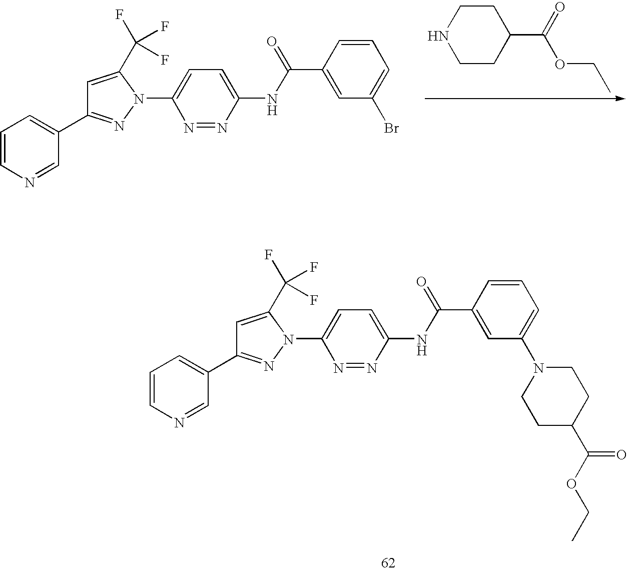

To a solution of 3-bromo-N-[6-(3-pyridin-3-yl-5-trifluoromethyl-pyrazol-1-yl)-pyridazin-3-yl]-benzamide (200 mg, 0.4 mmol), prepared according to example 45, in dimethylsulfoxide (4 mL) are added ethyl isonipecotate (0.13 mL, 0.8 mmol), copper (I) iodide (8 mg, 0.04 mmol), L-proline (9 mg, 0.08 mmol) and potassium carbonate (57 mg, 0.4 mmol) respectively. The solution mixture is heated up to 90° C. in a microwave reactor for 90 minutes. The resulting solution is cooled to room temperature and is washed with water (10 mL). The solution mixture is extracted with ethyl acetate (20 mL, 3×) and the combined organic layers are dried over magnesium sulfate and then filtered. The filtrate is concentrated under reduced pressure and the residue is purified by chromatography to afford the title compound (9 mg, 3%). LC-MS (M⁺+1): 566.26

The following compounds are prepared according to example 62 by replacing the amine with commercially available starting materials.

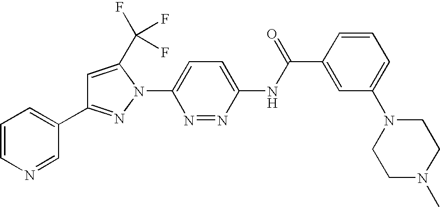

3-(4-Methyl-piperazin-1-yl)-N-[6-(3-pyridin-3-yl-5-trifluoromethyl-pyrazol-1-yl)-pyridazin-3-yl]-benzamide; LC-MS (M⁺+1): 509.28.

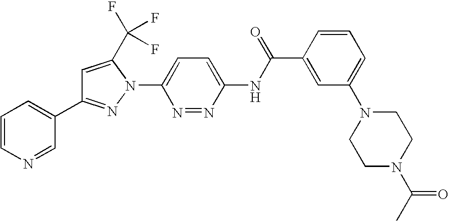

3-(4-Acetyl-piperazin-1-yl)-N-[6-(3-pyridin-3-yl-5-trifluoromethyl-pyrazol-1-yl)-pyridazin-3-yl]-benzamide; LC-MS (M⁺+1): 537.29.

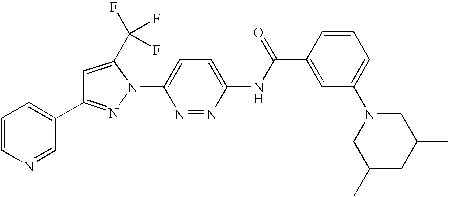

3-(3,5-Dimethyl-piperidin-1-yl)-N-[6-(3-pyridin-3-yl-5-trifluoromethyl-pyrazol-1-yl)-pyridazin-3-yl]-benzamide; LC-MS (M⁺+1): 522.34.

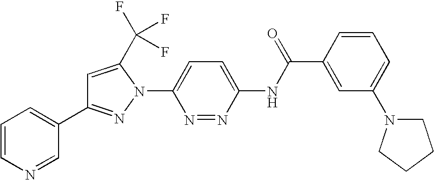

N-[6-(3-Pyridin-3-yl-5-trifluoromethyl-pyrazol-1-yl)-pyridazin-3-yl]-3-pyrrolidin-1-yl-benzamide; LC-MS (M⁺+1): 480.27.

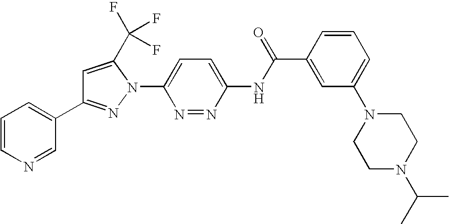

3-(4-Isopropyl-piperazin-1-yl)-N-[6-(3-pyridin-3-yl-5-trifluoromethyl-pyrazol-1-yl)-pyridazin-3-yl]-benzamide; LC-MS (M⁺+1): 537.29.

### Example 63

### Piperidine-3-carboxylic Acid [6-(3-pyridin-3-yl-5-trifluoromethyl-pyrazol-1-yl)-pyridazin-3-yl]-amide

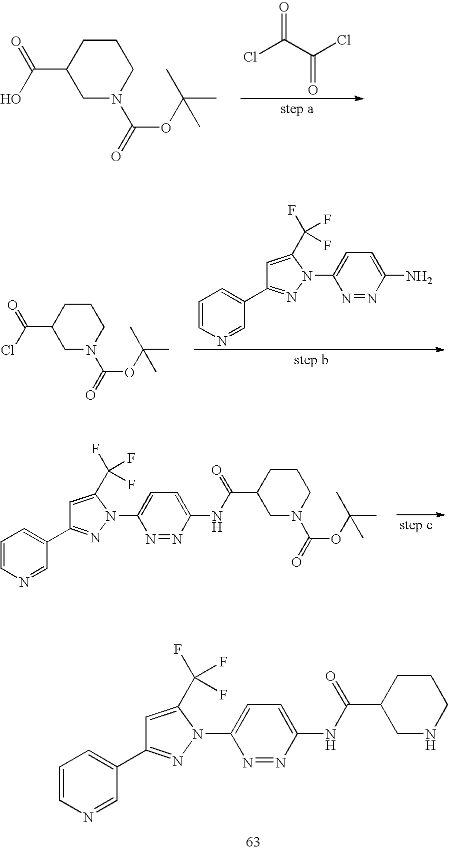

### Step a

To a solution of 1-(tert-butoxycarbonyl)-3-piperidinecarboxylic acid (300 mg, 1.31 mmol) in dichloromethane (20 mL) is added oxalyl chloride (0.23 mL, 2.6 mmol) at room temperature under nitrogen atmosphere. The solution is heated at reflux for 3 hours and then cooled to room temperature. The solution is concentrated under reduced pressure and the residue, 3-chlorocarbonyl-piperidine-1-carboxylic acid tert-butyl ester (250 mg, 77%), is used in the next step of the synthesis without further purification.

### Step b

6-(3-Pyridin-3-yl-5-trifluoromethyl-pyrazol-1-yl)-pyridazin-3-ylamine is prepared according to step b in example 45. To a solution of 6-(3-pyridin-3-yl-5-trifluoromethyl-pyrazol-1-yl)-pyridazin-3-ylamine (100 mg, 0.33 mmol) in tetrahydrofuran (10 mL) are added N,N-diisopropylethylamine (0.17 mL, 0.98 mmol) and 3-chlorocarbonyl-piperidine-1-carboxylic acid tert-butyl ester (162 mg, 0.65 mmol) respectively at 0° C. under nitrogen atmosphere. The solution is stirred at the same temperature for 1 hour. Saturated aqueous sodium bicarbonate solution (10 mL) is added and the solution is extracted with ethyl acetate (20 mL, 3×). The combined organic layers are dried over magnesium sulfate and then filtered. The filtrate is concentrated under reduced pressure and the residue is purified by chromatography to afford 3-[6-(3-pyridin-3-yl-5-trifluoromethyl-pyrazol-1-yl)-pyridazin-3-ylcarbamoyl]-piperidine-1-carboxylic acid tert-butyl ester (103 mg, 61%).

### Step c

To a solution of 3-[6-(3-pyridin-3-yl-5-trifluoromethyl-pyrazol-1-yl)-pyridazin-3-ylcarbamoyl]-piperidine-1-carboxylic acid tert-butyl ester (50 mg, 0.01 mmol) in dichloromethane (10 mL) is added trifluoromethansulfonic acid (0.1 mL) at room temperature. The solution is stirred at the same temperature for 1 hour. The solution is concentrated under reduced pressure and the residue is purified by chromatography to afford the title compound (28 mg, 70%). LC-MS (M⁺+1): 417.12.

### Example 64

### 3-Bromo-N-{6-[3-(6-methoxy-pyridin-3-yl)-5-trifluoromethyl-pyrazol-1-yl]-pyridazin-3-yl}-benzamide

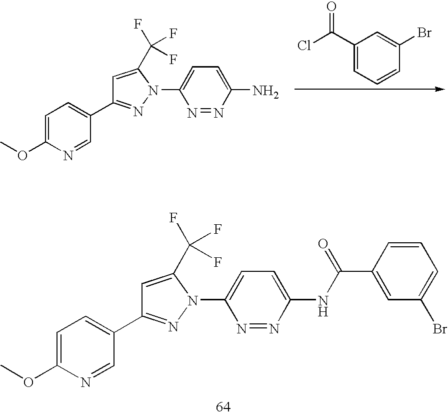

6-[3-(6-Methoxy-pyridin-3-yl)-5-trifluoromethyl-pyrazol-1-yl]-pyridazin-3-ylamine is prepared according to example 45 by replacing 3-acetylpyridine with 1-(6-methoxy-pyridin-3-yl)-ethanone (prepared according to step a in example 36). To a solution of 6-[3-(6-methoxy-pyridin-3-yl)-5-trifluoromethyl-pyrazol-1-yl]-pyridazin-3-ylamine (200 mg, 0.6 mmol) in tetrahydrofuran (10 mL) are added N,N-diisopropylethylamine (0.3 mL, 1.8 mmol) and 3-bromobenzoyl chloride (0.16 mL, 1.2 mmol) respectively at 0° C. under nitrogen atmosphere. The solution is stirred at the same temperature for 1 hour. Saturated aqueous sodium bicarbonate solution is added and the solution is extracted with ethyl acetate (20 mL, 3×). The combined organic layers are dried over magnesium sulfate and then filtered. The filtrate is concentrated under reduced pressure and the residue is purified by to afford the title compound (260 mg, 84%). LC-MS (M⁺+1): 521.18.

The following compound is prepared according to example 64 by replacing the acyl chloride with commercially available starting material.

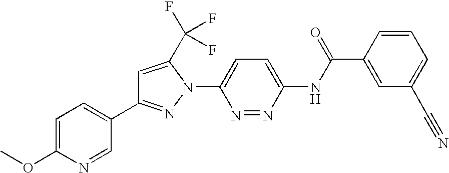

3-Cyano-N-{6-[3-(6-methoxy-pyridin-3-yl)-5-trifluoromethyl-pyrazol-1-yl]-pyridazin-3-yl}-benzamide; LC-MS (M⁺+1): 466.31.

### Example 65

### 3-(6-Fluoro-pyridin-3-yl)-N-{6-[3-(6-methoxy-pyridin-3-yl)-5-trifluoromethyl-pyrazol-1-yl]-pyridazin-3-yl}-benzamide

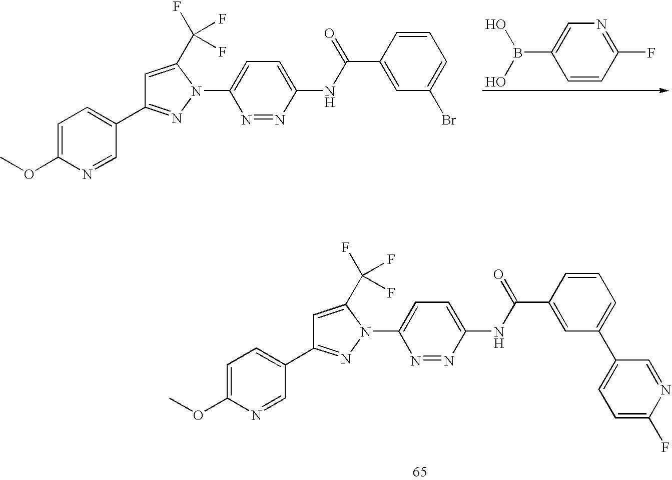

To a solution of 3-bromo-N-{6-[3-(6-methoxy-pyridin-3-yl)-5-trifluoromethyl-pyrazol-1-yl]-pyridazin-3-yl}-benzamide (100 mg, 0.19 mmol), prepared according to example 63, in dimethylformamide (5 mL) are added 2-fluoropyridine-5-boronic acid (54 mg, 0.39 mmol), tetrakis(triphenylphosphine)palladium(0) (22 mg, 0.019 mmol) and cesium carbonate (63 mg, 0.19 mmol). The solution is heated to 100° C. for 10 minutes in a microwave reactor. The reddish brown solution is cooled to room temperature and 3-mercaptopropyl-functionalized silica gel (500 mg) is added to the solution. The resulting solution is stirred for 30 minutes and is filtered. Water (20 mL) is added to the filtrate and the resulting mixture is extracted with ethyl acetate (20 mL, 3×). The combined organic layers are dried over magnesium sulfate and then filtered. The filtrate is concentrated under reduced pressure and the residue is purified by chromatography to afford the title compound (47 mg, 46%). LC-MS (M⁺+1): 536.25.

The following compound is prepared according to example 65 by replacing the boronic acid with commercially available starting material.

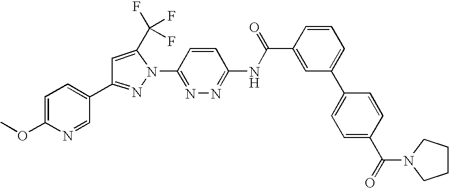

4′-(Pyrrolidine-1-carbonyl)-biphenyl-3-carboxylic acid {6-[3-(6-methoxy-pyridin-3-yl)-5-trifluoromethyl-pyrazol-1-yl]-pyridazin-3-yl}-amide; LC-MS (M⁺+1): 614.62.

### Example 66

### 3-Cyano-N-{6-[3-(6-oxo-1,6-dihydro-pyridin-3-yl)-5-trifluoromethyl-pyrazol-1-yl]-pyridazin-3-yl}-benzamide

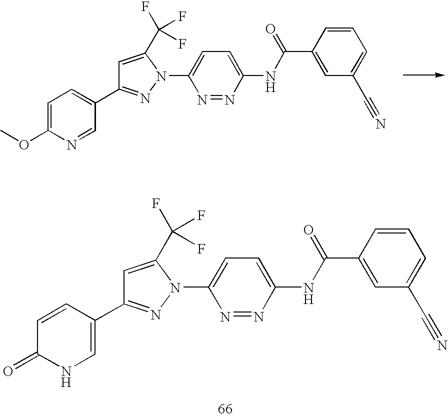

To a solution of sodium sulfide (28 mg, 0.36 mmol) in 1,3-dimethyl-2-imidazolinone (5 mL) is added trimethylsilyl chloride (0.05 mL, 0.36 mmol) at room temperature under nitrogen atmosphere. The solution is allowed to stir for 30 minutes at the same temperature prior to the addition of 3-cyano-N-{6-[3-(6-methoxy-pyridin-3-yl)-5-trifluoromethyl-pyrazol-1-yl]-pyridazin-3-yl}-benzamide (84 mg, 0.18 mmol), prepared according to example 63. The mixture is sealed and then heated up to 150° C. in a microwave reactor for 10 minutes. The solution is cooled to room temperature, diluted with water (10 mL) and extracted with dichloromethane (20 mL, 3×). The combined organic layers are dried over magnesium sulfate and then filtered. The filtrate is concentrated under reduced pressure and the residue is purified by chromatography to afford the title compound (6 mg, 7%). LC-MS (M⁺+1): 452.30.

### Example 67

### 3-Bromo-N-[6-(3-thiazol-2-yl-5-trifluoromethyl-pyrazol-1-yl)-pyridazin-3-yl]-benzamide

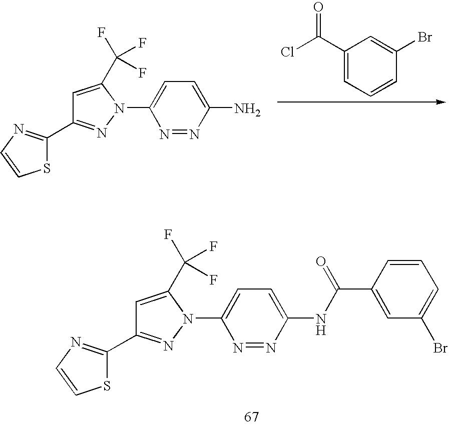

6-(3-Thiazol-2-yl-5-trifluoromethyl-pyrazol-1-yl)-pyridazin-3-ylamine is prepared according to example 45 by replacing 3-acetylpyridine with 1-thiazol-2-yl-ethanone (prepared according to step a in example 36). To a solution of 6-(3-thiazol-2-yl-5-trifluoromethyl-pyrazol-1-yl)-pyridazin-3-ylamine (200 mg, 0.6 mmol) in tetrahydrofuran (10 mL) are added N,N-diisopropylethylamine (0.3 mL, 1.8 mmol) and 3-bromobenzoyl chloride (0.16 mL, 1.2 mmol) respectively at 0° C. under nitrogen atmosphere. The solution is stirred at the same temperature for 1 hour. Saturated aqueous sodium bicarbonate solution, (10 mL) is added and the solution is extracted with ethyl acetate (20 mL, 3×). The combined organic layers are dried over magnesium sulfate and then filtered. The filtrate is concentrated under reduced pressure and the residue is purified by chromatography to afford the title compound (150 mg, 47%). LC-MS (M⁺+1): 497.02.

The following compound is prepared according to example 67 by replacing the acyl chloride with commercially available starting material.

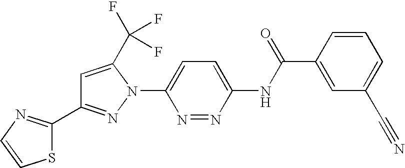

3-Cyano-N-[6-(3-thiazol-2-yl-5-trifluoromethyl-pyrazol-1-yl)-pyridazin-3-yl]-benzamide; LC-MS (M⁺+1): 442.23.

### Example 68

### 3-(6-Fluoro-pyridin-3-yl)-N-[6-(3-thiazol-2-yl-5-trifluoromethyl-pyrazol-1-yl)-pyridazin-3-yl]-benzamide

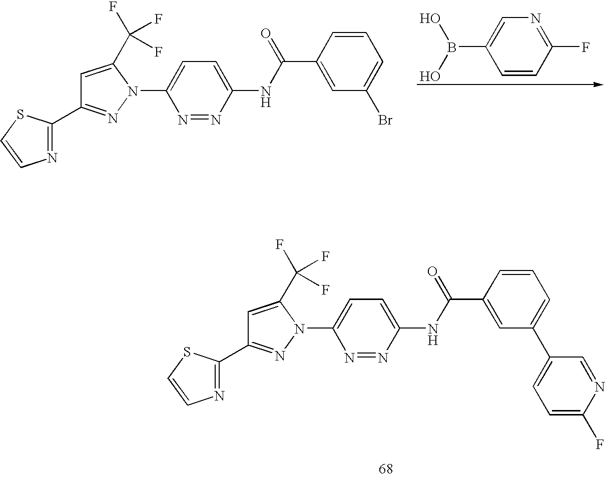

To a solution of 3-bromo-N-[6-(3-thiazol-2-yl-5-trifluoromethyl-pyrazol-1-yl)-pyridazin-3-yl]-benzamide (33) (100 mg, 0.2 mmol) in dimethylformamide (5 mL) are added 2-fluoropyridine-5-boronic acid (57 mg, 0.4 mmol), tetrakis(triphenylphosphine)-palladium(0) (23 mg, 0.02 mmol) and cesium carbonate (66 mg, 0.2 mmol). The solution is heated to 100° C. for 10 minutes in a microwave reactor. The reddish brown solution is cooled to room temperature and 3-mercaptopropyl-functionalized silica gel (500 mg) is added to the solution. The resulting solution is stirred for 30 minutes and then filtered. Water (20 mL) is added to the filtrate and the resulting mixture is extracted with ethyl acetate (20 mL, 3×). The combined organic layers are dried over magnesium sulfate and then filtered. The filtrate is concentrated under reduced pressure and the residue is purified by chromatography to afford the title compound (21 mg, 20%). LC-MS (M⁺+1): 512.21.

### Example 69

### 1-{3-[6-(3-Pyridin-3-yl-5-trifluoromethyl-pyrazol-1-yl)-pyridazin-3-ylcarbamoyl]-phenyl}-piperidine-4-carboxylic Acid

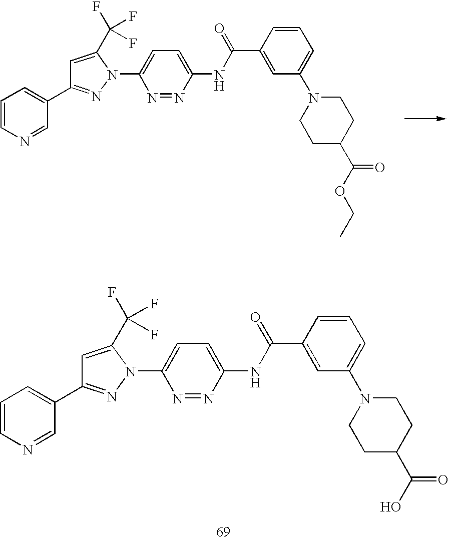

To a solution of 1-{3-[6-(3-pyridin-3-yl-5-trifluoromethyl-pyrazol-1-yl)-pyridazin-3-ylcarbamoyl]-phenyl}-piperidine-4-carboxylic acid ethyl ester (50 mg, 0.088 mmol), prepared according to example 61, in 1,4-dioxane (10 mL) and water (1 mL) is added lithium hydroxide (4 mg, 0.18 mmol) at room temperature. The solution is stirred at the same temperature for 6 hours. The solvent is removed under reduced pressure and the residue is purified by chromatography to afford the title compound (20 mg, 42%). LC-MS (M⁺+1): 538.22.

### Example 70

### 1-(2-Ethoxy-ethyl)-6-oxo-1,6-dihydro-pyridine-3-carboxylic Acid [6-(3-pyridin-3-yl-5-trifluoromethyl-pyrazol-1-yl)-pyridazin-3-yl]-amide

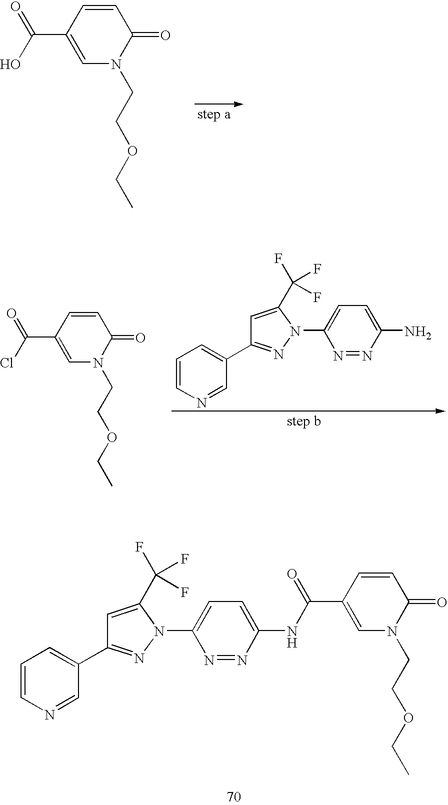

### Step a

To a solution of 1-(2-ethoxy-ethyl)-6-oxo-1,6-dihydro-pyridine-3-carboxylic acid (300 mg, 1.42 mmol), prepared according to step c in example 3, in dichloromethane (20 mL) is added oxalyl chloride (0.25 mL, 2.8 mmol) at room temperature under nitrogen atmosphere. The solution is heated at reflux for 3 hours and then cooled to room temperature. The solution is concentrated under reduced pressure and the residue, 1-(2-ethoxy-ethyl)-6-oxo-1,6-dihydro-pyridine-3-carbonyl chloride (250 mg, 77%), is used in the next step of the synthesis without further purification.

### Step b

6-(3-Pyridin-3-yl-5-trifluoromethyl-pyrazol-1-yl)-pyridazin-3-ylamine is prepared according to step b in example 45. To a solution of 6-(3-pyridin-3-yl-5-trifluoromethyl-pyrazol-1-yl)-pyridazin-3-ylamine (100 mg, 0.33 mmol) in tetrahydrofuran (20 mL) are added N,N-diisopropylethylamine (0.17 mL, 0.98 mmol) and 1-(2-ethoxy-ethyl)-6-oxo-1,6-dihydro-pyridine-3-carbonyl chloride (150 mg, 0.65 mmol) respectively at 0° C. under nitrogen atmosphere. The solution is stirred at the same temperature for 1 hour. Saturated aqueous sodium bicarbonate solution (10 mL) is added and the solution is extracted with ethyl acetate (20 mL, 3×). The combined organic layers are dried over magnesium sulfate and then filtered. The filtrate is concentrated under reduced pressure and the residue is purified by to afford the title compound (70 mg, 43%). LC-MS (M⁺+1): 500.30.

### Example 71

### 3-(6-Fluoro-pyridin-3-yl)-N-[5-(3-pyridin-3-yl-5-trifluoromethyl-pyrazol-1-yl)-pyridin-2-yl]-benzamide

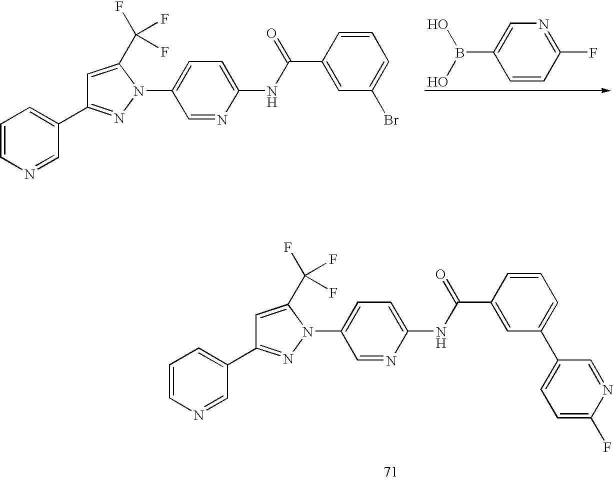

To a solution of 3-bromo-N-[5-(3-pyridin-3-yl-5-trifluoromethyl-pyrazol-1-yl)-pyridin-2-yl]-benzamide (100 mg, 0.2 mmol), prepared according to step a in example 8, in dimethylformamide (5 mL) are added 2-fluoropyridine-5-boronic acid (57 mg, 0.4 mmol), tetrakis(triphenylphosphine)palladium(0) (23 mg, 0.02 mmol) and cesium carbonate (66 mg, 0.2 mmol). The solution is heated to 100° C. for 10 minutes in a microwave reactor. The reddish brown solution is cooled to room temperature and 3-mercaptopropyl-functionalized silica gel (500 mg) is added to the solution. The resulting solution is stirred for 30 minutes and then filtered. Water (20 mL) is added to the filtrate and the resulting mixture is extracted with ethyl acetate (20 mL, 3×). The combined organic layers are dried over magnesium sulfate and then filtered. The filtrate is concentrated under reduced pressure and the residue is purified by to afford the title compound (51 mg, 49%). LC-MS (M⁺+1): 505.27.

### Example 72

### 1-{3-[5-(3-Pyridin-3-yl-5-trifluoromethyl-pyrazol-1-yl)-pyridin-2-ylcarbamoyl]-phenyl}-piperidine-4-carboxylic Acid Ethyl Ester

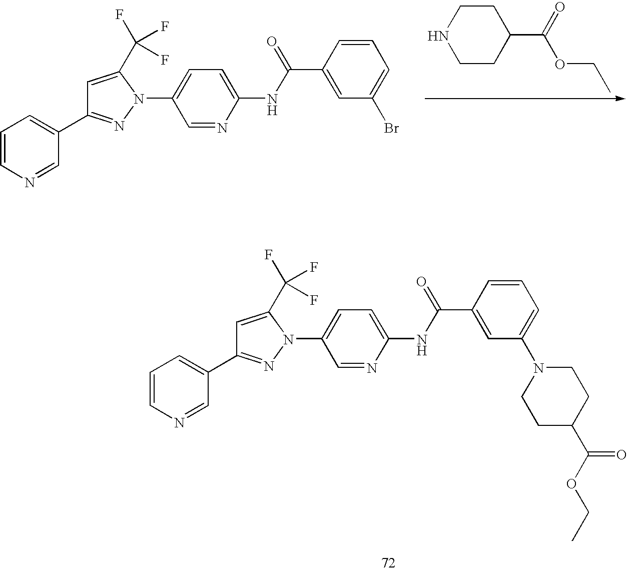

To a solution of 3-bromo-N-[5-(3-pyridin-3-yl-5-trifluoromethyl-pyrazol-1-yl)-pyridin-2-yl]-benzamide (100 mg, 0.21 mmol), prepared according to step a in example 8, in dimethylsulfoxide (4 mL) are added ethyl isonipecotate (0.06 mL, 0.4 mmol), Cu (I) iodide (4 mg, 0.02 mmol), L-proline (5 mg, 0.04 mmol) and potassium carbonate (28 mg, 0.21 mmol) respectively. The solution mixture is heated up to 90° C. in a microwave reactor for 90 minutes. The resulting solution is cooled to room temperature and then diluted with water (10 mL). The solution mixture is extracted with ethyl acetate (20 mL, 3×), the combined organic layers are dried over magnesium sulfate, and then filtered. The filtrate is concentrated under reduced pressure and the residue is purified by chromatography to afford the title compound (23 mg, 20%). LC-MS (M⁺+1): 565.30.

The following compounds are prepared according to example 72 by replacing the amine with commercially available starting materials.

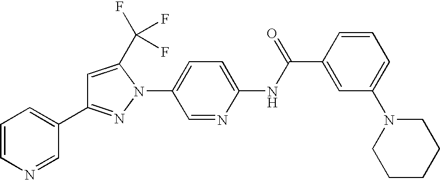

3-Piperidin-1-yl-N-[5-(3-pyridin-3-yl-5-trifluoromethyl-pyrazol-1-yl)-pyridin-2-yl]-benzamide; LC-MS (M⁺+1): 493.36.

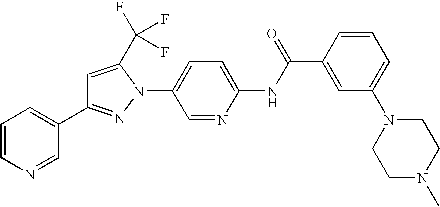

3-(4-Methyl-piperazin-1-yl)-N-[5-(3-pyridin-3-yl-5-trifluoromethyl-pyrazol-1-yl)-pyridin-2-yl]-benzamide; LC-MS (M⁺+1): 508.31.

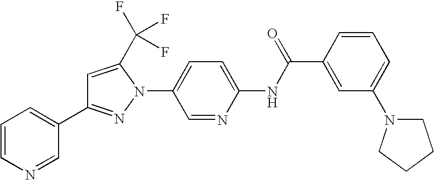

N-[5-(3-Pyridin-3-yl-5-trifluoromethyl-pyrazol-1-yl)-pyridin-2-yl]-3-pyrrolidin-1-yl-benzamide; LC-MS (M⁺+1): 479.27.

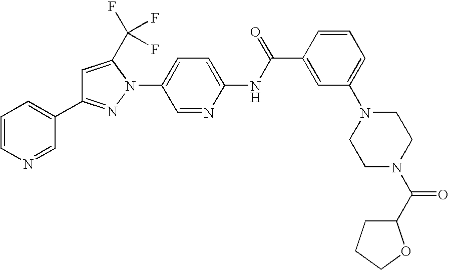

N-[5-(3-Pyridin-3-yl-5-trifluoromethyl-pyrazol-1-yl)-pyridin-2-yl]-3-[4-(tetrahydrofuran-2-carbonyl)-piperazin-1-yl]-benzamide; LC-MS (M⁺+1): 591.6.

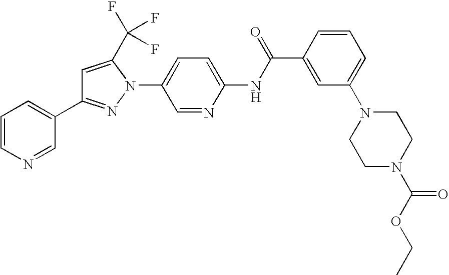

4-{3-[5-(3-Pyridin-3′-yl-5-trifluoromethyl-pyrazol-1-yl)-pyridin-2-ylcarbamoyl]-phenyl}-piperazine-1-carboxylic acid ethyl ester; LC-MS (M⁺+1): 565.48.

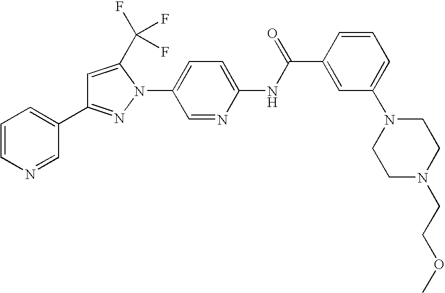

3-[4-(2-Methoxy-ethyl)-piperazin-1-yl]-N-[5-(3-pyridin-3-yl-5-trifluoromethyl-pyrazol-1-yl)-pyridin-2-yl]-benzamide, LC-MS (M⁺+1): 552.27.

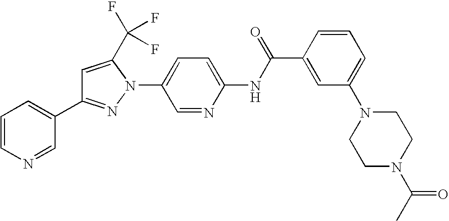

3-(4-Acetyl-piperazin-1-yl)-N-[5-(3-pyridin-3-yl-5-trifluoromethyl-pyrazol-1-yl)-pyridin-2-yl]-benzamide; LC-MS (M⁺+1): 536.25.

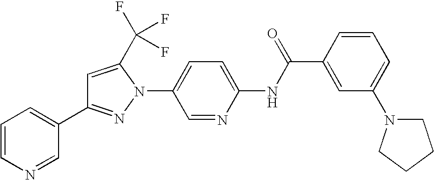

N-[5-(3-Pyridin-3-yl-5-trifluoromethyl-pyrazol-1-yl)-pyridin-2-yl]-3-pyrrolidin-1-yl-benzamide; LC-MS (M⁺+1): 479.27.

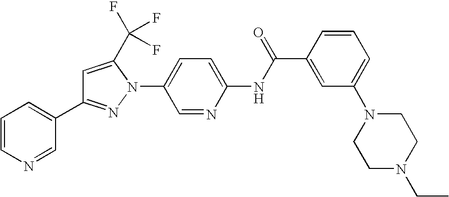

3-(4-Ethyl-piperazin-1-yl)-N-[5-(3-pyridin-3-yl-5-trifluoromethyl-pyrazol-1-yl)-pyridin-2-yl]-benzamide; LC-MS (M⁺+1): 522.3.

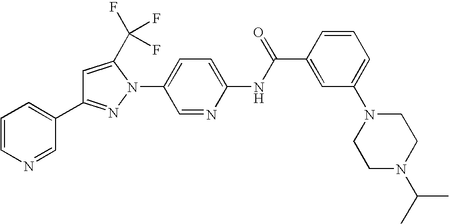

3-(4-Isopropyl-piperazin-1-yl)-N-[5-(3-pyridin-3-yl-5-trifluoromethyl-pyrazol-1-yl)-pyridin-2-yl]-benzamide; LC-MS (M⁺+1): 536.34.

### Example 73

### 1-{3-[5-(3-Pyridin-3-yl-5-trifluoromethyl-pyrazol-1-yl)-pyridin-2-ylcarbamoyl]-phenyl}-piperidine-4-carboxylic Acid

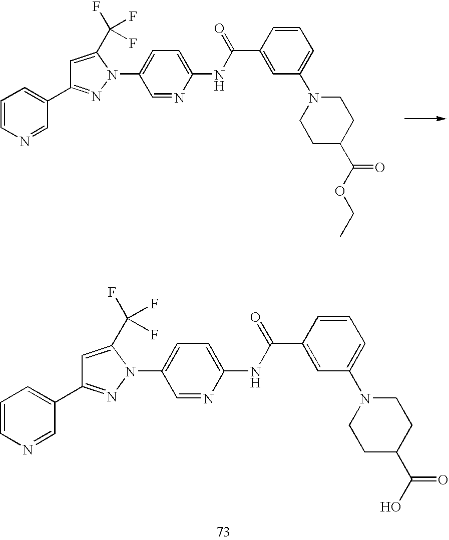

To a solution of 1-{3-[5-(3-pyridin-3-yl-5-trifluoromethyl-pyrazol-1-yl)-pyridin-2-ylcarbamoyl]-phenyl}-piperidine-4-carboxylic acid ethyl ester (50 mg, 0.089 mmol), prepared according to example 71, in 1,4-dioxane (10 mL) and water (1 mL) is added lithium hydroxide (4 mg, 0.18 mmol) at room temperature. The solution is stirred at the same temperature for 6 hours. The solvent is removed under reduced pressure and the residue is purified by chromatography to afford the title compound (19 mg, 40%). LC-MS (M¹+1): 536.50.

### Methods of Use

In accordance with the invention, there are provided methods of using the compounds as described herein and their pharmaceutically acceptable derivatives. The compounds used in the invention prevent the degradation of sEH substrates that have beneficial effects or prevent the formation of metabolites that have adverse effects. The inhibition of sEH is an attractive means for preventing and treating a variety of cardiovascular diseases or conditions e.g., endothelial dysfunction. Thus, the methods of the invention are useful for the treatment of such conditions. These encompass diseases including, but not limited to, type 1 and type 2 diabetes, insulin resistance syndrome, hypertension, atherosclerosis, coronary artery disease, angina, ischemia, ischemic stroke, Raynaud's disease and renal disease.

For therapeutic use, the compounds may be administered in any conventional dosage form in any conventional manner. Routes of administration include, but are not limited to, intravenously, intramuscularly, subcutaneously, intrasynovially, by infusion, sublingually, transdermally, orally, topically or by inhalation. The preferred modes of administration are oral and intravenous.

The compounds described herein may be administered alone or in combination with adjuvants that enhance stability of the inhibitors, facilitate administration of pharmaceutic compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies. Compounds of the invention may be physically combined with the conventional therapeutics or other adjuvants into a single pharmaceutical composition. Advantageously, the compounds may then be administered together in a single dosage form. In some embodiments, the pharmaceutical compositions comprising such combinations of compounds contain at least about 5%, but more preferably at least about 20%, of a compound (w/w) or a combination thereof. The optimum percentage (w/w) of a compound of the invention may vary and is within the purview of those skilled in the art. Alternatively, the compounds may be administered separately (either serially or in parallel). Separate dosing allows for greater flexibility in the dosing regime.

As mentioned above, dosage forms of the above-described compounds include pharmaceutically acceptable carriers and adjuvants known to those of ordinary skill in the art. These carriers and adjuvants include, for example, ion exchangers, alumina, aluminum stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known (scc, for example, H. C. Ansel and N. G. Popovish, Pharmaceutical Dosage Forms and Drug Delivery Systems, 5th ed., Lea and Febiger (1990)). Dosage levels and requirements are well-recognized in the art and may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

The term “patient” includes both human and non-human mammals.

The term “effective amount” means an amount of a compound according to the invention which, in the context of which it is administered or used, is sufficient to achieve the desired effect or result. Depending on the context, the term effective amount may include or be synonymous with a pharmaceutically effective amount or a diagnostically effective amount.

The terms “pharmaceutically effective amount” or “therapeutically effective amount” means an amount of a compound according to the invention which, when administered to a patient in need thereof, is sufficient to effect treatment for disease-states, conditions, or disorders for which the compounds have utility. Such an amount would be sufficient to elicit the biological or medical response of a tissue, system, or patient that is sought by a researcher or clinician. The amount of a compound of according to the invention which constitutes a therapeutically effective amount will vary depending on such factors as the compound and its biological activity, the composition used for administration, the time of administration, the route of administration, the rate of excretion of the compound, the duration of treatment, the type of disease-state or disorder being treated and its severity, drugs used in combination with or coincidentally with the compounds of the invention, and the age, body weight, general health, sex, and diet of the patient. Such a therapeutically effective amount can be determined routinely by one of ordinary skill in the art having regard to their own knowledge, the prior art, and this disclosure.

The term “diagnostically effective amount” means an amount of a compound according to the invention which, when used in a diagnostic method, apparatus, or assay, is sufficient to achieve the desired diagnostic effect or the desired biological activity necessary for the diagnostic method, apparatus, or assay. Such an amount would be sufficient to elicit the biological or medical response in a diagnostic method, apparatus, or assay, which may include a biological or medical response in a patient or in a in vitro or in vivo tissue or system, that is sought by a researcher or clinician. The amount of a compound according to the invention which constitutes a diagnostically effective amount will vary depending on such factors as the compound and its biological activity, the diagnostic method, apparatus, or assay used, the composition used for administration, the time of administration, the route of administration, the rate of excretion of the compound, the duration of administration, drugs and other compounds used in combination with or coincidentally with the compounds of the invention, and, if a patient is the subject of the diagnostic administration, the age, body weight, general health, sex, and diet of the patient. Such a diagnostically effective amount can be determined routinely by one of ordinary skill in the art having regard to their own knowledge, the prior art, and this disclosure.

The terms “treating” or “treatment” mean the treatment of a disease-state in a patient, and include:
(i) preventing the disease-state from occurring in a patient, in particular, when such patient is genetically or otherwise predisposed to the disease-state but has not yet been diagnosed as having it;
(ii) inhibiting or ameliorating the disease-state in a patient, i.e., arresting or slowing its development; or
(iii) relieving the disease-state in a patient, i.e., causing regression or cure of the disease-state.

### In Vitro Assay for Inhibition of hsEH

This high throughput screen identifies compounds that inhibit the interaction of human soluble epoxide hydrolase (sEH) with a tetramethyl rhodamine (TAMRA)-labeled probe. The UHTS employs the Zymark Allegro modular robotic system to dispense reagents, buffers, and test compounds into either 96-well or 384-well black microtiter plates (from Costar). The assay buffer is: 20 mM TES, 200 mM NaCl, 0.05% w/v CHAPS, 1 mM TCEP, pH=7.0. Test compounds dissolved in neat DMSO at 5 mg/mL are diluted to 0.5 mg/mL in neat DMSO. The 0.5 mg/mL solutions are further diluted to 30 μg/mL in assay buffer containing DMSO such that the final concentration of DMSO is 30%. For 384-well format, a mixture of 10.35 nM human sEH and 2.59 nM probe is prepared in assay buffer and 60 μL is added to each well for a final sEH concentration of 10 nM and a final probe concentration of 2.5 nM. 2.1 μL of diluted test compound is then added to each well, where the final assay concentration will be 1 μg/mL test compound and 1% DMSO. The final volume in each well is 62.1 μL. Positive controls are reaction mixtures containing no test compound; negative controls (blanks) are reaction mixtures containing 3 μM BI00611349XX. For 96-well format, the final concentration of all reaction components remains the same. 135 μL sEH/probe mixture is added to wells containing 15 μL test compound so that the final well volume is 150 mL. After incubating the reaction for 30 minutes at room temperature, the plates are read for fluorescence polarization in the LJL Analyst set to 530 nm excitation, 580 nm emission, using the Rh 561 dichroic mirror.

### In Vitro Assay for Inhibition of msEH

This screen identifies compounds that inhibit the interaction of rat soluble epoxide hydrolase (sEH) with a tetramethyl rhodamine (TAMRA)-labeled probe. The assay employs a Multimek, a Multidrop, and manual multi-channel pipettors to dispense reagents, buffers, and test compounds into 96-well black microtiter plates (Costar 3792). The assay buffer is: 20 mM TES, 200 mM NaCl, 0.05% w/v CHAPS, 1 mM TCEP, pH=7.0. Test compounds dissolved in neat DMSO at 10 mM are diluted to 1.5 mM in neat DMSO. The 1.5 mM solutions are serially diluted using 3-fold dilutions in neat DMSO in polypropylene plates. Assay buffer is added to the wells such that the compounds are diluted 10-fold and the DMSO concentration is 10%. A mixture of 11.1 nM rat sEH and 2.78 nM probe is prepared in assay buffer. 15 uL of diluted test compound is added to each well, where the final maximum assay concentration will be 3 uM test compound and 1% DMSO. 135 uL of sEH/probe mixture is added to each well for a final sEH concentration of 10 nM and a final probe concentration of 2.5 nM. The final volume in each well is 150 uL. Positive controls are reaction mixtures containing no test compound; negative controls (blanks) are reaction mixtures containing 3 uM BI00611349XX. After incubating the reaction for 30 minutes at room temperature, the plates are read for fluorescence polarization in the LJL Analyst set to 530 nm excitation, 580 nm emission, using the Rh 561 dichroic mirror.

### In Vivo Models of Hypertension

Compounds were administered to spontaneous hypertensive rats (SHR) or Dahl Salt Sensitive (D-SS) rats on a high salt diet. Tail plethysmography was used to record changes in systolic blood pressure (SBP) and heart rate (HR) at selected time points after dosing in conscious, resting rats. As an alternative, radio transmitters were surgically implanted in the abdominal aorta to facilitate continuous monitoring of SBP, diastolic blood pressure (DBP), mean blood pressure (MBP) and HR via telemetry in conscious, unrestrained rats. Efficacy was evaluated based on the ability of compound treatment to affect a statistically significant lowering of blood pressure compared to respective vehicle (placebo) control groups.

1. A compound of the formula (I) wherein: 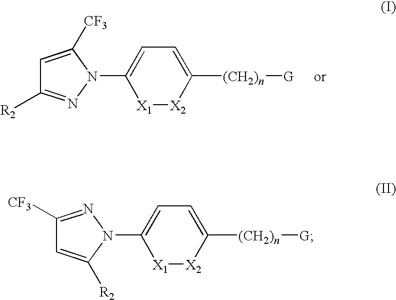
wherein G= 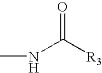
or 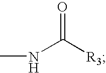
X₁-X₂ is —CH═CH—, —N═CH—, —C═N— or —N═N—;
R₂ is chosen from heteroaryl and carbocycle optionally substituted by C₁₋₁₀ alkyl, C₁₋₁₀ alkoxy each substituent of R₂ is optionally halogenated;
R₃ is chosen from heteroaryl, heterocycle, carbocycle, Ar₂—Ar₁- and an acyclic moiety chosen from: —NH—(CH₂)ₜ—Ar₁, —NH—(CH₂)ₜ—O—Ar₁, —NH—Ar₁, C₁₋₁₀ alkyl, —C₁₋₁₀ alkyl-Ar₁, O—C₁₋₁₀ alkyl-Ar₁, Ar₂-L-Ar₁- and —C₁₋₁₀ alkyl(phenyl)₂, or R₃ is L;
L is a C₁₋₁₀ alkyl chain optionally interrupted by O, S or NRₓ, and optionally substituted by oxo (═O);
Ar₁ and Ar₂ are each independently heteroaryl, heterocycle or carbocycle, each optionally substituted by one or more C₁₋₁₀ alkyl, C₁₋₁₀ alkoxy, —NRₓR_{y}, —C(O)—NRₓR_{y}, Rₓ—S(O)m-, Het-C(O)—, Het-S(O)ₘ—, NO₂, OH, halogen, C₁₋₁₀ alkoxycarbonyl, CO₂, CN, C₁₋₁₀ acyl, —S(O)ₘ—NRₓR_{y}, Rₓ—S(O)ₘ—NHR_{y}, —(CH₂)ₜ—OH wherein Het is pyrrolidinyl or morpholinyl;
m is 0-2;
n is 0-5;
t is 0-5;
or the pharmaceutically acceptable salts thereof.

2. The compound according to claim 1 wherein:
R₂ is chosen from pyridinyl, phenyl and cyclohexyl optionally substituted by C₁₋₁₀ alkyl, C₁₋₁₀ alkoxy each substituent of R₂ is optionally halogenated;
R₃ is chosen from phenyl, pyridinone, pyridinyl, —NH—(CH₂)ₜ—Ar₁, —NH—(CH₂)ₜ—O—Ar₁, —NH—Ar₁, C₁₋₁₀ alkyl, —C₁₋₁₀ alkyl-Ar₁ and —C₁₋₁₀ alkyl(phenyl)₂;
Ar₁ and Ar₂ are each independently phenyl, pyridinone, pyridinyl, morpholinyl, benzofuranyl, piperidinyl, cyclohexenyl, benzodioxolanyl, pyrrolidinyl, tetrazolyl, oxazolyl, isoxazolyl, pyrimidinyl or benzodioxolyl.

3. A compound of the formula (Ia): 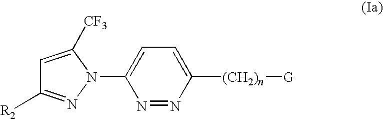
wherein for the Formula (Ia), the component R₂ is: 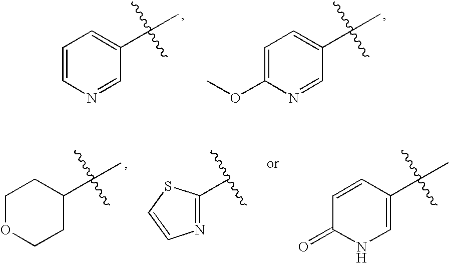
and the component 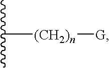
is chosen from those shown in the table I below;
TABLE I 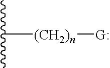 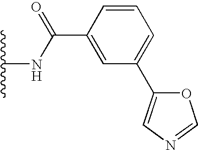 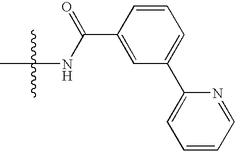 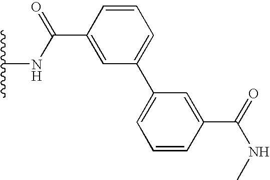 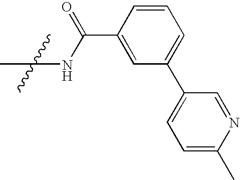 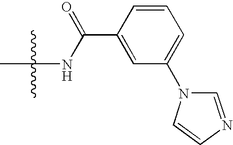 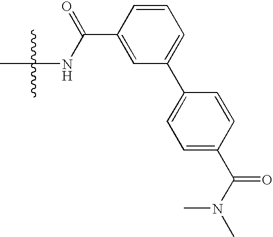 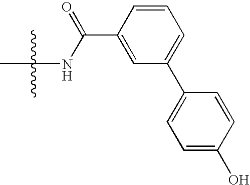 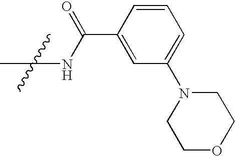 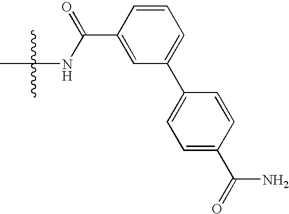 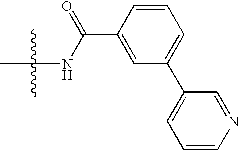 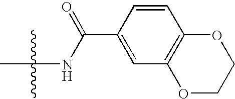 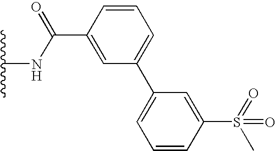 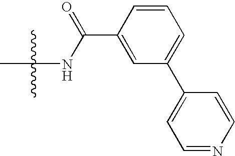 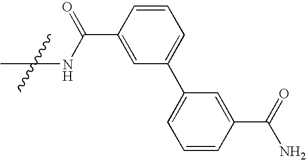 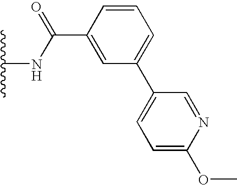 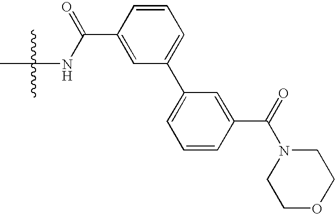 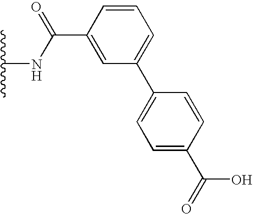 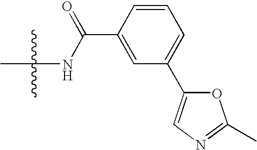 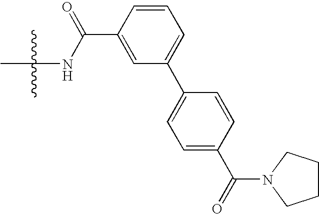 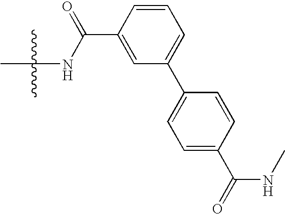 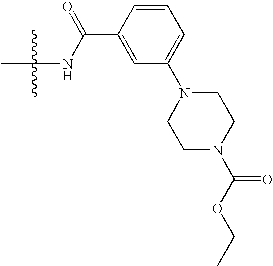 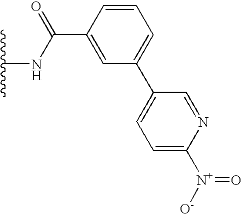 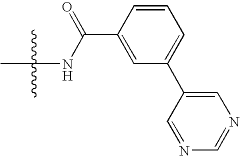 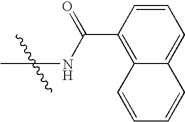 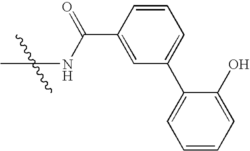 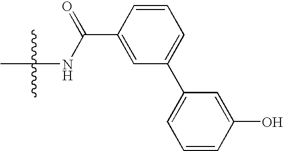 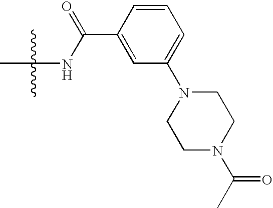 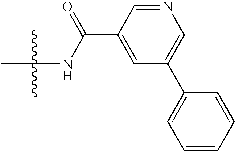 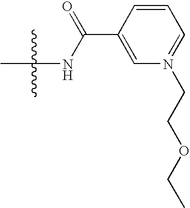 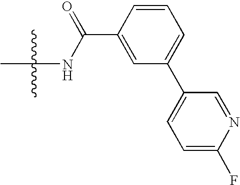 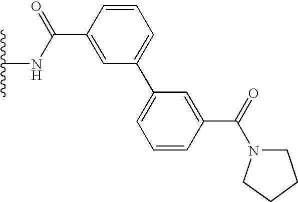 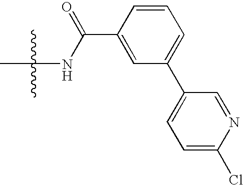 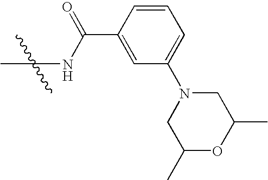 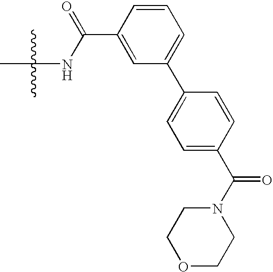 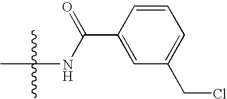 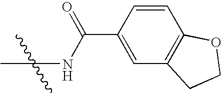 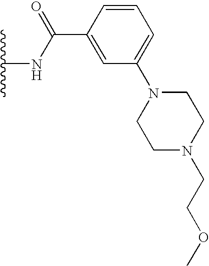 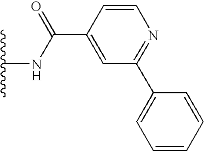 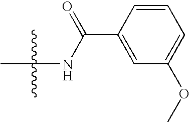 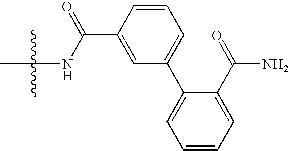 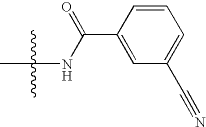 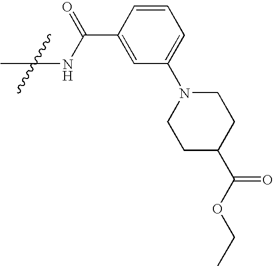 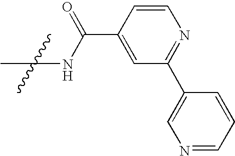 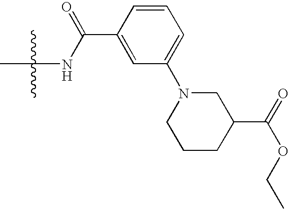 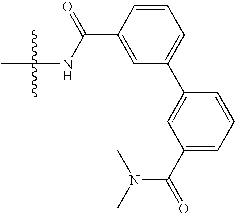 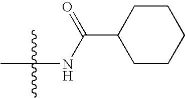 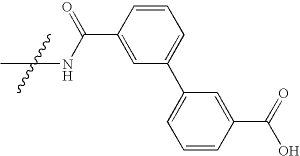 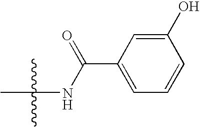 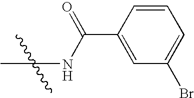 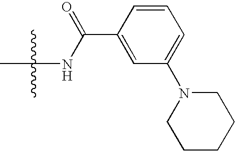 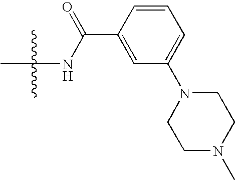 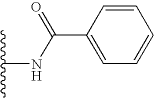 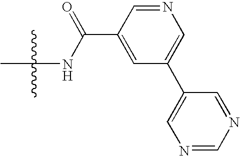 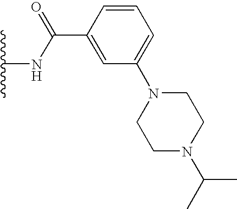 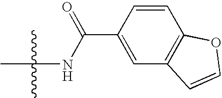 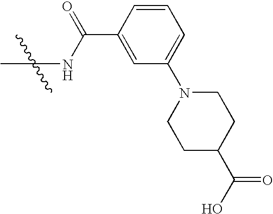 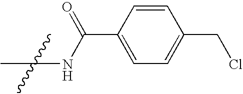 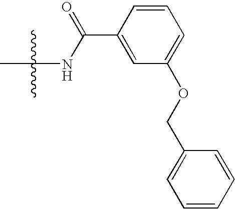 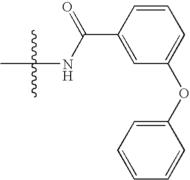 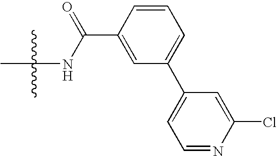 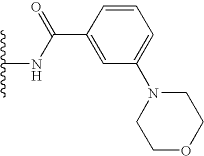 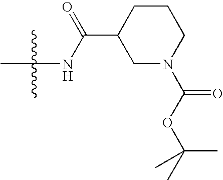 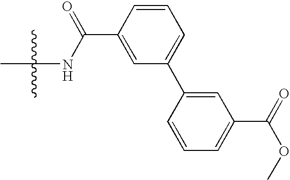 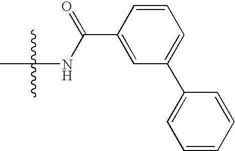 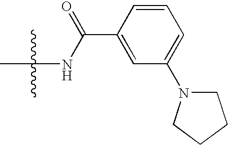 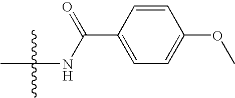 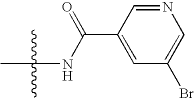 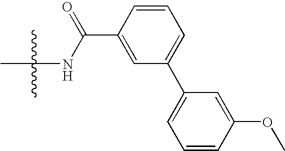 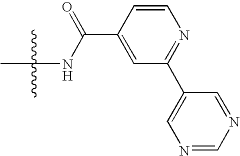 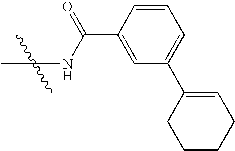 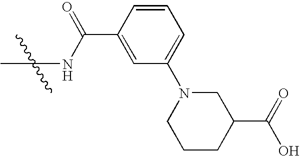 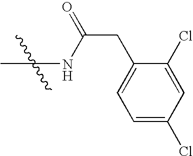 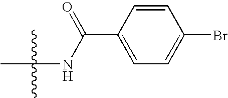 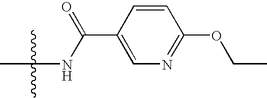 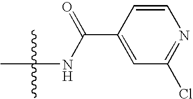 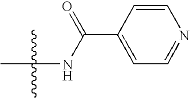 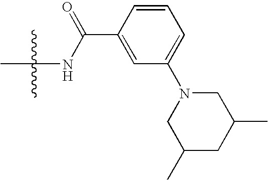 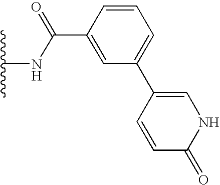 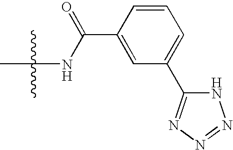 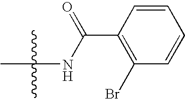 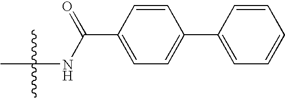 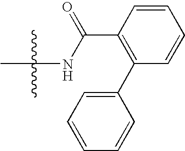 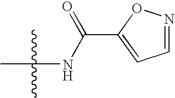 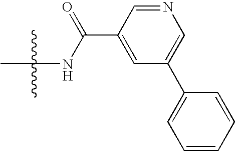
or the pharmaceutically acceptable salts thereof.

4. A compound of the formula (Ib): 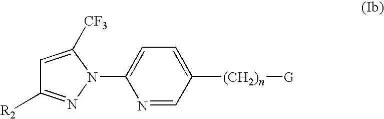
wherein for the Formula (Ib), the component R₂ is: 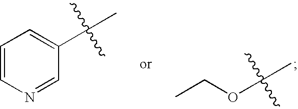
and the component 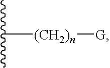
is chosen from those shown in the table II below;
TABLE II 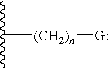 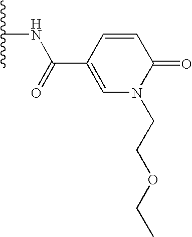 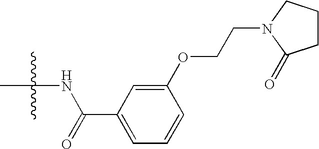 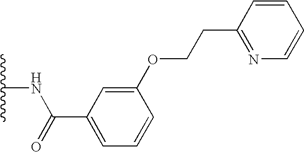 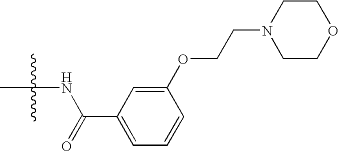 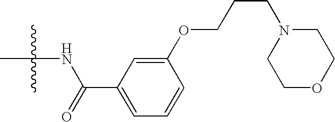 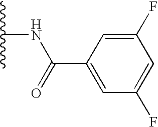 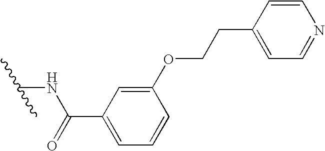 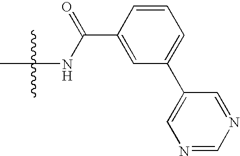 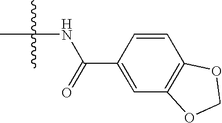 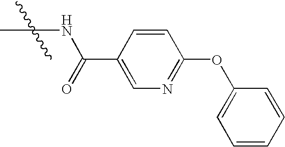 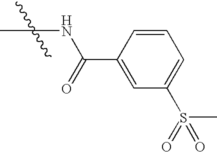 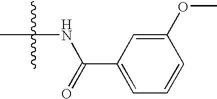 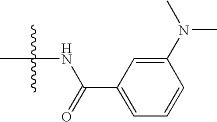 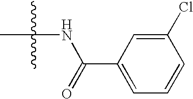 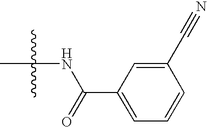 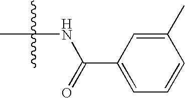 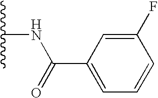 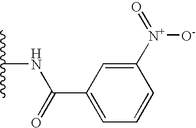 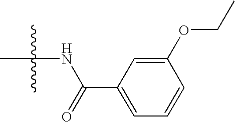 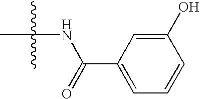 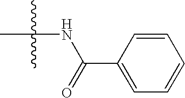 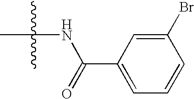 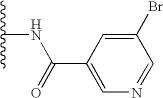 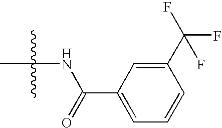 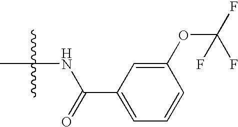 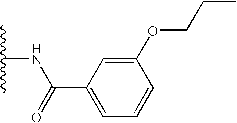 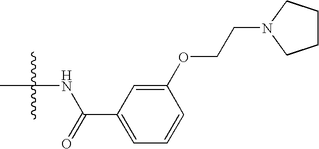 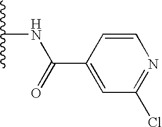 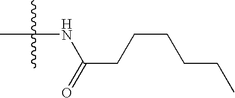 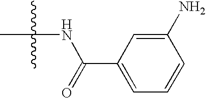 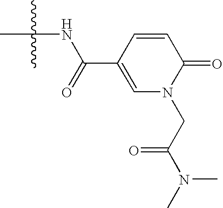 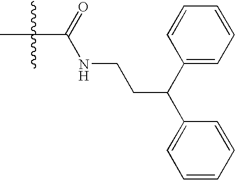 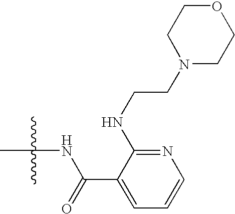 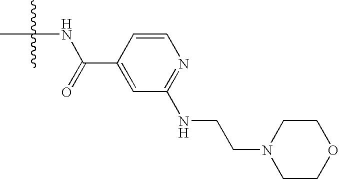 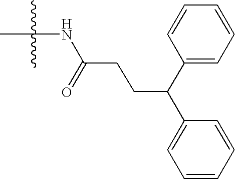 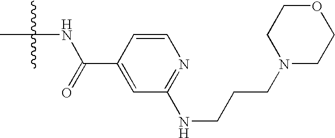 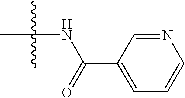 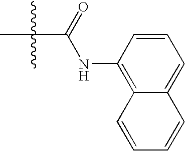 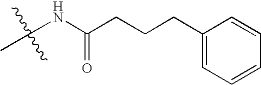 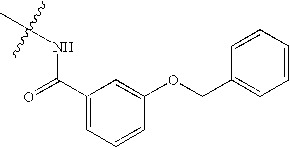 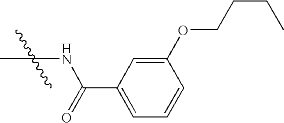 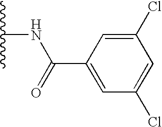 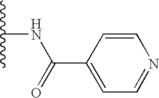 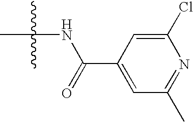 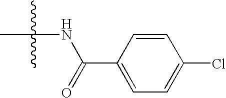 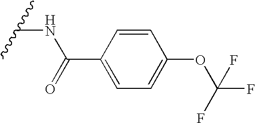 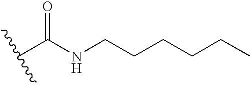 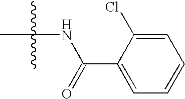 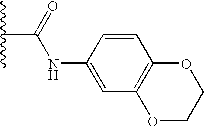 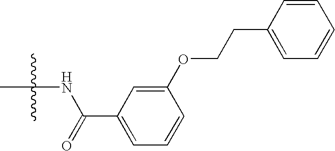 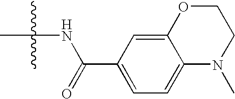 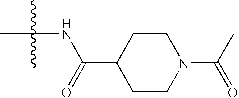 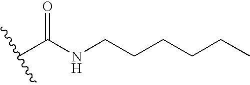 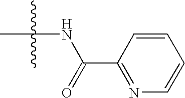 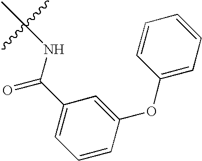 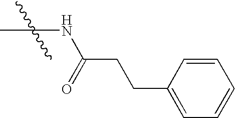 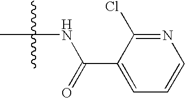 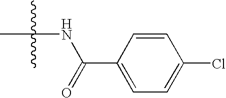 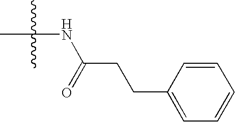
or the pharmaceutically acceptable salts thereof.

5. A compound of the formula (Ic) or (Id): 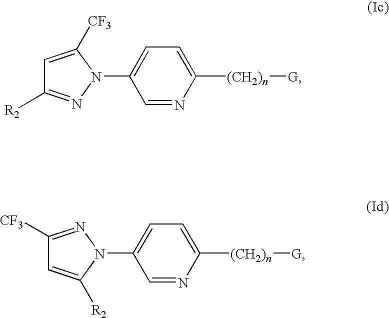
wherein for the Formula (Ic) or (Id), the component R₂ is: 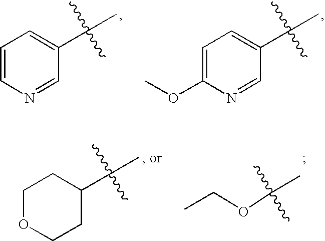
and the component 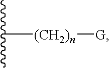
is chosen from those shown in the table III below;
TABLE III 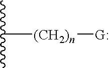 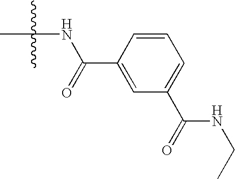 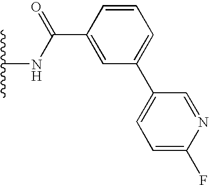 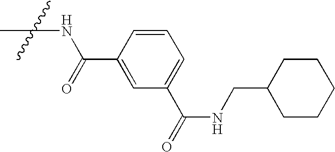 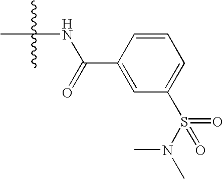 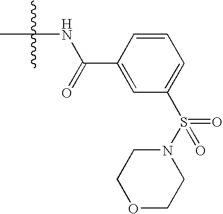 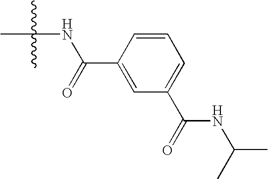 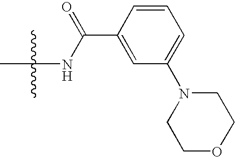 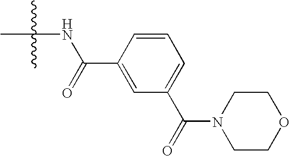 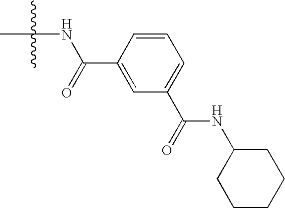 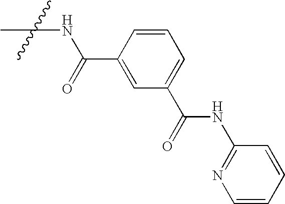 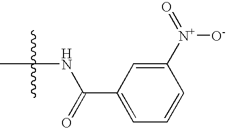 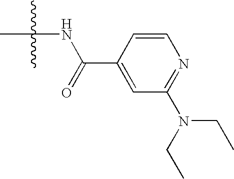 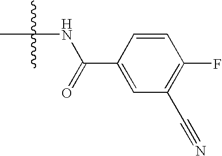 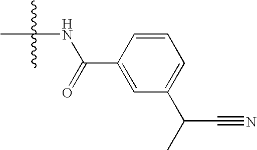 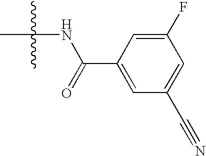 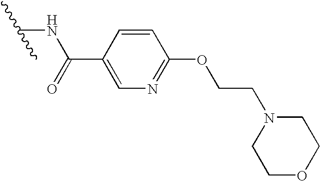 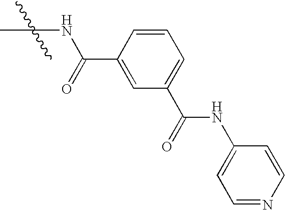 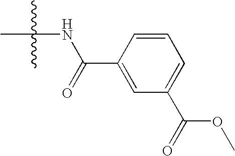 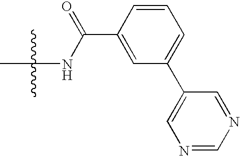 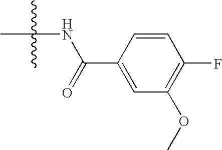 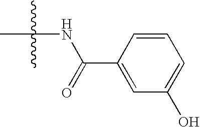 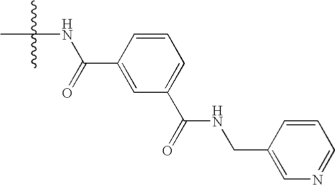 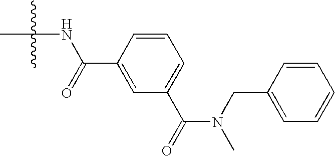 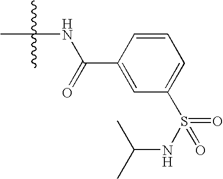 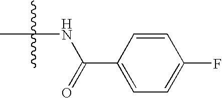 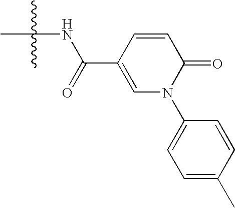 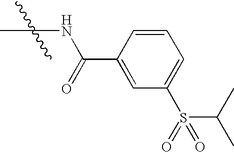 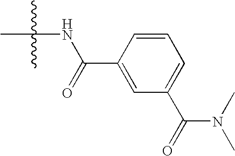 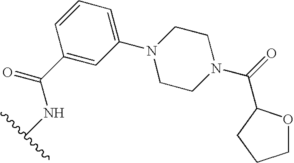 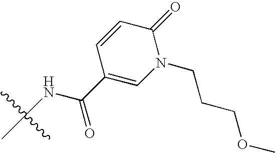 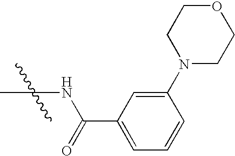 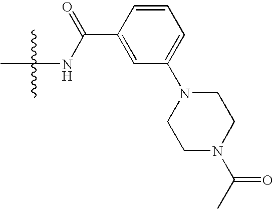 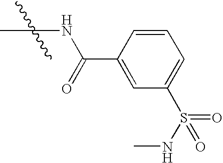 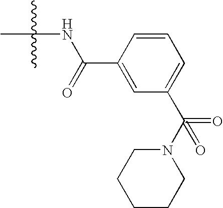 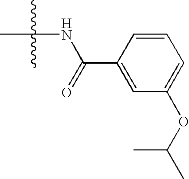 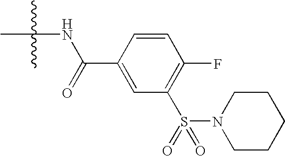 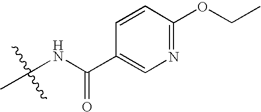 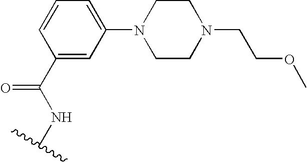 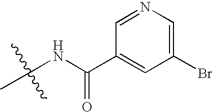 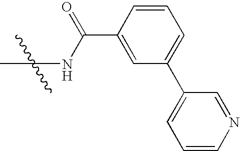 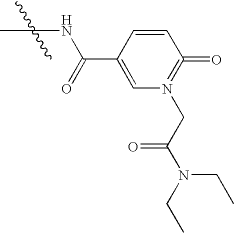 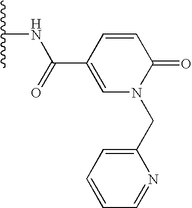 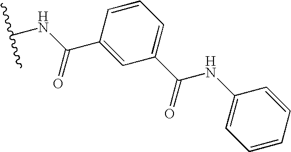 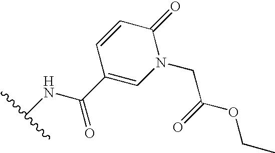 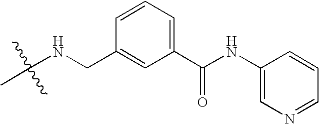 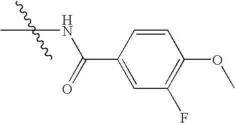 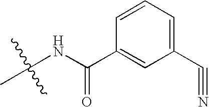 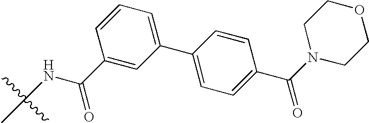 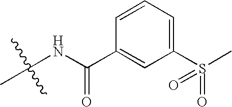 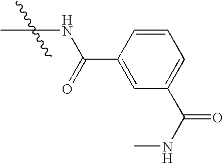 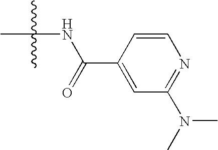 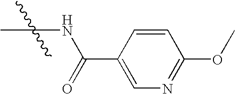 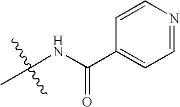 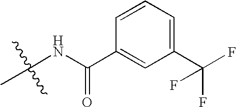 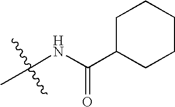 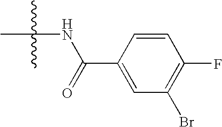 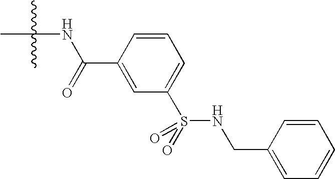 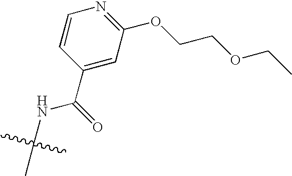 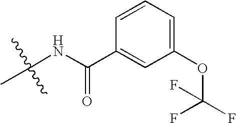 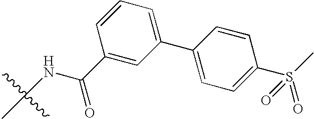 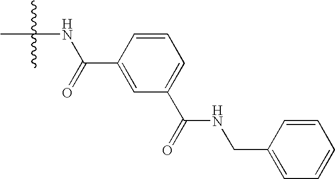 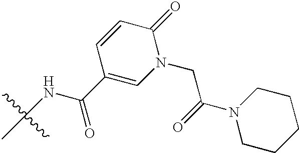 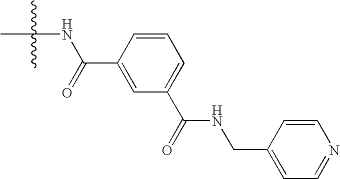 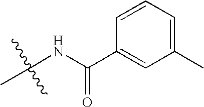 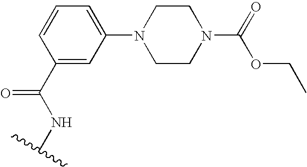 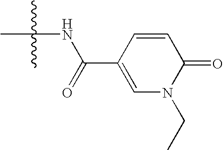 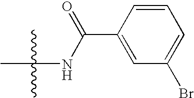 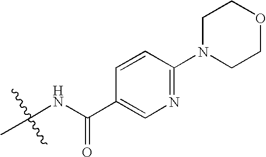 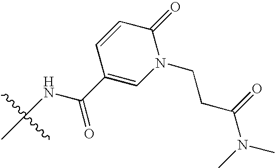 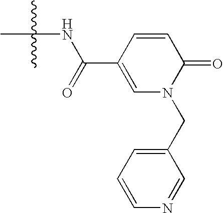 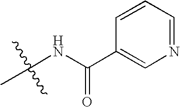 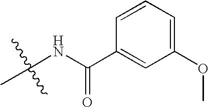 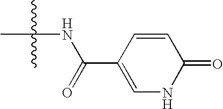 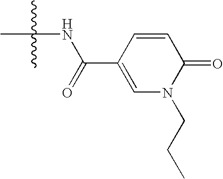 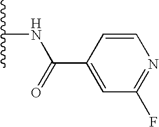 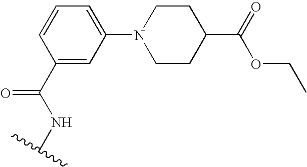 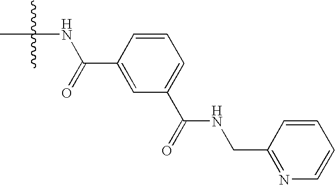 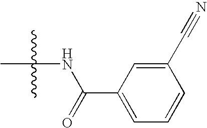 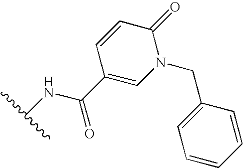 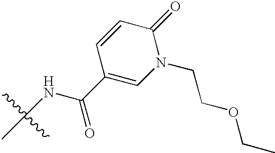 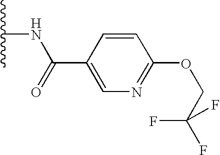 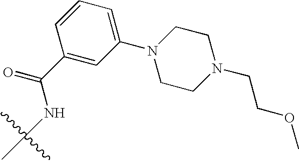 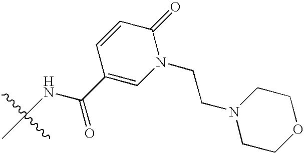 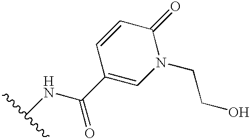 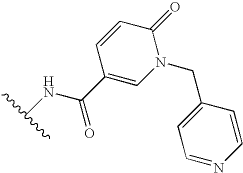 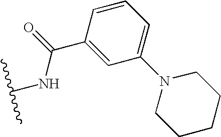 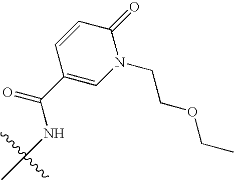 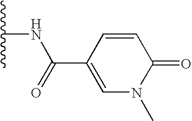 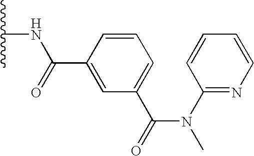 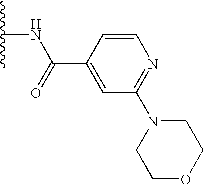 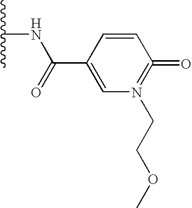 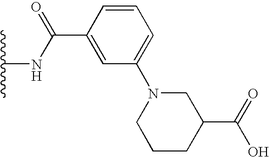 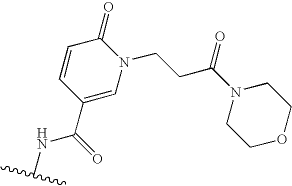 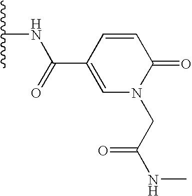 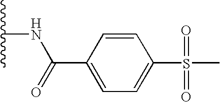 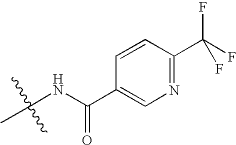 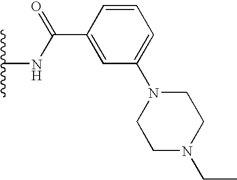 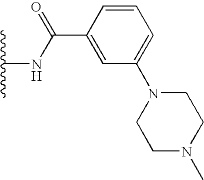 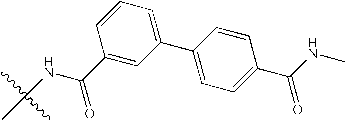 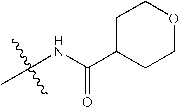 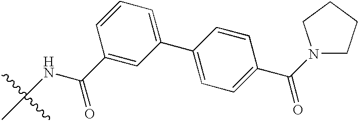 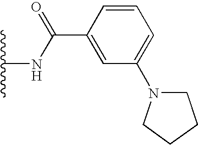 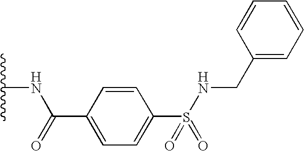 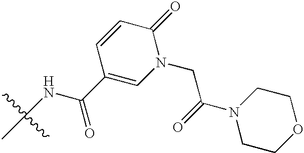 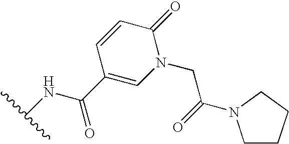 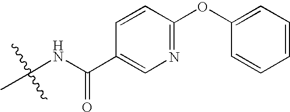 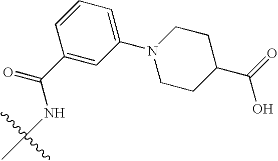 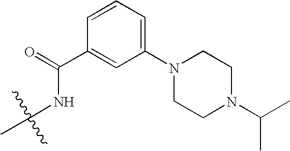 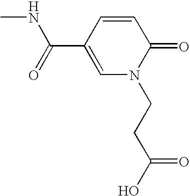 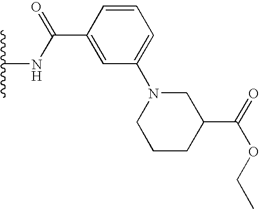 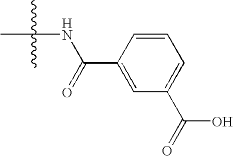 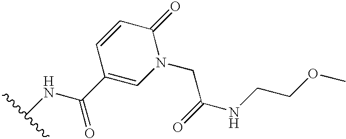 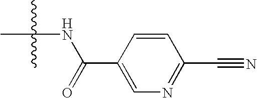 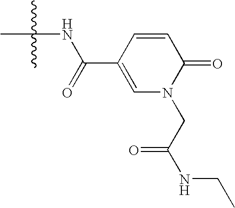 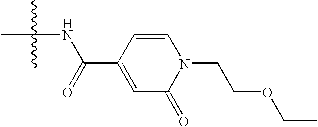 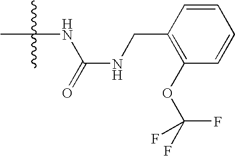 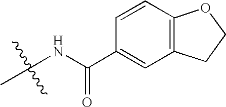 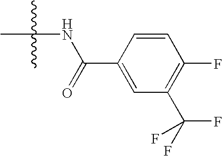 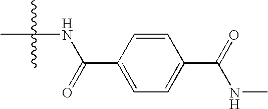 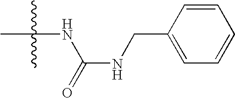 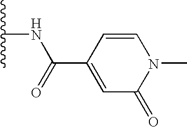 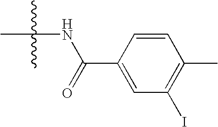 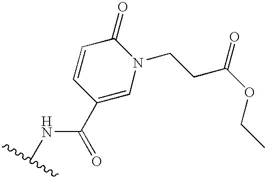 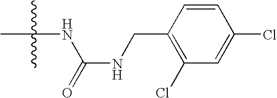 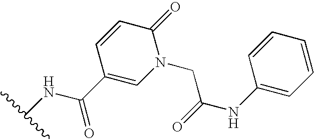 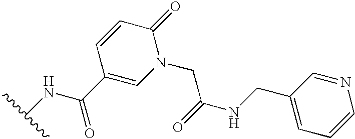 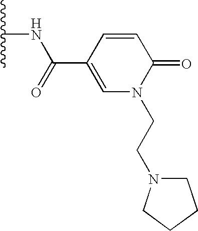 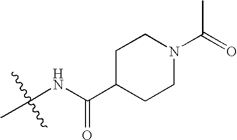 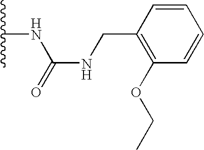 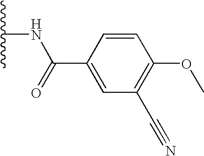 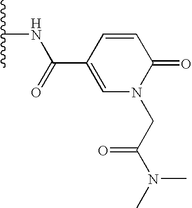 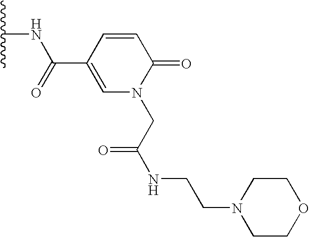 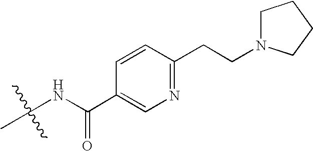 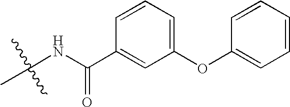 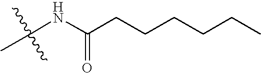 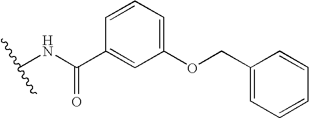 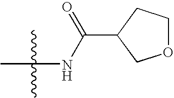 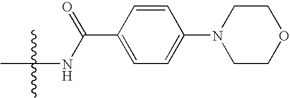 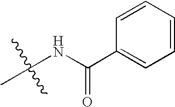 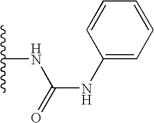 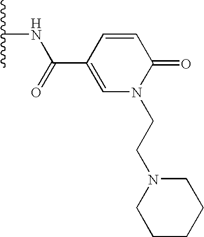 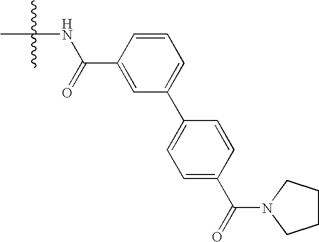 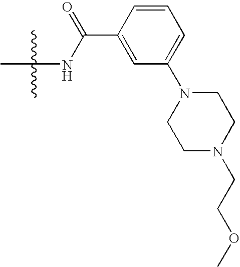 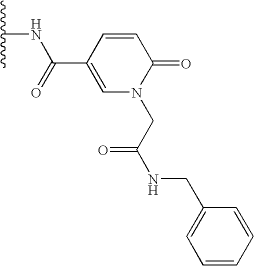 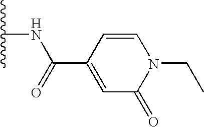
or the pharmaceutically acceptable salts thereof.

6. A compound of the formula (Ie), (If), (Ig) or (Ih): 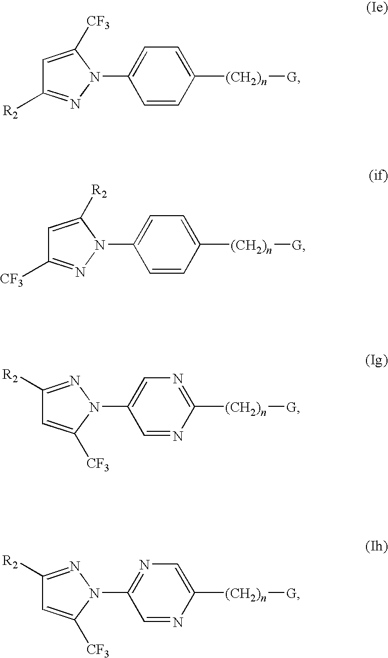
wherein for the Formula (Ie), (If), (Ig) or (Ih), the component R₂ is: 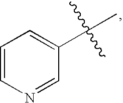
and the component 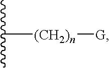
is chosen from those shown in the table IV below;
TABLE IV 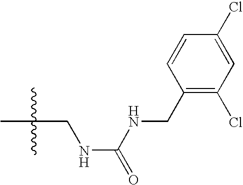 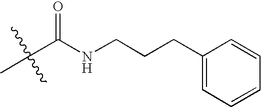 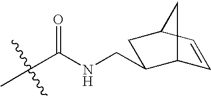 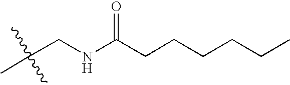 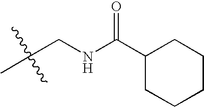 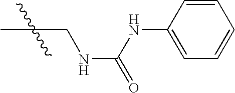 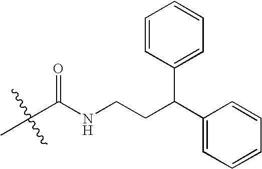 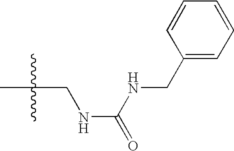 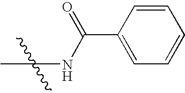 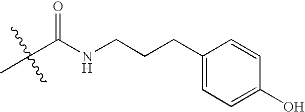 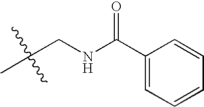 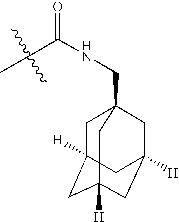 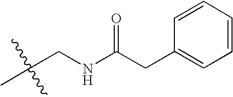 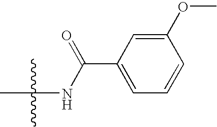 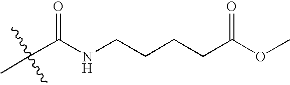 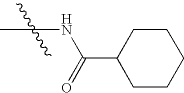 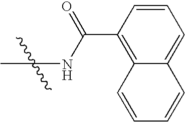 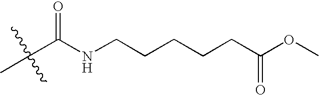 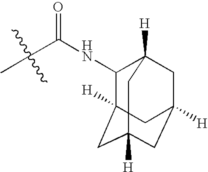 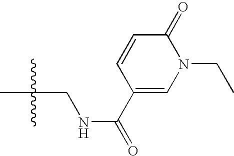
or the pharmaceutically acceptable salts thereof.

7. A compound chosen from: 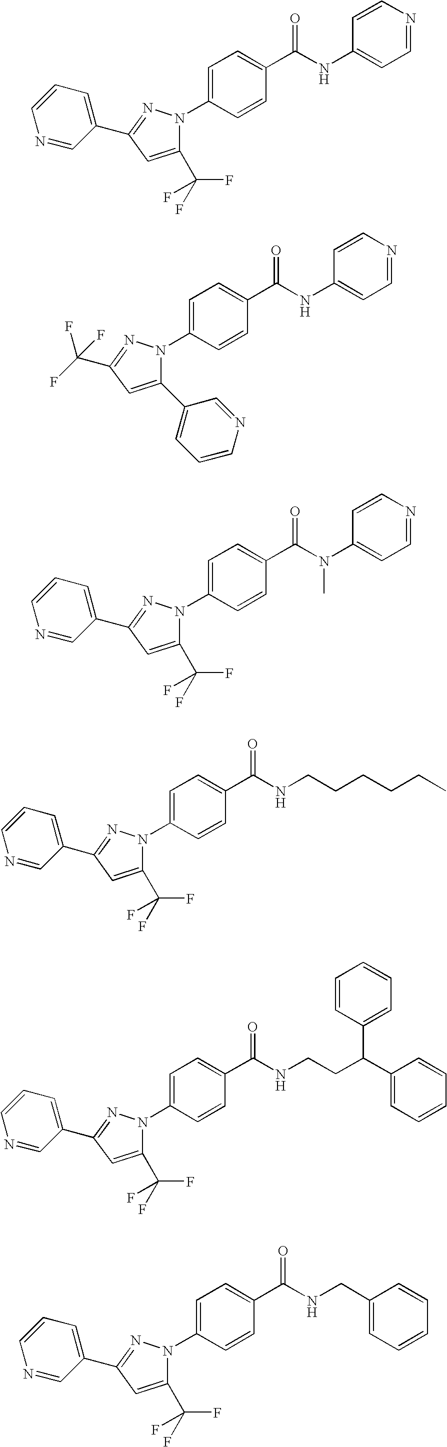 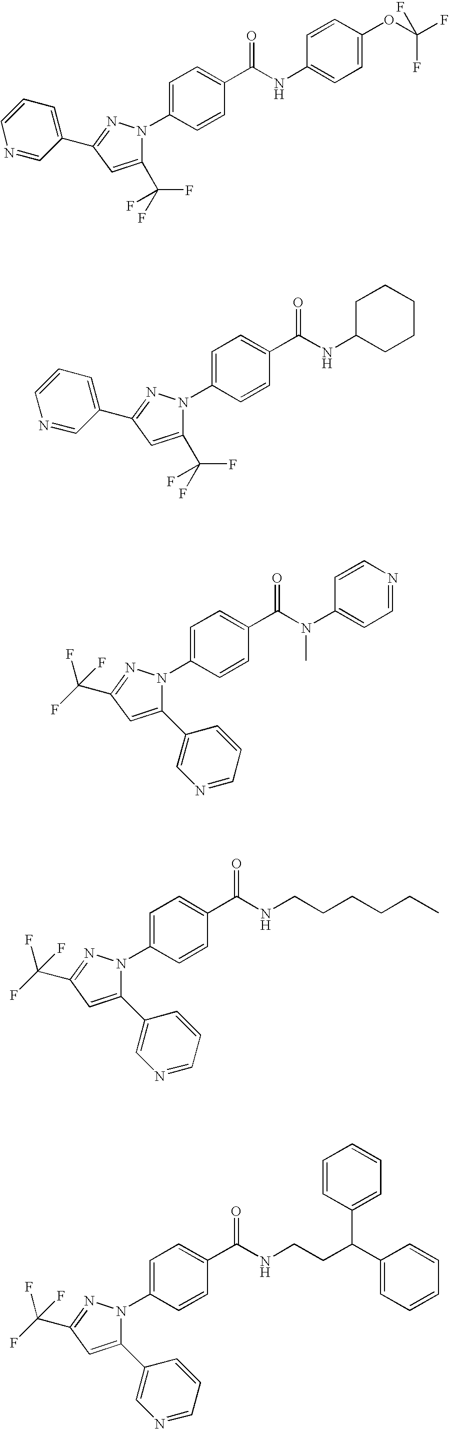 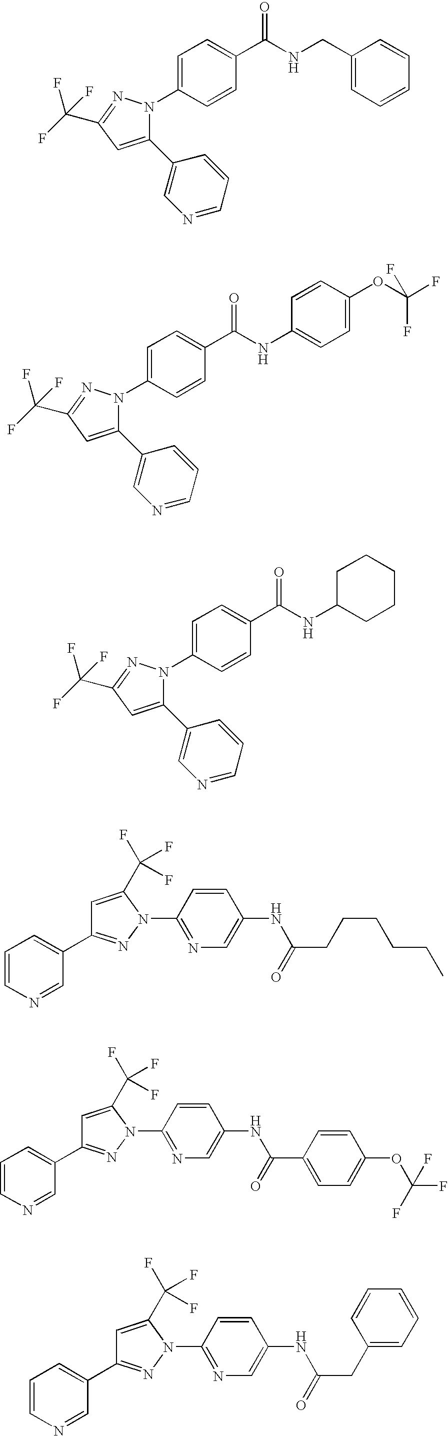 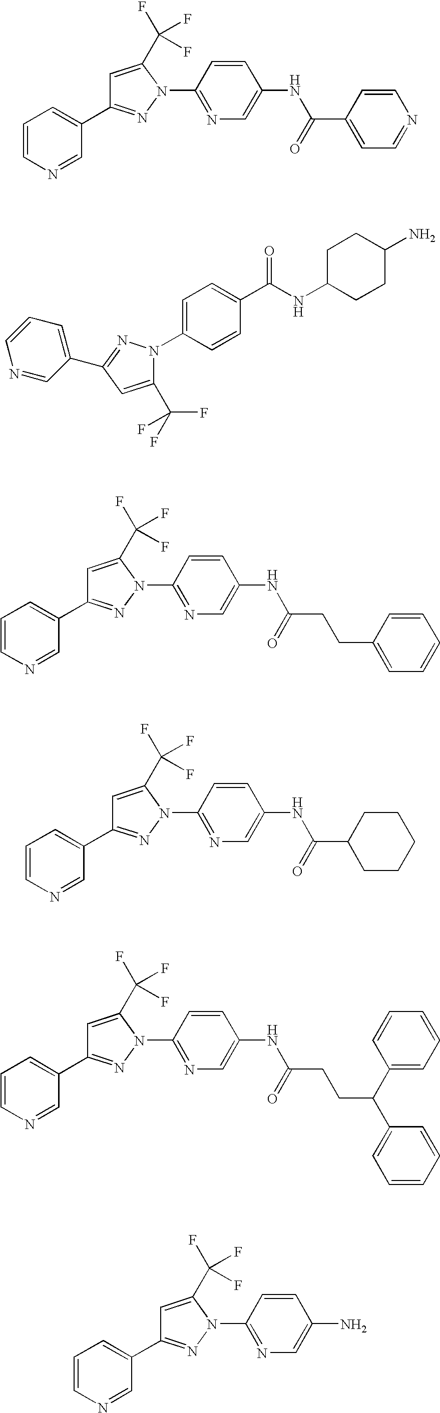 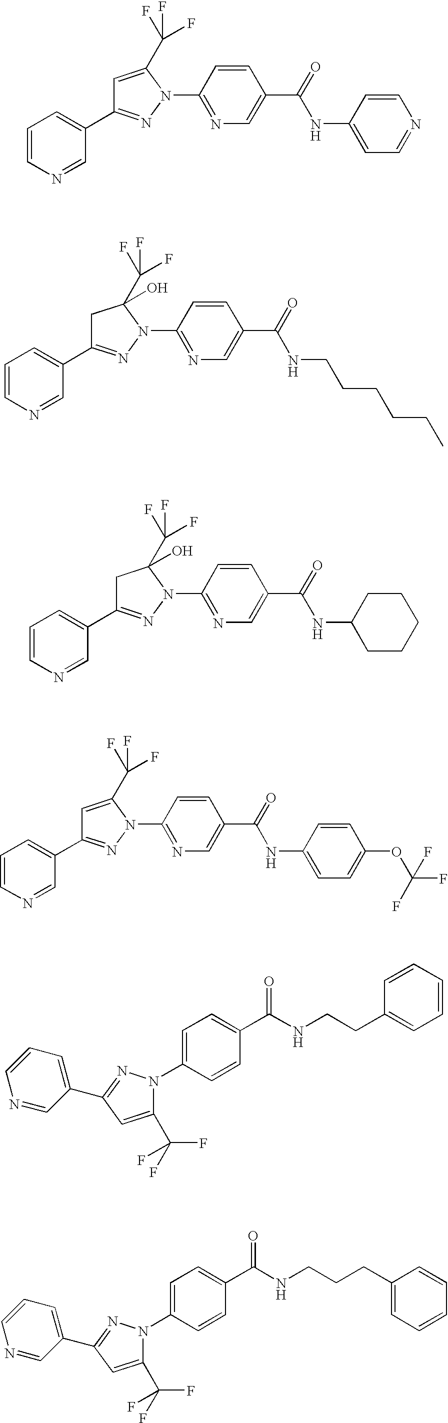 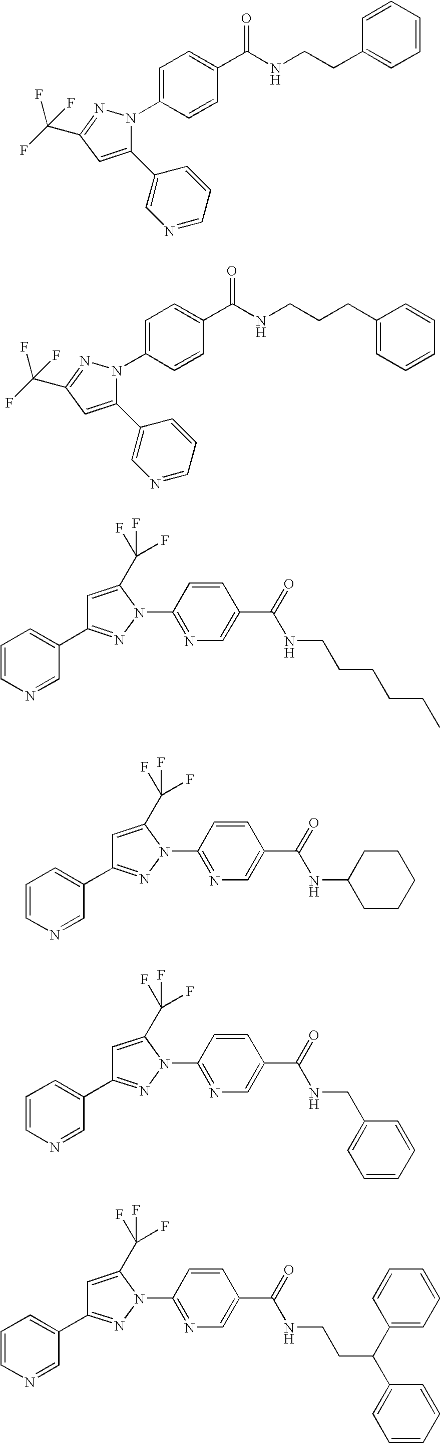 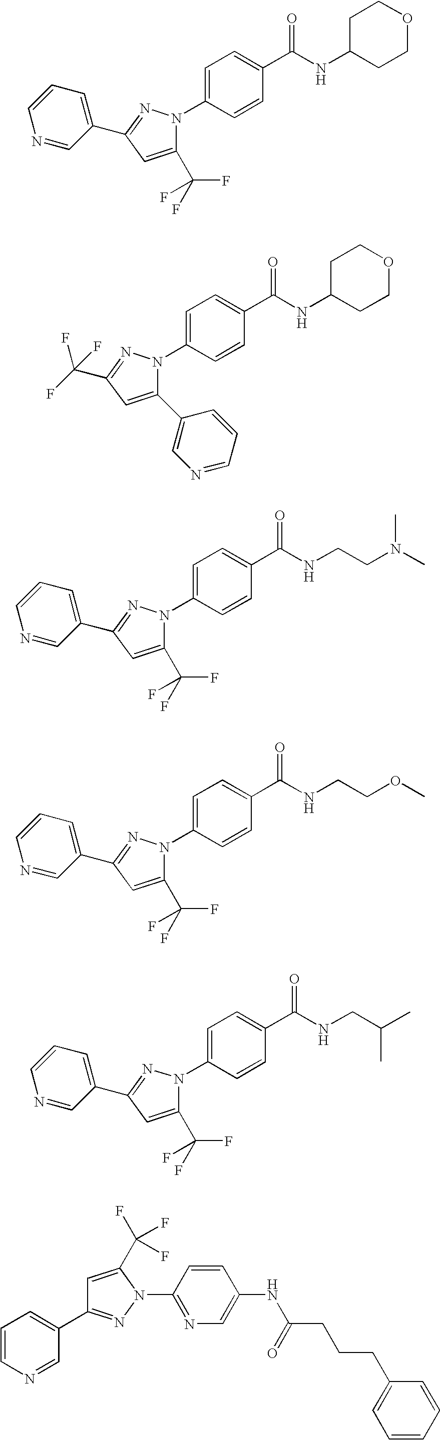 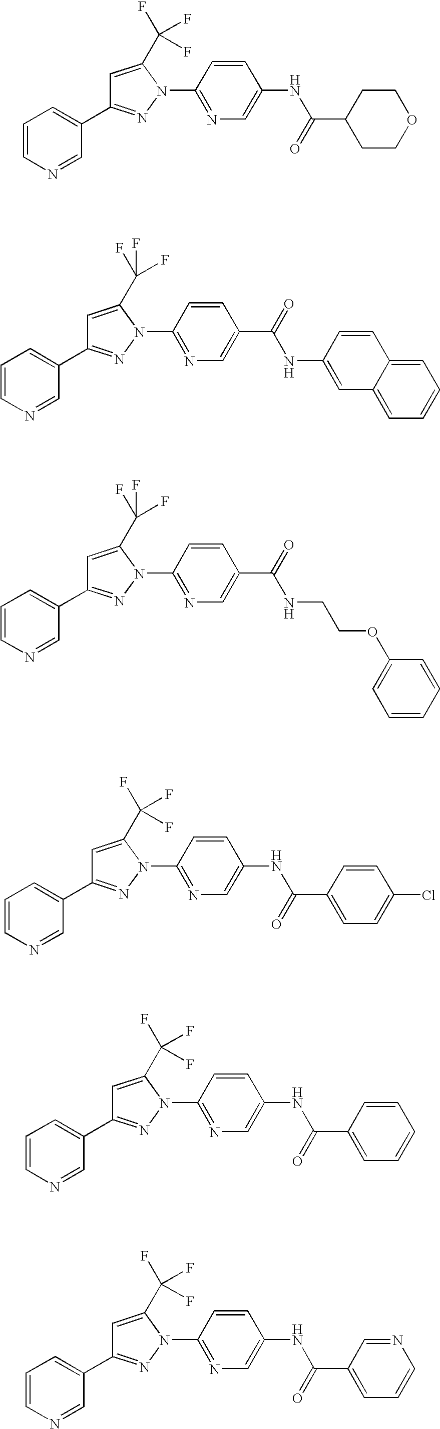 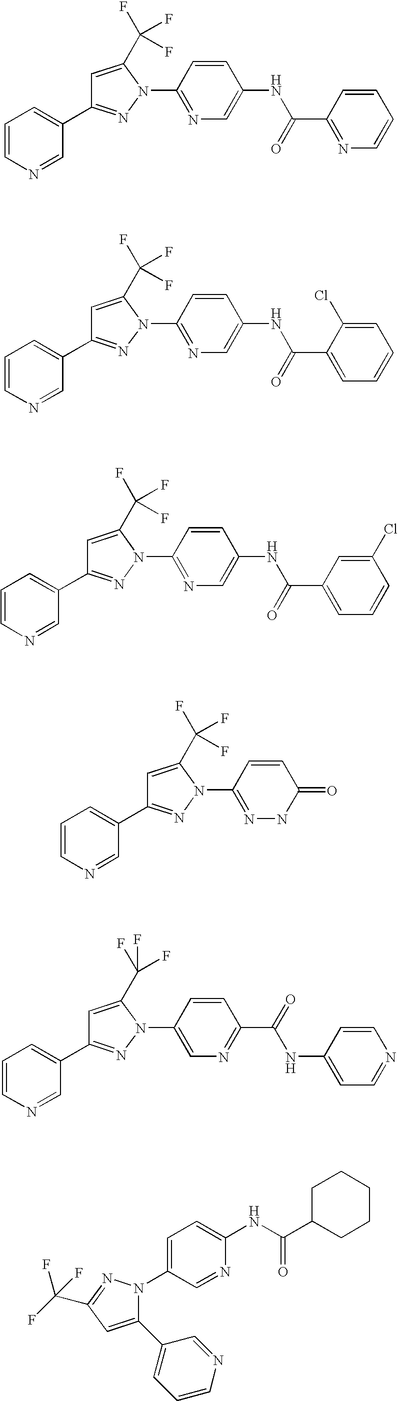 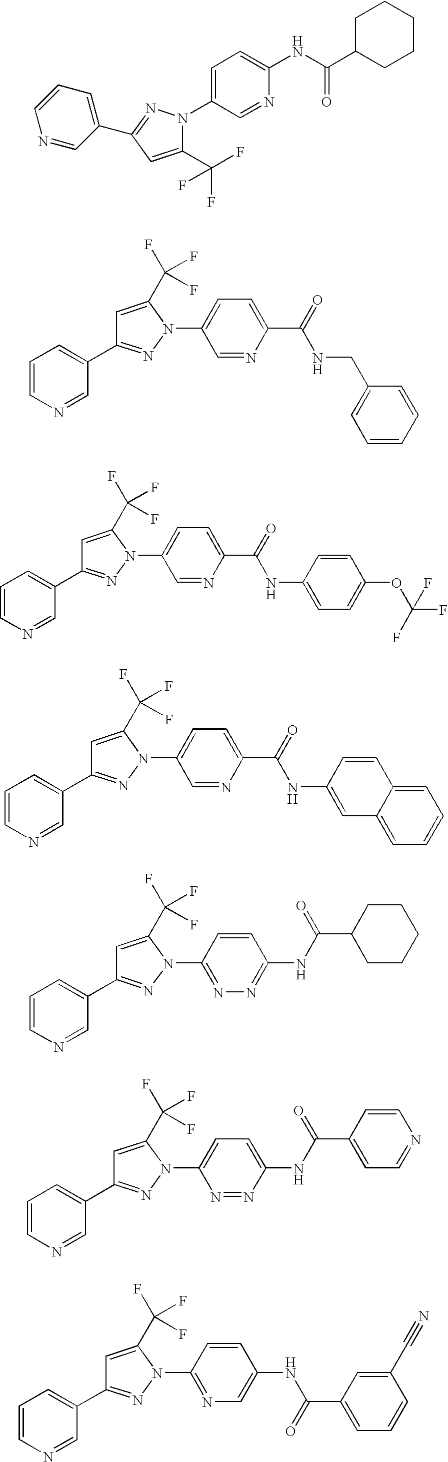 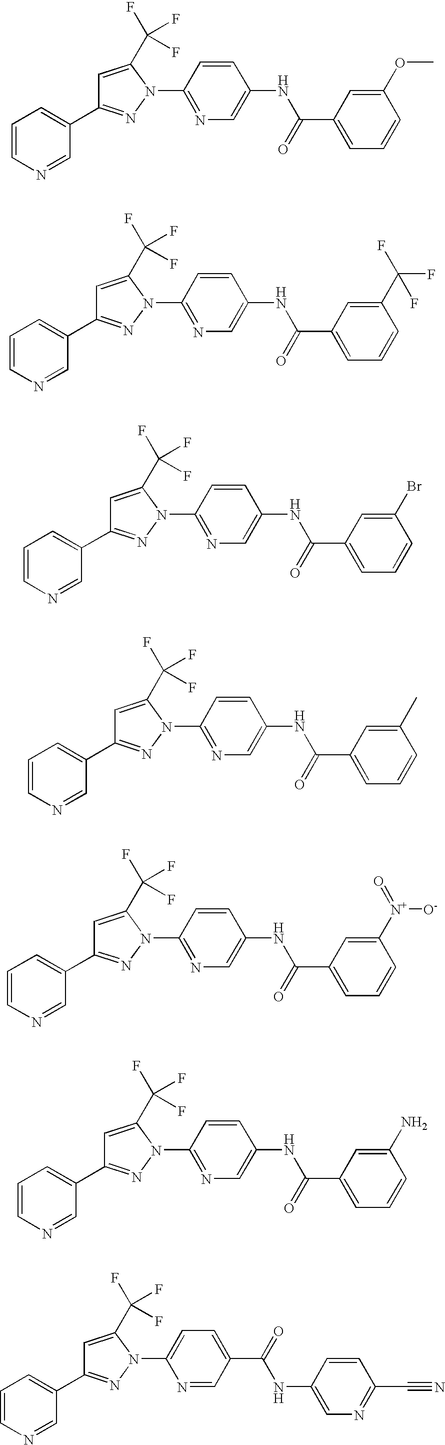 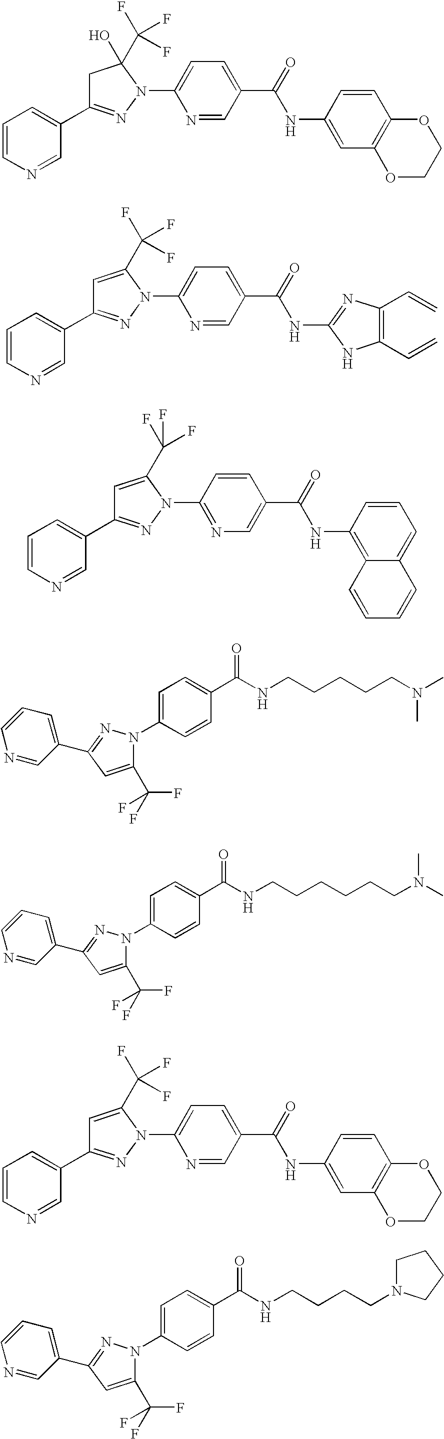 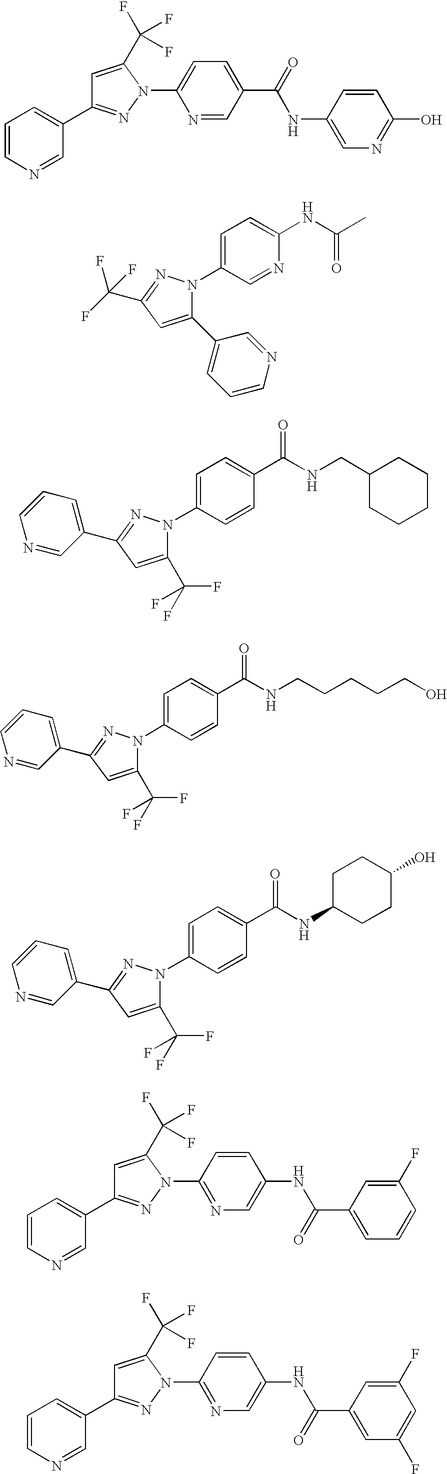 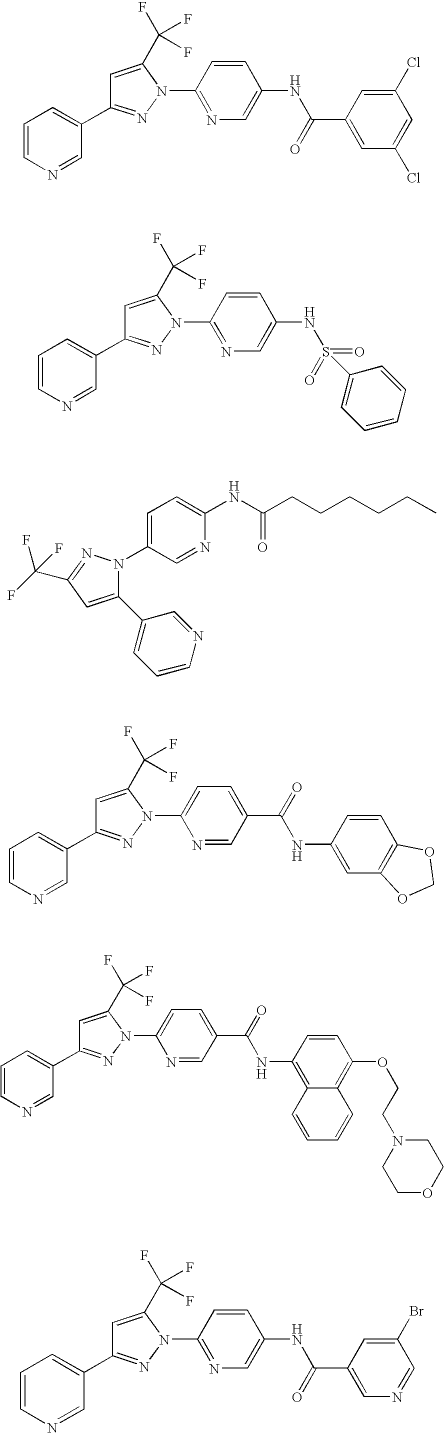 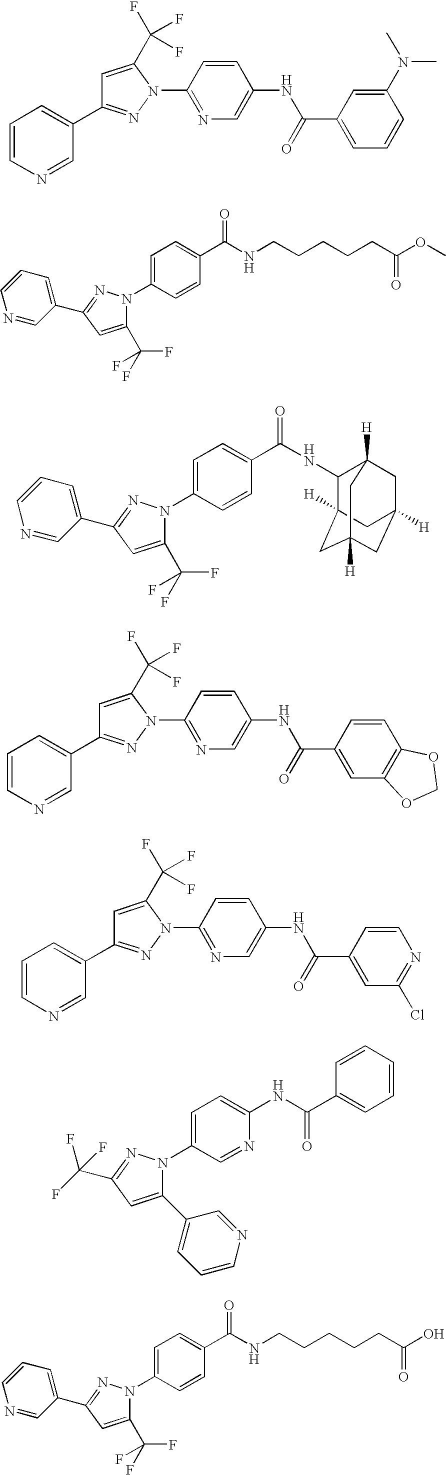 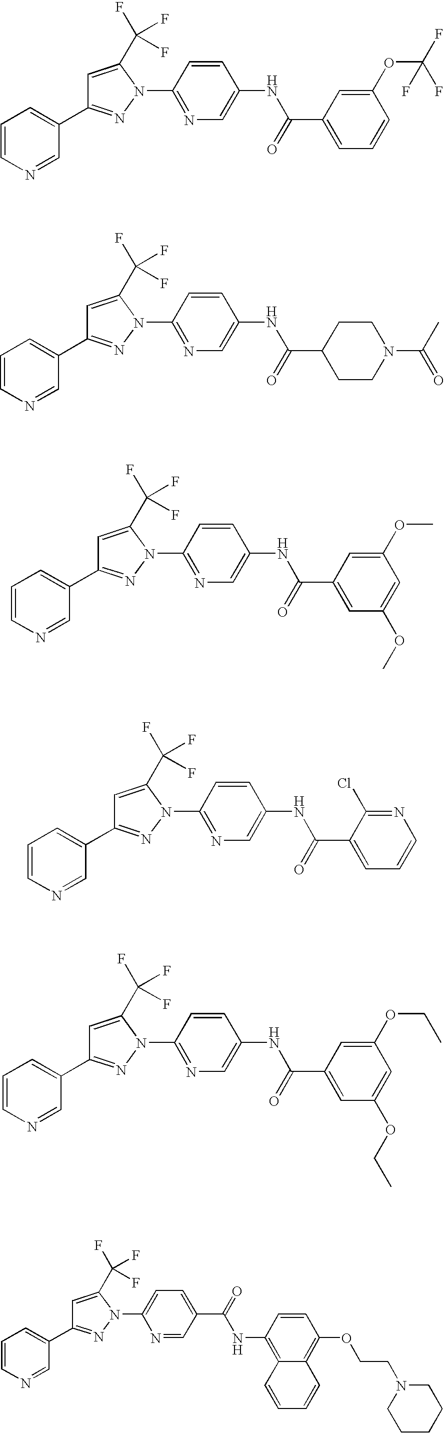 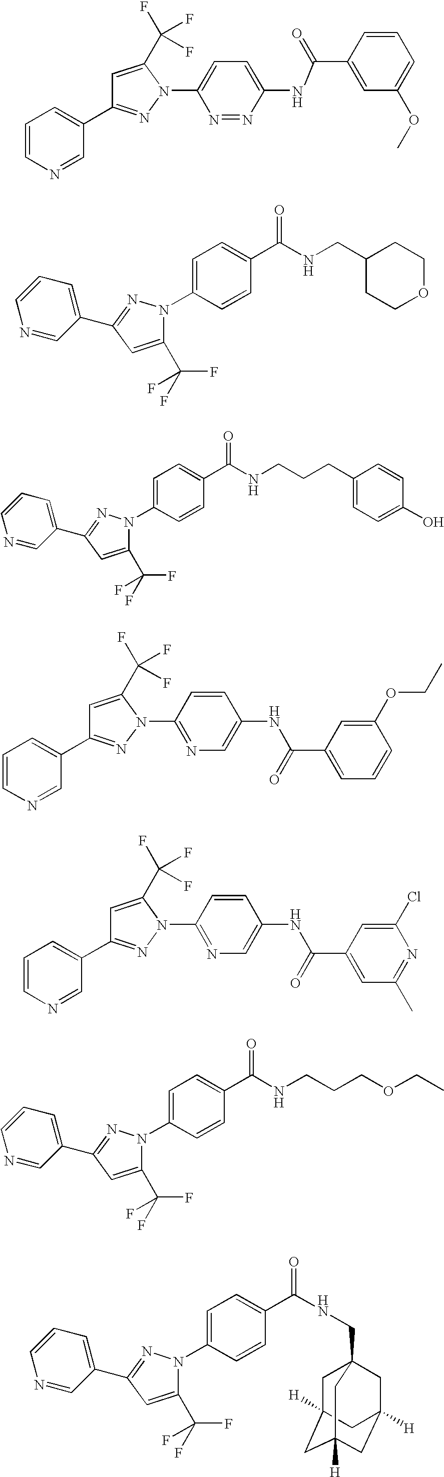 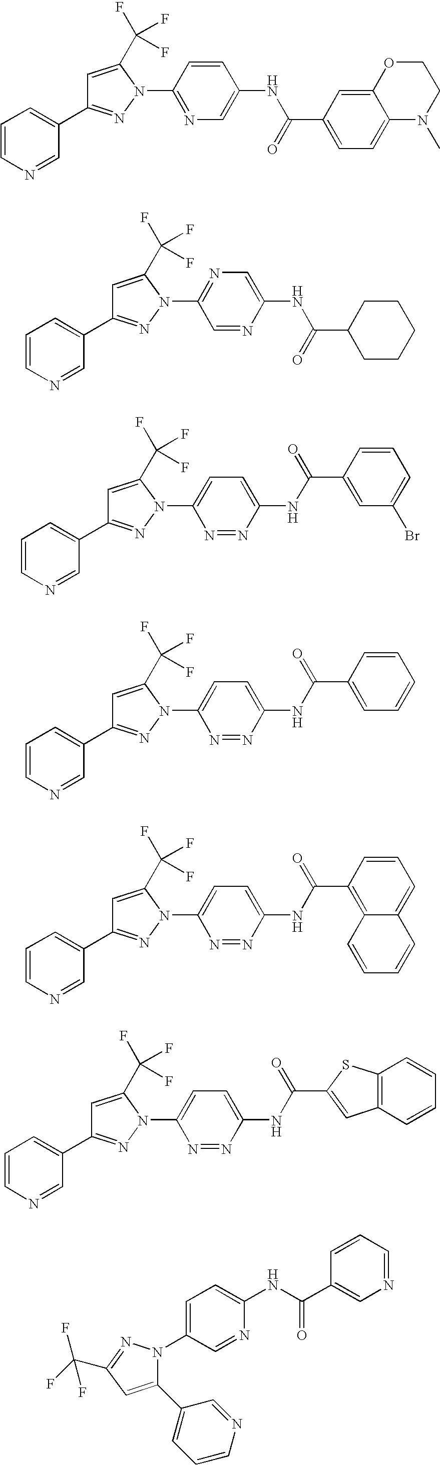 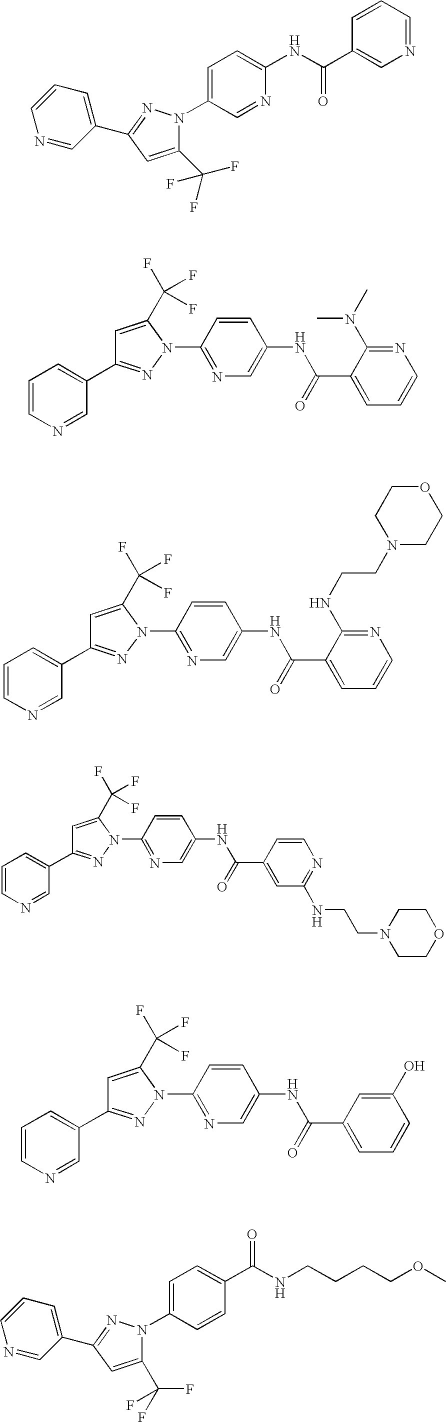 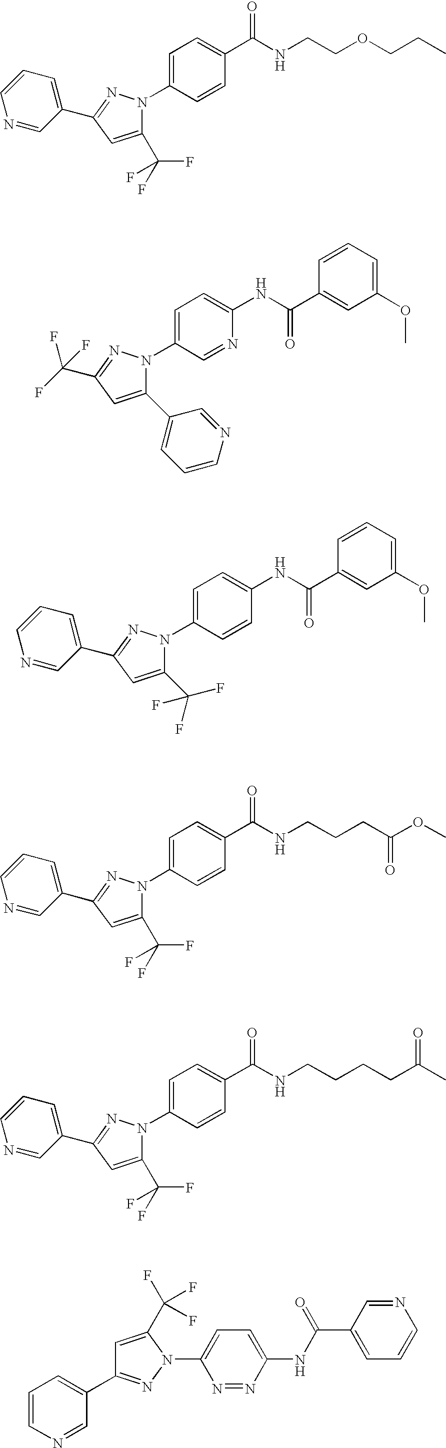 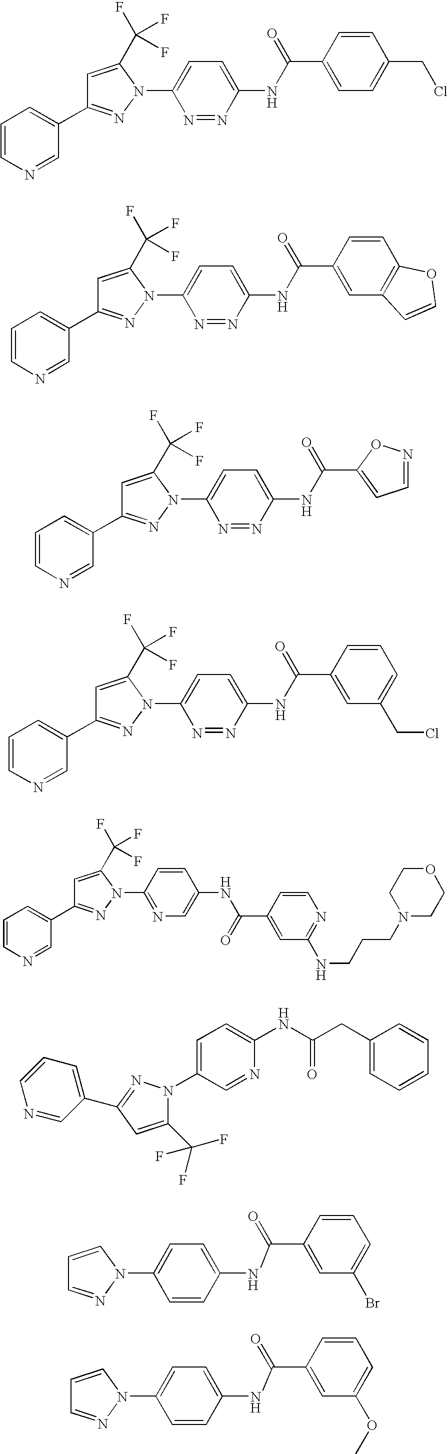 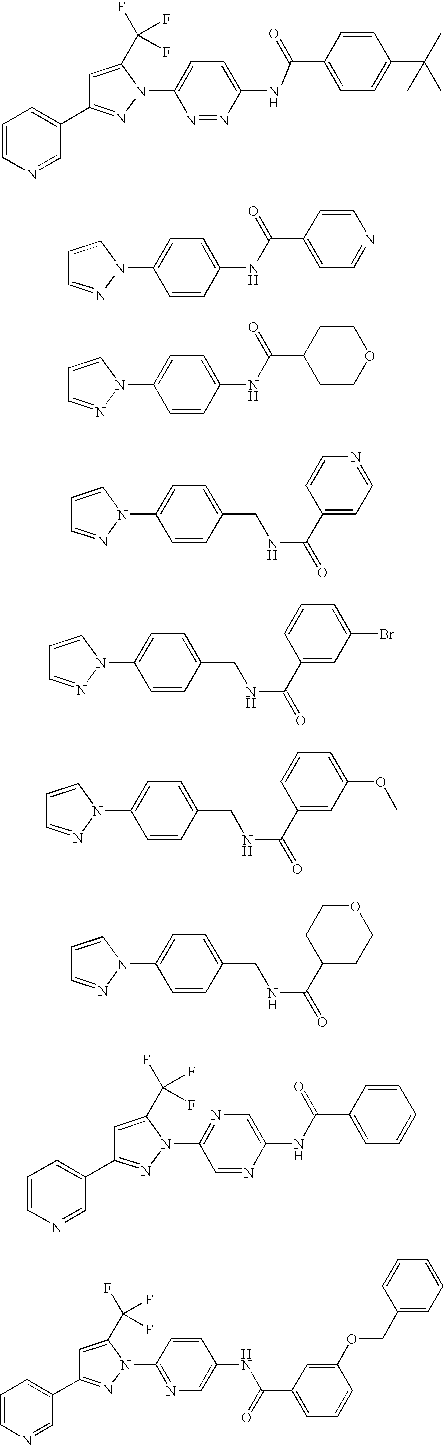 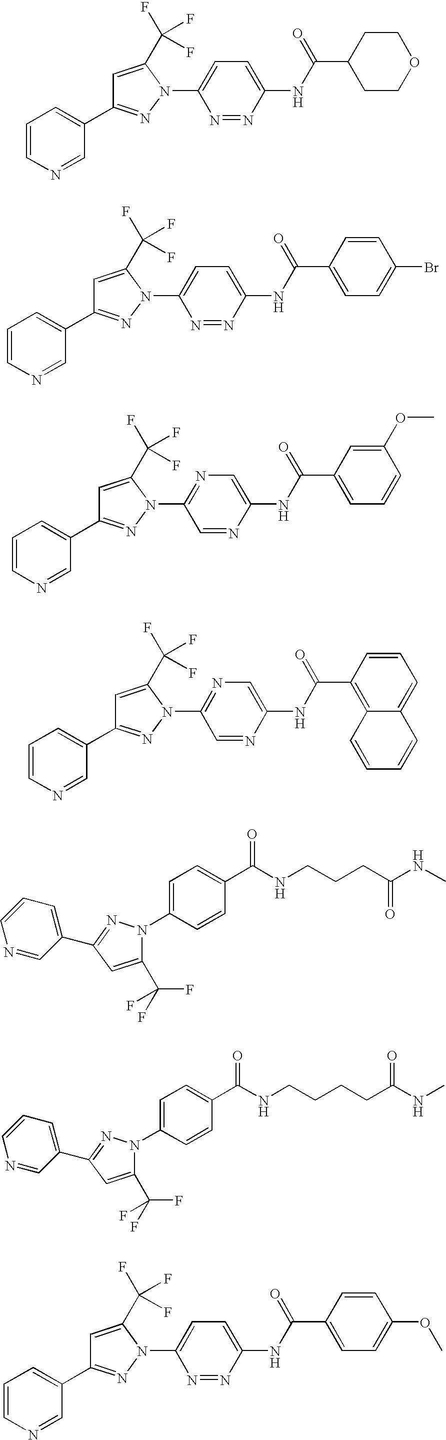 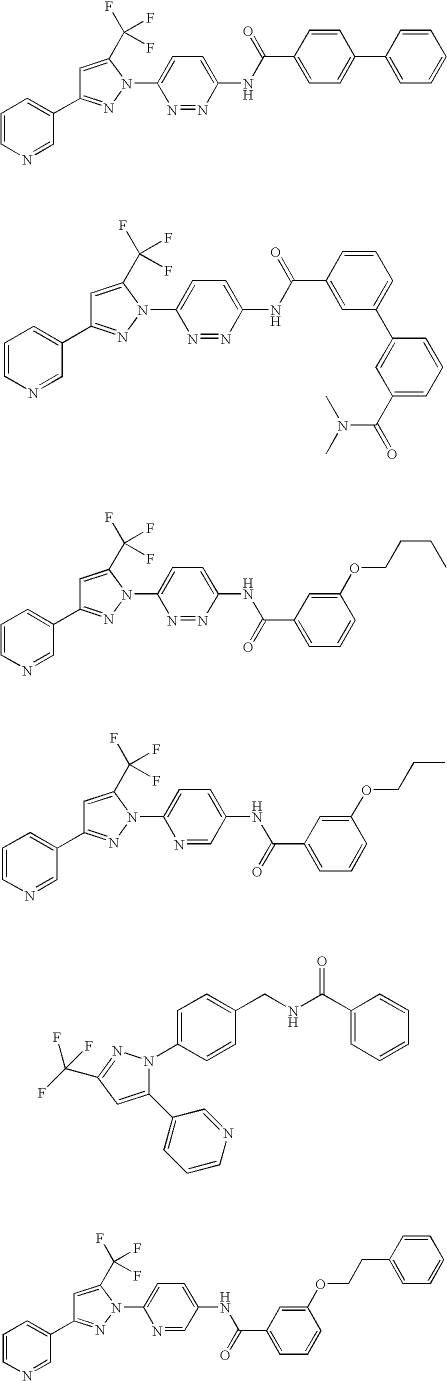 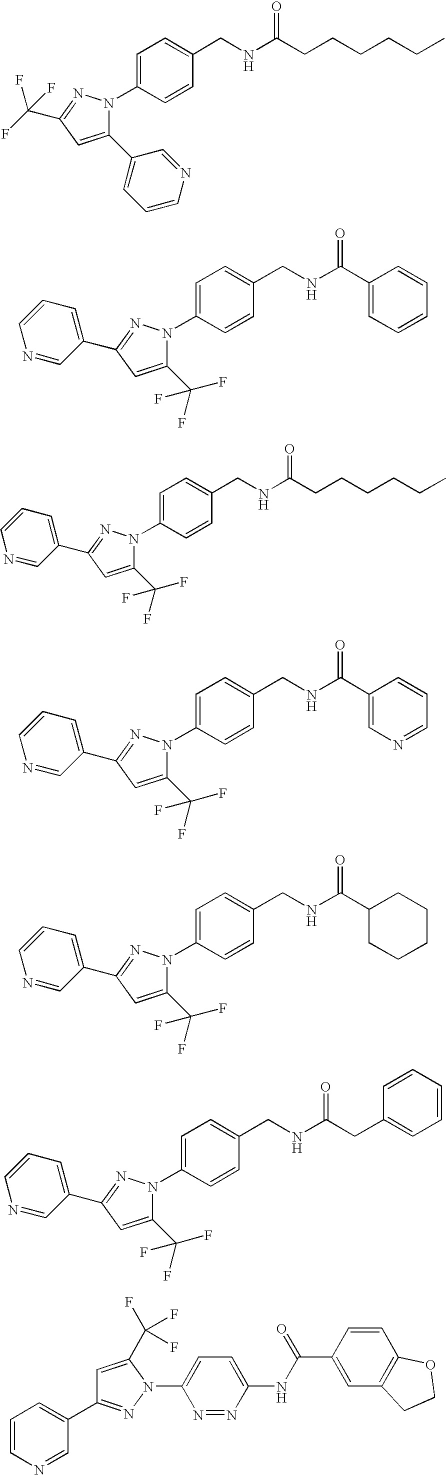 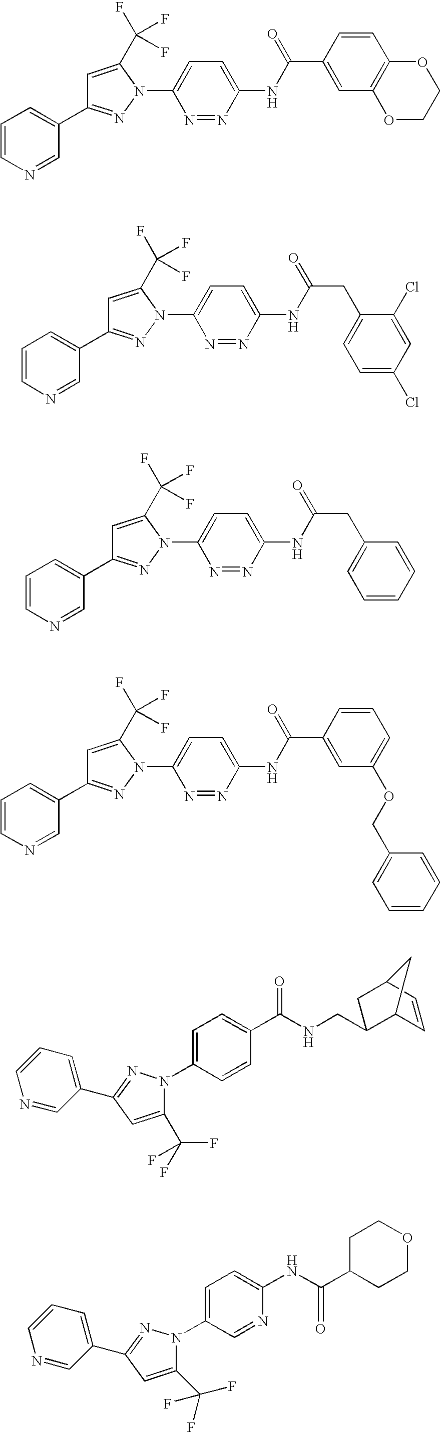 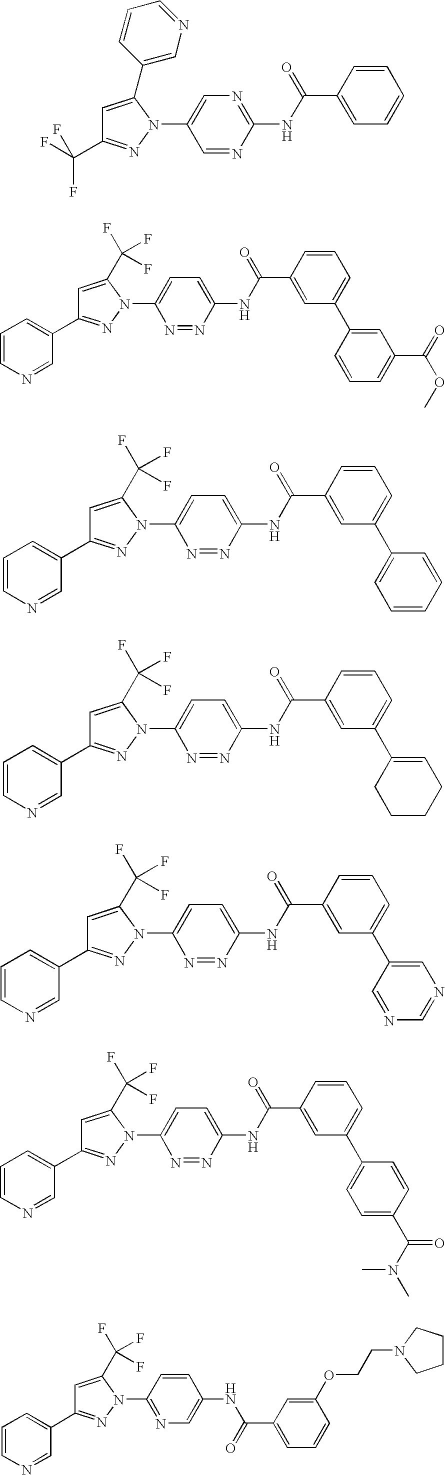 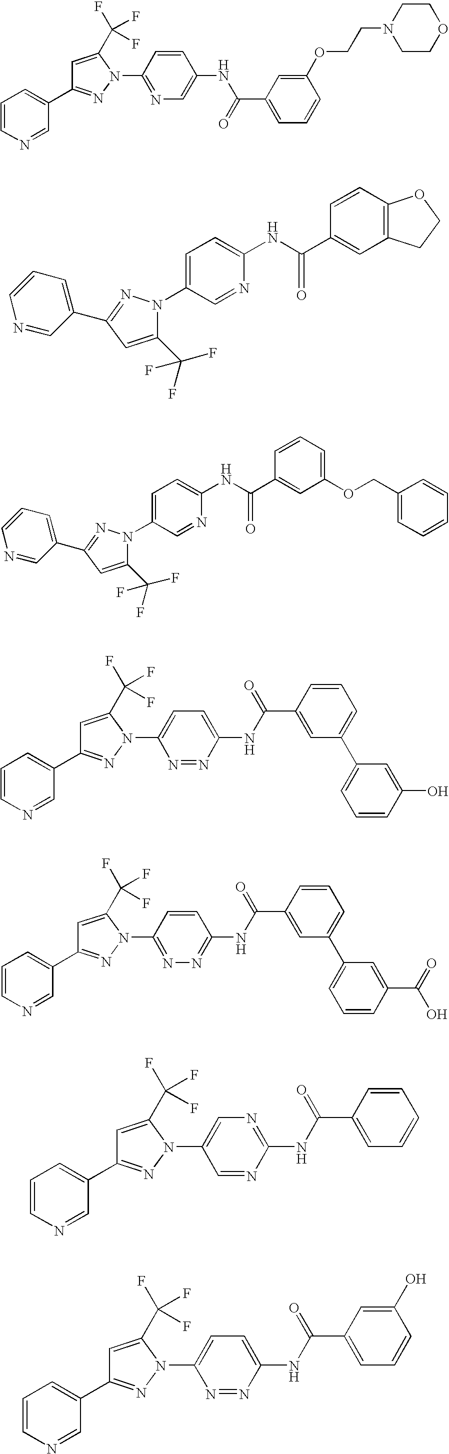 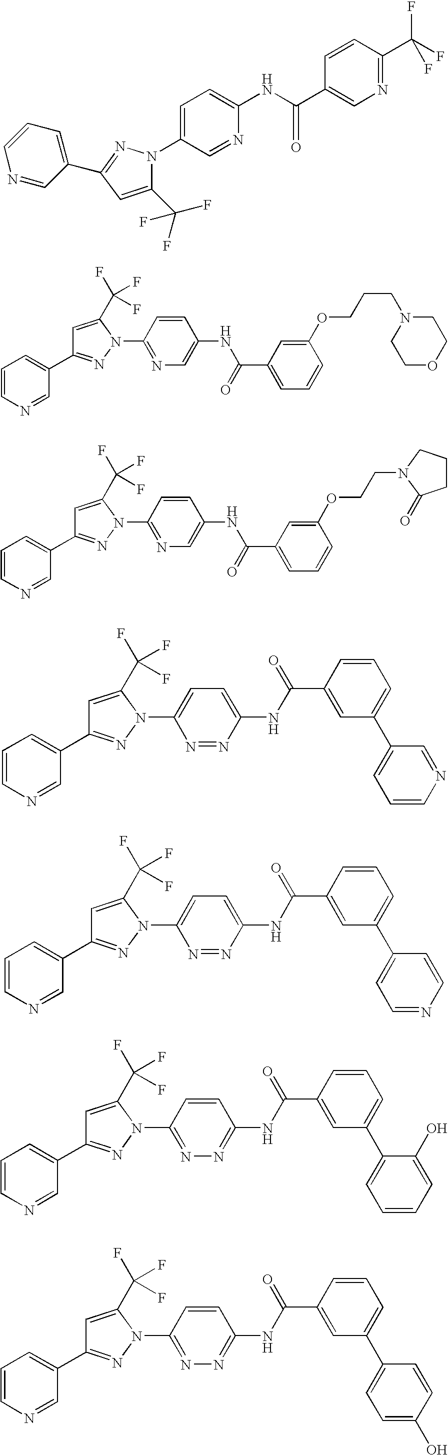 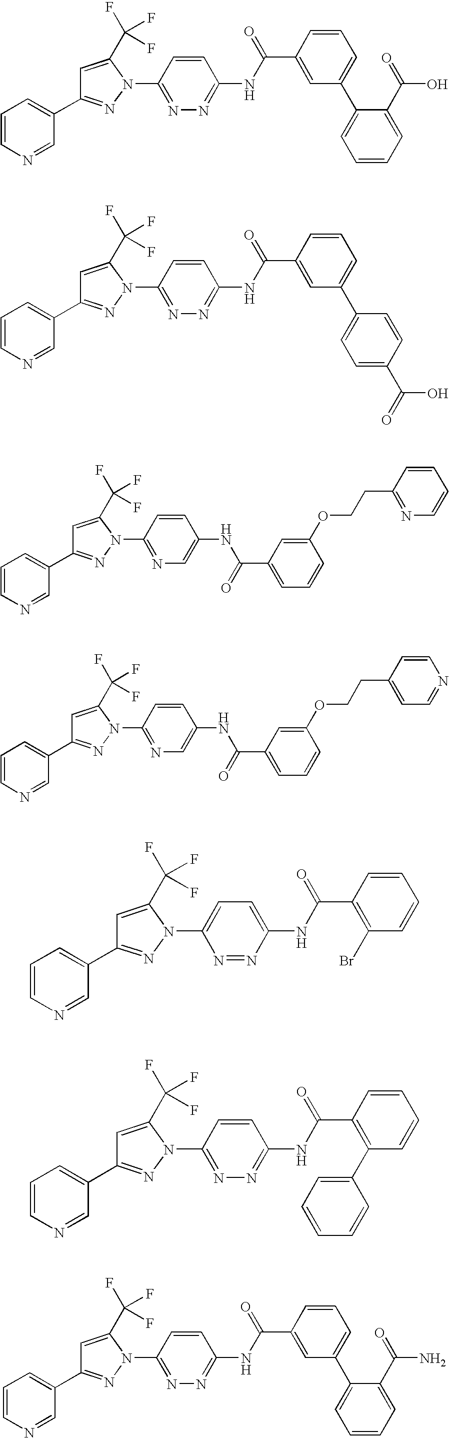 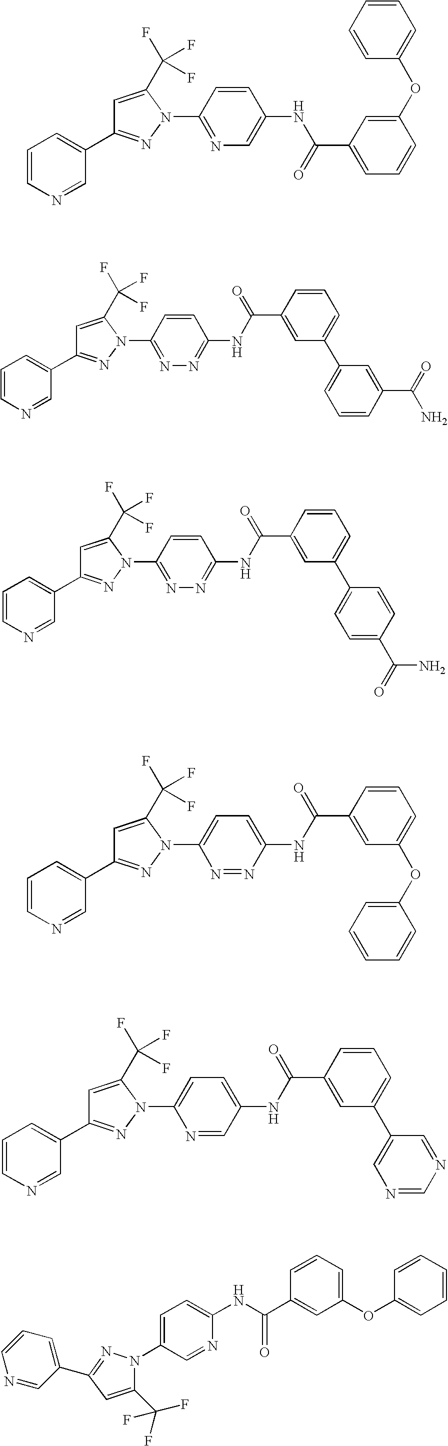 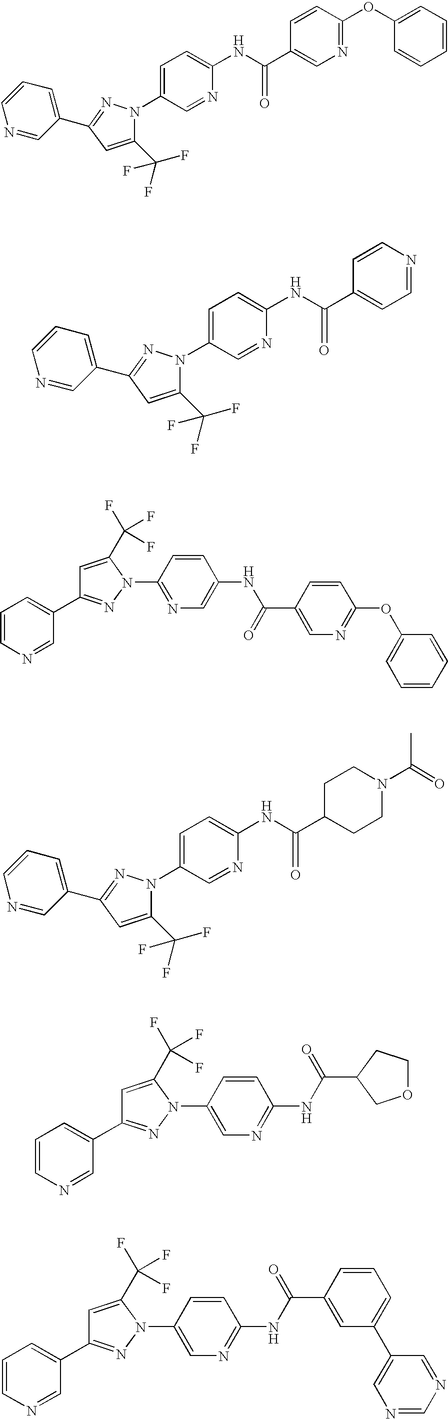 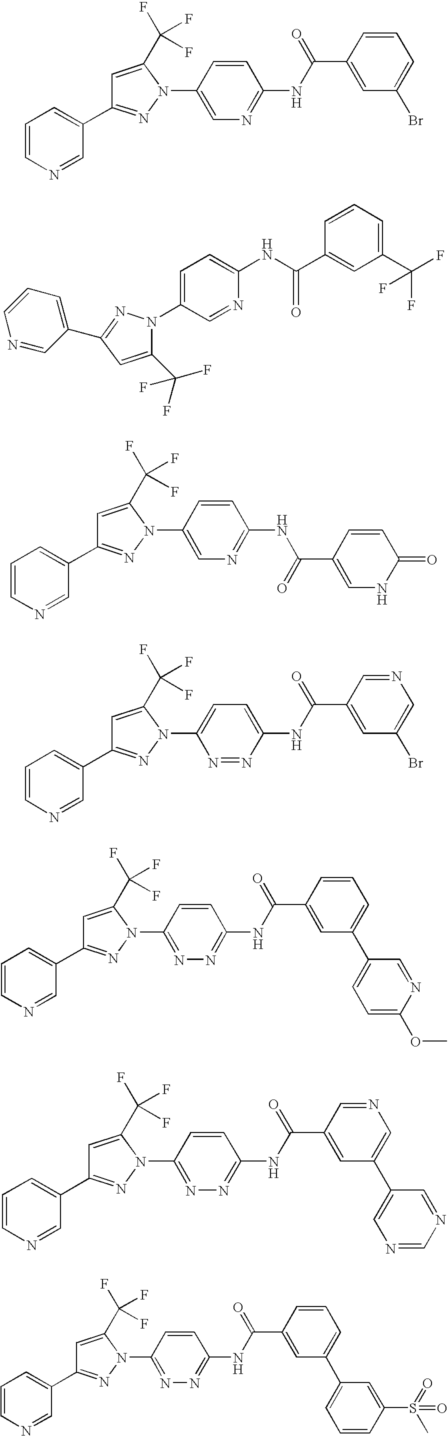 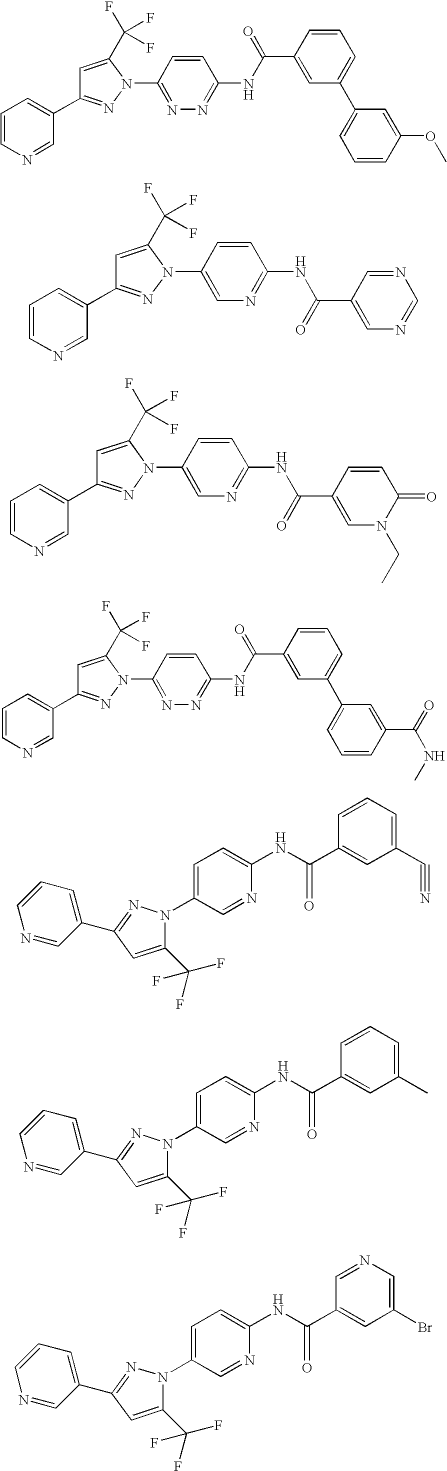 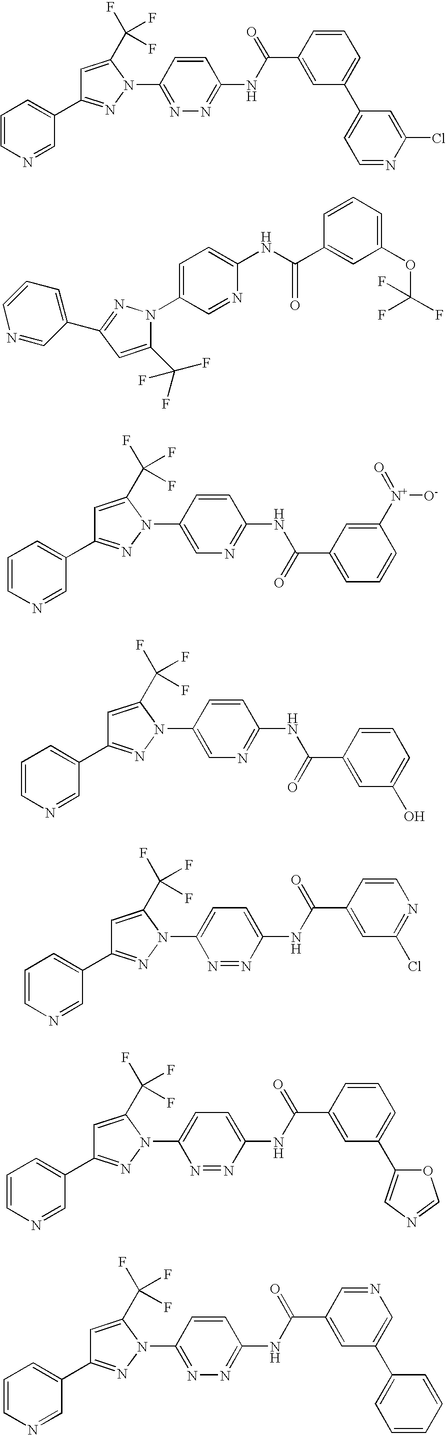 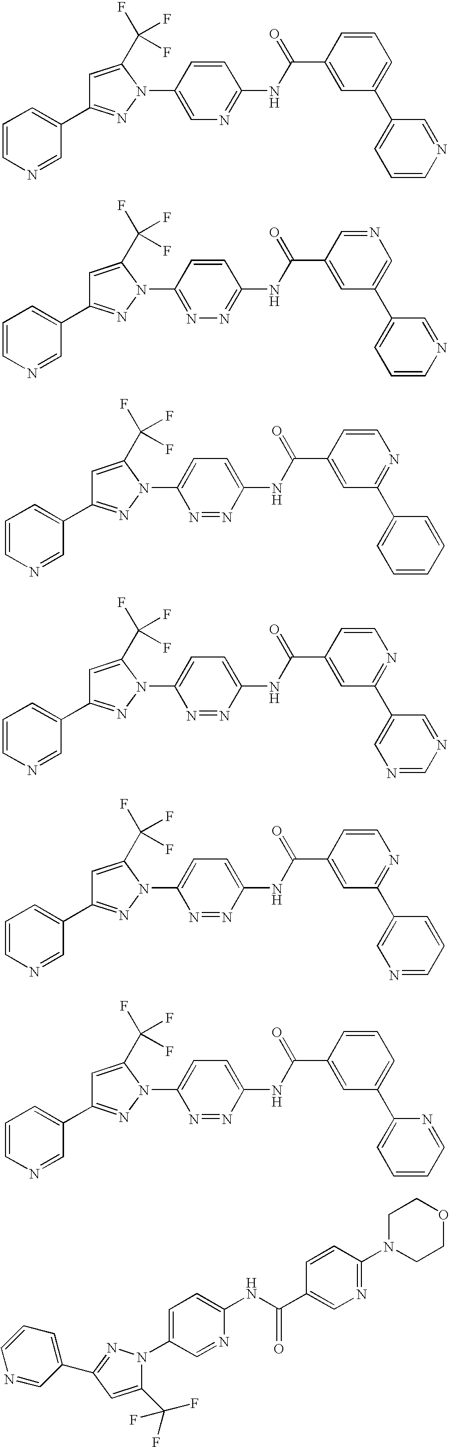 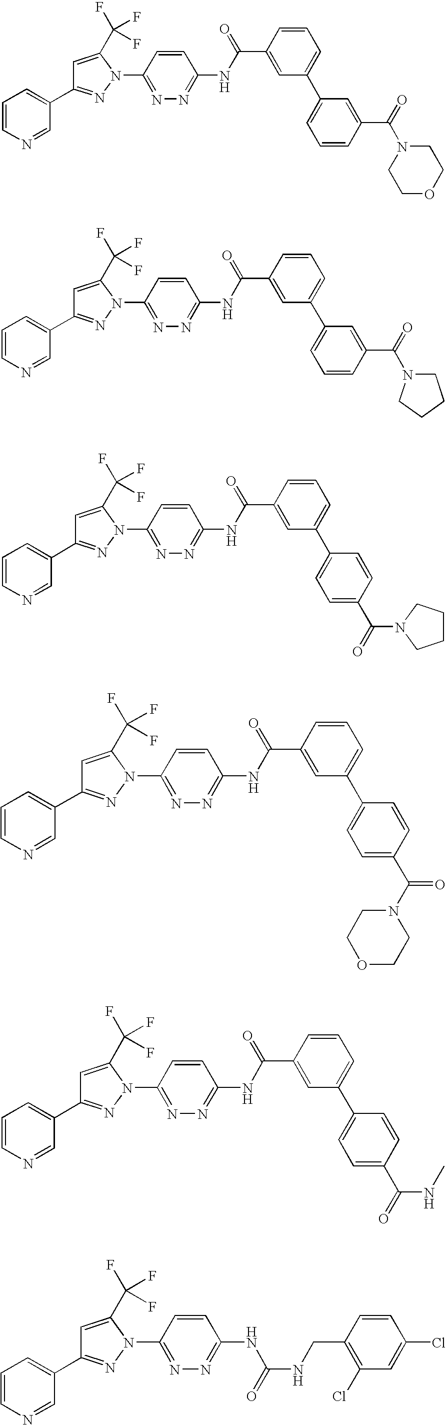 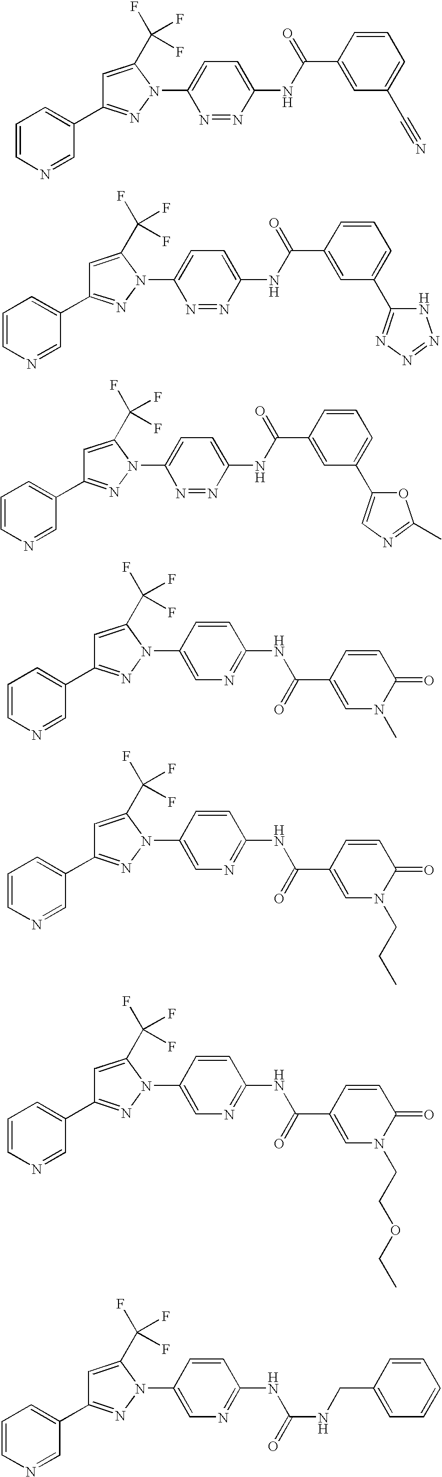 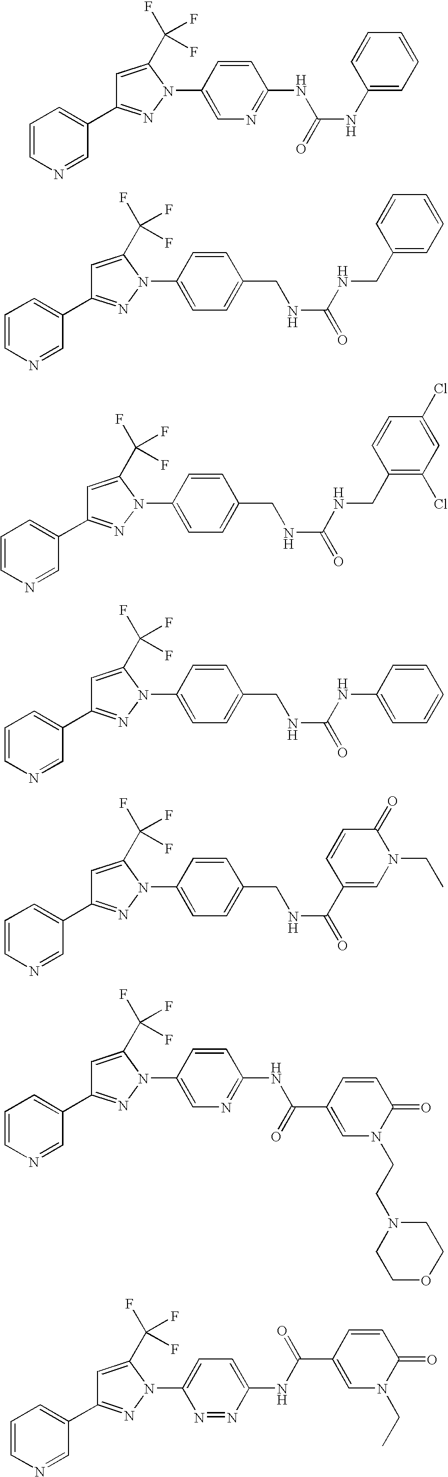 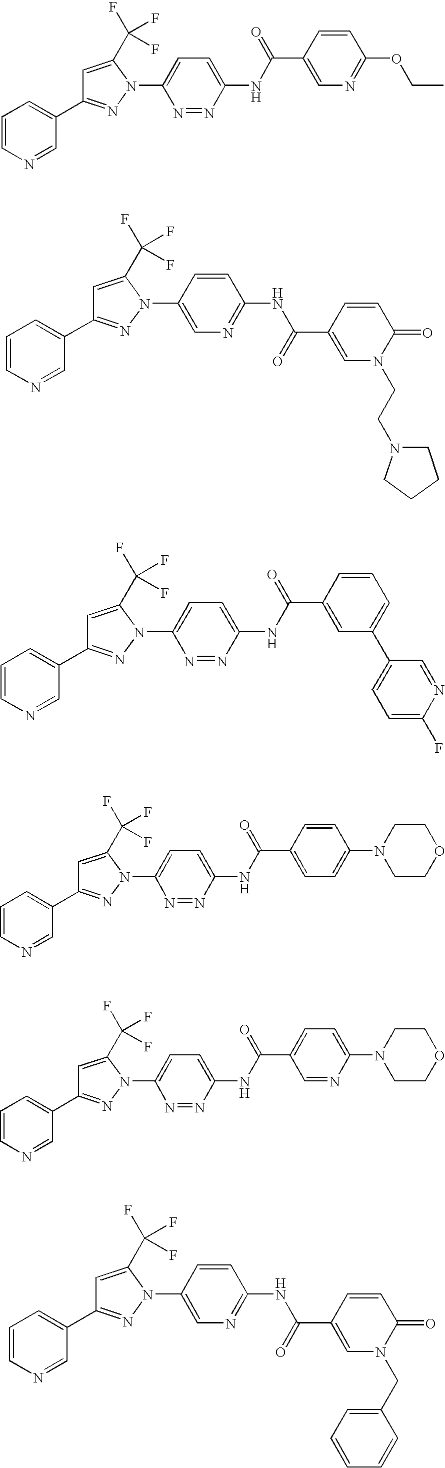 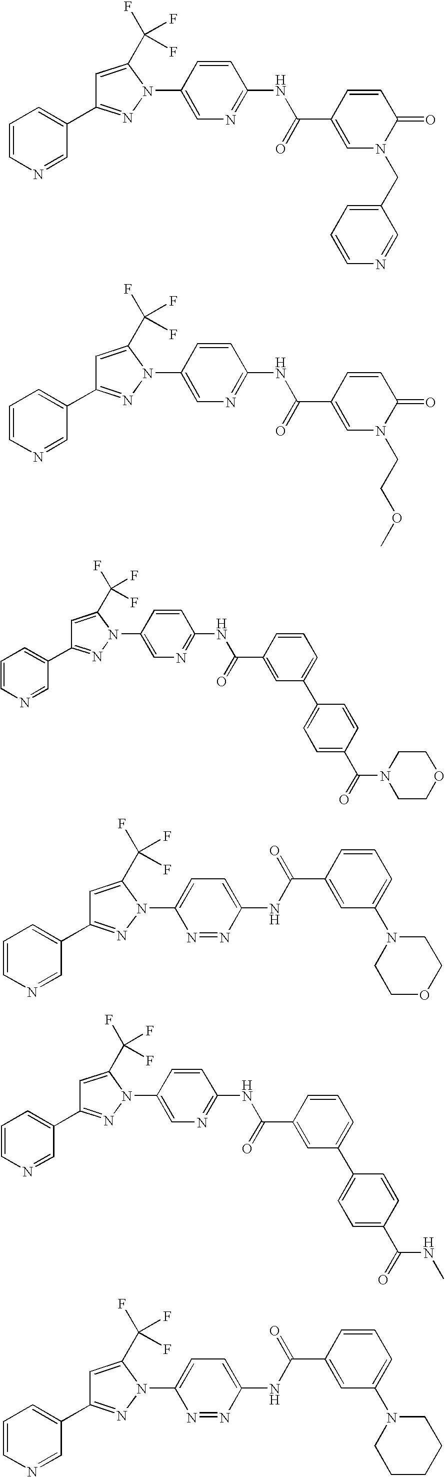 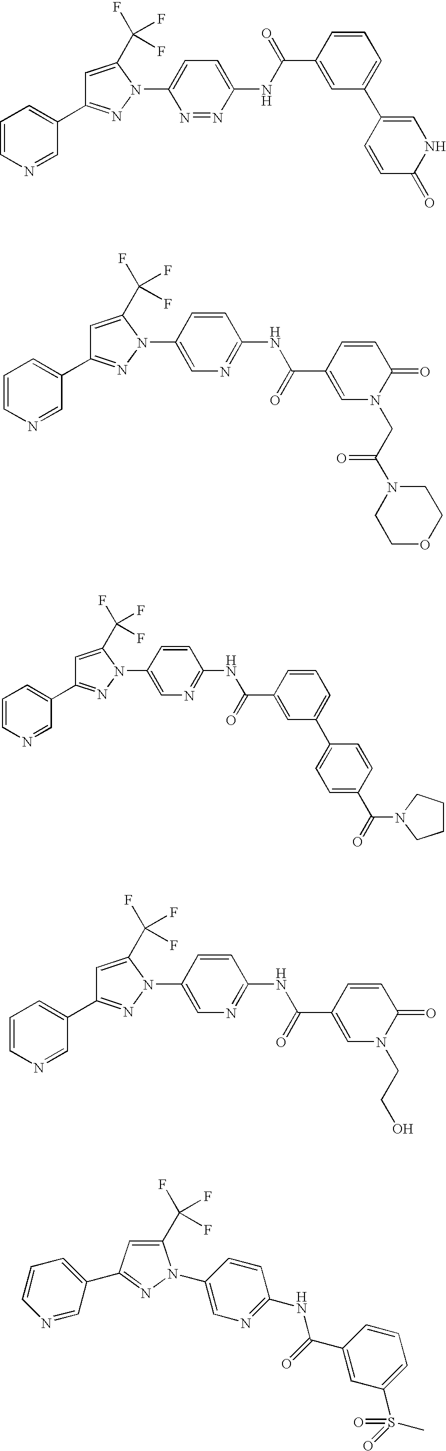 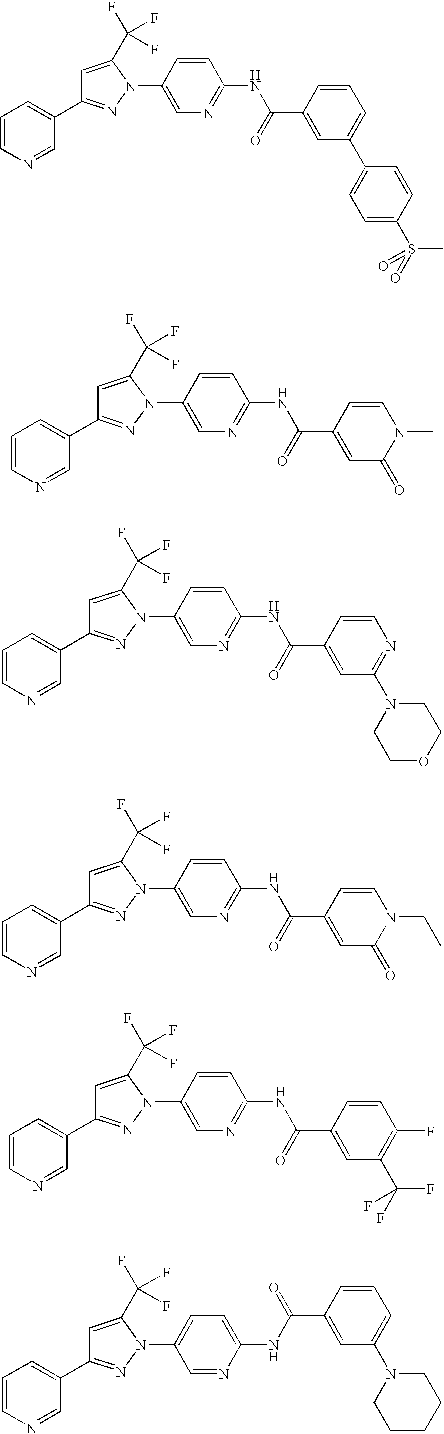 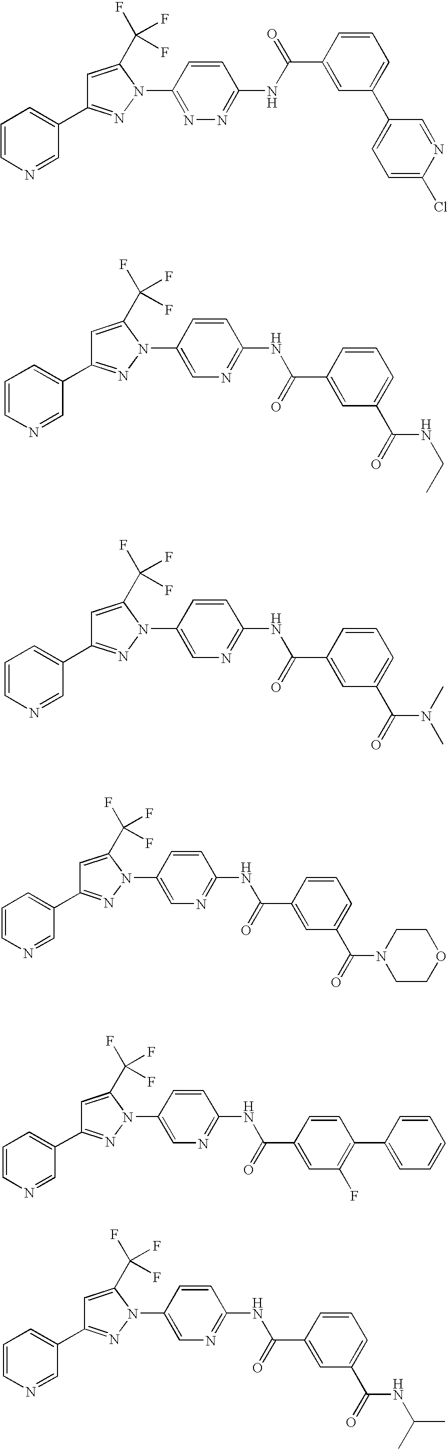 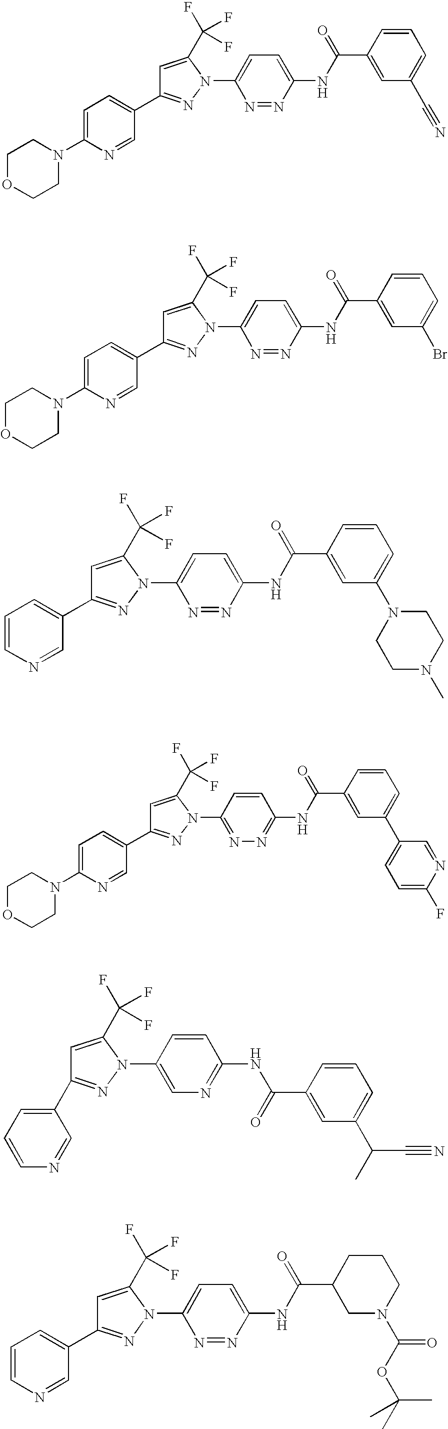 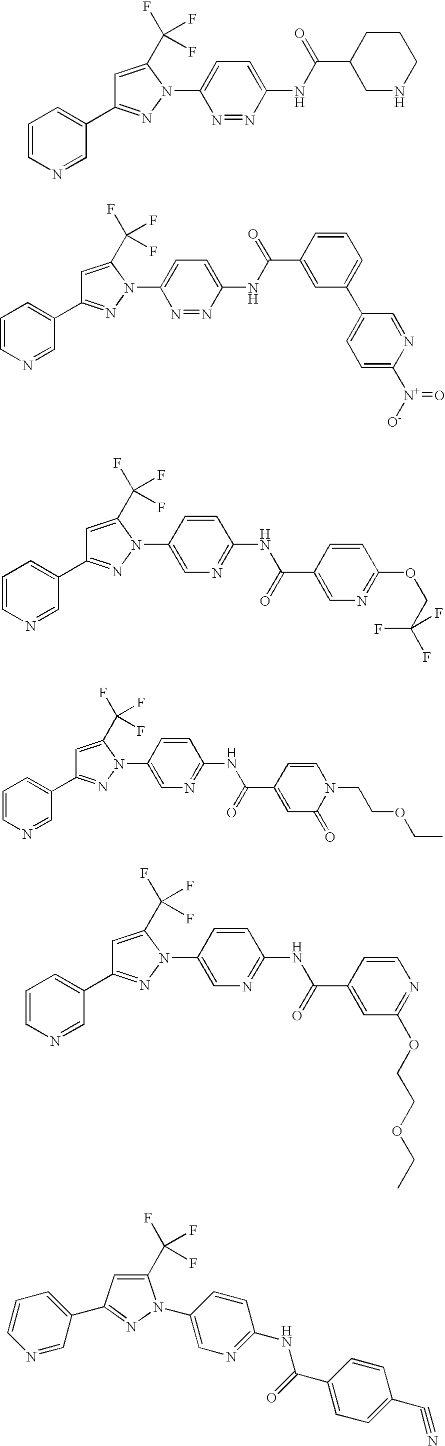 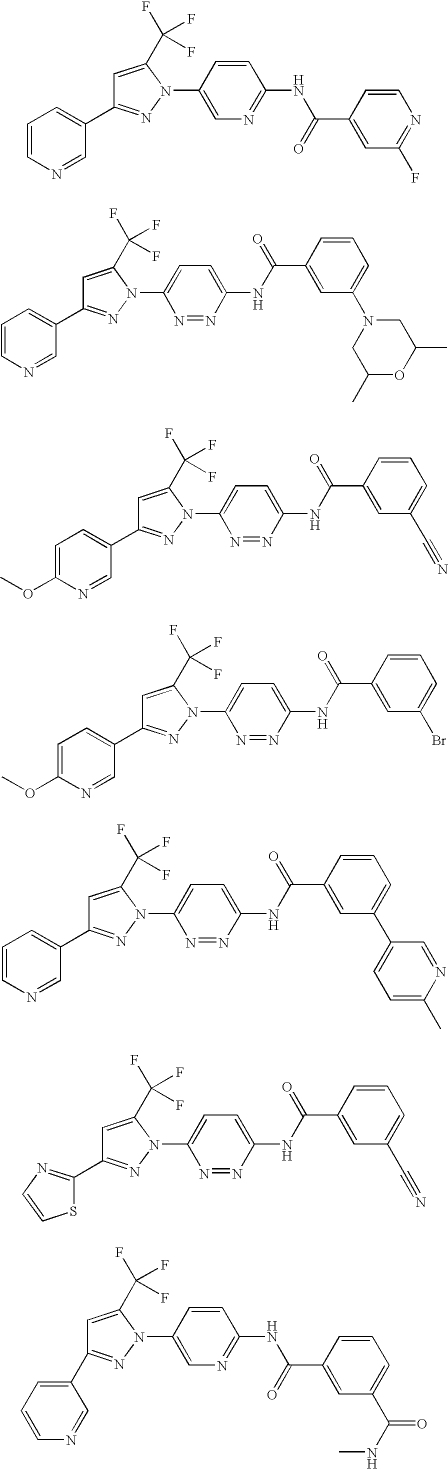 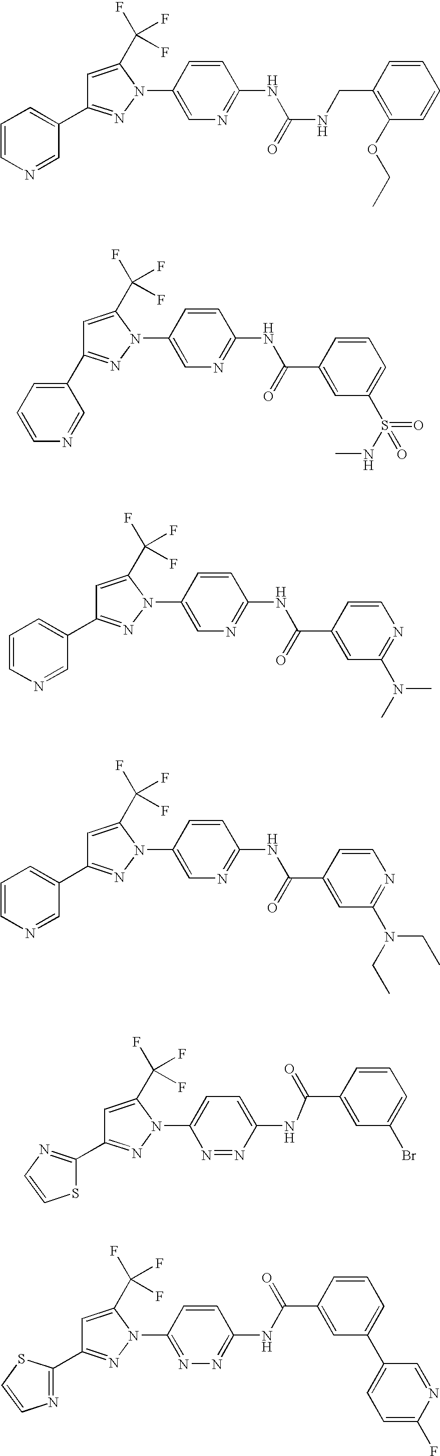 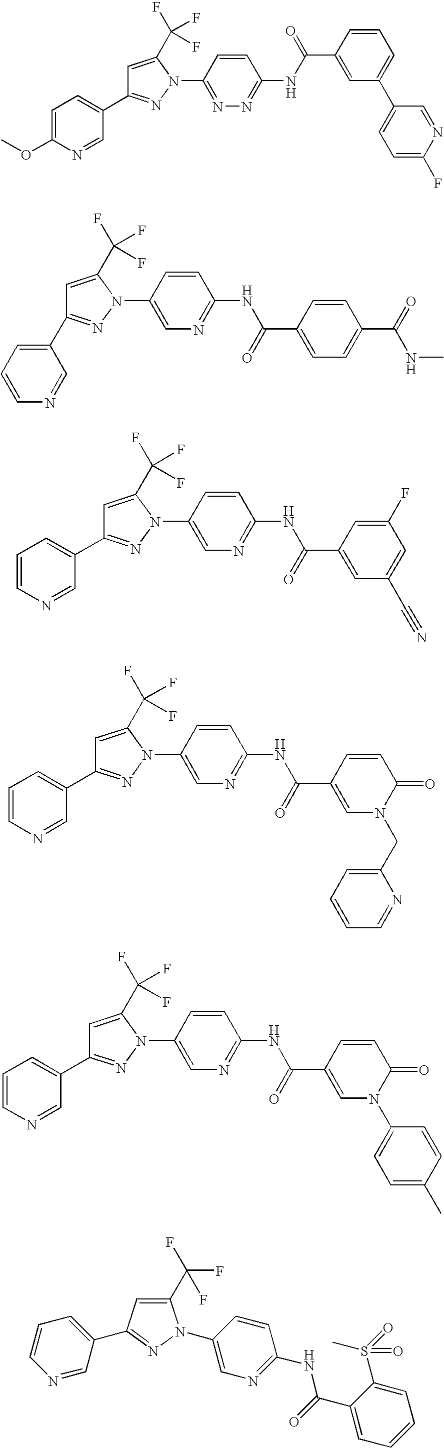 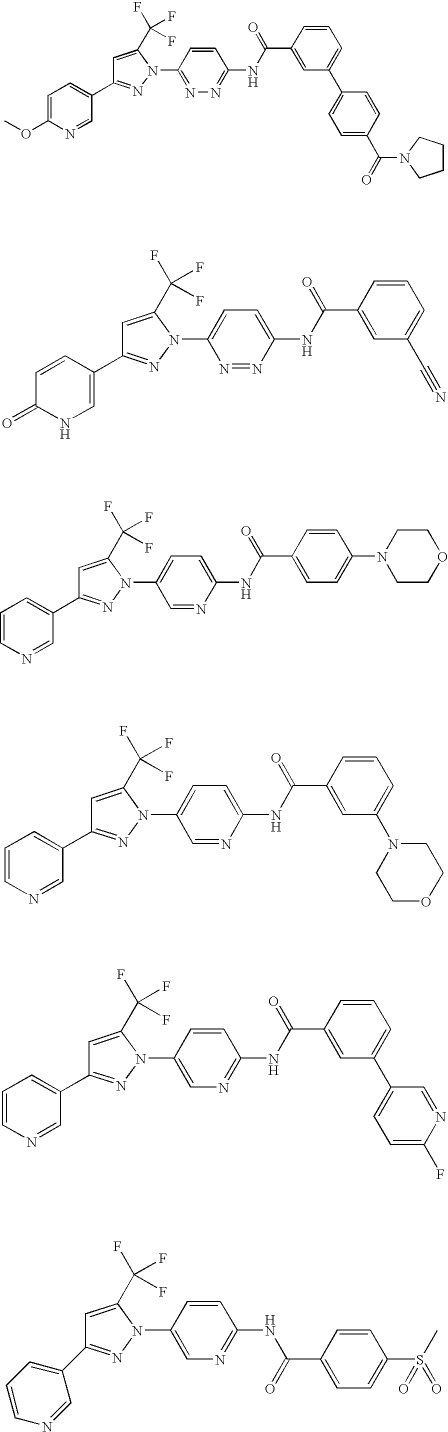 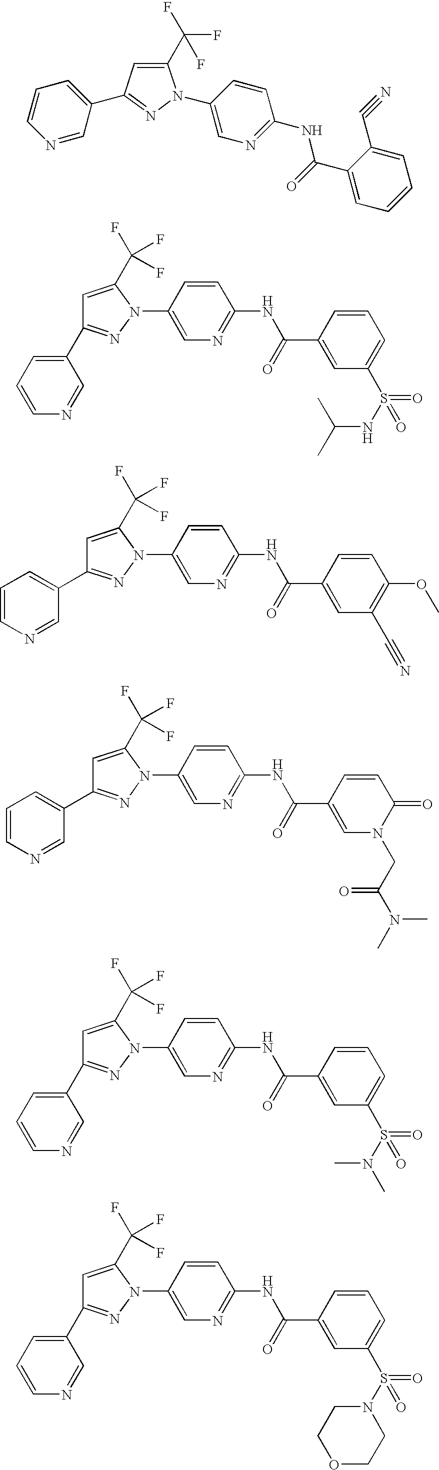 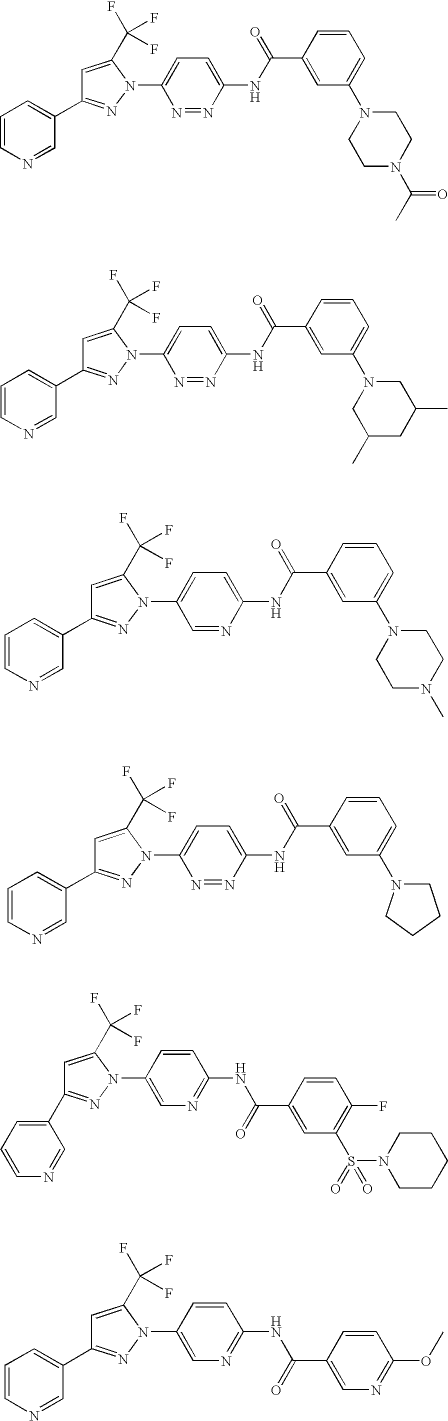 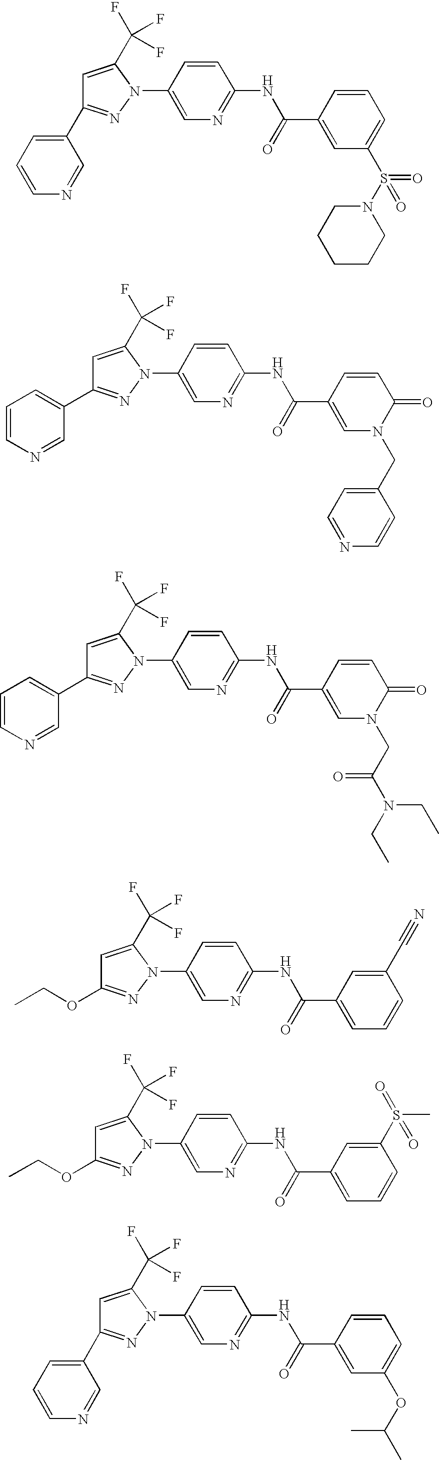 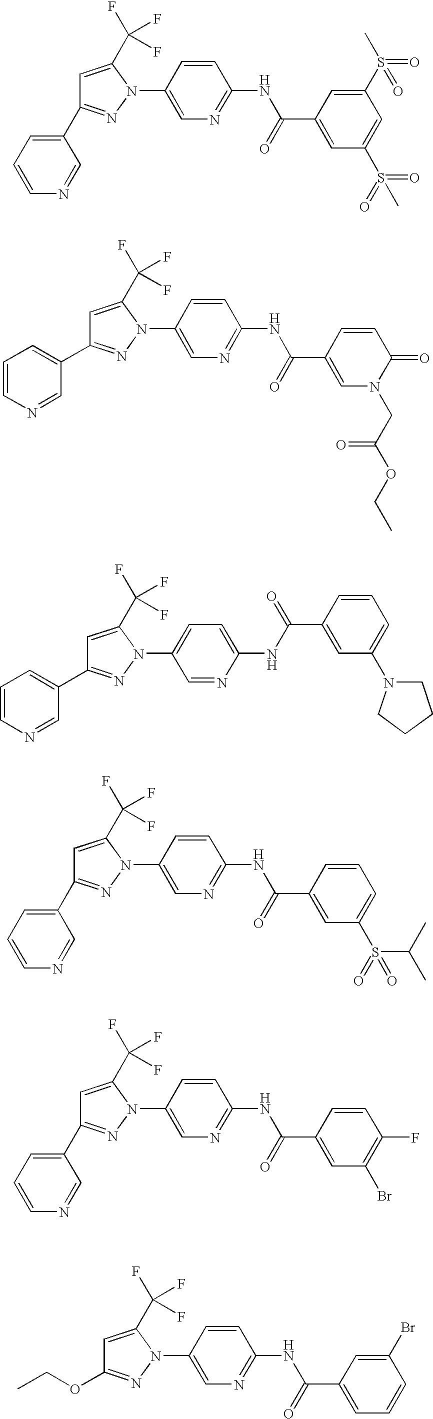 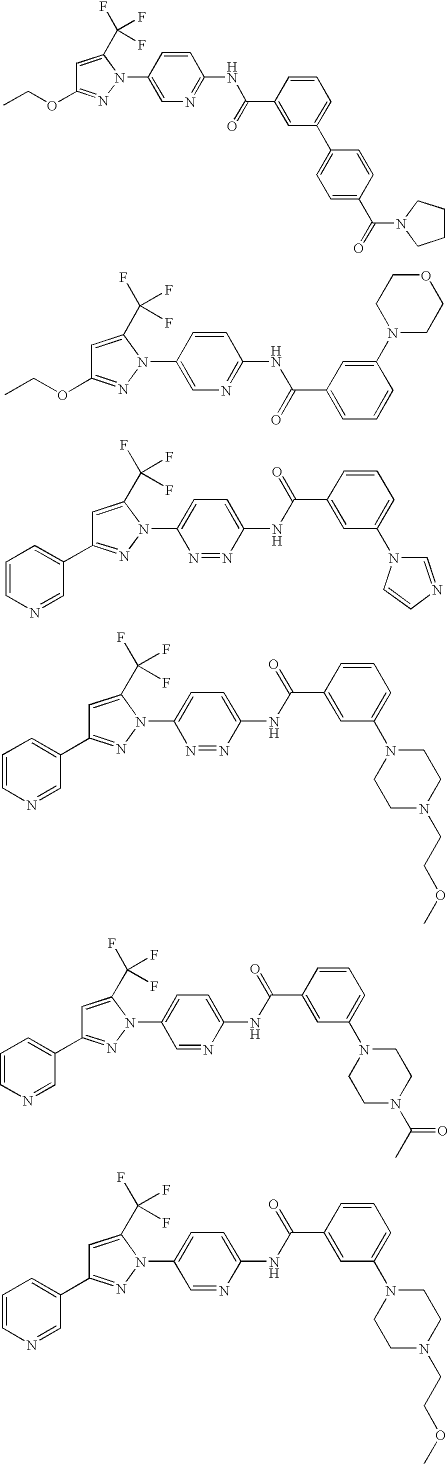 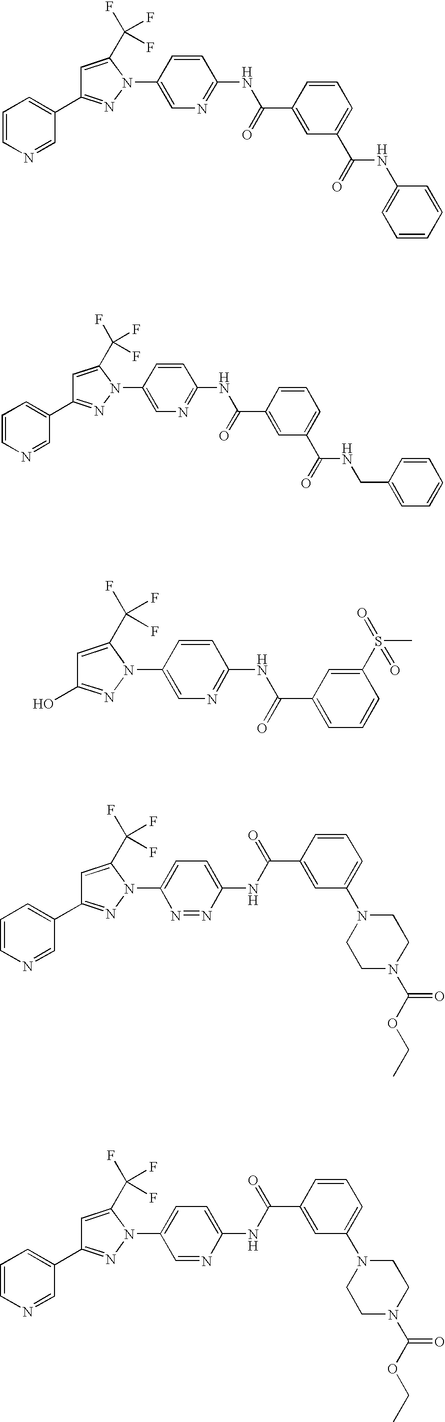 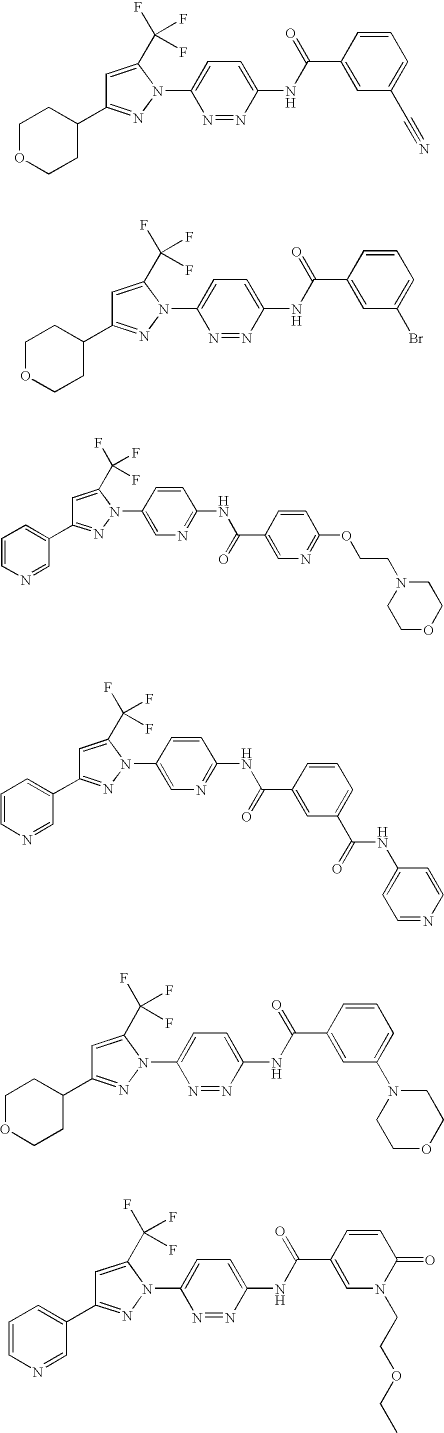 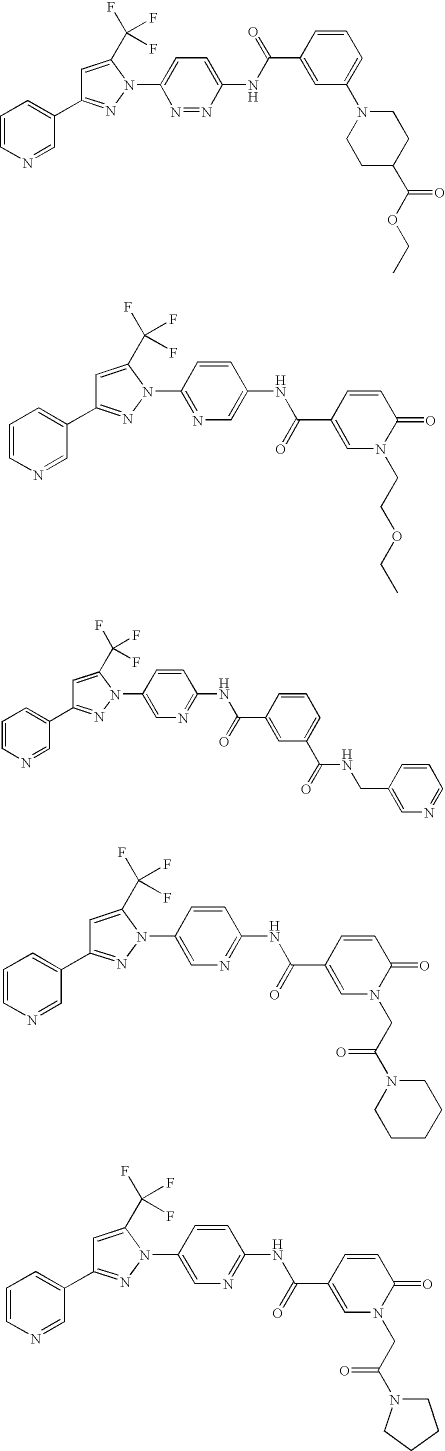 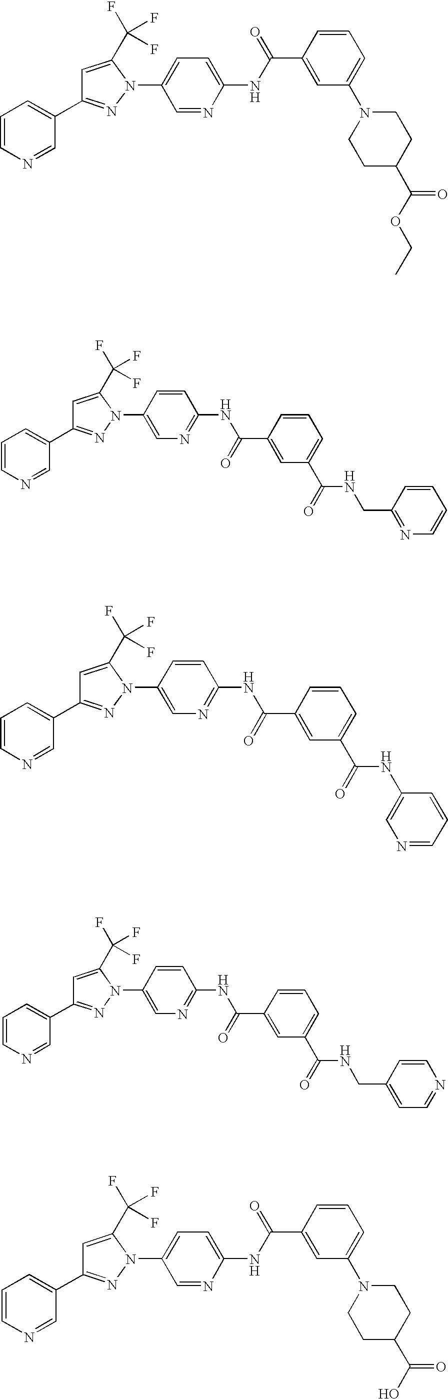 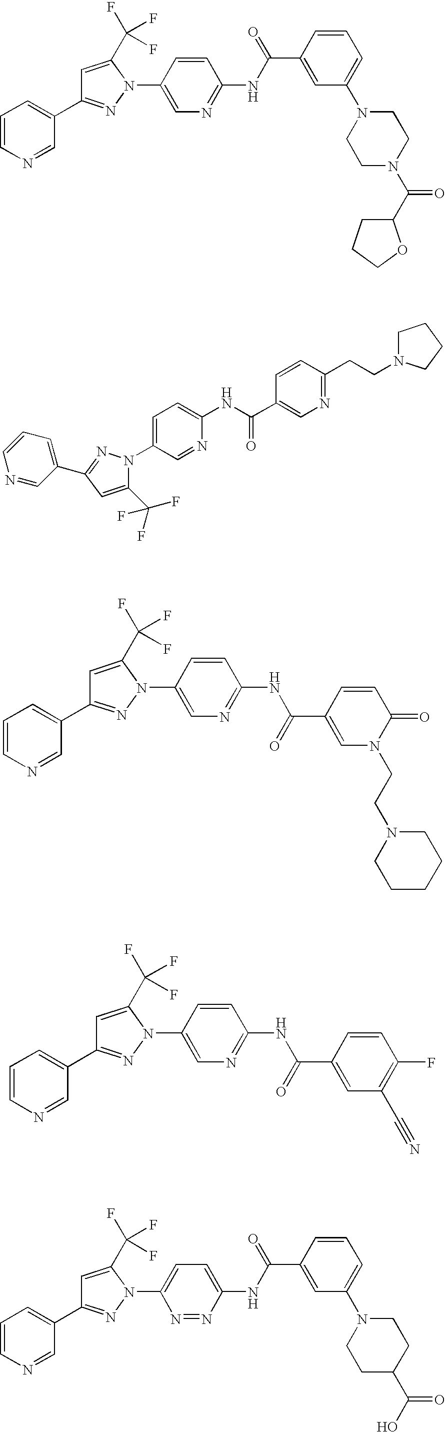 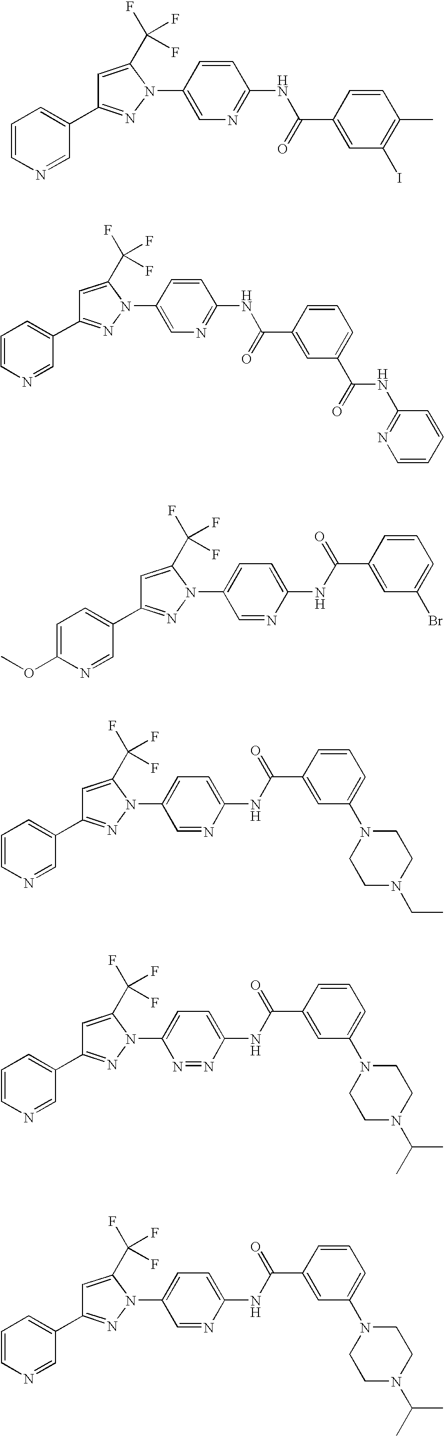 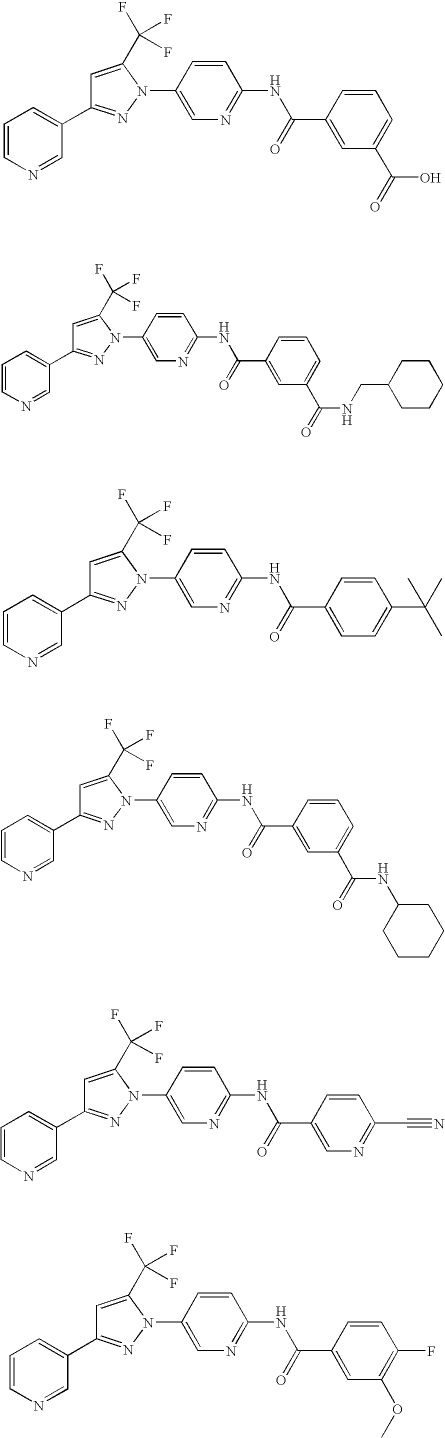 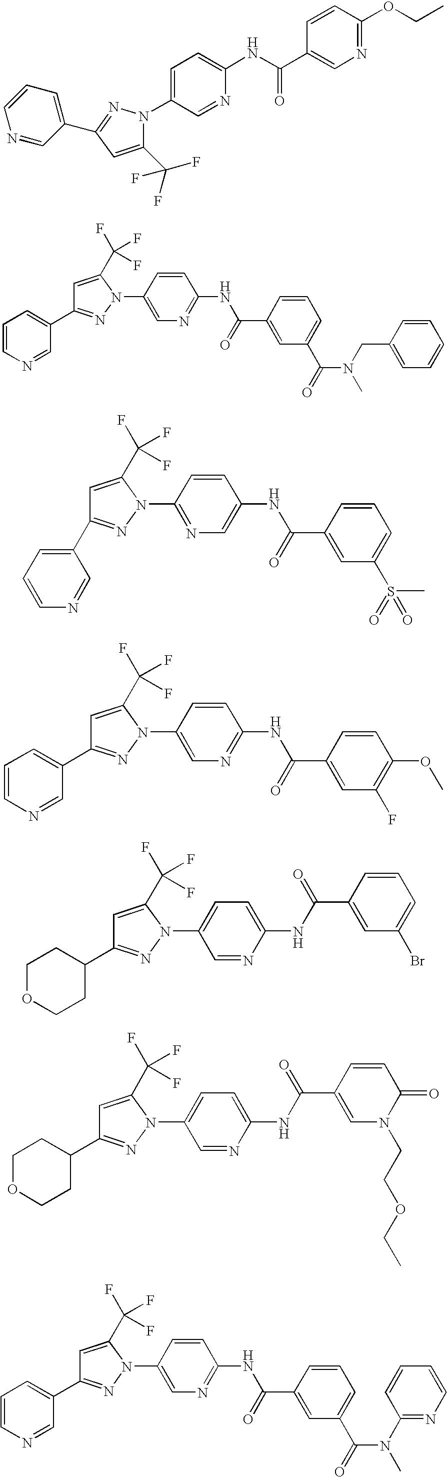 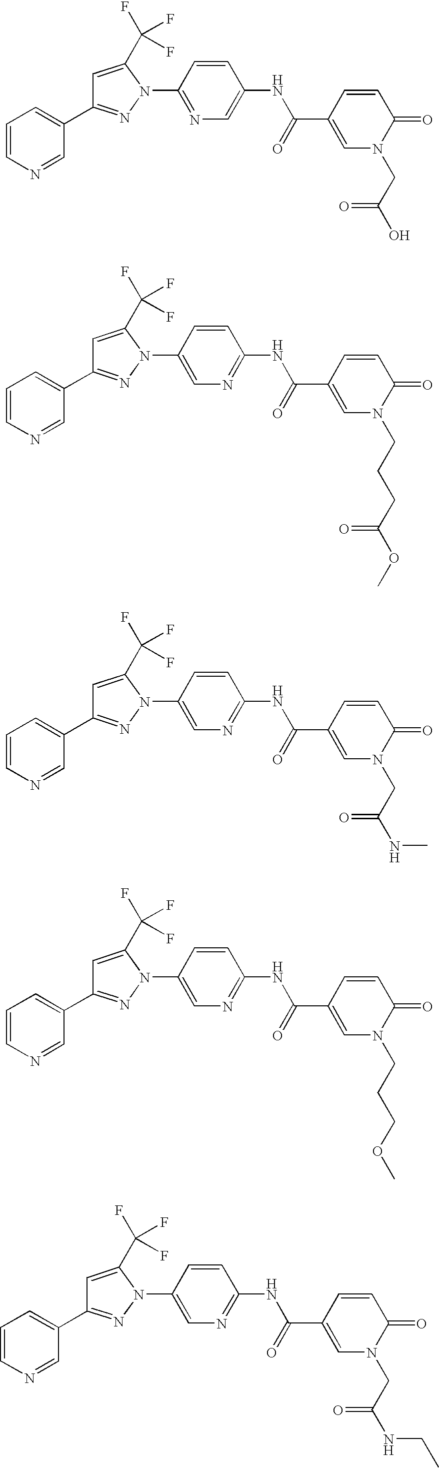 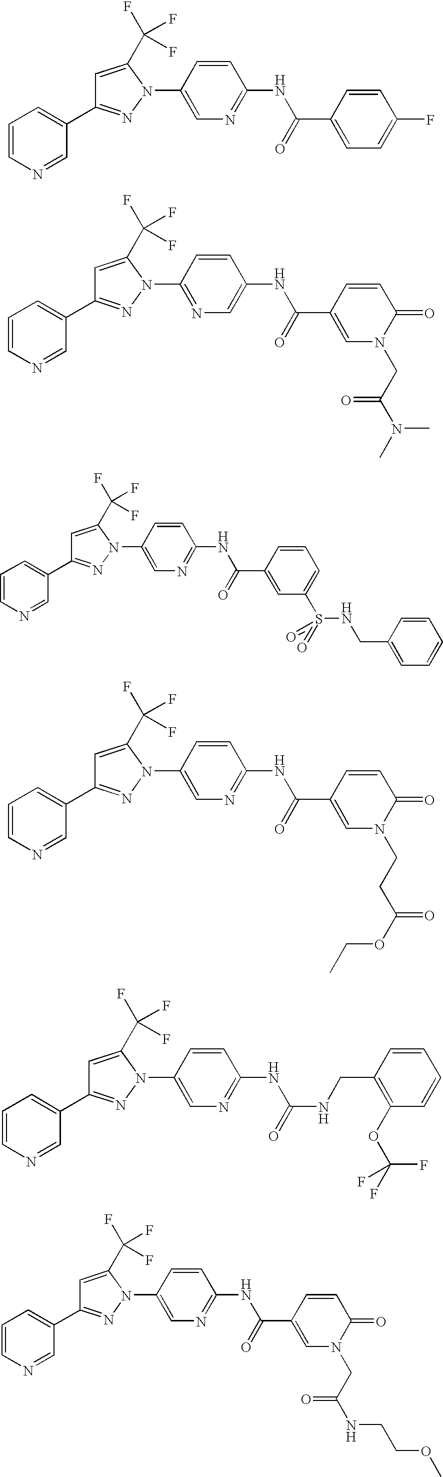 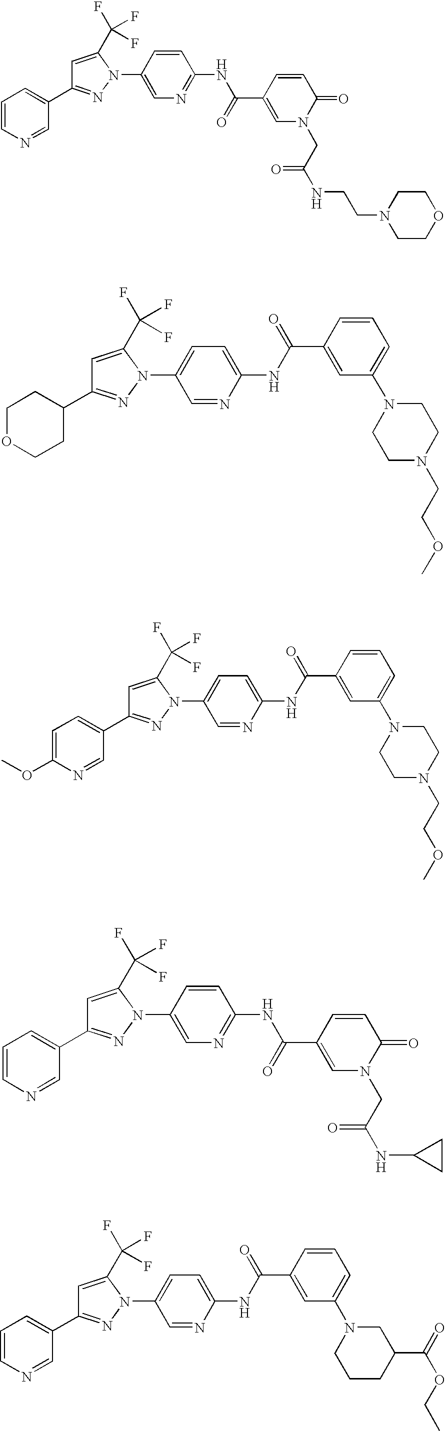 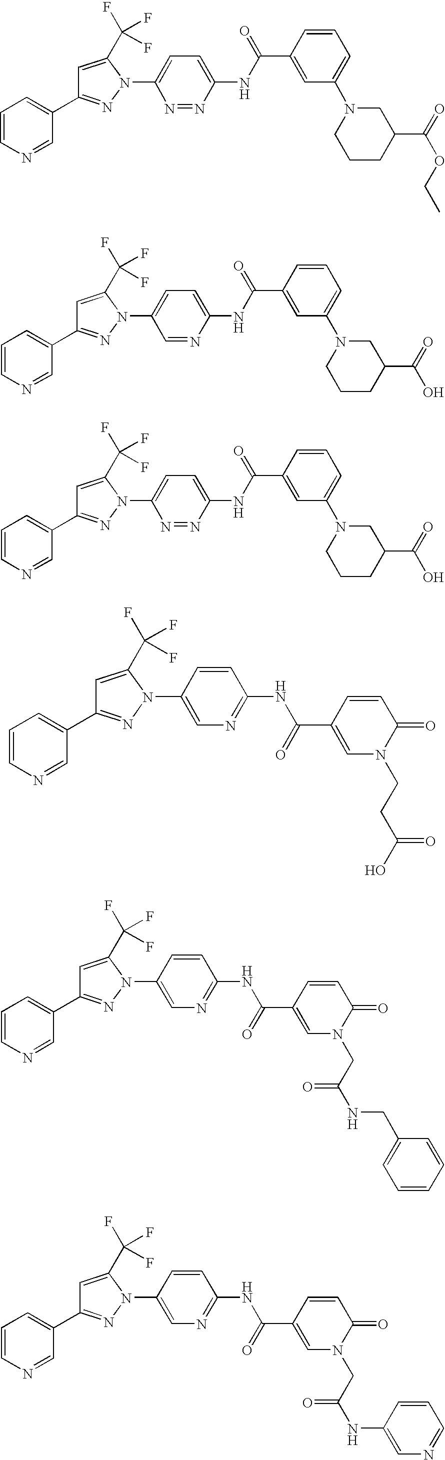 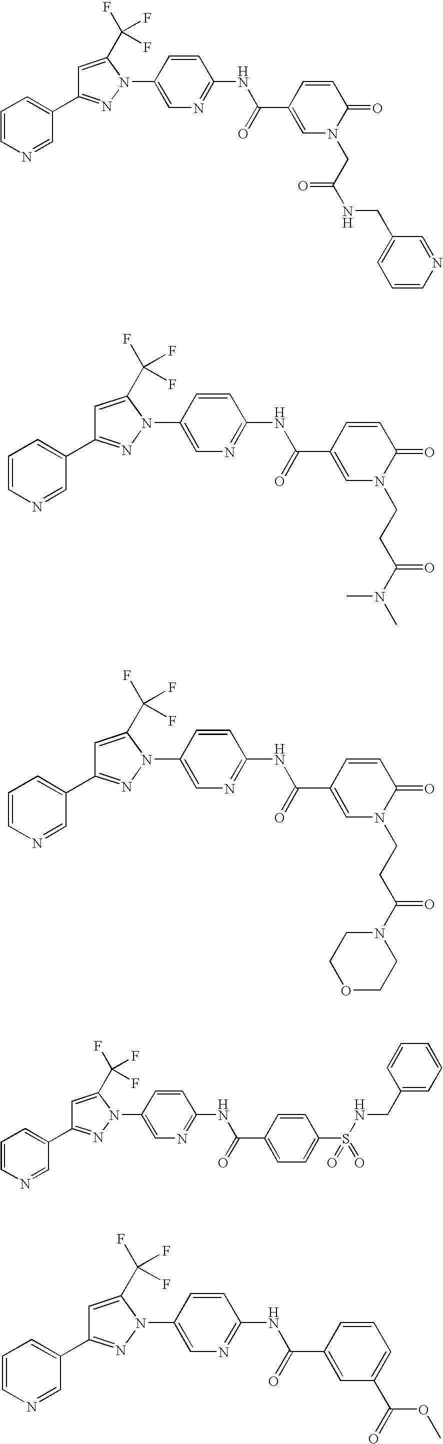 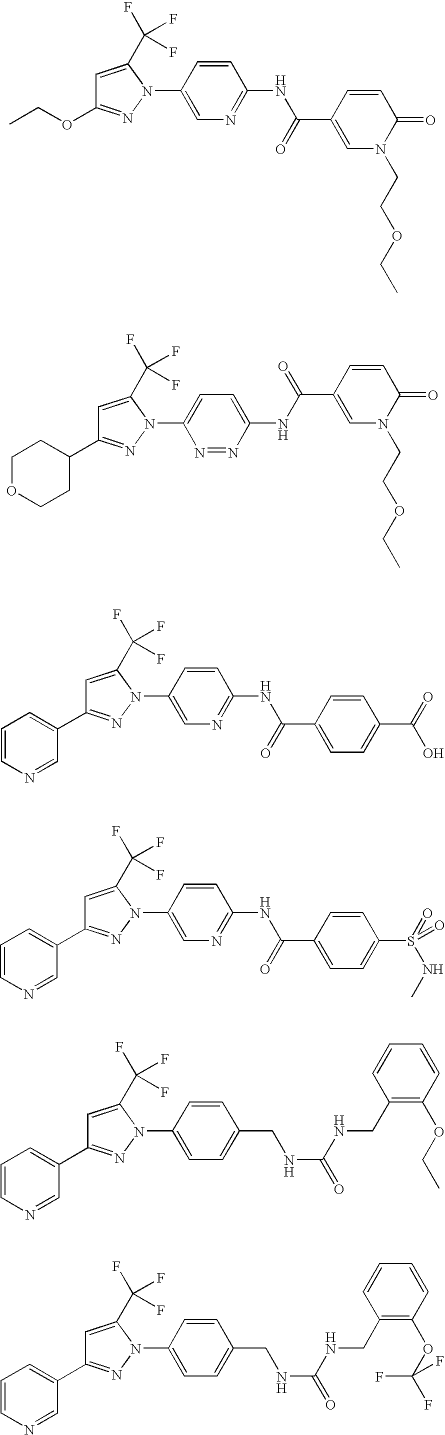 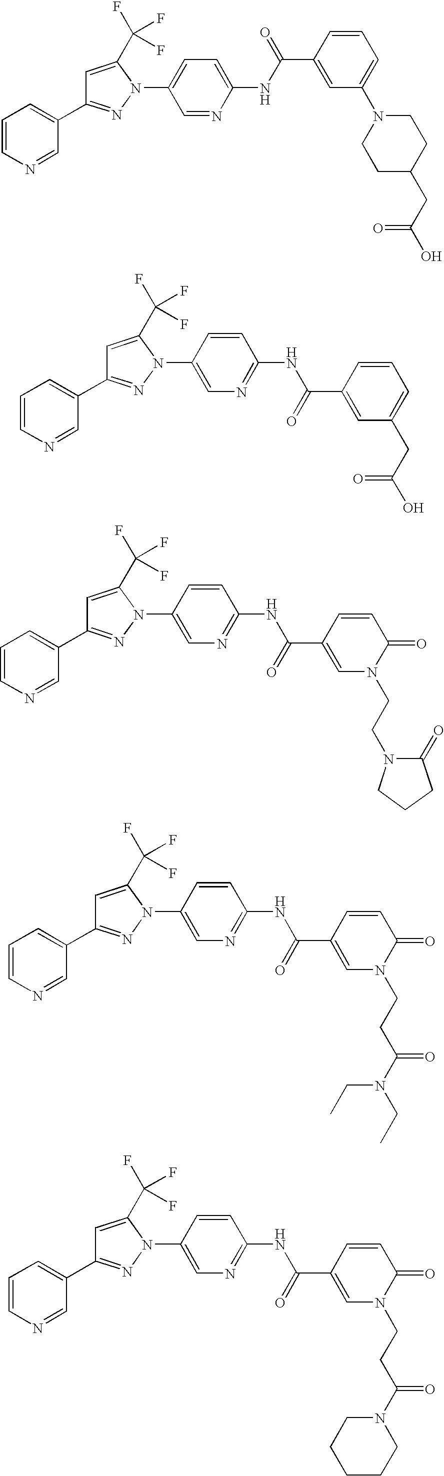 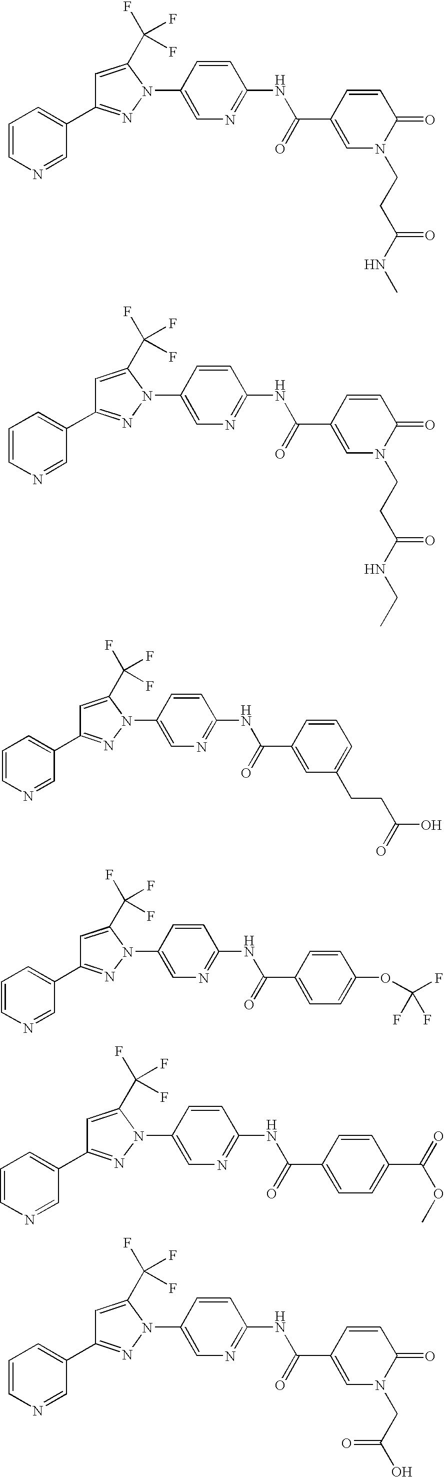 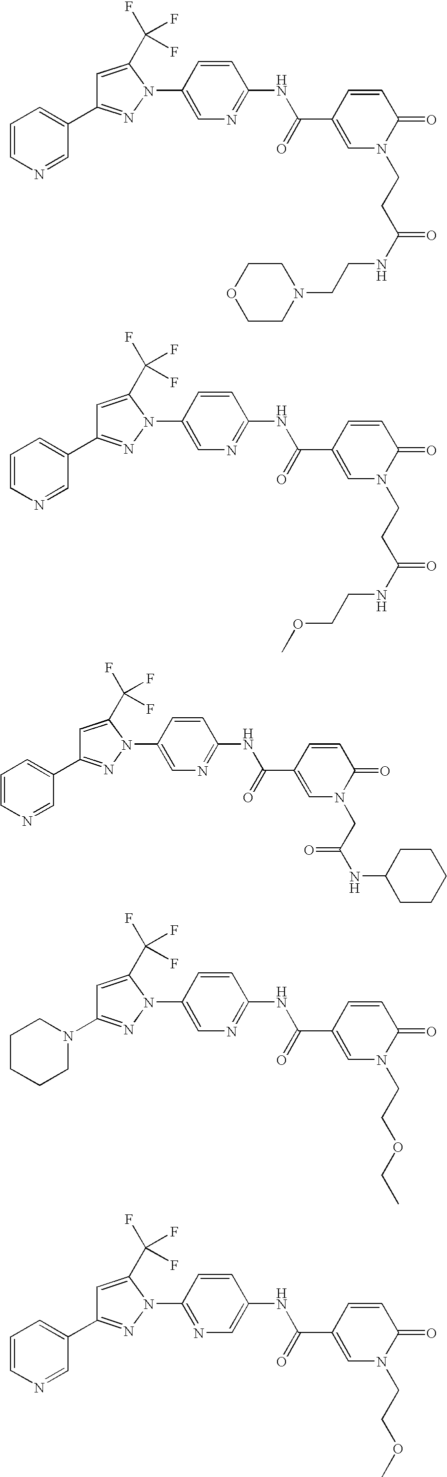
or the pharmaceutically acceptable salts thereof.

8. A method of treating a disease or condition chosen from type 1 and type 2 diabetes, insulin resistance syndrome, hypertension, atherosclerosis, coronary artery disease, angina, ischemia, ischemic stroke, Raynaud's disease and renal disease, said method comprising administering to a patient a pharmaceutically effective amount of a compound according to claim 1.

9. A pharmaceutical composition comprising a pharmaceutically effective amount of a compound according to claim 1 and one or more pharmaceutically acceptable carriers.
